(19) United States
(12) Patent Application Publication
(10) Pub. No.: US 2023/0357868 A1
(43) Pub. Date: Nov. 9, 2023

(54) COMPOSITIONS AND METHODS FOR OBTAINING PLANT CULTIVARS WITH DESIRED TERPENE PROFILES BASED ON TERPENE SYNTHASE GENE SELECTION

(71) Applicant: Front Range Biosciences, Inc., Boulder, CO (US)

(72) Inventors: Christopher Stephen PAULI, Boulder, CO (US); Anthony TORRES, Oakland, CA (US); Reginald GAUDINO, San Marcos, CA (US); Keith ALLEN, Port Ludlow, WA (US); Thomas BLANK, Palmetto, FL (US); Kymron DE CESARE, Los Gatos, CA (US)

(21) Appl. No.: 18/090,439

(22) Filed: Dec. 28, 2022

Related U.S. Application Data
(60) Provisional application No. 63/296,809, filed on Jan. 5, 2022.
(60) Provisional application No. 63/294,843, filed on Dec. 29, 2021.

(51) Int. Cl. A01H 1/00; C12Q 1/6895; C12Q 1/6851

(52) U.S. Cl. CPC C12Q 1/6895; A01H 1/127; C12Q 1/6851; C12Q 2600/13; C12Q 2600/156; C12Q 2600/158

(57) ABSTRACT

This technology relates in part to methods and compositions for identifying and/or selecting one or more terpene synthase genes and/or variants thereof in a plant, based on which a plant with a desired terpene profile can be obtained and/or used. A desired terpene profile of a plant (i.e., the types and, optionally, the relative amounts of terpenes in a plant) can include, for example, an energetic terpene profile, an anti-nociceptive terpene profile and an insecticidal terpene profile.

### RELATED PATENT APPLICATIONS

This patent application claims the benefit of U.S. Provisional Pat. Application No. 63/294,843, filed Dec. 29, 2021, entitled COMPOSITIONS AND METHODS FOR OBTAINING PLANT CULTIVARS WITH DESIRED TERPENE PROFILES BASED ON TERPENE SYNTHASE GENE SELECTION, naming Christopher Stephen PAULI et al. as inventors, and designated by Attorney Docket No. FRB-1004PROV1. This patent application also claims the benefit of U.S. Provisional Pat. Application No. 63/296,809, filed Jan. 5, 2022, entitled COMPOSITIONS AND METHODS FOR OBTAINING PLANT CULTIVARS WITH DESIRED TERPENE PROFILES BASED ON TERPENE SYNTHASE GENE SELECTION, naming Christopher Stephen PAULI et al. as inventors, and designated by Attorney Docket No. FRB-1004PROV2.

This patent application is related to PCT Application No. PCT/US2021/039461, filed on Jun. 28, 2021, entitled CHARACTERIZATION OF PLANT CULTIVARS BASED ON TERPENE SYNTHASE GENE PROFILES, naming Christopher Stephen PAULI et al. as inventors, and designated by Attorney Docket No. FRB-1002PCT, and also is related to U.S. Application No. 17/361,187, filed on Jun. 28, 2021, entitled CHARACTERIZATION OF PLANT CULTIVARS BASED ON TERPENE SYNTHASE GENE PROFILES, naming Christopher Stephen PAULI et al. as inventors, and designated by Attorney Docket No. FRB-1002T1. The entire content of each of the foregoing patent applications is incorporated herein by reference for all purposes, including all text, tables and drawings.

### SEQUENCE LISTING

The instant application contains a Sequence Listing which has been submitted electronically in XML file format and is hereby incorporated by reference in its entirety. Said XML copy, created on Jul. 19, 2023, is named FRB-1004_SL.xml and is 2,244,367 bytes in size.

### FIELD

The technology relates in part to methods and compositions for identifying and/or selecting one or more terpene synthase genes and/or variants thereof in a plant, based on which a plant with a desired terpene profile can be obtained and/or used. A desired terpene profile of a plant (i.e., the types and, optionally, the relative amounts of terpenes in a plant) can include, for example, an energetic terpene profile, an anti-nociceptive terpene profile and an insecticidal (insect deterrent) terpene profile.

### BACKGROUND

Terpenes are the largest and most structurally diverse class of natural compounds. They are produced by a large variety of plants, fungi, bacteria, and a few insects. To date, around 50,000 terpenoid metabolites, including monoterpenes, sesquiterpenes, and diterpenes, have been identified from higher plants, liverworts, and fungi. Terpenes play central roles in plant communication with the environment, including attracting beneficial organisms, repelling harmful ones, and facilitating communication between plants. The diversity of terpenoid compounds produced by plants plays an important role in mediating plant-herbivore, plant-pollinator, and plant-pathogen interactions. In plants such as Cannabis cultivars (e.g., Cannabis sativa), monoterpenes and sesquiterpenes are also responsible for most of their odor and flavor properties. Thus, variation in terpene content can be an important differentiator between cultivars of plants.

Terpene synthases (TPSs) are the key enzymes responsible for the biosynthesis of terpenes. Angiosperms, such as Cannabis, tend to have moderately large families of these enzymes, with both divergent and convergent evolution taking place. Thus, some TPS enzymes are highly divergent in sequence, while others differ from existing enzymes by just a few amino acids. Regardless, in general, the product profile of a given TPS enzyme cannot readily be determined from sequence similarity with or differences from other TPS gene family members. Therefore, for a given plant cultivar, there is a need to reliably identify all the members of the TPS gene family that are present, regardless of whether they are similar or different in sequence, in order to characterize the terpene production profile of a plant cultivar. There further is a need to identify plant cultivars, including cultivars of the family Rosidae and the sub-family Cannabis, that produce a terpene profile of interest, e.g., one or more of an energetic profile, an anti-nociceptive profile and an insecticidal (insect deterrent) profile.

### SUMMARY

Provided herein are methods and compositions for analyzing a plant cultivar, used interchangeably herein with plant, comprising identifying and/or selecting, in the plant cultivar, using at least one set of polynucleotide primers as provided herein, at least one terpene synthase gene that produces a terpene profile of interest, e.g., a terpene profile selected from among one or more of an energetic profile, an anti-nociceptive profile and an insecticidal profile. In certain embodiments, provided herein are compositions and methods for breeding plants with improved energetic, anti-nociceptive and/or insecticidal profiles by identifying/selecting parental plant cultivars that produce offspring having increased amounts of desired terpenes, or combinations of terpenes, that produce increased energetic, anti-nociceptive and/or insecticidal effects.

In certain embodiments of the methods provided herein, one or more of the polynucleotide primer pairs are selected from among a subset of those set forth in Table B (SEQ ID NOS: 1-1284, 1398 and 1399), in Primer Groups 1-19 as set forth in Tables 1-16 and in B3/F3 polynucleotide primer pairs set forth in Tables 17-35. In embodiments of the methods provided herein, one or more of the polynucleotide primer pairs are selected from among a subset of those set forth in SEQ ID NOS: 1-1284, 1398 and 1399, in Primer Groups 1-19 as set forth in Tables 1-16 and in B3/F3 polynucleotide primer pairs set forth in Tables 17-35, and/or from among sequences that share 90%, 91%, 92%, 93%, 94%, 95%, 96%, 97%, 98%, 99% or more identity with any of the sequences set forth in SEQ ID NOS: 1-1284, 1398 and 1399, in Primer Groups 1-19 as set forth in Tables 1-16 or in B3/F3 polynucleotide primer pairs set forth in Tables 17-35, or sequences that share 90%, 91%, 92%, 93%, 94%, 95%, 96%, 97%, 98%, 99% or more identity with any of the polynucleotide primer pair sequences set forth in in B3/F3 polynucleotide primer pairs set forth in Tables 17-35.

In embodiments of any of the methods provided herein, a plurality of plant cultivars can be analyzed. In certain embodiments, the plant cultivars are of the same species. In embodiments, the plurality of plant cultivars can be classified based on lineage and/or based on medicinal use.

In certain embodiments of the methods provided herein, one or more plant cultivars is/are a Cannabis cultivar. In aspects, the Cannabis cultivar is selected from among one or more of Type 1, Type 2, Type 3, Type 4 and Type 5 cultivars. In embodiments, one or more plant cultivars are of the family Rosidae.

In any of the methods provided herein, in certain embodiments, at least one plant cultivar that is analyzed produces one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A and Artemisinin.

In embodiments, the at least one plant cultivar includes terpene synthases that produce, singly or as combinations of terpene synthases, one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone,Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A and Artemisinin.

In certain embodiments of the methods provided herein, a terpene production profile is determined for one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A and Artemisinin.

In embodiments of the methods provided herein, at least one plant cultivar that is analyzed expresses one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, a terpene synthase expression profile is determined for one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, the one or more terpene synthases are selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

In certain embodiments of the methods provided herein, the plant cultivar is a Cannabis cultivar selected from among Cannabis sativa (Cs), a Cannabis indica, or Cannabis ruderalis, such as Sour Kusk (SK), Chocolate Kush (Choc), Black Label Kush (BL), BC Kush (BC), Lemon Skunk (LS), AK-47 (AK), Jamaican Lion (JL), Purple Kush (PK), CannaTsu (CT), Finola (FN), Valley Fire (VF), Cherry Chem (CC), LPA004 (L4), LPA005 (L5), and LPA021.3 (L21). Examples of Cannabis genomes include CS10, Arcata Trainwreck, Grape Stomper, Citrix, Black 84, Headcheese, Red Eye OG, Tahoe OG, Master Kush, Chem 91, Domnesia, Sour Tsunami, Sour Tsunami_x_CK, Tibor_1_2016, 80 E-1, 80 E-2, 80 E-3, Harlox, Saint Jack, Herijuana, Mothers Milk_5, Black Beauty, Sour Diesel, JL_1, JL_2, JL_3, JL_4, JL_5, JL_6, JL_father, BBCC_x_JL_father, JL_mother, JL_mother_p, IdaliaFT_1, Fedora17_6_1, Carmal_1_2016, CS_1_2016, EICam_1_2016, C3/USO-1, Carmagnola_3, and Merino_S_1.

In any of the methods provided herein, in certain embodiments, the methods further include, based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof, selecting a plant cultivar for in-breeding or out-crossing, or for cultivating as a crop. In certain embodiments, the plant cultivar is selected for its lineage that is assigned, and/or a medicinal use that is assigned based on identifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof. In embodiments, the plant cultivar is selected for resistance to an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof. In certain embodiments, the plant cultivar is selected for having an affinity towards an organism or situation that is identified based on identifying and/or quantifying one or more terpene synthase genes and/or paralogs thereof, determining the expression profile of one or more terpene synthase genes and/or paralogs thereof, determining the production profile of one or more terpenes, determining the production profile of one or more cannabinoids, determining the production profile of one or more flavonoids or a combination thereof. In embodiments, the organism or situation is selected from among insects, pests, mold, pesticides and other chemicals, mildew, fungi, bacteria, viruses and other pathogens, an environmental condition, such as climate or soil conditions, or a geographic location. In certain embodiments, the plant cultivar is selected for root-specific, stem-specific, leaf-specific or flower-specific expression of a terpene synthase gene and/or paralog thereof, a terpene, a cannabinoid or a flavonoid based on identifying one or more terpene synthase genes, determining the expression profile of one or more terpene synthase genes, determining the production profile of one or more terpenes, determining the production profile of one or more cannabinoids, determining the production profile of one or more flavonoids or a combination thereof. Also provided herein are methods of breeding any of the plant cultivars selected according to the methods provided herein. Also provided herein are methods of cultivating a crop of any of the plant cultivars selected according to the methods provided herein. Also provided herein are methods of treatment comprising administering a plant cultivar selected according to the methods provided herein, or a portion thereof (e.g., seed, root, stem, flower) or an extract thereof (e.g., extracts from tissues of the plant cultivar) to a subject having a condition or disease in need of such treatment, whereby the condition or disease, or symptoms thereof, are ameliorated or reduced.

In certain embodiments of the methods provided herein, the methods include, based on identifying one or more terpene synthase genes and/or paralogs thereof, determining the expression profile of the one or more terpene synthase genes and/or paralogs thereof, determining the production profile of one or more terpenes, determining the production profile of one or more cannabinoids, determining the production profile of one or more flavonoids or a combination thereof, genetically modifying a plant cultivar whereby the expression of at least one terpene synthase gene and/or paralog thereof is inhibited or increased in the plant cultivar. In certain embodiments, the genetic modification increases the production of at least one terpene or decreases the production of at least one terpene in the plant cultivar. In embodiments, the plant cultivar is of a Cannabis cultivar. In certain aspects, the Cannabis cultivar is selected from among one or more of Type 1, Type 2, Type 3, Type 4 and Type 5 cultivars. In embodiments, one or more plant cultivars are of the family Rosidae.

Also provided herein are methods of breeding daughter plant cultivars for improved energetic terpene profiles, anti-nociceptive terpene profiles and/or insecticidal terpene profiles based on the properties of the parent plant cultivars, analyzed according to the methods provided herein, that are selected for breeding the daughter plant cultivar.

Also provided herein is a method of treatment of a subject having a disease or condition, the method including:
analyzing at least one plant cultivar by any of the methods provided herein;
based on identifying one or more terpene synthase genes, determining the expression profile of one or more terpene synthase genes, determining the production profile of one or more terpenes, identifying the plant cultivar as having an energetic terpene profile, an anti-nociceptive terpene profile and/or an insecticidal terpene profile; and
administering the plant cultivar or a portion or extract thereof to the subject.

In certain embodiments, a terpene production profile is determined for one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone,Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A and Artemisinin. In certain embodiments, at least one plant cultivar that is analyzed expresses one or more terpene synthases selected from among TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, the one or more terpene synthases are selected from among TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

The plant cultivars analyzed and/or genetically modified according to the methods provided herein can be used in methods of breeding, of cultivating crops, of treatment and other uses as provided herein. In certain embodiments, when the plant cultivars are genetically modified, they can be screened for the existence of the genetic modification using any of the solid supports or collections of solid supports according to any of the methods provided herein. In embodiments, the existence of a mutation in a plant cultivar can be detected using any of the solid supports or collections of solid supports according to any of the methods provided herein.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale, and, in some instances, various aspects may be shown exaggerated or enlarged to facilitate an understanding of particular embodiments.

FIG. 1 provides examples of nucleic acid sequences encoding terpene synthases that can be detected to identify and/or select plant cultivars having one or more of an energetic profile, an anti-nociceptive profile and an insectical profile using the methods and compositions provided herein.

FIG. 2 depicts the terpene product profiles of alleles of the TPS20 terpene synthase gene.

FIG. 3 depicts a representative correlation between the detection of certain terpene synthase-specific exons using High Resolution Melt (HRM) analysis in Cannabis cultivars and the terpene distribution profiles (chemical phenotypes) of those cultivars.

### DETAILED DESCRIPTION

### Overview

Provided herein are methods and compositions for analyzing a plant cultivar comprising identifying and/or selecting, in the plant cultivar, at least one terpene synthase gene that produces a terpene product profile (used interchangeably herein with terpene profile) of interest, e.g., a terpene profile selected from among one or more of an energetic profile, an anti-nociceptive profile and an insecticidal profile. As used herein, an “energetic profile” means a terpene profile containing at least one terpene that imparts energy when administered to a subject, such as strength and/or vitality for sustained physical or mental activity, e.g., by mood elevation or by increased oxygenation of the brain, and/or a terpene profile that does not contain or contains a reduced amount of at least one terpene that has a sedative effect when administered to a subject, such as decreased physical or mental activity, e.g., by mood depression or calming. As used herein, an “antinociceptive profile” means a terpene profile containing at least one terpene that inhibits or blocks sensitivity to pain when administered to a subject. As used herein, an “insecticidal profile” means a terpene profile containing at least one terpene associated with one or more of insecticidal activity, such as a neurotoxic effect on the insect, oviposition deterrence, insect fumigant activity, contact toxicity to an insect and insect herbivore predator attractant.

### Terpenes

Terpenes are aromatic compounds that are a class of unsaturated compounds found in the essential oils of many plants. As used herein, the term “plant” or “plant cultivar” includes any and all plant species that produce terpenes, including for example, angiosperms, any species of woody, ornamental or decorative, crop or cereal, fruit or vegetable, fruit plant or vegetable plant, flower or tree, macroalga or microalga, phytoplankton and photosynthetic algae (e.g., green algae Chlamydomonas reinhardtii). A plant also refers to a unicellular plant (e.g. microalga) and a plurality of plant cells that are largely differentiated into a colony (e.g. volvox) or a structure that is present at any stage of a plant’s development. Such structures include, but are not limited to, a fruit, a flower, a seed, a shoot, a stem, a leaf, a root, plant tissue sand the like. As used herein, the term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue can be in planta, in organ culture, tissue culture, or cell culture. Any of the foregoing plant cultivars, portions thereof or extracts thereof are contemplated for use, e.g., as plant samples, in the methods provided herein.

The term “strain” is used interchangeably herein with “cultivar” (cultivated variety), “plant cultivar” or “variety” and refers to a species of a family of plants, such as a species of a Cannabis plant. A cultivar generally has been cultivated for desirable characteristics, such as color, shape, smell, medicinal use, etc., that are maintained during propagation. Phrases such as “plurality of strains of a plant” or “plurality of cultivars of a plant” or equivalent phrases, as used herein, refers to multiple species of the same plant, e.g., multiple species of Cannabis plant cultivars such as Jamaican Lion, Purple Kush, CannaTsu, Finola, Valley Fire, Cherry Chem and the like. The terms “strain,” “cultivar,” (cultivated variety), “plant cultivar” or “variety” also can be used interchangeably herein with “chemovar,” such as when the plant species is characterized by its chemical or biological profile, such as one or more of a terpene synthase gene profile, a terpene synthase expression profile, a terpene profile, a flavonoid profile, a cannabinoid profile or any combination thereof. The term “profile,” as used herein, can refer to the type and/or abundance (level of expression, in the case of a gene such as a terpene synthase) of each analyte of the group that is profiled, e.g., each terpene in a group of terpenes of a plant cultivar that are profiled, or each terpene synthase in a group of terpene synthasess of a plant cultivar that are profiled.

The molecular structures of terpenes consist of five carbon isoprene units. Mono terpenes contain 2 isoprene units, sesquiterpenes contain 3 isoprene units, and diterpenes contain 4 isoprene units. These aromatic compounds create the characteristic scent of many plants, such as Cannabis, pine, and lavender, as well as fresh orange peel. The fragrance of most plants is due to a combination of terpenes. Terpenes play central roles in plant communication with the environment, including attracting beneficial organisms, repelling harmful ones, and communication between plants. In nature, these terpenes can protect the plants from animal grazing or infectious germs.

Terpenes also can offer health benefits to animals, including humans. Terpenes and essential oils have been studied over decades as remedies for a variety of medical conditions and have been found to have a wide range of biological and therapeutic properties. For example, terpenes are known to have antioxidant, anti-inflammatory, antibacterial, antiviral, anti-anxiety, antinociceptive, analgesic, antihypertensive, sedative, antidepressant, neuro protective and gastro protective properties. More recently, researchers have looked at the individual terpenes in essential oils, to understand which terpenoids might be contributing to their overall biological and medical properties. Terpenes in essential oils can either exert their individual effects in the oil or they can operate synergistically or agonistically with other oil constituents.

In Cannabis plants, such as C. sativa, more than 100 terpenes have been identified. Monoterpenes and sesquiterpenes are responsible for most of the odor and flavor properties of C. sativa, meaning that variation in terpene content is an important differentiator between cultivars. Therefore, there has long been interest from breeders in creating cultivars with particular terpene profiles. Further, there is a growing body of preliminary evidence that terpenes play a role in the various effects of C. sativa on humans, either directly or by modulating the effect of the cannabinoids, implying that medical C. sativa breeding likely will include terpene targets.

Given the important role of terpenes in plants, there is a need for methodologies to reliably identify plants that have desired terpene production profiles (used interchangeably herein with terpene profiles) for agricultural, industrial or medicinal use. The terms “terpene (production) profile,” “cannabinoid (production) profile,” “flavonoid (production) profile,” as used herein, refers to the types and amounts of terpenes, cannabinoids or flavonoids, respectively, in a plant cultivar, and can also include ratios of the relative abundance of two or more terpenes, cannabinoids or flavonoids, respectively.

Terpene synthases (TPSs) are the key enzymes responsible for the biosynthesis of terpenes. Provided herein are molecular markers that permit reliable identification and/or quantitation of the TPS gene profile of a plant cultivar, thereby permitting the identification and selection of plants for use in methods of genetic modification, methods of screening, and methods of use in breeding, crop cultivating, therapeutic methods and other methods as provided herein.

### Terpene Synthases

The terpene synthase (TPS) family is a family of genes that encodes enzymes that use similar substrates and generate similar products but have diverged in different lineages to provide a wide variety of terpenes and mixtures of terpenes. Some estimates suggest that more than 25,000 terpene structures may exist in plants. Analysis of the several plant genomes that have been sequenced and annotated indicates that, with the exception of the moss Physcomitrella patens, which has a single functional TPS gene, the TPS gene family is a mid-size family, with gene numbers ranging from approximately 20 to 150 in sequenced plant genomes.

Isopentenyl diphosphate (IPP) is the common precursor of all terpenes. IPP is isomerized to give dimethylallyl pyrophosphate (DMAPP). DMAPP either serves as the substrate for hemiterpene biosynthesis or fuses with one IPP unit to form geranyl diphosphate (GPP). The condensation of one GPP molecule with one IPP molecule gives farnesyl diphosphate (FPP), and the condensation of one FPP molecule with one IPP molecule will give geranylgeranyl diphosphate (GGPP). GPP, FPP and GGPP are the precursors for monoterpenes, sesquiterpenes and diterpenes, respectively. While these prenyl diphosphates in the trans -configuration have been believed to be the ubiquitous natural substrates for terpene synthases, recent studies showed that two prenyl diphosphates in the cis-configuration, neryl diphosphate (NPP) and Z,Z-FPP, are also the naturally occurring substrates of terpene synthases. Isoprene synthase, monoterpene synthases, sesquiterpene synthases, and diterpene synthases convert DMAPP, GPP (or NPP), FPP (or Z,Z-FPP), and GGPP to isoprene, monoterpenes, sesquiterpenes and diterpenes, respectively.

Based on the reaction mechanism and products formed, plant TPSs can be classified into two groups: Class I and Class II. CPS is a representative of class II TPSs: it catalyzes the formation of CPP through protonation-induced cyclization of GGPP. However, most known plant TPSs are Class I TPSs. In the initial step of the enzymatic reactions catalyzed by Class I TPSs, the prenyl diphosphate is ionized and carbocation intermediates are formed.

A striking feature of class I TPS enzymes is that, because of the stochastic nature of bond rearrangements that follow the creation of the unusual carbocation intermediates, a single TPS enzyme using a single substrate often gives rise to multiple products. The central feature of this evolutionary plasticity is that change of just a single amino acid in the active site can lead to a different product profile. Most terpenes are secondary metabolites whose synthesis evolved in response to selection for increased fitness for some ecological niche. Consistent with the need (for terpene production profiles of a plant cultivar, e.g.) to be environmentally adaptive, the TPS family has evolved such that new TPS with differing product profiles can be derived from existing enzymes by changes to just a few amino acids.

Angiosperms, of which Cannabis is an example, tend to have moderately large families of these enzymes, with both divergent and convergent evolution taking place. Some of these TPS enzymes appear to be a result of recent duplications, i.e., they are paralogs of each other. Other TPS enzymes can be quite distant from each other: for example, there is a common “terpenoid synthase fold,” but sequence divergence across the family can be very high, just staying within the constraints of maintaining an overall fold and basic configuration of the active site.

Generally, the product profile of a given TPS enzyme cannot be determined from sequence similarity with other TPS enzymes. For example, two paralogous diterpene synthases in Norway spruce (Picea abies), isopimaradiene synthase and levopimaradiene/abietadiene synthase, although 91% identical at the amino acid level, differ in their terpene product profiles: one is a single-product enzyme, whereas the other is a multiproduct enzyme that forms completely different products. In addition, a one-amino acid mutation was found to switch the levopimaradiene/abietadiene synthase into producing isopimaradiene and sandaracopimaradiene and none of its normal products. Four mutations were sufficient to reciprocally reverse the product profiles for both of these paralogous enzymes, while maintaining catalytic efficiencies similar to the wild-type enzymes (Keeling et al., Proc. Natl. Acad. Sci. USA, 105(3):1085-1090 (2008).

Thus, given the widely differing terpene profiles of the TPS enzymes, even when there is a relatively high degree of overall sequence identity, there is a need to reliably identify the individual TPS genes that are present in the TPS gene profile of a plant cultivar. The methods provided herein are based on primer sets that amplify unique subsequences, such as exons or portions thereof, within each TPS gene, thereby providing a higher order differentiation that permits sensitive detection and/or quantification of each TPS gene in the plant cultivar, regardless of the overall sequence identity between the TPS genes.

Any terpene synthase, or combinations of terpene synthases, are contemplated for analysis and/or applications/uses according to the methods and compositions provided herein. In embodiments, terpene synthases contemplated herein for the compositions and methods of analyses/applications/uses include terpene synthases that produce, singly or in combinations of two or mor terpene synthases, one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, γ-Terpinene, α-Terpineol, Terpinolene, Valencene, γ-Elemene, Z-Ocimene, E-Ocimene, α-Thujone, Thujene, γ-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, γ-gurjunene, α-bulnesene, Bulnesol, α-eudesmol, β-eudesmol, Hedycaryol, γ-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A and Artemisinin.

In certain embodiments, the one or more terpene synthases are selected from among those designated as TPS11, TPS11-like, TPS12, TPS12-like, TPS13, TPS13-like, TPS13-like2, TPS14, TPS15, TPS16, TPS17, TPS18, TPS19, TPS1, TPS20, TPS23, TPS24, TPS2, TPS30, TPS30-like, TPS32, TPS33, TPS36, TPS37, TPS38, TPS39, TPS3, TPS40, TPS41, TPS42, TPS43, TPS44, TPS45, TPS46, TPS47, TPS48, TPS49, TPS4, TPS4-like, TPS50, TPS51, TPS52, TPS53, TPS54, TPS55, TPS56, TPS57, TPS58, TPS59, TPS5, TPS5, TPS60, TPS61, TPS62, TPS63, TPS64, TPS6, TPS6-like, TPS7, TPS8, TPS8, TPS8-like, TPS9, TPS9, TPS9-like and TPS9-like2. In certain embodiments, the one or more terpene synthases are selected from among those designated as TPS11JL, TPS11-likeJL, TPS12JL, TPS12-likeJL, TPS13JL, TPS13-likeJL, TPS13-like2JL, TPS14JL, TPS15JL, TPS16JL, TPS17JL, TPS18JL, TPS19JL, TPS1JL, TPS20JL, TPS23JL, TPS24JL, TPS2JL, TPS30JL, TPS30-likeJL, TPS32JL, TPS33JL, TPS36JL, TPS37JL, TPS38JL, TPS39JL, TPS3JL, TPS40JL, TPS41JL, TPS42JL, TPS43JL, TPS44JL, TPS45JL, TPS46JL, TPS47JL, TPS48JL, TPS49JL, TPS4JL, TPS4-likeJL, TPS50JL, TPS51JL, TPS52JL, TPS53JL, TPS54JL, TPS55JL, TPS56JL, TPS57JL, TPS58JL, TPS59JL, TPS5JL, TPS5JL, TPS60JL, TPS61JL, TPS62JL, TPS63JL, TPS64JL, TPS6JL, TPS6-likeJL, TPS7JL, TPS8JL, TPS8JL, TPS8-likeJL, TPS9JL, TPS9JL, TPS9-likeJL and TPS9-like2JL.

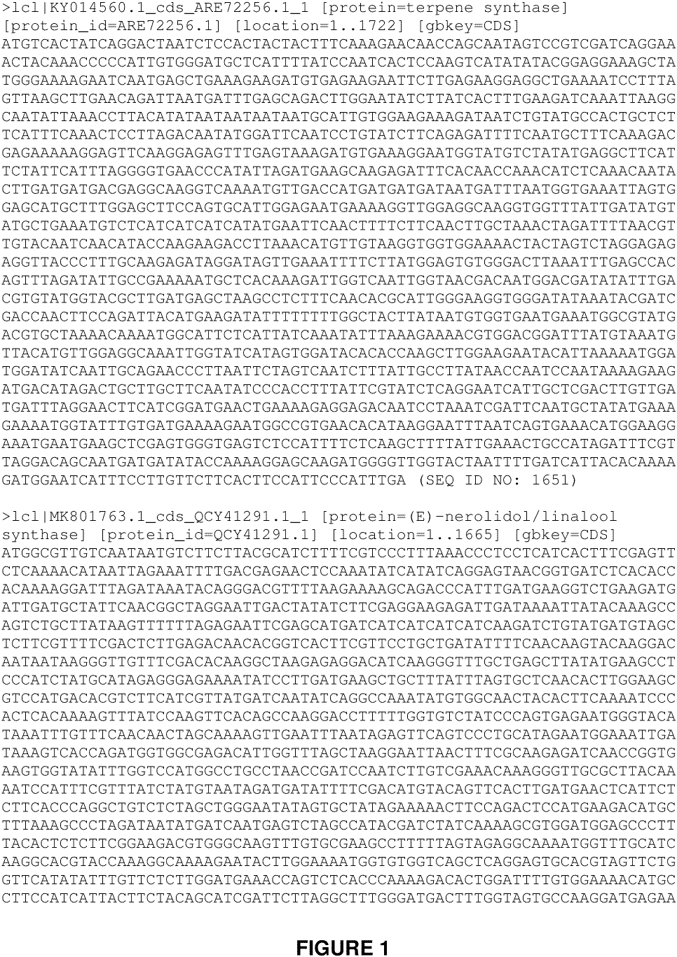

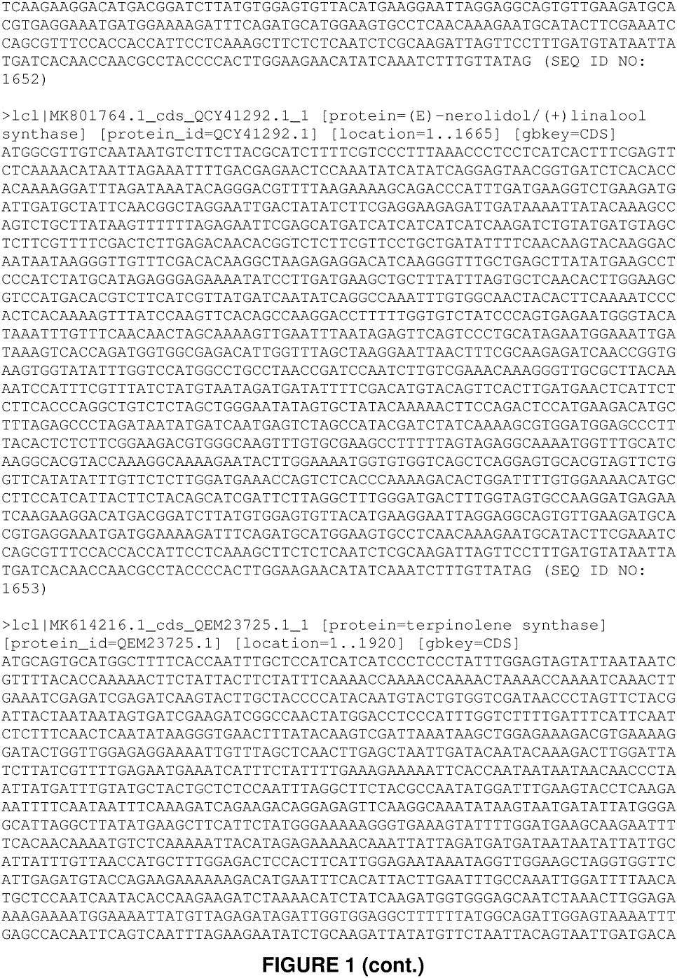

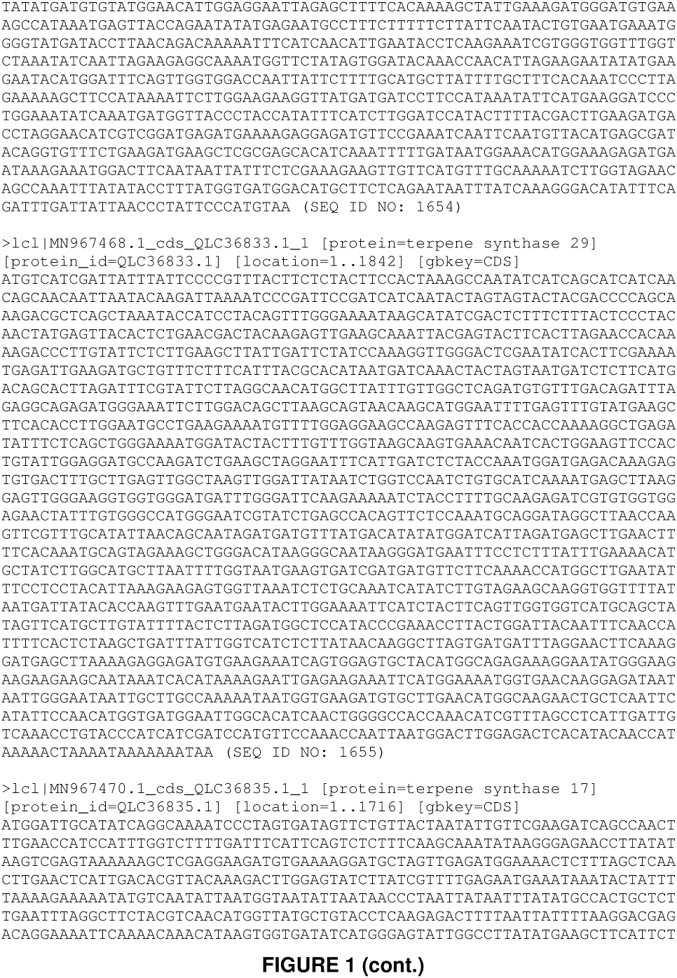

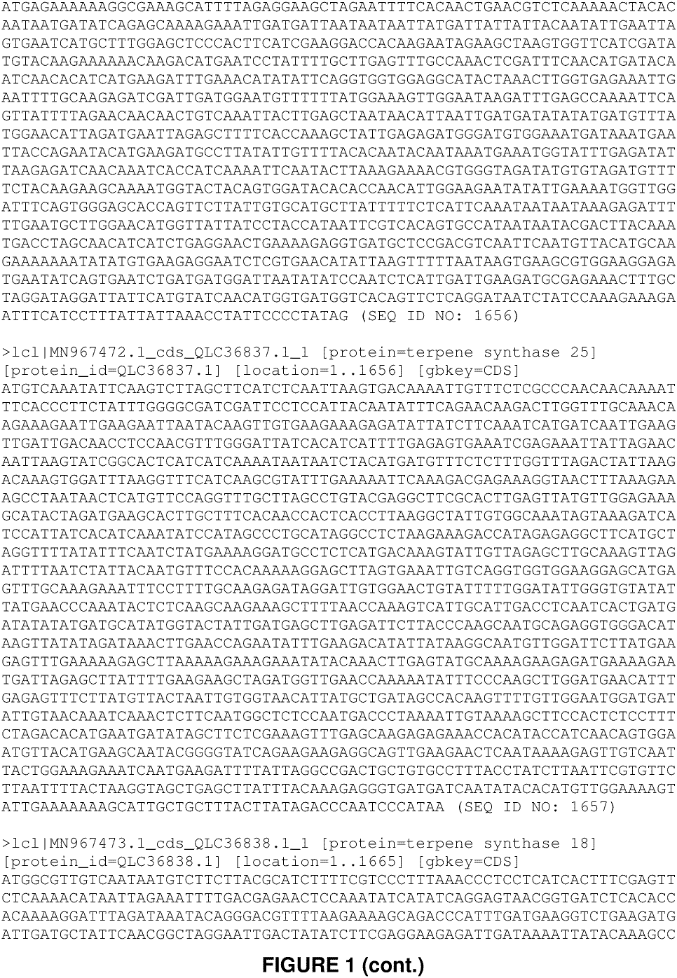

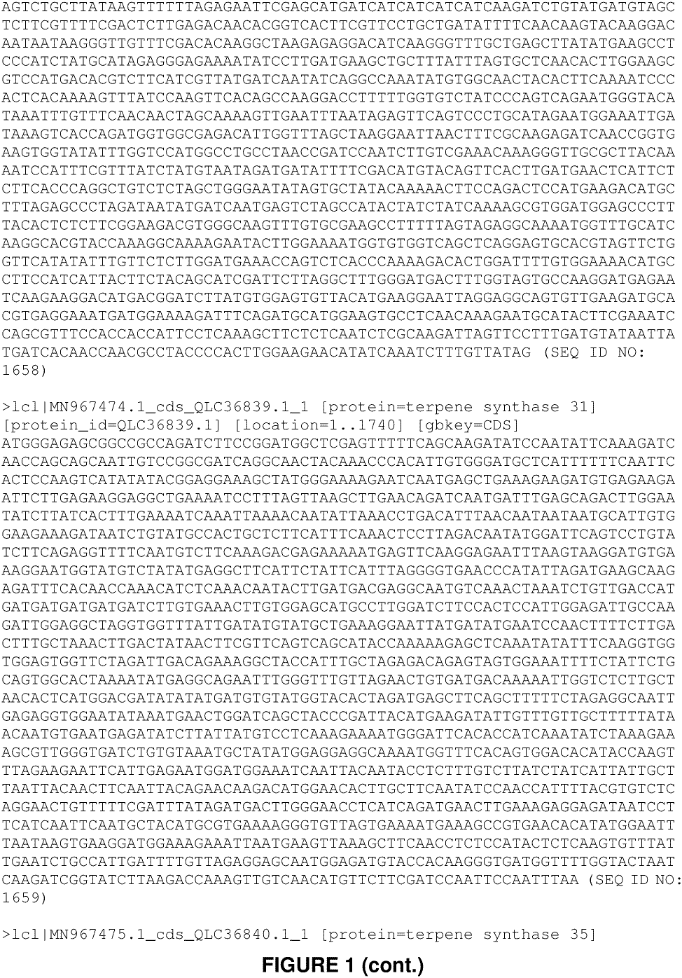

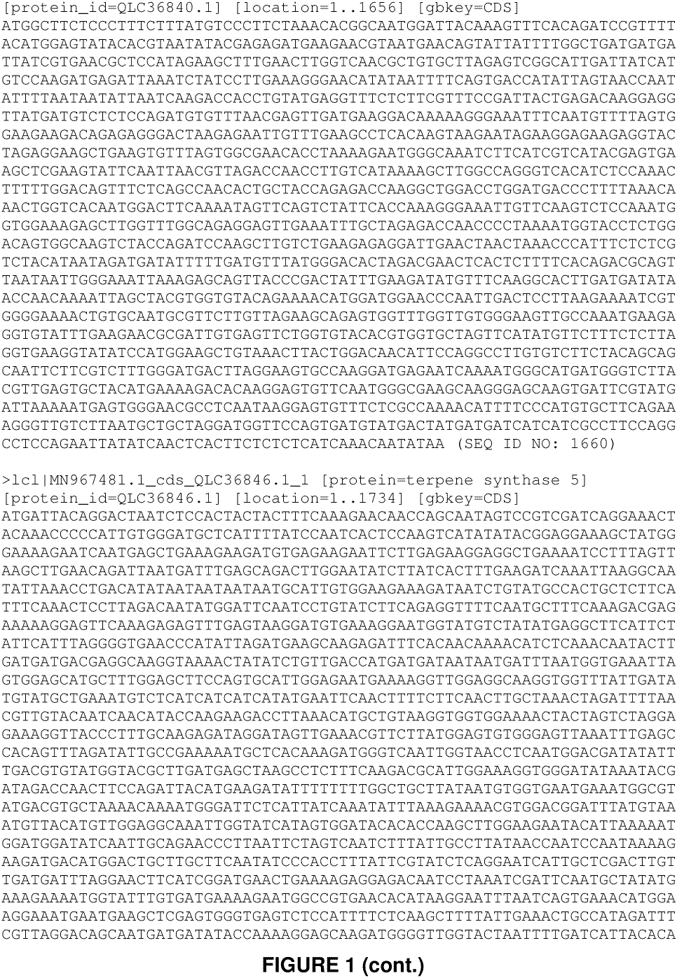

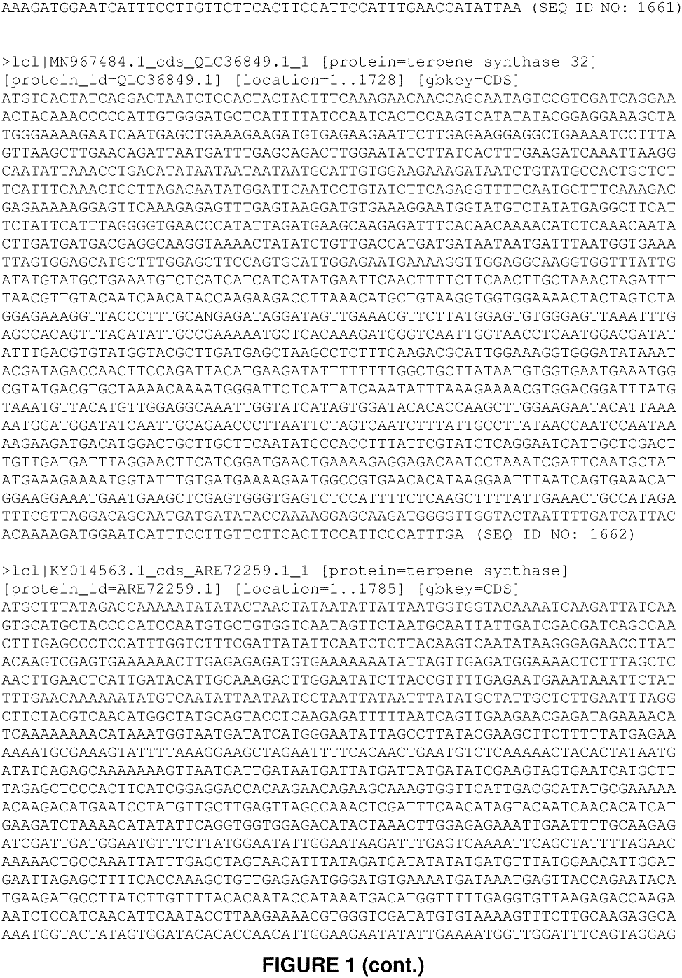

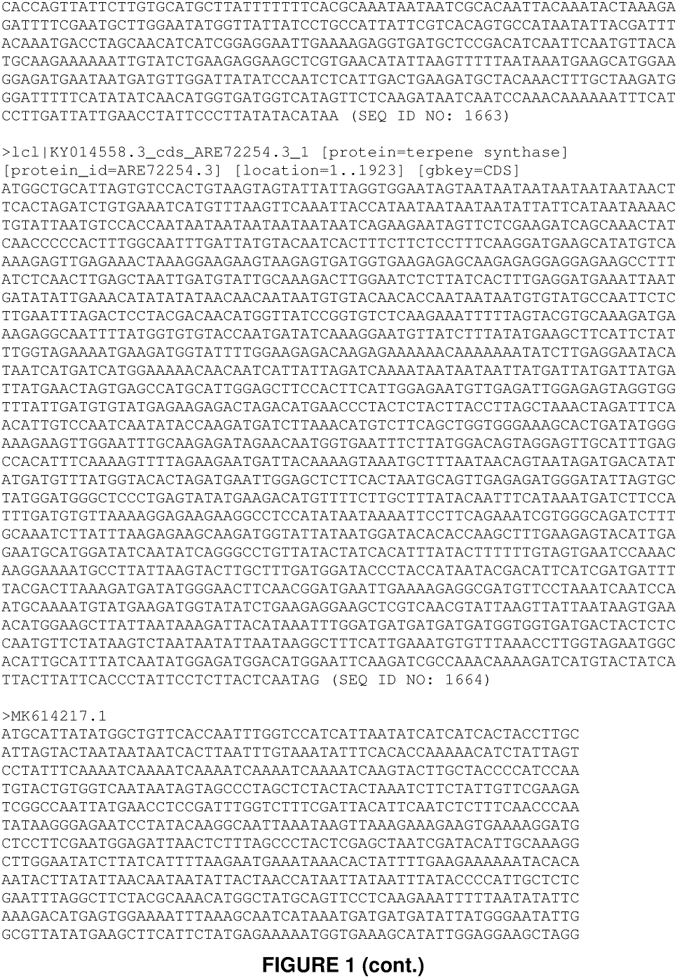

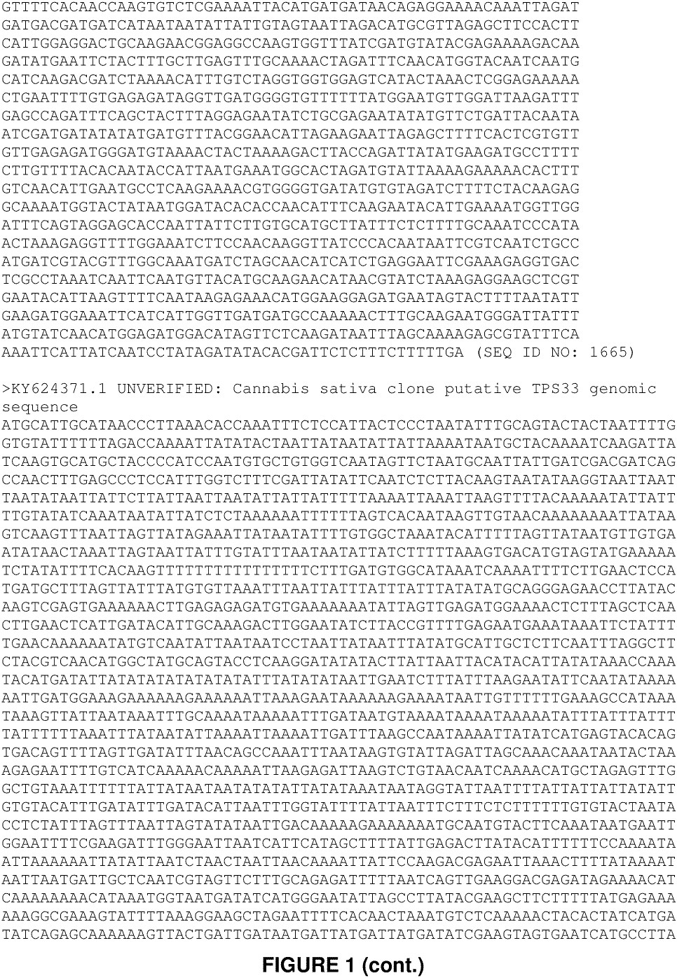

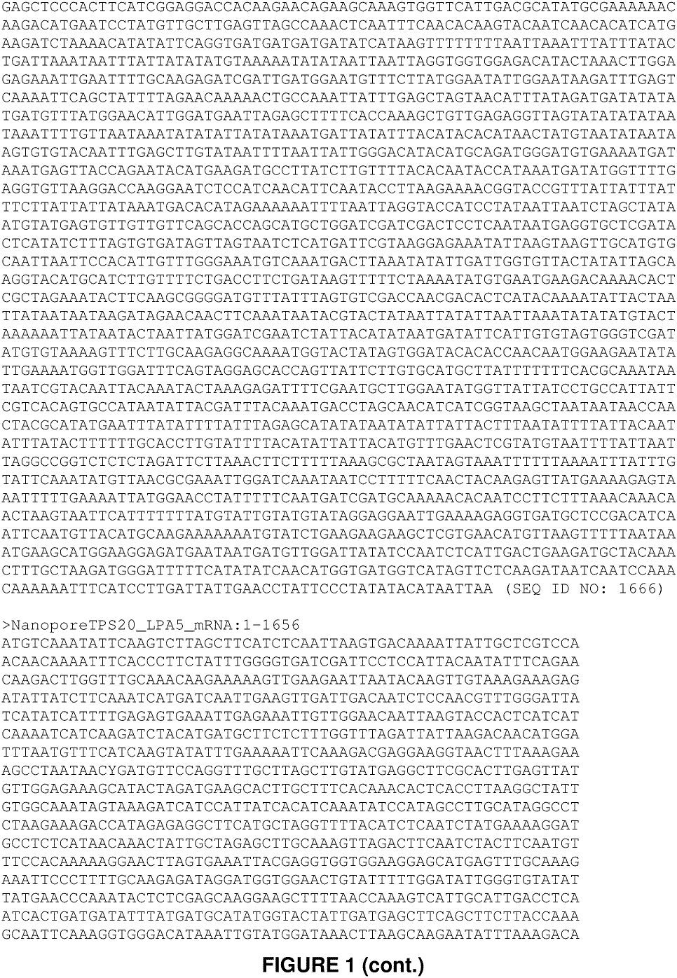

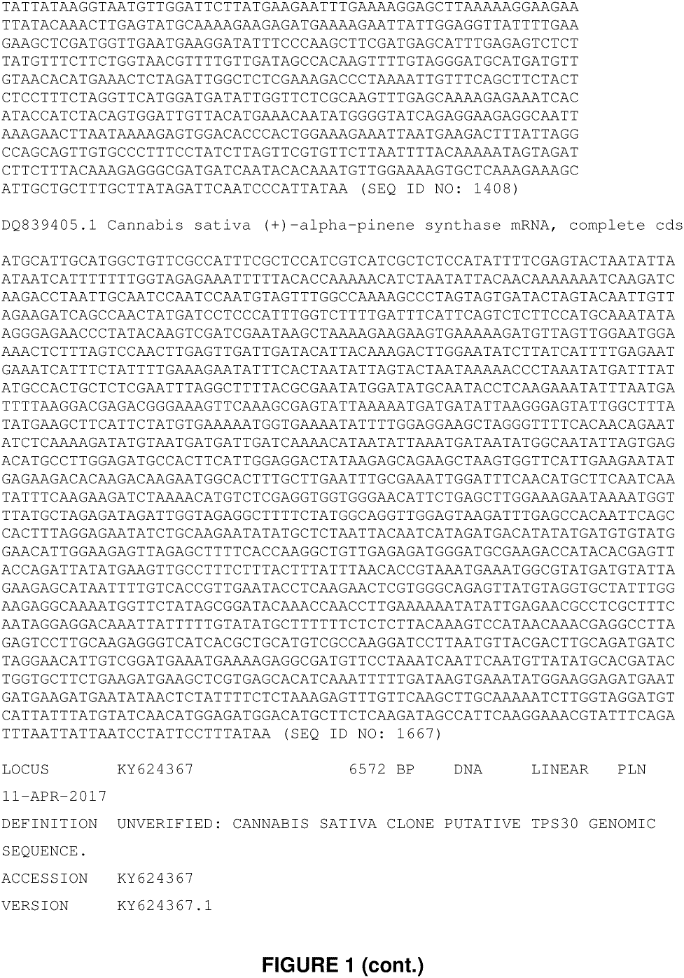

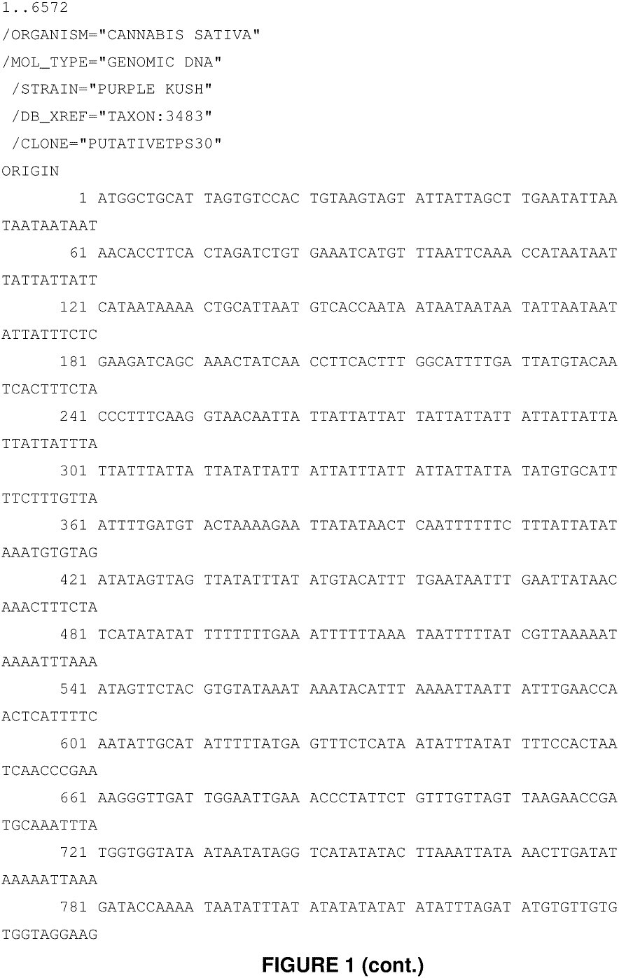

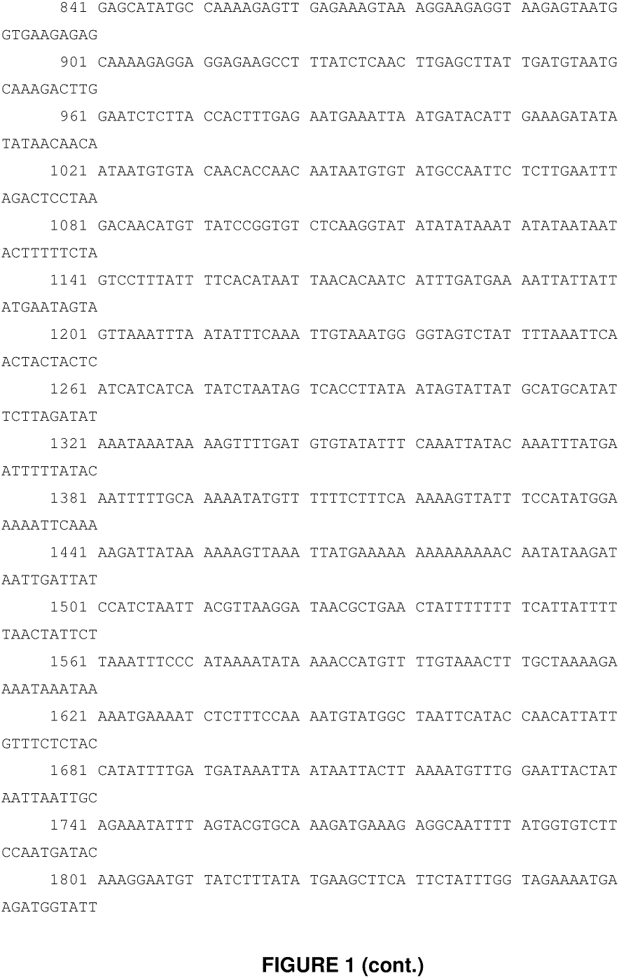

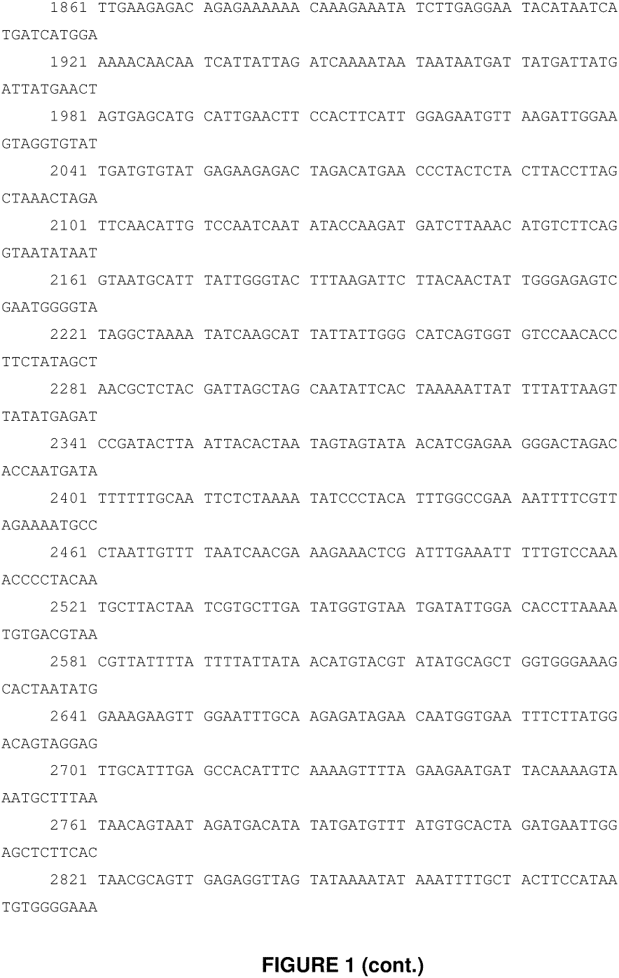

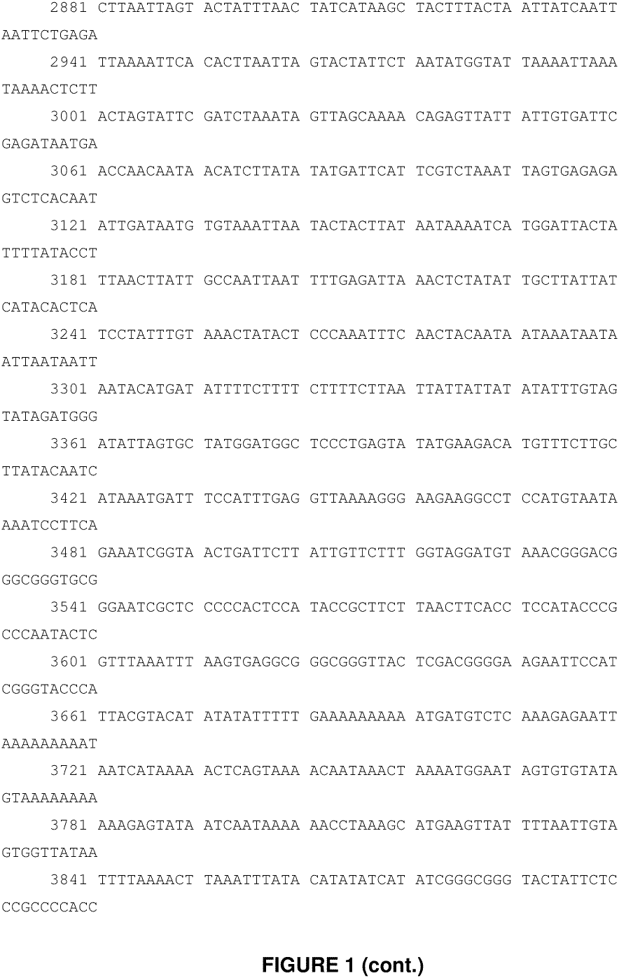

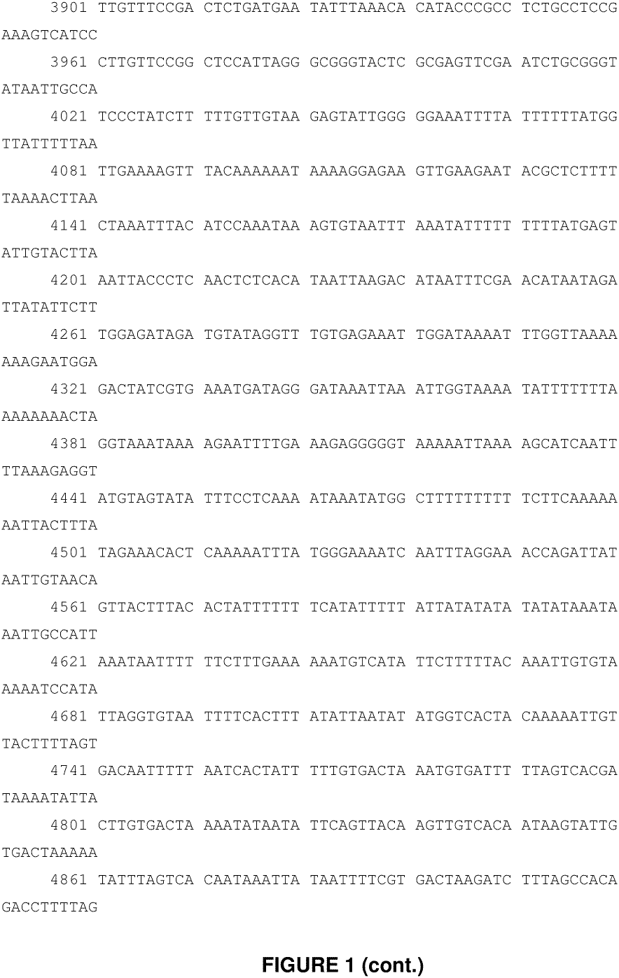

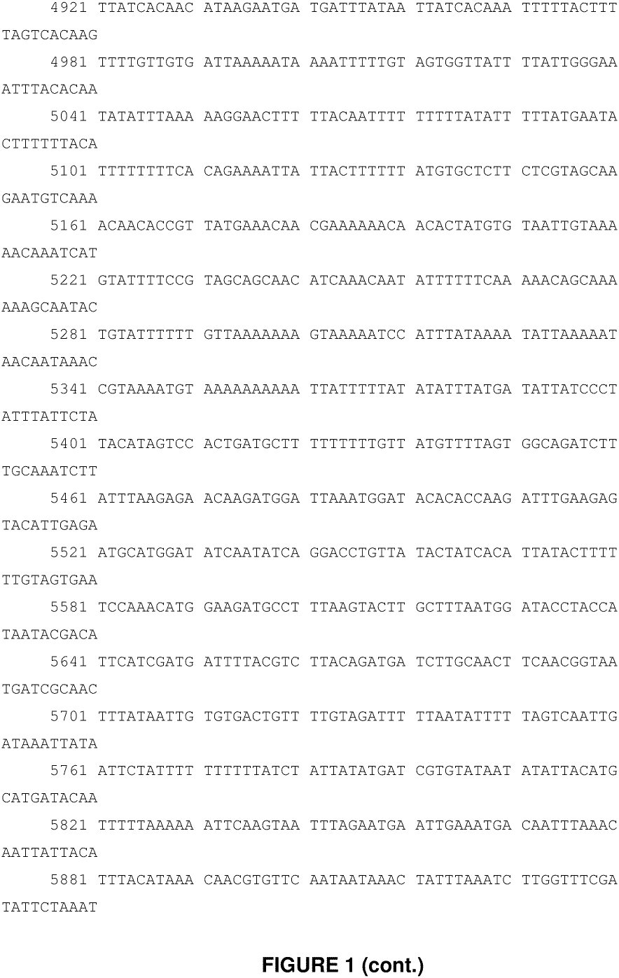

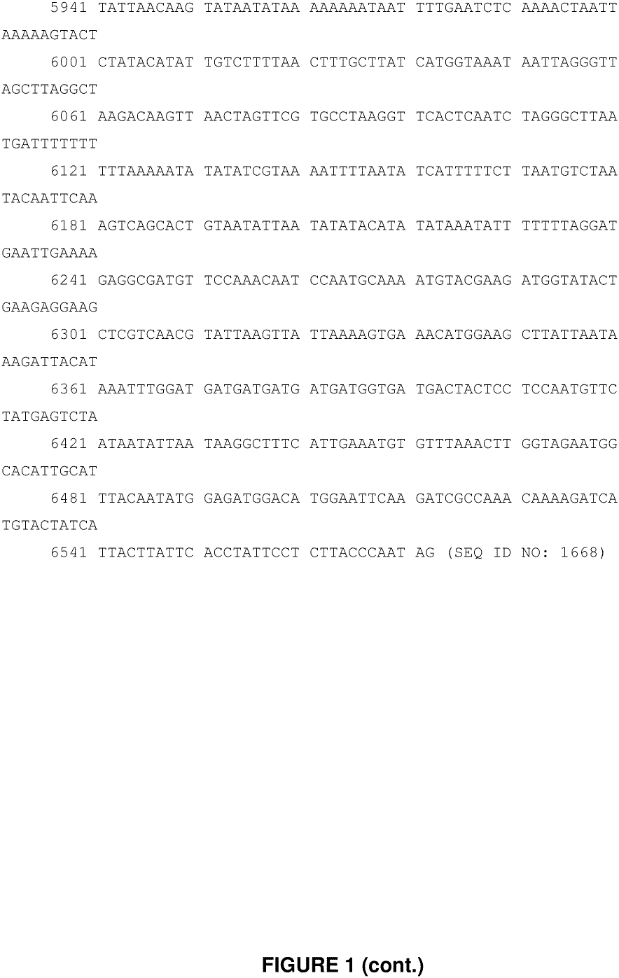

FIG. 1 provides certain mRNA coding sequences, genomic sequences (for Accession No. KY624367, genomic sequence fof CsTPS30PK that is analyzed using primer group 3 is provided) or scaffold sequences (For Accession No. KY624371, scaffold sequence that is analyzed using primer group 19 is provided) of terpene synthases that can be analyzed using the compositions and methods provided herein. Certain sequences encoding examples of terpene synthases that an be analyzed using the compositions and methods provided herein are set forth in SEQ ID NOS:1339-1397).

It is understood that for any of the terpene synthases described herein for compositions and methods of analyses/applications/uses as provided herein, suitable conservative substitutions of amino acids are known to those of skill in this art and can generally be made without altering the biological activity of the resulting terpene synthases. Those of skill in the art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224). Such substitutions can be made, for example, in accordance with those set forth in TABLE A as follows:

TABLE A

| | |
|---|---|
| Original residue | Conservative substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible, including more than one conservative substitution and, in some instances, such conservative substitutions in active sites of the enzyme, such as a terpene synthase. The substaitutions can be determined empirically or in accord with known conservative substitutions.

In embodiments of the methods provided herein, the identification of the presence or absence of a terpene synthase gene of interest is by a method selected from among one or more of high-resolution melting (HRM), quantitative PCR (qPCR), loop-mediated isothermal amplification (LAMP), restriction endonuclease digestion, gel electrophoresis and sequencing. Polynucleotide primer pairs for amplifying subsequences of terpene synthase genes of interest, e.g., by HRM, qPCR, etc. (to select plant cultivars expressing terpene synthase gene(s) that produce a desired energetic, anti-nociceptive and/or insecticidal terpene profile can be selected as a subset (one or more polyncleotide primer pairs) of the polynucleotide primer pairs (F: forward; R: reverse) listed in Table B below:

TABLE B

| Gene name | Exon # | Primer Name | Primer Seq (5′ -> 3′) | Seq ID# |
|---|---|---|---|---|
| TPS11JL | 1 | TPS11JL-1F | ATATTCAAAGATCAACCAGCAGC | SEQ ID NO: 1 |
| TPS11JL | 1 | TPS11JL-1R | TGTGGGTTTGTAGTTGCCTGAT | SEQ ID NO: 2 |
| TPS11JL | 2a | TPS11JL-2aF | AGCTATGGGAAAAGAATCAATGAGC | SEQ ID NO: 3 |
| TPS11JL | 2a | TPS11JL-2aR | GAGTTTGAAATGAAGAGCAGTGG | SEQ ID NO: 4 |
| TPS11JL | 2b | TPS11JL-2bF | GAGGCTGAAAATCCTTTAGTTAAGC | SEQ ID NO: 5 |
| TPS11JL | 2b | TPS11JL-2bR | ACAGGACTGAATCCATATTGTCTAAGG | SEQ ID NO: 6 |
| TPS11JL | 3a | TPS11JL-3aF | AGATGAAGCAAGAGATTTCACAACC | SEQ ID NO: 7 |
| TPS11JL | 3a | TPS11JL-3aR | AATGGAGTGGAAGATCCAAGGC | SEQ ID NO: 8 |
| TPS11JL | 3b | TPS11JL-3bF | ATGCCTTGGATCTTCCACTCC | SEQ ID NO: 9 |
| TPS11JL | 3b | TPS11JL-3bR | CTTTTTGGTATGCTGACTGAACG | SEQ ID NO: 10 |
| TPS11JL | 4 | TPS11JL-4F | CATTTGCTAGAGACAGAGTAGTGG | SEQ ID NO: 11 |
| TPS11JL | 4 | TPS11JL-4R | GCTGAAGCTCATCTAGTGTACC | SEQ ID NO: 12 |
| TPS11JL | 5 | TPS11JL-5F | TGAACTGGATCAGCTACCCG | SEQ ID NO: 13 |
| TPS11JL | 5 | TPS11JL-5R | GTGTGAATCCCATTTTCTTTGAGG | SEQ ID NO: 14 |
| TPS11JL | 6 | TPS11JL-6F | GTTGGGTGATCTGTGTAAATGC | SEQ ID NO: 15 |
| TPS11JL | 6 | TPS11JL-6R | CTGAGACACGTAAAATGGTTGG | SEQ ID NO: 16 |
| TPS11JL | 7 | TPS11JL-7F | TGCTACATGCGTGAAAAGGG | SEQ ID NO: 17 |
| TPS11JL | 7 | TPS11JL-7R | TGTGGTACATCTCCATTGCTCC | SEQ ID NO: 18 |
| TPS11-like1JL | 1 | TPS11-like1JL-1F | ATGTGCTGTGGTCAATAGTTCT | SEQ ID NO: 19 |
| TPS11-like1JL | 1 | TPS11-like1JL-1R | AAAGACCAAATGGAGGGCTCA | SEQ ID NO: 20 |
| TPS11-like1JL | 2 | TPS11-like1JL-2F | ACAGGTCGAGTCAAAGAATTGG | SEQ ID NO: 21 |
| TPS11-like1JL | 2 | TPS11-like1JL-2R | GCCATGTTGACGTAGAAGCC | SEQ ID NO: 22 |
| TPS11-like1JL | 3a | TPS11-like1JL-3aF | AGGAAGAGATAGGAAAAATCAAAGCG | SEQ ID NO: 23 |
| TPS11-like1JL | 3a | TPS11-like1JL-3aR | GCGTCGATGAACCACTTAGC | SEQ ID NO: 24 |
| TPS11-like1JL | 3b | TPS11-like1JL-3bF | TAAGGAAGAGATAGGAAAAATCAAAGC | SEQ ID NO: 25 |
| TPS11-like1JL | 3b | TPS11-like1JL-3bR | TTGTAGTCCTCCGATGAAGTGG | SEQ ID NO: 26 |
| TPS11-like1JL | 4 | TPS11-like1JL-4F | TGGAGGCATACTAAACTTGGGG | SEQ ID NO: 27 |
| TPS11-like1JL | 4 | TPS11-like1JL-4R | AAGCTCTAATTCATCCAATGTTCC | SEQ ID NO: 28 |
| TPS11-like1JL | 5 | TPS11-like1JL-5F | GATGGGATATGGAAATGATAAATGAGT | SEQ ID NO: 29 |
| TPS11-like1JL | 5 | TPS11-like1JL-5R | TGTTGATGGAGATGTGTTGGTCT | SEQ ID NO: 30 |
| TPS11-like1JL | 6 | TPS11-like1JL-6F | GGATTTCAGTGGGAGCACCG | SEQ ID NO: 31 |
| TPS11-like1JL | 6 | TPS11-like1JL-6R | GCACTATGACGAATTATGGCGG | SEQ ID NO: 32 |
| TPS11-like1JL | 7 | TPS11-like1JL-7F | TGAAAAGAGGTGATGCTCCG | SEQ ID NO: 33 |
| TPS11-like1JL | 7 | TPS11-like1JL-7R | CCATGCTGATACATGAAAAATCCC | SEQ ID NO: 34 |
| TPS12JL | 1 | TPS12JL-1F | ATCCTTAATACTACAAAATTAGCAAGAGC | SEQ ID NO: 35 |
| TPS12JL | 1 | TPS12JL-1R | AATGTAATCAAAAGACCAAATGGGAGG | SEQ ID NO: 36 |
| TPS12JL | 2 | TPS12JL-2F | AGTAGATTGAATGAGCTAGAGGC | SEQ ID NO: 37 |
| TPS12JL | 2 | TPS12JL-2R | AATTCGAGAGCTGTGGCG | SEQ ID NO: 38 |
| TPS12JL | 3 | TPS12JL-3F | AAGCAAGTTCAAGGCAAGAACG | SEQ ID NO: 39 |
| TPS12JL | 3 | TPS12JL-3R | GATGGTTTGTTTCGCTCCATCG | SEQ ID NO: 40 |
| TPS12JL | 4 | TPS12JL-4F | GGAGTGTTTCTTATGGACTGTGG | SEQ ID NO: 41 |
| TPS12JL | 4 | TPS12JL-4R | GAAAAGCTCTAATTCCTCCAATGTTCC | SEQ ID NO: 42 |
| TPS12JL | 5 | TPS12JL-5F | TGTGAAAGCAATGGATGATTTACC | SEQ ID NO: 43 |
| TPS12JL | 5 | TPS12JL-5R | AATTTTGGTGTCCTAACACATCG | SEQ ID NO: 44 |
| TPS12JL | 6a | TPS12JL-6aF | TCTGTAAACATCAATTACAAGAGGC | SEQ ID NO: 45 |
| TPS12JL | 6a | TPS12JL-6aR | TCGGTCCTGATACCGAAATCC | SEQ ID NO: 46 |
| TPS12JL | 6b | TPS12JL-6bF | ATGGATTTCGGTATCAGGACCG | SEQ ID NO: 47 |
| TPS12JL | 6b | TPS12JL-6bR | CATCTGTAAGTCGTAGAATCATGG | SEQ ID NO: 48 |
| TPS12JL | 7 | TPS12JL-7F | TAATGAAGATGAAAATATGGACTCTCC | SEQ ID NO: 49 |
| TPS12JL | 7 | TPS12JL-7R | GATAAACTATCCTGAGAAGCATGTCC | SEQ ID NO: 50 |
| TPS12-likeJL | 1a | TPS12-likeJL-1aF | TGTCCACTCAAATCTTAGCATCA | SEQ ID NO: 51 |
| TPS12-likeJL | 1a | TPS12-likeJL-1aR | TGAAATGTTTTTGTAGGACGAACA | SEQ ID NO: 52 |
| TPS12-likeJL | 1b | TPS12-likeJL-1bF | CAACCCAAATAAAATTGTTCGTCC | SEQ ID NO: 53 |
| TPS12-likeJL | 1b | TPS12-likeJL-1bR | TTGTGAAATGTTGTAATGTAAGAATCG | SEQ ID NO: 54 |
| TPS12-likeJL | 2a | TPS12-likeJL-2aF | TGTCAACAACAAAAGGTTGAGG | SEQ ID NO: 55 |
| TPS12-likeJL | 2a | TPS12-likeJL-2aR | AGTCTAAACCGTAAAGAAACATGG | SEQ ID NO: 56 |
| TPS12-likeJL | 2b | TPS12-likeJL-2bF | AAAGGTTGAGGAATTAAAGGAAGTGG | SEQ ID NO: 57 |
| TPS12-likeJL | 2b | TPS12-likeJL-2bR | TCAAAATGATAAGACAATCCCAAACG | SEQ ID NO: 58 |
| TPS12-likeJL | 3a | TPS12-likeJL-3aF | TGAAAAATTCAAAGACGAGGATGGG | SEQ ID NO: 59 |
| TPS12-likeJL | 3a | TPS12-likeJL-3aR | AAGGGCCTCTCTAGGGCTCG | SEQ ID NO: 60 |
| TPS12-likeJL | 3b | TPS12-likeJL-3bF | ACACTTAACCGAGTTTTTGGC | SEQ ID NO: 61 |
| TPS12-likeJL | 3b | TPS12-likeJL-3bR | TGCTGACTTCACAAAGCTCC | SEQ ID NO: 62 |
| TPS12-likeJL | 4 | TPS12-likeJL-4F | TGCAAGAGATAGGATTGTGGAAT | SEQ ID NO: 63 |
| TPS12-likeJL | 4 | TPS12-likeJL-4R | GCATGAGTTAGGATCTCAAGTTCTTC | SEQ ID NO: 64 |
| TPS12-likeJL | 5 | TPS12-likeJL-5F | TGGGATATAAATTGTGTGGATAAACTG | SEQ ID NO: 65 |
| TPS12-likeJL | 5 | TPS12-likeJL-5R | GCACTTGCTCAAACTCTTCATAACAA | SEQ ID NO: 66 |
| TPS12-likeJL | 6a | TPS12-likeJL-6aF | TGTTTGCATAGAGGACTCATCCC | SEQ ID NO: 67 |
| TPS12-likeJL | 6a | TPS12-likeJL-6aR | AGTTTTCATTCCAACCAAAGAGC | SEQ ID NO: 68 |
| TPS12-likeJL | 6b | TPS12-likeJL-6bF | TTTGGATGAAGCTCGATGTTTGC | SEQ ID NO: 69 |
| TPS12-likeJL | 6b | TPS12-likeJL-6bR | AGTAACAGATGCTCTAATAATCTTTCG | SEQ ID NO: 70 |
| TPS12-likeJL | 7a | TPS12-likeJL-7aF | ATGAGGAACATTCAGCAGTGG | SEQ ID NO: 71 |
| TPS12-likeJL | 7a | TPS12-likeJL-7aR | AGCACGAAGTAATATAGGTGAAGC | SEQ ID NO: 72 |
| TPS12-likeJL | 7b | TPS12-likeJL-7bF | ATGGCGTTTGTGAAGAGGAAGC | SEQ ID NO: 73 |
| TPS12-likeJL | 7b | TPS12-likeJL-7bR | AATTGGATGAATAAGCAAAGCAGC | SEQ ID NO: 74 |
| TPS13JL | 1 | TPS13JL-1F | TCCTTAGTTCTACAAAATTAGCAAGAGC | SEQ ID NO: 75 |
| TPS13JL | 1 | TPS13JL-1R | GTAATCAAAAGACCAAATGGGAGG | SEQ ID NO: 76 |
| TPS13JL | 2 | TPS13JL-2F | ACAAGTAGATTGAATGAGCTAGAAGC | SEQ ID NO: 77 |
| TPS13JL | 2 | TPS13JL-2R | TCATAACCATGTTGTCGTAGAAGC | SEQ ID NO: 78 |
| TPS13JL | 3a | TPS13JL-3aF | GTTCAAGGCAAGAAGGAGTAGC | SEQ ID NO: 79 |
| TPS13JL | 3a | TPS13JL-3aR | TTTGCTCCATCGTCATCGTCG | SEQ ID NO: 80 |
| TPS13JL | 3b | TPS13JL-3bF | ATTATGCAGCGACGATGACG | SEQ ID NO: 81 |
| TPS13JL | 3b | TPS13JL-3bR | CCTTGCTTCTGATCTTGTTATTCTCC | SEQ ID NO: 82 |
| TPS13JL | 4 | TPS13JL-4F | TGGAGTGTTTCTTATGGACTGTGG | SEQ ID NO: 83 |
| TPS13JL | 4 | TPS13JL-4R | CTCTCAACAGCATTAGTGAAAAGC | SEQ ID NO: 84 |
| TPS13JL | 5 | TPS13JL-5F | TGTGAAAGCAATGGATGATTTACC | SEQ ID NO: 85 |
| TPS13JL | 5 | TPS13JL-5R | AATTTTGGTCTCCTAACACATCG | SEQ ID NO: 86 |
| TPS13JL | 6 | TPS13JL-6F | GGCGAAATGGTTTCACAGTGG | SEQ ID NO: 87 |
| TPS13JL | 6 | TPS13JL-6R | CGTAGAATCATGGATGCGTGG | SEQ ID NO: 88 |
| TPS13JL | 7 | TPS13JL-7F | AAATAATGAAGATGAAAATATGGACTCTCC | SEQ ID NO: 89 |
| TPS13JL | 7 | TPS13JL-7R | GATAAACTATCCTGAGAAGCATGTCC | SEQ ID NO: 90 |
| TPS13-like1JL | 1 | TPS13-like1JL-1F | AAACTATCAACCTTCACTTTGGC | SEQ ID NO: 91 |
| TPS13-like1JL | 1 | TPS13-like1JL-1R | TTCCTCTTTTGCTCTCTTCACC | SEQ ID NO: 92 |
| TPS13-like1JL | 2 | TPS13-like1JL-2F | TGCAAAGACTTGGAATCTCTTATCAC | SEQ ID NO: 93 |
| TPS13-like1JL | 2 | TPS13-like1JL-2R | GAGACACCGGATAACCATGTTG | SEQ ID NO: 94 |
| TPS13-like1JL | 3 | TPS13-like1JL-3F | GAGGCAATTTTATGGTGTGTACC | SEQ ID NO: 95 |
| TPS13-like1JL | 3 | TPS13-like1JL-3R | TCTTTGTTGTTTCTCTTGTCTCTTCC | SEQ ID NO: 96 |
| TPS13-like1JL | 4a | TPS13-like1JL-4aF | TGGGAAAGCACTGGTATGGG | SEQ ID NO: 97 |
| TPS13-like1JL | 4a | TPS13-like1JL-4aR | AGCTCCAATTCATCTAGTGTACC | SEQ ID NO: 98 |
| TPS13-like1JL | 4b | TPS13-like1JL-4bF | GTGGGAAAGCACTGGTATGGG | SEQ ID NO: 99 |
| TPS13-like1JL | 4b | TPS13-like1JL-4bR | AATTTTTGTAATGTGGCTCAAATGC | SEQ ID NO: 100 |
| TPS13-like1JL | 5 | TPS13-like1JL-5F | TGGGATATTAGTGCTATGGATGGG | SEQ ID NO: 101 |
| TPS13-like1JL | 5 | TPS13-like1JL-5R | TTCTGAAGGAATTTTATTATATGGAGGC | SEQ ID NO: 102 |
| TPS13-like1JL | 6 | TPS13-like1JL-6F | TAGTGAATCCAAACAAGGAAGATGC | SEQ ID NO: 103 |
| TPS13-like1JL | 6 | TPS13-like1JL-6R | TCATCTGTAAGACGTAAAATCATCG | SEQ ID NO: 104 |
| TPS13-like1JL | 7 | TPS13-like1JL-7F | GATGGCGTGTTTGAAGAAGAAGC | SEQ ID NO: 105 |
| TPS13-like1JL | 7 | TPS13-like1JL-7R | TGATAGTACATGATCTTTTGTTTGGC | SEQ ID NO: 106 |
| TPS13-like2JL | 1 | TPS13-like2JL-1F | TTCTCGAAGATCAGCAAACTATCAAC | SEQ ID NO: 107 |
| TPS13-like2JL | 1 | TPS13-like2JL-1R | TTGAAAGGGGTAGAAAGTGATTGTA | SEQ ID NO: 108 |
| TPS13-like2JL | 2a | TPS13-like2JL-2aF | AGGTAAGAGTAATGGTGAAGAGAGC | SEQ ID NO: 109 |
| TPS13-like2JL | 2a | TPS13-like2JL-2aR | CATTCTCAAAGTGGTAAGAGATTCC | SEQ ID NO: 110 |
| TPS13-like2JL | 2b | TPS13-like2JL-2bF | GAGCAAAAGAGGAGGAGAAGCC | SEQ ID NO: 111 |
| TPS13-like2JL | 2b | TPS13-like2JL-2bR | GGCATAGACATTTTTGTTGGTGTTGC | SEQ ID NO: 112 |
| TPS13-like2JL | 3 | TPS13-like2JL-3F | GAGGCAATTTTATGGTGTCTTCC | SEQ ID NO: 113 |
| TPS13-like2JL | 3 | TPS13-like2JL-3R | TGTTGTGTGTCTTGTCTCTTCC | SEQ ID NO: 114 |
| TPS13-like2JL | 4 | TPS13-like2JL-4F | TGGGAAAGCACTGATATGGG | SEQ ID NO: 115 |
| TPS13-like2JL | 4 | TPS13-like2JL-4R | AGCTCCAATTCATCTAGTGTACC | SEQ ID NO: 116 |
| TPS13-like2JL | 5 | TPS13-like2JL-5F | TGGGATATTAGTGCTATGGATGGG | SEQ ID NO: 117 |
| TPS13-like2JL | 5 | TPS13-like2JL-5R | TTCTGAAGGAATTTTATTATATGGAGGC | SEQ ID NO: 118 |
| TPS13-like2JL | 6 | TPS13-like2JL-6F | AGCTTTGAAGAGTACATTGAGAATGC | SEQ ID NO: 119 |
| TPS13-like2JL | 6 | TPS13-like2JL-6R | ATCATCTGTAAGTCGTAAAATCATCG | SEQ ID NO: 120 |
| TPS13-like2JL | 7a | TPS13-like2JL-7aF | GGCGATGTTCCCAAATCAATCC | SEQ ID NO: 121 |
| TPS13-like2JL | 7a | TPS13-like2JL-7aR | AACATTGGAGAGTAGTCATCACC | SEQ ID NO: 122 |
| TPS13-like2JL | 7b | TPS13-like2JL-7bF | GATGATGATGGTGATGACTACTCTCC | SEQ ID NO: 123 |
| TPS13-like2JL | 7b | TPS13-like2JL-7bR | GATAGTACATGATCTTTTGTATGGCG | SEQ ID NO: 124 |
| TPS13PK | 2 | TPS13PK-2F | GATCAGCAAACTATCAACCCCC | SEQ ID NO: 125 |
| TPS13PK | 2 | TPS13PK-2R | TCACCATCACTCTTACTTCTTCC | SEQ ID NO: 126 |
| TPS13PK | 3 | TPS13PK-3F | ACTTGGAATCTCTTATCACTTTGAGG | SEQ ID NO: 127 |
| TPS13PK | 3 | TPS13PK-3R | AGAGAATTGGCATACACATTATTATTGG | SEQ ID NO: 128 |
| TPS13PK | 4 | TPS13PK-4F | GAGGCAATTTTATGGTGTGTACC | SEQ ID NO: 129 |
| TPS13PK | 4 | TPS13PK-4R | TTTCTCTTGTCTCTTCCAAAATACC | SEQ ID NO: 130 |
| TPS13PK | 5a | TPS13PK-5aF | TGGTGGGAAAGCACTGATATGG | SEQ ID NO: 131 |
| TPS13PK | 5a | TPS13PK-5aR | AATGTGGCTCAAATGCAACTCC | SEQ ID NO: 132 |
| TPS13PK | 5b | TPS13PK-5bF | TGGGAAAGCACTGATATGGG | SEQ ID NO: 133 |
| TPS13PK | 5b | TPS13PK-5bR | AGCTCCAATTCATCTAGTGTACC | SEQ ID NO: 134 |
| TPS13PK | 6 | TPS13PK-6F | TGGGATATTAGTGCTATGGATGGG | SEQ ID NO: 135 |
| TPS13PK | 6 | TPS13PK-6R | TTCTGAAGGAATTTTATTATATGGAGGC | SEQ ID NO: 136 |
| TPS13PK | 7a | TPS13PK-7aF | GCTTTGAAGAGTACATTGAGAATGC | SEQ ID NO: 137 |
| TPS13PK | 7a | TPS13PK-7aR | CGATGAATGTCGTATTATGGTAGGG | SEQ ID NO: 138 |
| TPS13PK | 7b | TPS13PK-7bF | GTGAATCCAAACAAGGAAAATGCC | SEQ ID NO: 139 |
| TPS13PK | 7b | TPS13PK-7bR | TGAAGTTCCCATATCATCTTTAAGTCG | SEQ ID NO: 140 |
| TPS13PK | 8a | TPS13PK-8aF | TGAATTGAAAAGAGGCGATGTTCC | SEQ ID NO: 141 |
| TPS13PK | 8a | TPS13PK-8aR | ACATTGGAGAGTAGTCATCACC | SEQ ID NO: 142 |
| TPS13PK | 8b | TPS13PK-8bF | TGGTATATCTGAAGAGGAAGCTCG | SEQ ID NO: 143 |
| TPS13PK | 8b | TPS13PK-8bR | AGAACATTGGAGAGTAGTCATCACC | SEQ ID NO: 144 |
| TPS14CT | 1 | TPS14CT-1F | GCATAGCTTTTCACCAATTTGC | SEQ ID NO: 145 |
| TPS14CT | 1 | TPS14CT-1R | GGGAGGGATGATGATGAAGCA | SEQ ID NO: 146 |
| TPS14CT | 2 | TPS14CT-2F | CAATGTACTGTGGTCGATAACCC | SEQ ID NO: 147 |
| TPS14CT | 2 | TPS14CT-2R | GAACAAAATCAAAAGACCAAATGGG | SEQ ID NO: 148 |
| TPS14CT | 3 | TPS14CT-3F | TGGAGAAAGATGTGAAAAGGATGC | SEQ ID NO: 149 |
| TPS14CT | 3 | TPS14CT-3R | ATTGGCGTAGAAGCCTAAATTGG | SEQ ID NO: 150 |
| TPS14CT | 4 | TPS14CT-4F | TCACAAGACAGGAGAGTTCAAGG | SEQ ID NO: 151 |
| TPS14CT | 4 | TPS14CT-4R | TGAAGTGGCATCTCCAAAGC | SEQ ID NO: 152 |
| TPS14CT | 5a | TPS14CT-5aF | TGGTGGAAGGATTCTAAACTTGG | SEQ ID NO: 153 |
| TPS14CT | 5a | TPS14CT-5aR | CTCCAATGTTCCATAAATGTCATGC | SEQ ID NO: 154 |
| TPS14CT | 5b | TPS14CT-5bF | GCAAGTTGGAGTAAGATTTGAGC | SEQ ID NO: 155 |
| TPS14CT | 5b | TPS14CT-5bR | ATTAGTGAAAAGTTGTAGTTCCTCC | SEQ ID NO: 156 |
| TPS14CT | 7a | TPS14CT-7aF | AAGAGGCAAAATGGTTTTATAGTGG | SEQ ID NO: 157 |
| TPS14CT | 7a | TPS14CT-7aR | TTGGTAACAGGATTTGTAAAAGCG | SEQ ID NO: 158 |
| TPS14CT | 7b | TPS14CT-7bF | GGATGGTTGTCTGTGGGAGG | SEQ ID NO: 159 |
| TPS14CT | 7b | TPS14CT-7bR | TGCATGGCGAACTATGTTAGG | SEQ ID NO: 160 |
| TPS14CT | 8 | TPS14CT-8F | TGCAAAAATCTTGGTAGAGCG | SEQ ID NO: 161 |
| TPS14CT | 8 | TPS14CT-8R | TGGGGATAGGAGTAATAATCAACCC | SEQ ID NO: 162 |
| TPS14JL | 1 | TPS14JL-1F | ATATTCAAGTCTTAGCTTCATCTCAATTA | SEQ ID NO: 163 |
| TPS14JL | 1 | TPS14JL-1R | ATCGCCCCAAATAGAAGGGTG | SEQ ID NO: 164 |
| TPS14JL | 2 | TPS14JL-2F | TTTGAGAGTGAAATTGAGAAATTGTTGG | SEQ ID NO: 165 |
| TPS14JL | 2 | TPS14JL-2R | TAAATCCATGTTGTCTTAATAATCTAAACC | SEQ ID NO: 166 |
| TPS14JL | 3 | TPS14JL-3F | GGTTTGCTTAGCTTGTATGAGGC | SEQ ID NO: 167 |
| TPS14JL | 3 | TPS14JL-3R | GCCTCTCTATGGTCTTTCTTAGAGG | SEQ ID NO: 168 |
| TPS14JL | 4 | TPS14JL-4F | TGGTGGAAAGAGCATGAGTTTGC | SEQ ID NO: 169 |
| TPS14JL | 4 | TPS14JL-4R | ATTGAGGCTAATGCAATGACTTTGG | SEQ ID NO: 170 |
| TPS14JL | 6 | TPS14JL-6F | AGCTCGATGGTTGAGTGAAGG | SEQ ID NO: 171 |
| TPS14JL | 6 | TPS14JL-6R | CTAGCAAGGAGAGTGGAAGC | SEQ ID NO: 172 |
| TPS14JL | 7a | TPS14JL-7aF | TGAGCAAAAGAGAAATCACATACC | SEQ ID NO: 173 |
| TPS14JL | 7a | TPS14JL-7aR | TTTCTTTCCAGTGGGTGTCC | SEQ ID NO: 174 |
| TPS14JL | 7b | TPS14JL-7bF | TGAAACAATATGGGGTATCAGAGG | SEQ ID NO: 175 |
| TPS14JL | 7b | TPS14JL-7bR | AATGCTTTCTTTGAGCACTTTTCC | SEQ ID NO: 176 |
| TPS15CT | 1a | TPS15CT-1aF | GCATTGTATGGCTGTTCACC | SEQ ID NO: 177 |
| TPS15CT | 1a | TPS15CT-1aR | TCGAAAGACCAAATCGGAGG | SEQ ID NO: 178 |
| TPS15CT | 1b | TPS15CT-1bF | TCACACCAAAAACATCTATTAGTCC | SEQ ID NO: 179 |
| TPS15CT | 1b | TPS15CT-1bR | CAAATCGGAGGTTCATAATTGGC | SEQ ID NO: 180 |
| TPS15CT | 2a | TPS15CT-2aF | AAGAAAGAAGTGACAAGAATGCTCC | SEQ ID NO: 181 |
| TPS15CT | 2a | TPS15CT-2aR | ACTGTATAGCCATGTTGGCG | SEQ ID NO: 182 |
| TPS15CT | 2b | TPS15CT-2bF | GAAATTAACTCTTTAGCCCTACTCG | SEQ ID NO: 183 |
| TPS15CT | 2b | TPS15CT-2bR | AGCCTAAATTCGAGAGCAATGG | SEQ ID NO: 184 |
| TPS15CT | 3a | TPS15CT-3aF | TGCTTTCAAGGATAAGAGAGGG | SEQ ID NO: 185 |
| TPS15CT | 3a | TPS15CT-3aR | TCTTCCTCATTCTCCATTTTCTCC | SEQ ID NO: 186 |
| TPS15CT | 3b | TPS15CT-3bF | CATGGAGAAAATGGAGAATGAGG | SEQ ID NO: 187 |
| TPS15CT | 3b | TPS15CT-3bR | TCGCAAACTCGAACAAAGTCG | SEQ ID NO: 188 |
| TPS15CT | 4 | TPS15CT-4F | TTGCTAGAGATAGATTGATGGAAGC | SEQ ID NO: 189 |
| TPS15CT | 4 | TPS15CT-4R | AAAGCTCTAATTCCTCCAAAGTTCC | SEQ ID NO: 190 |
| TPS15CT | 5 | TPS15CT-5F | ACCAGATTACATGAAGATGCCTT | SEQ ID NO: 191 |
| TPS15CT | 5 | TPS15CT-5R | CTAATACATCGAACCCCATCTCA | SEQ ID NO: 192 |
| TPS15CT | 6a | TPS15CT-6aF | GGTATTATAGTGGATACCAACCAAC | SEQ ID NO: 193 |
| TPS15CT | 6a | TPS15CT-6aR | AAGCCAACCCAACTCAGTGT | SEQ ID NO: 194 |
| TPS15CT | 6b | TPS15CT-6bF | ACACTGAGTTGGGTTGGCTTT | SEQ ID NO: 195 |
| TPS15CT | 6b | TPS15CT-6bR | GTTCCTAAATCATCTGCAAGCCT | SEQ ID NO: 196 |
| TPS15CT | 7 | TPS15CT-7F | TTGAATAGAGGCGACGTTCC | SEQ ID NO: 197 |
| TPS15CT | 7 | TPS15CT-7R | TGCTGTTCTAGCCATATTTTTGC | SEQ ID NO: 198 |
| TPS16CC | 1 | TPS16CC-1F | TCTAGTCAAGTGTTAGCTTCATCTC | SEQ ID NO: 199 |
| TPS16CC | 1 | TPS16CC-1R | TTTGTTGTTGGTCGAATGATGT | SEQ ID NO: 200 |
| TPS16CC | 2 | TPS16CC-2F | AGGGACAAGTTGAAGAATTGAAAGA | SEQ ID NO: 201 |
| TPS16CC | 2 | TPS16CC-2R | CATGGTTGGAGTAGTAGTAGTGT | SEQ ID NO: 202 |
| TPS16CC | 3a | TPS16CC-3aF | GAGAGTGGTAAGTTTAAGGAAAGC | SEQ ID NO: 203 |
| TPS16CC | 3a | TPS16CC-3aR | CCTAGCATAAAGCCTCACTAGG | SEQ ID NO: 204 |
| TPS16CC | 3b | TPS16CC-3bF | GGCTCTTGCTTTCACTACCACC | SEQ ID NO: 205 |
| TPS16CC | 3b | TPS16CC-3bR | CCTCACTAGGGTTTTTCTCAAAGG | SEQ ID NO: 206 |
| TPS16CC | 4 | TPS16CC-4F | GGTGGAAAGAATTAGACTTGGC | SEQ ID NO: 207 |
| TPS16CC | 4 | TPS16CC-4R | ATGTCCCACCTGAGAATTGC | SEQ ID NO: 208 |
| TPS16CC | 5 | TPS16CC-5F | ACTTAGTCCAGATTATTTGAAGACATATT | SEQ ID NO: 209 |
| TPS16CC | 5 | TPS16CC-5R | TGCATAGTGAAGTTTGTATCTCTCT | SEQ ID NO: 210 |
| TPS16CC | 6 | TPS16CC-6F | TTGAGACCTCTTTTGTTGGAATGC | SEQ ID NO: 211 |
| TPS16CC | 6 | TPS16CC-6R | AAAATCTTTGGTTGTGTAGAGAGC | SEQ ID NO: 212 |
| TPS16CC | 7a | TPS16CC-7aF | ATGGTGTATCGGAACAAGAGG | SEQ ID NO: 213 |
| TPS16CC | 7a | TPS16CC-7aR | AACGCAGCAACACTGTCC | SEQ ID NO: 214 |
| TPS16CC | 7b | TPS16CC-7bF | AAGTGCTTGACATTATCTACAAAGAAGG | SEQ ID NO: 215 |
| TPS16CC | 7b | TPS16CC-7bR | TAATGGGATGGGATCTATAAGCAACGC | SEQ ID NO: 216 |
| TPS17JL | 1a | TPS17JL-1aF | ATGGCTTTTCACCAATTTGCTCC | SEQ ID NO: 217 |
| TPS17JL | 1a | TPS17JL-1aR | TCGTAGAACTAGGGTTATCGACC | SEQ ID NO: 218 |
| TPS17JL | 1b | TPS17JL-1bF | TTTGCTCCATCATCATCCCTCCC | SEQ ID NO: 219 |
| TPS17JL | 1b | TPS17JL-1bR | GAGGTCCATAGTTGGCTGATCTTCG | SEQ ID NO: 220 |
| TPS17JL | 2 | TPS17JL-2F | TTGAAGAAAGAAGTGACAAGAATGG | SEQ ID NO: 221 |
| TPS17JL | 2 | TPS17JL-2R | GGTAAGATATTCCAAGCCTTTGC | SEQ ID NO: 222 |
| TPS17JL | 4a | TPS17JL-4aF | TACATGGAGAAAATGGAGAATGAGG | SEQ ID NO: 223 |
| TPS17JL | 4a | TPS17JL-4aR | TGAAGCGGAAGCTCGAAAGC | SEQ ID NO: 224 |
| TPS17JL | 4b | TPS17JL-4bF | ATCACGCTTTCGAGCTTCCG | SEQ ID NO: 225 |
| TPS17JL | 4b | TPS17JL-4bR | AAATCTTCTTGGTGTGTTGATTGC | SEQ ID NO: 226 |
| TPS17JL | 5 | TPS17JL-5F | TTGCTAGAGATAGATTGATGGAAGC | SEQ ID NO: 227 |
| TPS17JL | 5 | TPS17JL-5R | AAAGCTCTAATTCCTCCAAAGTTCC | SEQ ID NO: 228 |
| TPS17JL | 6 | TPS17JL-6F | GTTACCAGATTACATGAAGATGCC | SEQ ID NO: 229 |
| TPS17JL | 6 | TPS17JL-6R | ACTAATACATCGAACCCCATCTC | SEQ ID NO: 230 |
| TPS17JL | 7 | TPS17JL-7F | AGTTGGGTTGGCTTTCAATAGG | SEQ ID NO: 231 |
| TPS17JL | 7 | TPS17JL-7R | ATGTTCCTAAATCATCTGCAAGC | SEQ ID NO: 232 |
| TPS17JL | 8 | TPS17JL-8F | TAGAGGCGACGTTCCTAAATCG | SEQ ID NO: 233 |
| TPS17JL | 8 | TPS17JL-8R | TGCTGTTCTAGCCATATTTTTGC | SEQ ID NO: 234 |
| TPS18JL | 1 | TPS18JL-1F | TCTAGTCAAGTGTTAGCTTCATCTC | SEQ ID NO: 235 |
| TPS18JL | 1 | TPS18JL-1R | TGTTGTTGGTCGAATGATGTTTTG | SEQ ID NO: 236 |
| TPS18JL | 2 | TPS18JL-2F | AAGAAGTTGTTAGGAAAGAGATATTCC | SEQ ID NO: 237 |
| TPS18JL | 2 | TPS18JL-2R | AGATGAAATGTTAAATCCATGTTGTCG | SEQ ID NO: 238 |
| TPS18JL | 3a | TPS18JL-3aF | TGAGAGTGGTAAGTTTAAGGAAAGC | SEQ ID NO: 239 |
| TPS18JL | 3a | TPS18JL-3aR | GGTGGTAGTGAAAGCAAGAGC | SEQ ID NO: 240 |
| TPS18JL | 3b | TPS18JL-3bF | GGCTCTTGCTTTCACTACCACC | SEQ ID NO: 241 |
| TPS18JL | 3b | TPS18JL-3bR | CCTCACTAGGGTTTTTCTCAAAGG | SEQ ID NO: 242 |
| TPS18JL | 3c | TPS18JL-3cF | TGAGAAAAACCCTAGTGAGGC | SEQ ID NO: 243 |
| TPS18JL | 3c | TPS18JL-3cR | TTGTAGTAGATTGAAGTCCAACTTTGC | SEQ ID NO: 244 |
| TPS18JL | 4 | TPS18JL-4F | TTAGACTTGGCAAACAAACTACC | SEQ ID NO: 245 |
| TPS18JL | 4 | TPS18JL-4R | TATGTCCCACCTGAGAATTGC | SEQ ID NO: 246 |
| TPS18JL | 5 | TPS18JL-5F | ACTTAGTCCAGATTATTTGAAGACATATTA | SEQ ID NO: 247 |
| TPS18JL | 5 | TPS18JL-5R | TTGCATAGTGAAGTTTGTATCTCTCT | SEQ ID NO: 248 |
| TPS18JL | 6a | TPS18JL-6aF | TTCCATGAAGCACAATGGTTGA | SEQ ID NO: 249 |
| TPS18JL | 6a | TPS18JL-6aR | GCATTCCAACAAAAGAGGTCTCA | SEQ ID NO: 250 |
| TPS18JL | 6b | TPS18JL-6bF | GGTTACCCAATGTTGATTGAGACC | SEQ ID NO: 251 |
| TPS18JL | b | TPS18JL-6bR | GGTTGTGTAGAGAGCCATTCAAATA | SEQ ID NO: 252 |
| TPS18JL | 7 | TPS18JL-7F | TGGTGTATCGGAACAAGAGGC | SEQ ID NO: 253 |
| TPS18JL | 7 | TPS18JL-7R | CAACGCAGCAACACTGTCC | SEQ ID NO: 254 |
| TPS18VF | 1 | TPS18VF-1F | ACGCATCTTTTCGTCCCTTT | SEQ ID NO: 255 |
| TPS18VF | 1 | TPS18VF-1R | TGAGATCACCGTTACTCCTGATA | SEQ ID NO: 256 |
| TPS18VF | 2 | TPS18VF-2F | AGCAGACCCATTTGATGAAGG | SEQ ID NO: 257 |
| TPS18VF | 2 | TPS18VF-2R | CAGCAGGAACGAAGAGACCG | SEQ ID NO: 258 |
| TPS18VF | 3a | TPS18VF-3aF | TCGACACAAGGCTAAGAGAGG | SEQ ID NO: 259 |
| TPS18VF | 3a | TPS18VF-3aR | GTCCTTGGCTGTGAACTTGG | SEQ ID NO: 260 |
| TPS18VF | 3b | TPS18VF-3bF | CAAGTTCACAGCCAAGGACC | SEQ ID NO: 261 |
| TPS18VF | 3b | TPS18VF-3bR | TTATCAATTTCCATTCTATGCAGGG | SEQ ID NO: 262 |
| TPS18VF | 4 | TPS18VF-4F | GGTGGCGAGACATTGGTTTAGC | SEQ ID NO: 263 |
| TPS18VF | 4 | TPS18VF-4R | ATGGATTTTGTAAGCGCAACCC | SEQ ID NO: 264 |
| TPS18VF | 5 | TPS18VF-5F | TGCTATACAAAAACTTCCAGACTCC | SEQ ID NO: 265 |
| TPS18VF | 5 | TPS18VF-5R | TGTAAAGGGCTCCATCCACG | SEQ ID NO: 266 |
| TPS18VF | 6a | TPS18VF-6aF | GGGCAAGTTTGTGCGAAGC | SEQ ID NO: 267 |
| TPS18VF | 6a | TPS18VF-6aR | TGGCACTACCAAAGTCATCCC | SEQ ID NO: 268 |
| TPS18VF | 6b | TPS18VF-6bF | ATGGTGTGGTCAGCTCAGG | SEQ ID NO: 269 |
| TPS18VF | 6b | TPS18VF-6bR | TGGCACTACCAAAGTCATCCC | SEQ ID NO: 270 |
| TPS18VF | 7a | TPS18VF-7aF | AGAATCAAGAAGGACATGACGG | SEQ ID NO: 271 |
| TPS18VF | 7a | TPS18VF-7aR | TTTGTTGAGGCACTTCCATGC | SEQ ID NO: 272 |
| TPS18VF | 7b | TPS18VF-7bF | AAGTGCCTCAACAAAGAATGC | SEQ ID NO: 273 |
| TPS18VF | 7b | TPS18VF-7bR | GTTCTTCCAAGTGGGGTAGG | SEQ ID NO: 274 |
| TPS19BL | 1 | TPS19BL-1F | ATGGCGTTGTCAATAATGTCTTCTTACG | SEQ ID NO: 275 |
| TPS19BL | 1 | TPS19BL-1R | GAGAACTCGAAAGTGATGAGGAGG | SEQ ID NO: 276 |
| TPS19BL | 2a | TPS19BL-2aF | AGCAGACCCATTTGATGAAGG | SEQ ID NO: 277 |
| TPS19BL | 2a | TPS19BL-2aR | AGCAGGAACGAAGTGACCG | SEQ ID NO: 278 |
| TPS19BL | 2b | TPS19BL-2bF | GATAAATACAGGGACGTTTTAAGAAAAGC | SEQ ID NO: 279 |
| TPS19BL | 2b | TPS19BL-2bR | CCTCGAAGATATAGTCAATTCCTAGC | SEQ ID NO: 280 |
| TPS19BL | 3 | TPS19BL-3F | ATATGAAGCCTCCCATCTATGC | SEQ ID NO: 281 |
| TPS19BL | 3 | TPS19BL-3R | CCATTCTCACTGGGATAGACACC | SEQ ID NO: 282 |
| TPS19BL | 4 | TPS19BL-4F | GGTGGCGAGACATTGGTTTAGC | SEQ ID NO: 283 |
| TPS19BL | 4 | TPS19BL-4R | ATGGATTTTGTAAGCGCAACCC | SEQ ID NO: 284 |
| TPS19BL | 5a | TPS19BL-5aF | AGAAAAACTTCCAGACTCCATGA | SEQ ID NO: 285 |
| TPS19BL | 5a | TPS19BL-5aR | GCTCCATCCACGCTTTTGAT | SEQ ID NO: 286 |
| TPS19BL | 5b | TPS19BL5bF | TCAATGAGTCTAGCCATACGATCT | SEQ ID NO: 287 |
| TPS19BL | 5b | TPS19BL-5bR | TGTAAAGGGCTCCATCCACG | SEQ ID NO: 288 |
| TPS19BL | 6 | TPS19BL-6F | GGGCAAGTTTGTGCGAAGC | SEQ ID NO: 289 |
| TPS19BL | 6 | TPS19BL-6R | TGGCACTACCAAAGTCATCCC | SEQ ID NO: 290 |
| TPS19BL | 7a | TPS19BL-7aF | GGACATGACGGATCTTATGTGG | SEQ ID NO: 291 |
| TPS19BL | 7a | TPS19BL-7aR | TTGTTGAGGCACTTCCATGC | SEQ ID NO: 292 |
| TPS19BL | 7b | TPS19BL-7bF | ATCCAGCGTTTCCACCACC | SEQ ID NO: 293 |
| TPS19BL | 7b | TPS19BL-7bR | GTTCTTCCAAGTGGGGTAGGC | SEQ ID NO: 294 |
| TPS1JL | 1 | TPS1JL-1F | ACCAATTTGCTTCATCATCATCCC | SEQ ID NO: 295 |
| TPS1JL | 1 | TPS1JL-1R | AAATGGGAGGTCCATAGTTGGC | SEQ ID NO: 296 |
| TPS1JL | 2 | TPS1JL-2F | TGAAAAGGATGCTAATTGGAGTGG | SEQ ID NO: 297 |
| TPS1JL | 2 | TPS1JL-2R | CGTAGAAGCCTAAATTGGAGAGC | SEQ ID NO: 298 |
| TPS1JL | 3 | TPS1JL-3F | ATCACAAGACAGGAGAGTTCAAGGC | SEQ ID NO: 299 |
| TPS1JL | 3 | TPS1JL-3R | ATTCTCCATTGAAGTGGCATCTCC | SEQ ID NO: 300 |
| TPS1JL | 4a | TPS1JL-4aF | ACTTGGAGAGAAATTGCCTTTCG | SEQ ID NO: 301 |
| TPS1JL | 4a | TPS1JL-4aR | TCCAATGTTCCATAAATGTCATGC | SEQ ID NO: 302 |
| TPS1JL | 4b | TPS1JL-4bF | GCAAGTTGGAGTAAGATTTGAGC | SEQ ID NO: 303 |
| TPS1JL | 4b | TPS1JL-4bR | ATTAGTGAAAAGTTGTAGTTCCTCC | SEQ ID NO: 304 |
| TPS1JL | 5 | TPS1JL-5F | TAAGTTACCAGATTATATGAAGACAGC | SEQ ID NO: 305 |
| TPS1JL | 5 | TPS1JL-5R | TTTGTGAAATTGTATGTAAAGTAGAAAGC | SEQ ID NO: 306 |
| TPS1JL | 6 | TPS1JL-6F | AGAATGGATGGTTGTCTGTGGG | SEQ ID NO: 307 |
| TPS1JL | 6 | TPS1JL-6R | GGCGAACTATGTTAGGATGACC | SEQ ID NO: 308 |
| TPS1JL | 7 | TPS1JL-7F | CAATTCAATGTTATATGCACGATACTGG | SEQ ID NO: 309 |
| TPS1JL | 7 | TPS1JL-7R | CTGAGAAGCATGTCCATCGCC | SEQ ID NO: 310 |
| TPS20CT | 1 | TPS20CT-1F | ATTCAAGTCTTAGCTTCATCTCAATTA | SEQ ID NO: 311 |
| TPS20CT | 1 | TPS20CT-1R | CGATCACCCCAAATAGAAGGGT | SEQ ID NO: 312 |
| TPS20CT | 2 | TPS20CT-2F | TTTGAGAGTGAAATTGAGAAATTGTTGG | SEQ ID NO: 313 |
| TPS20CT | 2 | TPS20CT-2R | TAAATCCATGTTGTCTTAATAATCTAAACC | SEQ ID NO: 314 |
| TPS20CT | 3a | TPS20CT-3aF | GCCTAATAACCGATGTTCCAGG | SEQ ID NO: 315 |
| TPS20CT | 3a | TPS20CT-3aR | GAGATGTAAAACCTAGCATGAAGCC | SEQ ID NO: 316 |
| TPS20CT | 3b | TPS20CT-3bF | AGACCATAGAGAGGCTTCATGC | SEQ ID NO: 317 |
| TPS20CT | 3b | TPS20CT-3bR | CGTAATTTCACTAAGTTCCTTTTTGTGG | SEQ ID NO: 318 |
| TPS20CT | 4 | TPS20CT-4F | GGTGGAAGGAGCATGAGTTTGC | SEQ ID NO: 319 |
| TPS20CT | 4 | TPS20CT-4R | ACCTTTGAATTGCTTTGGTAAGAAGC | SEQ ID NO: 320 |
| TPS20CT | 6 | TPS20CT-6F | GAGTCTCTTATGTTTCTTCTGGTAACG | SEQ ID NO: 321 |
| TPS20CT | 6 | TPS20CT-6R | CATGAACCTAGAAAGGAGAGTAGAAGC | SEQ ID NO: 322 |
| TPS20CT | 7a | TPS20CT-7aF | TGAGCAAAAGAGAAATCACATACC | SEQ ID NO: 323 |
| TPS20CT | 7a | TPS20CT-7aR | TTTCTTTCCAGTGGGTGTCC | SEQ ID NO: 324 |
| TPS20CT | 7b | TPS20CT-7bF | CCAGCAGTTGTGCCCTTTCC | SEQ ID NO: 325 |
| TPS20CT | 7b | TPS20CT-7bR | CAATGCTTTCTTTGAGCACTTTTCC | SEQ ID NO: 326 |
| TPS20JL | 1 | TPS20JL-1F | TCAAGTCTTAGCTTCATCTCAATTATGTG | SEQ ID NO: 327 |
| TPS20JL | 1 | TPS20JL-1R | GATCACCCCAAATAGAAGGGT | SEQ ID NO: 328 |
| TPS20JL | 2 | TPS20JL-2F | TTCGAGAGTGAAATCGAGAAATTATTGG | SEQ ID NO: 329 |
| TPS20JL | 2 | TPS20JL-2R | ATCCACTTTGTCTTAATAGTCTAAACCG | SEQ ID NO: 330 |
| TPS20JL | 3a | TPS20JL-3aF | CTTGCTTTCACAACCACTCACC | SEQ ID NO: 331 |
| TPS20JL | 3a | TPS20JL-3aR | CCTCTCTATGGTCTTTCTTAGAGGC | SEQ ID NO: 332 |
| TPS20JL | 3b | TPS20JL-3bF | ATAACCGATGTTTCAGGTTTGC | SEQ ID NO: 333 |
| TPS20JL | 3b | TPS20JL-3bR | TAAGGTGAGTGGTTGTGAAAGC | SEQ ID NO: 334 |
| TPS20JL | 4 | TPS20JL-4F | GTGTATATTATGAACCCAAATACTCTCG | SEQ ID NO: 335 |
| TPS20JL | 4 | TPS20JL-4R | CCTTTGAATTGCTTTGGTAAGAAGC | SEQ ID NO: 336 |
| TPS20JL | 6 | TPS20JL-6F | GAACTATTGGAGGTTATTTTGAAGAAGC | SEQ ID NO: 337 |
| TPS20JL | 6 | TPS20JL-6R | GAGTGGAAGCTGAAACAATCTTAGGG | SEQ ID NO: 338 |
| TPS20JL | 7 | TPS20JL-7F | TGAGCAAGAGAGAAATCACATACC | SEQ ID NO: 339 |
| TPS20JL | 7 | TPS20JL-7R | AGAACACGAAGTAAGATAGGAAAAGG | SEQ ID NO: 340 |
| TPS23JL | 1 | TPS23JL-1F | CACACAAATCTTAGTATCATTATCTTCAAA | SEQ ID NO: 341 |
| TPS23JL | 1 | TPS23JL-1R | CAAATCTTGTTGTGAAATGTTGTAATGT | SEQ ID NO: 342 |
| TPS23JL | 2 | TPS23JL-2F | TGTCAACAACAAAAGGTTGAGG | SEQ ID NO: 343 |
| TPS23JL | 2 | TPS23JL-2R | TCAAAATGATAAGACAATCCCAAACG | SEQ ID NO: 344 |
| TPS23JL | 3a | TPS23JL-3aF | GAACACCATGAAGACGATGATCC | SEQ ID NO: 345 |
| TPS23JL | 3a | TPS23JL-3aR | TGCTGACTTCACTAAGCTCC | SEQ ID NO: 346 |
| TPS23JL | 3b | TPS23JL-3bF | TGCTTGGTAAGTGATACCCTTGG | SEQ ID NO: 347 |
| TPS23JL | 3b | TPS23JL-3bR | AGGGCCTCTCTAGGGCTCG | SEQ ID NO: 348 |
| TPS23JL | 4 | TPS23JL-4F | TGCAAGAGATAGGATTGTGGAATTG | SEQ ID NO: 349 |
| TPS23JL | 4 | TPS23JL-4R | ACTCAAGTTCTTCAAGTGTACCATA | SEQ ID NO: 350 |
| TPS23JL | 5 | TPS23JL-5F | TTGTTATGAAGAGTTTGAGCAACTG | SEQ ID NO: 351 |
| TPS23JL | 5 | TPS23JL-5R | ACTCTGTATGTTTCTTCTTTTTCTAGCAG | SEQ ID NO: 352 |
| TPS23JL | 6 | TPS23JL-6F | TTTGAATGAAGCTCGATGTTTGC | SEQ ID NO: 353 |
| TPS23JL | 6 | TPS23JL-6R | CCATTAGCCTACAAATAGTAACAGATGC | SEQ ID NO: 354 |
| TPS23JL | 7a | TPS23JL-7aF | GCGTTTGTGAAGAGGAAGCC | SEQ ID NO: 355 |
| TPS23JL | 7a | TPS23JL-7aR | ACCCTTGAAAAGTTAAGAGCACG | SEQ ID NO: 356 |
| TPS23JL | 7b | TPS23JL-7bF | AGAAATAAATGAAGAGTTTTTGAAGCC | SEQ ID NO: 357 |
| TPS23JL | 7b | TPS23JL-7bR | TGGATGAATAAGCAAAGCAGC | SEQ ID NO: 358 |
| TPS24JL | 10a | TPS24JL-10aF | GCTCAAGTCTATGTTGAAGGAAGC | SEQ ID NO: 359 |
| TPS24JL | 10a | TPS24JL-10aR | AACATCCTCAGAAAGCATAGGTCC | SEQ ID NO: 360 |
| TPS24JL | 10b | TPS24JL-10bF | TCAATGGTTGAAATACGAGACCG | SEQ ID NO: 361 |
| TPS24JL | 10b | TPS24JL-10bR | ATCGTTGAGAAGTCGTCCG | SEQ ID NO: 362 |
| TPS24JL | 11 | TPS24JL-11F | CGCGTGGTTTTGGCTCTGG | SEQ ID NO: 363 |
| TPS24JL | 11 | TPS24JL-11R | GGTGAATCCGTCGTCATTGGC | SEQ ID NO: 364 |
| TPS24JL | 1 | TPS24JL-1F | TGCATTCCAACATTAGGTGCTC | SEQ ID NO: 365 |
| TPS24JL | 1 | TPS24JL-1R | TGCTTTTCCTTGCACCATTTAGT | SEQ ID NO: 366 |
| TPS24JL | 2a | TPS24JL-2aF | TTATGACACTGCCTGGGTGG | SEQ ID NO: 367 |
| TPS24JL | 2a | TPS24JL-2aR | GAGGAAGACCCCATGAACCG | SEQ ID NO: 368 |
| TPS24JL | 2b | TPS24JL-2bF | AGAGAATCAGCACTCTGACGG | SEQ ID NO: 369 |
| TPS24JL | 2b | TPS24JL-2bR | CACCCCATCGCTTCATTGC | SEQ ID NO: 370 |
| TPS24JL | 4 | TPS24JL-4F | TCAGGAAGCTACTCAGAGGG | SEQ ID NO: 371 |
| TPS24JL | 4 | TPS24JL-4R | ACAGAGAACCATTTTTCCTTTGG | SEQ ID NO: 372 |
| TPS24JL | 5 | TPS24JL-5F | TATGCTCGTCTCTCAATGGTGG | SEQ ID NO: 373 |
| TPS24JL | 5 | TPS24JL-5R | AGAACACACTCCTCTCCTTGC | SEQ ID NO: 374 |
| TPS24JL | 6 | TPS24JL-6F | TCCGAGATCATCATACACCCA | SEQ ID NO: 375 |
| TPS24JL | 6 | TPS24JL-6R | TCTCCCCTAGTTGAAATGCTGT | SEQ ID NO: 376 |
| TPS24JL | 7 | TPS24JL-7F | GAAAACTTCATATTGTTCCTCAAATGTTCG | SEQ ID NO: 377 |
| TPS24JL | 7 | TPS24JL-7R | GAGTTCTTCACGGTGTATTGATTGG | SEQ ID NO: 378 |
| TPS24JL | 8a | TPS24JL-8aF | GTGGGTTGTAGAGAATAGGTTGG | SEQ ID NO: 379 |
| TPS24JL | 8a | TPS24JL-8aR | GCGGGCATCAGATAATTCAGG | SEQ ID NO: 380 |
| TPS24JL | 8b | TPS24JL-8bF | CCTGAATTATCTGATGCCCGC | SEQ ID NO: 381 |
| TPS24JL | 8b | TPS24JL-8bR | CTAGTTCCTCTTCTGAACCTCC | SEQ ID NO: 382 |
| TPS24JL | 9 | TPS24JL-9F | CAGTGTTGATTGTTTGTCGGAGC | SEQ ID NO: 383 |
| TPS24JL | 9 | TPS24JL-9R | TTGTCACACTACGTCCTTGC | SEQ ID NO: 384 |
| TPS2FN | 1a | TPS2FN-1aF | AAGATCAGCCAACTATGATCCTCCC | SEQ ID NO: 385 |
| TPS2FN | 1a | TPS2FN-1aR | ATGGAAGAGACTGAATGAAATCAAAAGACC | SEQ ID NO: 386 |
| TPS2FN | 1b | TPS2FN-1bF | TTAGAAGATCAGCCAACTATGATCC | SEQ ID NO: 387 |
| TPS2FN | 1b | TPS2FN-1bR | GAATGAAATCAAAAGACCAAATGGG | SEQ ID NO: 388 |
| TPS2FN | 2 | TPS2FN-2F | AAAGAAGAAGTGAAAAAGATGTTAGTTGG | SEQ ID NO: 389 |
| TPS2FN | 2 | TPS2FN-2R | AAAAGCCTAAATTCGAGAGCAGTGG | SEQ ID NO: 390 |
| TPS2FN | 3a | TPS2FN-3aF | GAGACGGGAAAGTTCAAAGCG | SEQ ID NO: 391 |
| TPS2FN | 3a | TPS2FN-3aR | TGAAGTGGCATCTCCAAGGC | SEQ ID NO: 392 |
| TPS2FN | 3b | TPS2FN-3bF | CGCAAATTCAAGCAAAGTGC | SEQ ID NO: 394 |
| TPS2FN | 3b | TPS2FN-3bR | GCTTCATTCTATGTGAAAAATGGCG | SEQ ID NO: 393 |
| TPS2FN | 4a | TPS2FN-4aF | AAATGGTTTATGCTAGAGATAGATTGG | SEQ ID NO: 395 |
| TPS2FN | 4a | TPS2FN-4aR | CTTGCAGATATTCTCCTAAAGTGGC | SEQ ID NO: 396 |
| TPS2FN | 4b | TPS2FN-4bF | AGAGGCTTTTCTATGGCAGG | SEQ ID NO: 397 |
| TPS2FN | 4b | TPS2FN-4bR | AAGCTCTAATTCTTCCAATGTTCC | SEQ ID NO: 398 |
| TPS2FN | 5a | TPS2FN-5aF | TGCCTTTCTTTACTTTATTTAACACCG | SEQ ID NO: 399 |
| TPS2FN | 5a | TPS2FN-5aR | GCTCTTCTAACACATCATACGCC | SEQ ID NO: 400 |
| TPS2FN | 5b | TPS2FN-5bF | ACACCGTAAATGAAATGGCG | SEQ ID NO: 401 |
| TPS2FN | 5b | TPS2FN-5bR | CGAGTTCTTGAGGTATTCAACGC | SEQ ID NO: 402 |
| TPS2FN | 6a | TPS2FN-6aF | TGGGCAGAGTTATGTAGATGC | SEQ ID NO: 403 |
| TPS2FN | 6a | TPS2FN-6aR | TCCTATTGAAAGCGAGGCG | SEQ ID NO: 404 |
| TPS2FN | 6b | TPS2FN-6bF | ACGCCTCGCTTTCAATAGG | SEQ ID NO: 405 |
| TPS2FN | 6b | TPS2FN-6bR | CTGCAAGTCGTAACATCAAGG | SEQ ID NO: 406 |
| TPS2FN | 6c | TPS2FN-6cF | TGGAAGAGGCAAAATGGTTTTATAGC | SEQ ID NO: 407 |
| TPS2FN | 6c | TPS2FN-6cR | GATCATCTGCAAGTCGTAACATCAAGG | SEQ ID NO: 408 |
| TPS2FN | 7a | TPS2FN-7aF | AAAGAGGTGACATTCTTAAATCGG | SEQ ID NO: 409 |
| TPS2FN | 7a | TPS2FN-7aR | AGTTATATTCATCTTCATCATTCATCTCC | SEQ ID NO: 410 |
| TPS2FN | 7b | TPS2FN-7bF | GGTGTTTCTGAAGATGAAGCTCG | SEQ ID NO: 411 |
| TPS2FN | 7b | TPS2FN-7bR | CTGAAATACGTTTCCTTGAATGGC | SEQ ID NO: 412 |
| TPS30JL | 2 | TPS30JL-2F | TCTCGAAGATCAGCAAACTATCAACC | SEQ ID NO: 413 |
| TPS30JL | 2 | TPS30JL-2R | ACATAATCAAATTGCCAAAGTGGG | SEQ ID NO: 414 |
| TPS30JL | 4a | TPS30JL-4aF | AGACTTGGAATCTCTTATCACTTTGAG | SEQ ID NO: 415 |
| TPS30JL | 4a | TPS30JL-4aR | GGCATACACATTATTTTTGTTGGTGTT | SEQ ID NO: 416 |
| TPS30JL | 4b | TPS30JL-4bF | GTACAACACCAACAAAAATAATGTGT | SEQ ID NO: 417 |
| TPS30JL | 4b | TPS30JL-4bR | TCGTAGGAGTCTAAATTCAAGAGAA | SEQ ID NO: 418 |
| TPS30JL | 5 | TPS30JL-5F | GAGGCAATTTTATGGTGTGTACC | SEQ ID NO: 419 |
| TPS30JL | 5 | TPS30JL-5R | TTGTTTTGTGTCTTGTCTCTTCC | SEQ ID NO: 420 |
| TPS30JL | 6 | TPS30JL-6F | TGGGAAAGCACTGATATGGG | SEQ ID NO: 421 |
| TPS30JL | 6 | TPS30JL-6R | AGCTCCAATTCATCTAGTGTACC | SEQ ID NO: 422 |
| TPS30JL | 7 | TPS30JL-7F | TGGGACATTAGTGCTATGGATGG | SEQ ID NO: 423 |
| TPS30JL | 7 | TPS30JL-7R | TCTGAAGGAATTTTATTATATGGAGGC | SEQ ID NO: 424 |
| TPS30JL | 8 | TPS30JL-8F | AGATTTGAAGAGTACATTGAGAATGC | SEQ ID NO: 425 |
| TPS30JL | 8 | TPS30JL-8R | CGATGAATGTCGTATTATGGTAGGG | SEQ ID NO: 426 |
| TPS30JL | 9 | TPS30JL-9F | AGAGGAAGCTCGTCAACGTAT | SEQ ID NO: 427 |
| TPS30JL | 9 | TPS30JL-9R | ATGTGCCATTCTACCAAGGTT | SEQ ID NO: 428 |
| TPS30-likeJL | 1 | TPS30-likeJL-1F | GACTTGGAATCTCTTACCACTTTGA | SEQ ID NO: 429 |
| TPS30-likeJL | 1 | TPS30-likeJL-1R | ACCATGTTGTCTTAGGAGTCTAAAT | SEQ ID NO: 430 |
| TPS30-likeJL | 2 | TPS30-likeJL-2F | GAATAGTTCTCGAAGATCAGCAAAC | SEQ ID NO: 431 |
| TPS30-likeJL | 2 | TPS30-likeJL-2R | CAAATTGCCAAAGTGGGGGT | SEQ ID NO: 432 |
| TPS30-likeJL | 3 | TPS30-likeJL-3F | GCAAAGACTTGGAATCTCTTACCA | SEQ ID NO: 433 |
| TPS30-likeJL | 3 | TPS30-likeJL-3R | GAGACACCGGATAACCATGTTG | SEQ ID NO: 434 |
| TPS30-likeJL | 4 | TPS30-likeJL-4F | GAGGCAATTTTATGGTGTCTTCC | SEQ ID NO: 435 |
| TPS30-likeJL | 4 | TPS30-likeJL-4R | TGTTGTGTGTCTTGTCTCTTCC | SEQ ID NO: 436 |
| TPS30-likeJL | 5 | TPS30-likeJL-5F | GTGGGAAAGCACTGGTATGG | SEQ ID NO: 437 |
| TPS30-likeJL | 5 | TPS30-likeJL-5R | AGCTCCAATTCATCTAGTGTACC | SEQ ID NO: 438 |
| TPS30-likeJL | 6 | TPS30-likeJL-6F | TTAGTGCTATGGATGGGCTCC | SEQ ID NO: 439 |
| TPS30-likeJL | 6 | TPS30-likeJL-6R | CGATTTCTGAAGGACTTTTATTATATGG | SEQ ID NO: 440 |
| TPS30-likeJL | 7 | TPS30-likeJL-7F | AGAGAAGCAAGATGGTATTATGATGG | SEQ ID NO: 441 |
| TPS30-likeJL | 7 | TPS30-likeJL-7R | CGATGAATGTCGTATTATGGTAGGG | SEQ ID NO: 442 |
| TPS30-likeJL | 8a | TPS30-likeJL-8aF | TGGCGTGTCTGAAGAAGAAGC | SEQ ID NO: 443 |
| TPS30-likeJL | 8a | TPS30-likeJL-8aR | AACATTGGAGAGTAGTCATCACC | SEQ ID NO: 444 |
| TPS30-likeJL | 8b | TPS30-likeJL-8bF | CATGATGGTGATGACTACTCTCC | SEQ ID NO: 445 |
| TPS30-likeJL | 8b | TPS30-likeJL-8bR | AGTACATGATCTTTTGTTTGGCG | SEQ ID NO: 446 |
| TPS32JL | 1 | TPS32JL-1F | TGCAAAGAAGAACGAGTGAAGG | SEQ ID NO: 447 |
| TPS32JL | 1 | TPS32JL-1R | TGTTTGTAGTAGTTTAGATCATGTTTTTCC | SEQ ID NO: 448 |
| TPS32JL | 2 | TPS32JL-2F | ATAACCGATGTTTCGGGTTTGC | SEQ ID NO: 449 |
| TPS32JL | 2 | TPS32JL-2R | CCTCTCTATGGTCTTTCTTAATGGC | SEQ ID NO: 450 |
| TPS32JL | 3 | TPS32JL-3F | GAAAGCTAACAACCAAAATCTCTGC | SEQ ID NO: 451 |
| TPS32JL | 3 | TPS32JL-3R | TTGCATGGCTTGGGTAAGAAGC | SEQ ID NO: 452 |
| TPS32JL | 5 | TPS32JL-5F | AGAAGAAGCTCGATGGTTGAACG | SEQ ID NO: 453 |
| TPS32JL | 5 | TPS32JL-5R | CATGAACCTAGCAAGTAGAGTGG | SEQ ID NO: 454 |
| TPS32JL | 6 | TPS32JL-6F | GCCAACTGTTATGCCTTTCCC | SEQ ID NO: 455 |
| TPS32JL | 6 | TPS32JL-6R | GGGATTGGATCTATAAGTAAAGCAGC | SEQ ID NO: 456 |
| TPS33JL | 1 | TPS33JL-1F | ATGCTACCCCATCCAATGTGC | SEQ ID NO: 457 |
| TPS33JL | 1 | TPS33JL-1R | AATATAATCGAAAGACCAAATGGAGGGC | SEQ ID NO: 458 |
| TPS33JL | 2 | TPS33JL-2F | ATTAGTTGAGATGGAAAACTCTTTAGC | SEQ ID NO: 459 |
| TPS33JL | 2 | TPS33JL-2R | ATAGCCATGTTGACGTAGAAGCC | SEQ ID NO: 460 |
| TPS33JL | 3 | TPS33JL-3F | AGCTCCCACTTCATCGGAGG | SEQ ID NO: 461 |
| TPS33JL | 3 | TPS33JL-3R | TTTGGCTAACTCAAGCAACATAGG | SEQ ID NO: 462 |
| TPS33JL | 4a | TPS33JL-4aF | AGACATACTAAACTTGGAGAGAAATTGA | SEQ ID NO: 463 |
| TPS33JL | 4a | TPS33JL-4aR | TATTCCATAAGAAACATTCCATCAATCG | SEQ ID NO: 464 |
| TPS33JL | 4b | TPS33JL-4bF | GGAGACATACTAAACTTGGAGAGA | SEQ ID NO: 465 |
| TPS33JL | 4b | TPS33JL-4bR | GCTTTGGTGAAAAGCTCTAATTCAT | SEQ ID NO: 466 |
| TPS33JL | 5 | TPS33JL-5F | AGTTACCAGAATACATGAAGATGCC | SEQ ID NO: 467 |
| TPS33JL | 5 | TPS33JL-5R | TGAATGTTGATGGAGATTTCTTGG | SEQ ID NO: 468 |
| TPS33JL | 6 | TPS33JL-6F | AAATGGTTGGATTTCAGTAGGAGC | SEQ ID NO: 469 |
| TPS33JL | 6 | TPS33JL-6R | GAAAATCTCTTTAGTATTTGTAATTGTGCC | SEQ ID NO: 470 |
| TPS33JL | 7 | TPS33JL-7F | AATTGAAAAGAGGTGATGCTCCG | SEQ ID NO: 471 |
| TPS33JL | 7 | TPS33JL-7R | TTTGGATTGATTATCTTGAGAACTATGACC | SEQ ID NO: 472 |
| TPS36JL | 1 | TPS36JL-1F | AAAGATCAACCAGCAGCAATCG | SEQ ID NO: 473 |
| TPS36JL | 1 | TPS36JL-1R | GTGGGTTTGTAGTTTCCTGATCG | SEQ ID NO: 474 |
| TPS36JL | 2 | TPS36JL-2F | AAGGAGAGTGAAAATCCTTTAGTTAAGC | SEQ ID NO: 475 |
| TPS36JL | 2 | TPS36JL-2R | GAGTTTGAAATGAAGAGCAGTGGC | SEQ ID NO: 476 |
| TPS36JL | 3 | TPS36JL-3F | AATGCCTTCAAAAACGAGCAAAAGG | SEQ ID NO: 477 |
| TPS36JL | 3 | TPS36JL-3R | GTCATCAAGTATTGTTTGAGATGTTTGG | SEQ ID NO: 478 |
| TPS36JL | 4 | TPS36JL-4F | TGGCATTGAAATATGAGGCGG | SEQ ID NO: 479 |
| TPS36JL | 4 | TPS36JL-4R | GCTGAAGCTCATCTAGTGTACC | SEQ ID NO: 480 |
| TPS36JL | 5 | TPS36JL-5F | ATAAATGAACTGGATCAGCTACCCG | SEQ ID NO: 481 |
| TPS36JL | 5 | TPS36JL-5R | GATGGTGTGAATCCCATTTTCTTTGAGG | SEQ ID NO: 482 |
| TPS36JL | 6 | TPS36JL-6F | TTGGGGGATCTGTGTAAATGC | SEQ ID NO: 483 |
| TPS36JL | 6 | TPS36JL-6R | CAGTTCCTGAGACACGTAAAATGG | SEQ ID NO: 484 |
| TPS36JL | 7 | TPS36JL-7F | TGCTACATGC GTGAAAAGGG | SEQ ID NO:1398 |
| TPS36JL | 7 | TPS36JL-7R | TGTGATACAT CTCCATTGCTCC | SEQ ID NO: 1399 |
| TPS37FN | 1 | TPS37FN-1F | GCAGTGCATGGCTTTTCACC | SEQ ID NO: 485 |
| TPS37FN | 1 | TPS37FN-1R | TCCAAATAGGGAGGGATGATGA | SEQ ID NO: 486 |
| TPS37FN | 2 | TPS37FN-2F | ACAATGTACTGTGGTCGATAACCC | SEQ ID NO: 487 |
| TPS37FN | 2 | TPS37FN-2R | AATGAAATCAAAAGACCAAATGGGAGG | SEQ ID NO: 488 |
| TPS37FN | 3 | TPS37FN-3F | GAGAAAGACGTGAAAAGGATACTGG | SEQ ID NO: 489 |
| TPS37FN | 3 | TPS37FN-3R | TCAAATCCATATTGGCGTAGAAGC | SEQ ID NO: 490 |
| TPS37FN | 4 | TPS37FN-4F | AGCTTCATTCTATGGGAAAAAGGG | SEQ ID NO: 491 |
| TPS37FN | 4 | TPS37FN-4R | TGAACCACCTAGCTTCCAACC | SEQ ID NO: 492 |
| TPS37FN | 5 | TPS37FN-5F | GCAATCTAAACTTGGAGAAAAGAAAATGG | SEQ ID NO: 493 |
| TPS37FN | 5 | TPS37FN-5R | TTTGTGAAAAGCTCTAATTCCTCC | SEQ ID NO: 494 |
| TPS37FN | 7 | TPS37FN-7F | CATGCTTATTTTGCTTTCACAAATCCC | SEQ ID NO: 495 |
| TPS37FN | 7 | TPS37FN-7R | TAGGTCATCTTCAAGTCGTAAAAGTATGG | SEQ ID NO: 496 |
| TPS37FN | 8a | TPS37FN-8aF | GAGATGAAAAGAGGAGATGTTCCG | SEQ ID NO: 497 |
| TPS37FN | 8a | TPS37FN-8aR | AAGCATGTCCATCACCATAAAGG | SEQ ID NO: 498 |
| TPS37FN | 8b | TPS37FN-8bF | AAACATGGAAAGAGATGAATAAAGAAATGG | SEQ ID NO: 499 |
| TPS37FN | 8b | TPS37FN-8bR | GAGAAGCATGTCCATCACCATAAAGG | SEQ ID NO: 500 |
| TPS37JL | 1 | TPS37JL-1F | ATTCAAAGAACAACCGGAAGC | SEQ ID NO: 501 |
| TPS37JL | 1 | TPS37JL-1R | AAAAATGAGCATCCCACAACG | SEQ ID NO: 502 |
| TPS37JL | 2 | TPS37JL-2F | AAAGCTATGGGAAAAGAATCAATGAGC | SEQ ID NO: 503 |
| TPS37JL | 2 | TPS37JL-2R | GGAGTTTGAAATGAAGAGCAGTGG | SEQ ID NO: 504 |
| TPS37JL | 3 | TPS37JL-3F | GAGGCTTCATTCTATTCATTTAGGGG | SEQ ID NO: 505 |
| TPS37JL | 3 | TPS37JL-3R | TCAACAGATTTAGTTTGACATTGCC | SEQ ID NO: 506 |
| TPS37JL | 4 | TPS37JL-4F | TCTAGGTTGACAGAAAGGCTACC | SEQ ID NO: 507 |
| TPS37JL | 4 | TPS37JL-4R | GTCCATGAGTGTTAGCAAGAGACC | SEQ ID NO: 508 |
| TPS37JL | 5 | TPS37JL-5F | TATAAATGAATTGGATCAGCTACCCG | SEQ ID NO: 509 |
| TPS37JL | 5 | TPS37JL-5R | TATAAAAAGCAACAAACAATATCTTCATGT | SEQ ID NO: 510 |
| TPS37JL | 6a | TPS37JL-6aF | GTTGGGGGATCTGTGTAAATGC | SEQ ID NO: 511 |
| TPS37JL | 6a | TPS37JL-6aR | AGTTGGATATTGAAGCAAGTGTTCC | SEQ ID NO: 512 |
| TPS37JL | 6b | TPS37JL-6bF | TGTGTAAATGCTATATGGAGGAGGC | SEQ ID NO: 513 |
| TPS37JL | 6b | TPS37JL-6bR | TCGAAAGACAGTTCCTGAGACACG | SEQ ID NO: 514 |
| TPS37JL | 7 | TPS37JL-7F | TGCTACATGCGTGAAAAGGG | SEQ ID NO: 515 |
| TPS37JL | 7 | TPS37JL-7R | AGTACCAAAGCCATCACCC | SEQ ID NO: 516 |
| TPS38FN | 1 | TPS38FN-1F | TTAATATCATCATCACTACCTTGCATT | SEQ ID NO: 517 |
| TPS38FN | 1 | TPS38FN-1R | GGACTAATAGATGTTTTTGGTGTGAA | SEQ ID NO: 518 |
| TPS38FN | 2 | TPS38FN-2F | CAATGTACTGTGGTCAATAATAGTAGCC | SEQ ID NO: 519 |
| TPS38FN | 2 | TPS38FN-2R | AATCGAAAGACCAAATCGGAGG | SEQ ID NO: 520 |
| TPS38FN | 3 | TPS38FN-3F | ATAAGGGAGAATCCTATACAAGGC | SEQ ID NO: 521 |
| TPS38FN | 3 | TPS38FN-3R | CCTAAATTCGAGAGCAATGGGG | SEQ ID NO: 522 |
| TPS38FN | 4 | TPS38FN-4F | AGCATATTGGAGGAAGCTAGGG | SEQ ID NO: 523 |
| TPS38FN | 4 | TPS38FN-4R | TCGATAAACCACTTGGCCTCC | SEQ ID NO: 524 |
| TPS38FN | 5 | TPS38FN-5F | GATCTAAAACATTTGTCTAGGTGGTGG | SEQ ID NO: 525 |
| TPS38FN | 5 | TPS38FN-5R | ATTCTCCTAAAGTAGCTGAAATCTGG | SEQ ID NO: 526 |
| TPS38FN | 6 | TPS38FN-6F | TCGTGTTGTTGAGAGATGGG | SEQ ID NO: 527 |
| TPS38FN | 6 | TPS38FN-6R | AAGTGTTTTTCTTTTAATACATCTAGTGCC | SEQ ID NO: 528 |
| TPS38FN | 7 | TPS38FN-7F | TGGTTGGATTTCAGTAGGAGC | SEQ ID NO: 529 |
| TPS38FN | 7 | TPS38FN-7R | ACGATCATGGCAGATTGACG | SEQ ID NO: 530 |
| TPS38FN | 8 | TPS38FN-8F | CATAACGTATCTAAAGAGGAAGCTCG | SEQ ID NO: 531 |
| TPS38FN | 8 | TPS38FN-8R | TGCAAAGTTTTTGGCATCATCAACC | SEQ ID NO: 532 |
| TPS38JL | 1 | TPS38JL-1F | AAAGATCAACCAGCAGCAATCG | SEQ ID NO: 533 |
| TPS38JL | 1 | TPS38JL-1R | GTGGGTTTGTAGTTTCCTGATCG | SEQ ID NO: 534 |
| TPS38JL | 2a | TPS38JL-2aF | AAGCTATGGGAAAAGAATCAATGAGC | SEQ ID NO: 535 |
| TPS38JL | 2a | TPS38JL-2aR | GAGTTTGAAATGAAGAGCAGTGG | SEQ ID NO: 536 |
| TPS38JL | 2b | TPS38JL-2bF | AGGCTGAAAATCCTTTAGTTAAGC | SEQ ID NO: 537 |
| TPS38JL | 2b | TPS38JL-2bR | GGACTGAATCCATATTGTCGAAGG | SEQ ID NO: 538 |
| TPS38JL | 3 | TPS38JL-3F | TGCCTTCAAAAACGAGAAAAAGG | SEQ ID NO: 539 |
| TPS38JL | 3 | TPS38JL-3R | AGTCTCTTGCTTCATCTAATATGGG | SEQ ID NO: 540 |
| TPS38JL | 4 | TPS38JL-4F | TGCAGTGGCATTGAAATATGAGG | SEQ ID NO: 541 |
| TPS38JL | 4 | TPS38JL-4R | GCTGAAGCTCATCTAGTGTACC | SEQ ID NO: 542 |
| TPS38JL | 5 | TPS38JL-5F | ATAAATGAACTGGATCAGCTACCCG | SEQ ID NO: 543 |
| TPS38JL | 5 | TPS38JL-5R | TATAAAAAGCAACAAACAATATCTTCATGT | SEQ ID NO: 544 |
| TPS38JL | 6 | TPS38JL-6F | TGCTATATGGAGGAGGCAAAATGG | SEQ ID NO: 545 |
| TPS38JL | 6 | TPS38JL-6R | TCGAAAAACAGTTCCTGAGACACG | SEQ ID NO: 546 |
| TPS38JL | 7 | TPS38JL-7F | TGCTACATGCGTGAAAAGGG | SEQ ID NO: 547 |
| TPS38JL | 7 | TPS38JL-7R | TGTGGTACATCTCCATTGCTCC | SEQ ID NO: 548 |
| TPS39JL | 1 | TPS39JL-1F | TGGCTTTTCACCAATTTGCTCC | SEQ ID NO: 549 |
| TPS39JL | 1 | TPS39JL-1R | AAATGGGAGGTCCATAGTTGGC | SEQ ID NO: 550 |
| TPS39JL | 2 | TPS39JL-2F | TTGAAGAAAGAAGTGACAAGATGGC | SEQ ID NO: 551 |
| TPS39JL | 2 | TPS39JL-2R | GGCGTAGAAGCCTAAATTCGC | SEQ ID NO: 552 |
| TPS39JL | 3 | TPS39JL-3F | ACATGGAGAAAATGGAGAATGAGG | SEQ ID NO: 553 |
| TPS39JL | 3 | TPS39JL-3R | CGCAACCTCGAAACAAGTCG | SEQ ID NO: 554 |
| TPS39JL | 4 | TPS39JL-4F | AGGGGATTGTAAACTTGGTGG | SEQ ID NO: 555 |
| TPS39JL | 4 | TPS39JL-4R | TTCTCTAAAATAGCTGAAATTCTCCTCG | SEQ ID NO: 556 |
| TPS39JL | 5 | TPS39JL-5F | TGAGTTACCAGATTACATGAAGATGCC | SEQ ID NO: 557 |
| TPS39JL | 5 | TPS39JL-5R | ACTAATACATCGAACCCCATCTCA | SEQ ID NO: 558 |
| TPS39JL | 6 | TPS39JL-6F | ACAAGAAGCAAAATGGTATTATAGTGG | SEQ ID NO: 559 |
| TPS39JL | 6 | TPS39JL-6R | TTTTGTTATAGGATTTGTGAAACAATAAGC | SEQ ID NO: 560 |
| TPS39JL | 7 | TPS39JL-7F | ATAATGCTACCGAAGACGAAGC | SEQ ID NO: 561 |
| TPS39JL | 7 | TPS39JL-7R | TCTGAGAACCATGTCCATCTCC | SEQ ID NO: 562 |
| TPS3JL | 1 | TPS3JL-1F | AATCACTTTTGTAGATTTTTCACACC | SEQ ID NO: 563 |
| TPS3JL | 1 | TPS3JL-1R | GTAATCGAAAGACCAAATCGGAGG | SEQ ID NO: 564 |
| TPS3JL | 2 | TPS3JL-2F | AGAAAGAAGTGACAAGAATGCTCC | SEQ ID NO: 565 |
| TPS3JL | 2 | TPS3JL-2R | AGCCATGTTGGCGTAGAAGC | SEQ ID NO: 566 |
| TPS3JL | 3a | TPS3JL-3aF | TGCTTTCAAGGATAAGAGAGGG | SEQ ID NO: 567 |
| TPS3JL | 3a | TPS3JL-3aR | TCTTCCTCATTCTCCATTTTCTCC | SEQ ID NO: 568 |
| TPS3JL | 3b | TPS3JL-3bF | AATACATGGAGAAAATGGAGAATGAGG | SEQ ID NO: 569 |
| TPS3JL | 3b | TPS3JL-3bR | CGCAAACTCGAACAAAGTCG | SEQ ID NO: 570 |
| TPS3JL | 4 | TPS3JL-4F | TTTGCTAGAGATAGATTGATGGAAGC | SEQ ID NO: 571 |
| TPS3JL | 4 | TPS3JL-4R | AAAAGCTCTAATTCCTCCAAAGTTCC | SEQ ID NO: 572 |
| TPS3JL | 5 | TPS3JL-5F | ATCAATGAGTTACCAGATTACATGAAG | SEQ ID NO: 573 |
| TPS3JL | 5 | TPS3JL-5R | ACTAATACATCGAACCCCATCTCAT | SEQ ID NO: 574 |
| TPS3JL | 6 | TPS3JL-6F | ATACACTGAGTTGGGTTGGC | SEQ ID NO: 575 |
| TPS3JL | 6 | TPS3JL-6R | TGTTCCTAAATCATCTGCAAGCC | SEQ ID NO: 576 |
| TPS3JL | 7 | TPS3JL-7F | GAATTGAATAGAGGCGACGTTCC | SEQ ID NO: 577 |
| TPS3JL | 7 | TPS3JL-7R | ATAGTGCTGTTCTAGCCATATTTTTGC | SEQ ID NO: 578 |
| TPS40JL | 1a | TPS40JL-1aF | CAATGTACTGTGGTCAATAATAGTAGCC | SEQ ID NO: 579 |
| TPS40JL | 1a | TPS40JL-1aR | AATCGAAAGACCAAATCGGAGG | SEQ ID NO: 580 |
| TPS40JL | 1b | TPS40JL-1bF | ACTGTGGTCAATAATAGTAGCCC | SEQ ID NO: 581 |
| TPS40JL | 1b | TPS40JL-1bR | TGAATGTAATCGAAAGACCAAATCGG | SEQ ID NO: 582 |
| TPS40JL | 2 | TPS40JL-2F | AATATAAGGGAGAATCCTATACAAGGC | SEQ ID NO: 583 |
| TPS40JL | 2 | TPS40JL-2R | ATAGCCATGTTGGCGTAGAAGC | SEQ ID NO: 584 |
| TPS40JL | 3a | TPS40JL-3aF | TGAGAAAAATGGTGAAAGCATATTGG | SEQ ID NO: 585 |
| TPS40JL | 3a | TPS40JL-3aR | TCCGTTCTTGCAGTCCTCC | SEQ ID NO: 586 |
| TPS40JL | 3b | TPS40JL-3bF | GAGGACTGCAAGAACGGAGG | SEQ ID NO: 587 |
| TPS40JL | 3b | TPS40JL-3bR | ACAAATGTTTTAGATCGTCTTGATGC | SEQ ID NO: 588 |
| TPS40JL | 4 | TPS40JL-4F | TTTGTGAGAGATAGGTTGATGGGG | SEQ ID NO: 589 |
| TPS40JL | 4 | TPS40JL-4R | AAAGCTCTAATTCTTCTAATGTTCCG | SEQ ID NO: 590 |
| TPS40JL | 6 | TPS40JL-6F | ATGGTTGGATTTCAGTAGGAGC | SEQ ID NO: 591 |
| TPS40JL | 6 | TPS40JL-6R | GTACGATCATGGCAGATTGACG | SEQ ID NO: 592 |
| TPS40JL | 7 | TPS40JL-7F | ATGCAAGACCATAATGTATCTAAAGAGG | SEQ ID NO: 593 |
| TPS40JL | 7 | TPS40JL-7R | TGCAAAGTTTTTGGCATCATCAACC | SEQ ID NO: 594 |
| TPS41JL | 1 | TPS41JL-1F | TCAATGTACTGTAGTCGATAGTTCTAATCC | SEQ ID NO: 595 |
| TPS41JL | 1 | TPS41JL-1R | GACCAAATGGATGGTTCATAGTTGC | SEQ ID NO: 596 |
| TPS41JL | 2a | TPS41JL-2aF | TAGAAGTCGGGTGAAAGAGATCG | SEQ ID NO: 597 |
| TPS41JL | 2a | TPS41JL-2aR | AGAAGTCTAAATTCAAGAGCAATGG | SEQ ID NO: 598 |
| TPS41JL | 2b | TPS41JL-2bF | ATAGAAGTCGGGTGAAAGAGATCG | SEQ ID NO: 599 |
| TPS41JL | 2b | TPS41JL-2bR | AAGCGATAAGATACTCCAAGTCTTTGC | SEQ ID NO: 600 |
| TPS41JL | 3 | TPS41JL-3F | ACGAGACAGGAAAATTCAAGGC | SEQ ID NO: 601 |
| TPS41JL | 3 | TPS41JL-3R | TCCTCCAATGAAGTGGGAGC | SEQ ID NO: 602 |
| TPS41JL | 4 | TPS41JL-4F | GTGGAGGCATACTAAACTTGGG | SEQ ID NO: 603 |
| TPS41JL | 4 | TPS41JL-4R | AAAAGCTCTAATTCATCCAATGTTCC | SEQ ID NO: 604 |
| TPS41JL | 5 | TPS41JL-5F | ACCAGAATACATGAAGATGCCT | SEQ ID NO: 605 |
| TPS41JL | 5 | TPS41JL-5R | TGTTGATGGAGATGTCTTTGTCTCT | SEQ ID NO: 606 |
| TPS41JL | 6 | TPS41JL-6F | GTGGATAGATATGTGTAGAGGTTTTCT | SEQ ID NO: 607 |
| TPS41JL | 6 | TPS41JL-6R | AAGAACTGGTGCTCCCACTG | SEQ ID NO: 608 |
| TPS41JL | 7 | TPS41JL-7F | TGTTCGGATGAAATGAAAAGAGGC | SEQ ID NO: 609 |
| TPS41JL | 7 | TPS41JL-7R | TGTGACGCTCTACCAAGATTTTTGC | SEQ ID NO: 610 |
| TPS42JL | 1 | TPS42JL-1F | CAAATGTTCTGTGGTCCATAACCC | SEQ ID NO: 611 |
| TPS42JL | 1 | TPS42JL-1R | GACCAAATGGGAGGTTCATAGTTTCC | SEQ ID NO: 612 |
| TPS42JL | 2 | TPS42JL-2F | GAGAAAGATGTGAAAATGATGCTACTTGG | SEQ ID NO: 613 |
| TPS42JL | 2 | TPS42JL-2R | TGAGGTACTACAAATCCATACTCCC | SEQ ID NO: 614 |
| TPS42JL | 3 | TPS42JL-3F | GATGATGAAACAGGAGAGTTCAAGG | SEQ ID NO: 615 |
| TPS42JL | 3 | TPS42JL-3R | GACATTTGGTTGTGAAAATTCTTGC | SEQ ID NO: 616 |
| TPS42JL | 4 | TPS42JL-4F | TGGATCATGCTTTGGAAATGCC | SEQ ID NO: 617 |
| TPS42JL | 4 | TPS42JL-4R | AGCTCTTCTTGATATGTTGATTGC | SEQ ID NO: 618 |
| TPS42JL | 5a | TPS42JL-5aF | TGGTGGAAGCATTCTAAACTTGG | SEQ ID NO: 619 |
| TPS42JL | 5a | TPS42JL-5aR | AGCATTAGCGAAGAGTTGTAGTTCC | SEQ ID NO: 620 |
| TPS42JL | 5b | TPS42JL-5bF | AGTGTTTCATGTGGCAAGTTGG | SEQ ID NO: 621 |
| TPS42JL | 5b | TPS42JL-5bR | AGCGAAGAGTTGTAGTTCCTCC | SEQ ID NO: 622 |
| TPS42JL | 6 | TPS42JL-6F | GGGATTTGAAAGTAATAGATGAGTTACCG | SEQ ID NO: 623 |
| TPS42JL | 6 | TPS42JL-6R | AGAAAGCCATCTTCATGTAATCCG | SEQ ID NO: 624 |
| TPS42JL | 7 | TPS42JL-7F | AGTGGATACCAACCAACATTGC | SEQ ID NO: 625 |
| TPS42JL | 7 | TPS42JL-7R | TGTTATGAGATTTGTAAAACAGAAATAAGC | SEQ ID NO: 626 |
| TPS42JL | 8a | TPS42JL-8aF | GATGAAATGAAAAGAGGCGATGTTCC | SEQ ID NO: 627 |
| TPS42JL | 8a | TPS42JL-8aR | TCAAATGCTTGATGTGCTCACG | SEQ ID NO: 628 |
| TPS42JL | 8b | TPS42JL-8bF | TGGAAGGAGATGAATAATGAAAATGG | SEQ ID NO: 629 |
| TPS42JL | 8b | TPS42JL-8bR | GATCTTTTGATAGAGTATTTTGAGAAGC | SEQ ID NO: 630 |
| TPS43JL | 1 | TPS43JL-1F | TCACTTAGAACCACAAAAGACCC | SEQ ID NO: 631 |
| TPS43JL | 1 | TPS43JL-1R | GCCAACAAATAAGCCATGTTGC | SEQ ID NO: 632 |
| TPS43JL | 2a | TPS43JL-2aF | ACAGATTTAGAGGCAGAGATGGG | SEQ ID NO: 633 |
| TPS43JL | 2a | TPS43JL-2aR | GTGGTGAAACTCTTGGCTTCC | SEQ ID NO: 634 |
| TPS43JL | 2b | TPS43JL-2bF | AGCTTCACACCTTGGAATGG | SEQ ID NO: 635 |
| TPS43JL | 2b | TPS43JL-2bR | ATCCAACTTAGCCAACTCAAGC | SEQ ID NO: 636 |
| TPS43JL | 3 | TPS43JL-3F | TGCAAGAGATCGTGTGGTGG | SEQ ID NO: 637 |
| TPS43JL | 3 | TPS43JL-3R | ACTGCATTTGTGAAAAGTTCAAGC | SEQ ID NO: 638 |
| TPS43JL | 4 | TPS43JL-4F | TGGGACATAAGGGCAATAAGGG | SEQ ID NO: 639 |
| TPS43JL | 4 | TPS43JL-4R | CGATCACTTCATTACCAAAATTAAGCA | SEQ ID NO: 640 |
| TPS43JL | 5 | TPS43JL-5F | TCAGTTGGTGGTCATGCAGC | SEQ ID NO: 641 |
| TPS43JL | 5 | TPS43JL-5R | TGAAGTTCCTAAATCATCACTAAGCC | SEQ ID NO: 642 |
| TPS43JL | 6 | TPS43JL-6F | TCAGTGGAGTGCTACATGGC | SEQ ID NO: 643 |
| TPS43JL | 6 | TPS43JL-6R | TCACCATTATTTTTGGCAGGC | SEQ ID NO: 644 |
| TPS44JL | 1a | TPS44JL-1aF | ACAAAGAGAGTTATGTGAAGATTATTGAGC | SEQ ID NO: 645 |
| TPS44JL | 1a | TPS44JL-1aR | TCAAGTGTCTCTAAAGGGTTTTCC | SEQ ID NO: 646 |
| TPS44JL | 1b | TPS44JL-1bF | ACTTCATGGTCTTCATCCTTTGG | SEQ ID NO: 647 |
| TPS44JL | 1b | TPS44JL-1bR | TTCCCTAAGCAACCGAAAGC | SEQ ID NO: 648 |
| TPS44JL | 2 | TPS44JL-2F | GCCCTAATGCACCCTATTCG | SEQ ID NO: 649 |
| TPS44JL | 2 | TPS44JL-2R | TAATTCTTTTTGATGTAGTTGTTGAAGC | SEQ ID NO: 650 |
| TPS44JL | 3 | TPS44JL-3F | TGATGACCAAGTTGATTTCTTTGC | SEQ ID NO: 651 |
| TPS44JL | 3 | TPS44JL-3R | TTTCTAACTGCTTCAGTAAAAGGC | SEQ ID NO: 652 |
| TPS44JL | 4 | TPS44JL-4F | ACTAGAGTACATGAAAGTATGTTACAAGA | SEQ ID NO: 653 |
| TPS44JL | 4 | TPS44JL-4R | ACATAGCTCGCACAGTATGGA | SEQ ID NO: 654 |
| TPS44JL | 5 | TPS44JL-5F | AGGAAGCCCAATGGTTACACA | SEQ ID NO: 655 |
| TPS44JL | 5 | TPS44JL-5R | AGTTCGTATAATCTTAGGTTGGGGT | SEQ ID NO: 656 |
| TPS44JL | 6 | TPS44JL-6F | GTAGTGGCTTCCGCTGTGG | SEQ ID NO: 657 |
| TPS44JL | 6 | TPS44JL-6R | TCGCCTTCTCTATACATCTCATGG | SEQ ID NO: 658 |
| TPS45JL | 1 | TPS45JL-1F | TGGTCTTCATCCTTTGGAAAACCC | SEQ ID NO: 659 |
| TPS45JL | 1 | TPS45JL-1R | TTCCCTAAGCAACCGAAAGCG | SEQ ID NO: 660 |
| TPS45JL | 2 | TPS45JL-2F | GTGAAACATGCCCTAATGCACC | SEQ ID NO: 661 |
| TPS45JL | 2 | TPS45JL-2R | ATCTAGTTTGGCAGTTAAAAGAAGC | SEQ ID NO: 662 |
| TPS45JL | 3 | TPS45JL-3F | ACAGAATAGTGGAGTGTTACATTTGG | SEQ ID NO: 663 |
| TPS45JL | 3 | TPS45JL-3R | ATAATAGTAAGCAAAGAAATCAACTTGG | SEQ ID NO: 664 |
| TPS45JL | 5 | TPS45JL-5F | TTCAAAACTCCCAAAAAGCGAAA | SEQ ID NO: 665 |
| TPS45JL | 5 | TPS45JL-5R | TGTCACGTCAGCATACACTCC | SEQ ID NO: 666 |
| TPS45JL | 6a | TPS45JL-6aF | TCAAAAGATGTAGTGGCTTCTGC | SEQ ID NO: 667 |
| TPS45JL | 6a | TPS45JL-6aR | ATTAAGAGTGGTCTAGGAATAGCG | SEQ ID NO: 668 |
| TPS45JL | 6b | TPS45JL-6bF | CAATATGGTGTAACAGATGAAGAAGC | SEQ ID NO: 669 |
| TPS45JL | 6b | TPS45JL-6bR | TGAGAGTATCAATCAAATTCTTGAGC | SEQ ID NO: 670 |
| TPS46JL | 1 | TPS46JL-1F | CATCGAGACAAACTGCGAATACT | SEQ ID NO: 671 |
| TPS46JL | 1 | TPS46JL-1R | ATATGTGATCTCCATTTTCTTCCATTG | SEQ ID NO: 672 |
| TPS46JL | 2 | TPS46JL-2F | CATGGATGGCCCGATTGGA | SEQ ID NO: 673 |
| TPS46JL | 2 | TPS46JL-2R | TCGAAAATGATTCTTTCCAAGCCAT | SEQ ID NO: 674 |
| TPS46JL | 4 | TPS46JL-4F | GATTGGGGACTTAGTGAAATGGG | SEQ ID NO: 675 |
| TPS46JL | 4 | TPS46JL-4R | GTAAAGTTTTCTAACTCGGTGATTAAGG | SEQ ID NO: 676 |
| TPS46JL | 5 | TPS46JL-5F | GGTGGGATGGTGAAGGATTGA | SEQ ID NO: 677 |
| TPS46JL | 5 | TPS46JL-5R | TGATGTCACTTGTTTCTTGTTGAT | SEQ ID NO: 678 |
| TPS46JL | 6 | TPS46JL-6F | TCACACCTTGCTTCTTCCAGC | SEQ ID NO: 679 |
| TPS46JL | 6 | TPS46JL-6R | TCATTCAACAAACGACAAATAATCATAACC | SEQ ID NO: 680 |
| TPS46JL | 7 | TPS46JL-7F | ACTTGAAGAACAATCCCGAGG | SEQ ID NO: 681 |
| TPS46JL | 7 | TPS46JL-7R | AACCCTAACATCATCATCGTCG | SEQ ID NO: 682 |
| TPS47JL | 10a | TPS47JL-10aF | CAGACACTGAAATGCTTGACG | SEQ ID NO: 683 |
| TPS47JL | 10a | TPS47JL-10aR | TAGCGGTTAGACATTTAGAGGG | SEQ ID NO: 684 |
| TPS47JL | 10b | TPS47JL-10bF | CAACCCTCTAAATGTCTAACCGC | SEQ ID NO: 685 |
| TPS47JL | 10b | TPS47JL-10bR | AAATTGAGTTTTGAAGGCATAGTAGG | SEQ ID NO: 686 |
| TPS47JL | 1a | TPS47JL-1aF | ATCTTTGCCCCAAACTCAGG | SEQ ID NO: 687 |
| TPS47JL | 1a | TPS47JL-1aR | GGTTAGGATGTGGTATCATGGC | SEQ ID NO: 688 |
| TPS47JL | 1b | TPS47JL-1bF | TACCACATCCTAACCAACCTTCG | SEQ ID NO: 689 |
| TPS47JL | 1b | TPS47JL-1bR | CAACAACACAAGCCAGAGTGG | SEQ ID NO: 690 |
| TPS47JL | 2 | TPS47JL-2F | TATTCATTCATCTAATGCAAAAAGGC | SEQ ID NO: 691 |
| TPS47JL | 2 | TPS47JL-2R | AATATGTCCGAAACGAAAACGG | SEQ ID NO: 692 |
| TPS47JL | 3a | TPS47JL-3aF | TGTTATCATATCTTGAAGTGTTGCC | SEQ ID NO: 693 |
| TPS47JL | 3a | TPS47JL-3aR | CTGTTGCCGATGGAGATTGG | SEQ ID NO: 694 |
| TPS47JL | 3b | TPS47JL-3bF | CAATCTCCATCGGCAACAGC | SEQ ID NO: 695 |
| TPS47JL | 3b | TPS47JL-3bR | TGTTATTATTGGAAAACTTGTGAACTAGG | SEQ ID NO: 696 |
| TPS47JL | 4 | TPS47JL-4F | AGGGTTAGCTGAGCATTTCGC | SEQ ID NO: 697 |
| TPS47JL | 4 | TPS47JL-4R | CTAGGAAAAACTTTGTAGCCATGC | SEQ ID NO: 698 |
| TPS47JL | 5a | TPS47JL-5aF | ACTATGAATGCTTTTCGGTTACG | SEQ ID NO: 699 |
| TPS47JL | 5a | TPS47JL-5aR | AAAATAGACTTTTGAAGTAGTTTTCTCG | SEQ ID NO: 700 |
| TPS47JL | 5b | TPS47JL-5bF | TTGCTTTTCATGGTGAATTTGAGC | SEQ ID NO: 701 |
| TPS47JL | 5b | TPS47JL-5bR | TTTGTTAAAAGGATTTGTATGTGGAGC | SEQ ID NO: 702 |
| TPS47JL | 6 | TPS47JL-6F | TGGCTCGACTAGATCACTTGG | SEQ ID NO: 703 |
| TPS47JL | 6 | TPS47JL-6R | GTGAAACCTCGACAACCTTTGG | SEQ ID NO: 704 |
| TPS47JL | 7 | TPS47JL-7F | GGGGACTTAATGAAATGGGATTTGG | SEQ ID NO: 705 |
| TPS47JL | 7 | TPS47JL-7R | TCATAAGGCAAAGAACAACAAGC | SEQ ID NO: 706 |
| TPS47JL | 9 | TPS47JL-9F | CCATAGCAACTCACACCTTGC | SEQ ID NO: 707 |
| TPS47JL | 9 | TPS47JL-9R | ACACTCTCACATTGAATTGGTCG | SEQ ID NO: 708 |
| TPS48JL | 1 | TPS48JL-1F | CTTGCTTTTCATGGTGAATTTGAGC | SEQ ID NO: 709 |
| TPS48JL | 1 | TPS48JL-1R | CAGTTTCTTAAAGGGATTTGTATGTTGAGC | SEQ ID NO: 710 |
| TPS48JL | 2 | TPS48JL-2F | GATGGCTCGACTAGATCACC | SEQ ID NO: 711 |
| TPS48JL | 2 | TPS48JL-2R | AGATGTCTTTCCCATCCATAAAGC | SEQ ID NO: 712 |
| TPS48JL | 4 | TPS48JL-4F | AGAATGGGGACTTAGTGAAATGGG | SEQ ID NO: 713 |
| TPS48JL | 4 | TPS48JL-4R | TGGAGAATCATAAGGCAAATAACAGC | SEQ ID NO: 714 |
| TPS48JL | 5 | TPS48JL-5F | TGGAATAATGAAGGGTTGAGTGGT | SEQ ID NO: 715 |
| TPS48JL | 5 | TPS48JL-5R | TCTTATGTAGCTGGTTATGTCTTCA | SEQ ID NO: 716 |
| TPS48JL | 6a | TPS48JL-6aF | TGAATCTGAGTGGAGTAAAAGTGG | SEQ ID NO: 717 |
| TPS48JL | 6a | TPS48JL-6aR | AGAAGAAATGAAGCTGGAAGAAGC | SEQ ID NO: 718 |
| TPS48JL | 6b | TPS48JL-6bF | GCAACTCATACCTTGCTTCTTCC | SEQ ID NO: 719 |
| TPS48JL | 6b | TPS48JL-6bR | GAACTTTGTAAGTCATTCAACAAACG | SEQ ID NO: 720 |
| TPS48JL | 7a | TPS48JL-7aF | GAGGGAAAGAGAAGAAGGCAAACC | SEQ ID NO: 721 |
| TPS48JL | 7a | TPS48JL-7aR | ACTTTTAAGCAAGATAAGTGTAGAAGCC | SEQ ID NO: 722 |
| TPS48JL | 7b | TPS48JL-7bF | AATGACAATGGTTTTACCAATCTTCC | SEQ ID NO: 723 |
| TPS48JL | 7b | TPS48JL-7bR | AATCGCTTTGTTAATGTCTTCAAGC | SEQ ID NO: 724 |
| TPS49JL | 1 | TPS49JL-1F | TCGGCCAATTTTCATCCTAGT | SEQ ID NO: 725 |
| TPS49JL | 1 | TPS49JL-1R | GGTATATTTGAGAAAGTGATCTCCCC | SEQ ID NO: 726 |
| TPS49JL | 2 | TPS49JL-2F | GGATGCTAACTGCTGCCCC | SEQ ID NO: 727 |
| TPS49JL | 2 | TPS49JL-2R | AAAGGGAGACCGTATGAAGGG | SEQ ID NO: 728 |
| TPS49JL | 3a | TPS49JL-3aF | GAAATCCCAGTTGACCATGAGC | SEQ ID NO: 729 |
| TPS49JL | 3a | TPS49JL-3aR | GATGACTGTGGTTAGGGTCG | SEQ ID NO: 730 |
| TPS49JL | 3b | TPS49JL-3bF | CACAACTCATTTGGTGGAGGC | SEQ ID NO: 731 |
| TPS49JL | 3b | TPS49JL-3bR | AGATGACTGTGGTTAGGGTCG | SEQ ID NO: 732 |
| TPS49JL | 4a | TPS49JL-4aF | GGAAAGACTTGGACTTTGCATCG | SEQ ID NO: 733 |
| TPS49JL | 4a | TPS49JL-4aR | CTAGCAGCACTGTACTTTGGC | SEQ ID NO: 734 |
| TPS49JL | 4b | TPS49JL-4bF | GGAAAGACTTGGACTTTGCATCG | SEQ ID NO: 735 |
| TPS49JL | 4b | TPS49JL-4bR | TTGCGTCTGTGAAGAGTTCC | SEQ ID NO: 736 |
| TPS49JL | 5 | TPS49JL-5F | TATACGATGAAATTGAGGAGGAGTTGGC | SEQ ID NO: 737 |
| TPS49JL | 5 | TPS49JL-5R | AGGCCATCCGATAAGTTCTGCC | SEQ ID NO: 738 |
| TPS49JL | 6a | TPS49JL-6aF | TACTACGTGGAAGCTCAATGG | SEQ ID NO: 739 |
| TPS49JL | 6a | TPS49JL-6aR | AAGCCAATGAAAGACCTCAGC | SEQ ID NO: 740 |
| TPS49JL | 6b | TPS49JL-6bF | ACTACATGCTCACTGCCACG | SEQ ID NO: 741 |
| TPS49JL | 6b | TPS49JL-6bR | TCTGCAAACTACTGCTGATGC | SEQ ID NO: 742 |
| TPS49JL | 7a | TPS49JL-7aF | GTTTGAGCAAGATAGAGGACACG | SEQ ID NO: 743 |
| TPS49JL | 7a | TPS49JL-7aR | GCATGAGGACAGACATGGGC | SEQ ID NO: 744 |
| TPS49JL | 7b | TPS49JL-7bF | TGAATGAGGAGTGCATGGAGC | SEQ ID NO: 745 |
| TPS49JL | 7b | TPS49JL-7bR | TCCGGCATGGGTGTAACCG | SEQ ID NO: 746 |
| TPS4FN | 1 | TPS4FN-1F | AGCCTCATCACAAAATGACAAAGT | SEQ ID NO: 747 |
| TPS4FN | 1 | TPS4FN-1R | GGTTGATAAGTTGTTGTTGGACGA | SEQ ID NO: 748 |
| TPS4FN | 2 | TPS4FN-2F | AAGGAAGTGGTAAGGAGAGAGG | SEQ ID NO: 749 |
| TPS4FN | 2 | TPS4FN-2R | GAAACCATAAATCCATGTTGGCG | SEQ ID NO: 750 |
| TPS4FN | 3a | TPS4FN-3aF | AGCAAGGGAATTTTAAGGAGTGC | SEQ ID NO: 751 |
| TPS4FN | 3a | TPS4FN-3aR | AGGTGAGTGGTGGTGAAAGC | SEQ ID NO: 752 |
| TPS4FN | 3b | TPS4FN-3bF | TTGCTTTCACCACCACTCACC | SEQ ID NO: 753 |
| TPS4FN | 3b | TPS4FN-3bR | GAAATGTAATGCCTGGCGTGG | SEQ ID NO: 754 |
| TPS4FN | 4a | TPS4FN-4aF | TGCAAGAGATAGGATTGTGGAGC | SEQ ID NO: 755 |
| TPS4FN | 4a | TPS4FN-4aR | TCTGCAACTGAGGCCAATGC | SEQ ID NO: 756 |
| TPS4FN | 4b | TPS4FN-4bF | TGAACCTGAATTGTCACTGGC | SEQ ID NO: 757 |
| TPS4FN | 4b | TPS4FN-4bR | GCTCAAGCTCTTCAAATGTACC | SEQ ID NO: 758 |
| TPS4FN | 5 | TPS4FN-5F | AATTGTGCGGATCAACTTCG | SEQ ID NO: 759 |
| TPS4FN | 5 | TPS4FN-5R | TGTAACTTTCCTCCTTTCCAAGC | SEQ ID NO: 760 |
| TPS4FN | 6 | TPS4FN-6F | AGCGATGAAAAGATTACTTGGAGC | SEQ ID NO: 761 |
| TPS4FN | 6 | TPS4FN-6R | CATCCATGAACCTACAAAGGATATTGC | SEQ ID NO: 762 |
| TPS4FN | 7a | TPS4FN-7aF | AGAGATCATTCACCGTCTACCG | SEQ ID NO: 763 |
| TPS4FN | 7a | TPS4FN-7aR | AAACTGGATAAGGCACATTAGAAGGC | SEQ ID NO: 764 |
| TPS4FN | 7b | TPS4FN-7bF | GGCCTTCTAATGTGCCTTATCC | SEQ ID NO: 765 |
| TPS4FN | 7b | TPS4FN-7bR | TCATGGGATTTGATCTATAAGTAACGC | SEQ ID NO: 766 |
| TPS4JL | 1a | TPS4JL-1aF | AGTTTTAGCCTCATCACAAAATGACA | SEQ ID NO: 767 |
| TPS4JL | 1a | TPS4JL-1aR | GTTGATAAGTTGTTGTTGGACGAA | SEQ ID NO: 768 |
| TPS4JL | 1b | TPS4JL-1bF | TAGCCTCATCACAAAATGACAAAG | SEQ ID NO: 769 |
| TPS4JL | 1b | TPS4JL-1bR | CCCCCAAATAGAAGGTTGATAAGT | SEQ ID NO: 770 |
| TPS4JL | 2a | TPS4JL-2aF | TAAAAAGCAGCGAGTTGACG | SEQ ID NO: 771 |
| TPS4JL | 2a | TPS4JL-2aR | AATGATACGACAATCCCAAACG | SEQ ID NO: 772 |
| TPS4JL | 2b | TPS4JL-2bF | AAGGAAGTGGTAAGGAGAGAGG | SEQ ID NO: 773 |
| TPS4JL | 2b | TPS4JL-2bR | GAAACCATAAATCCATGTTGGCG | SEQ ID NO: 774 |
| TPS4JL | 3a | TPS4JL-3aF | AGCAAGGGAATTTTAAGGAGTGC | SEQ ID NO: 775 |
| TPS4JL | 3a | TPS4JL-3aR | AGGTGAGTGGTGGTGAAAGC | SEQ ID NO: 776 |
| TPS4JL | 3b | TPS4JL-3bF | CTTGCTTTCACCACCACTCACC | SEQ ID NO: 777 |
| TPS4JL | 3b | TPS4JL-3bR | GTAATGCCTGGCGTGGAGC | SEQ ID NO: 778 |
| TPS4JL | 4a | TPS4JL-4aF | GCAAGAGATAGGATTGTGGAGC | SEQ ID NO: 779 |
| TPS4JL | 4a | TPS4JL-4aR | ATCTGCAACTGAGGCCAAGG | SEQ ID NO: 780 |
| TPS4JL | 4b | TPS4JL-4bF | TGAACCTGAATTGTCACTGGC | SEQ ID NO: 781 |
| TPS4JL | 4b | TPS4JL-4bR | GCTCAAGCTCTTCAAATGTACC | SEQ ID NO: 782 |
| TPS4JL | 5 | TPS4JL-5F | AATTGTGCGGATCAACTTCG | SEQ ID NO: 783 |
| TPS4JL | 5 | TPS4JL-5R | TGTAACTTTCCTCCTTTCCAAGC | SEQ ID NO: 784 |
| TPS4JL | 6 | TPS4JL-6F | CAGTGAAGCTCGATGGTTGC | SEQ ID NO: 785 |
| TPS4JL | 6 | TPS4JL-6R | TCCATGAACCTACAAAGGATATTGC | SEQ ID NO: 786 |
| TPS4JL | 7a | TPS4JL-7aF | CCGTTGAGAGTTACATGAGGC | SEQ ID NO: 787 |
| TPS4JL | 7a | TPS4JL-7aR | ACTGGATAAGGCACATTAGAAGG | SEQ ID NO: 788 |
| TPS4JL | 7b | TPS4JL-7bF | ACAAGAGGCATGTGATGAGC | SEQ ID NO: 789 |
| TPS4JL | 7b | TPS4JL-7bR | GGGATTTGATCTATAAGTAACGCAGC | SEQ ID NO: 790 |
| TPS4-likeJL | 1 | TPS4-likeJL-1F | TGTCGTCTCAAATCTTAGCAACC | SEQ ID NO: 791 |
| TPS4-likeJL | 1 | TPS4-likeJL-1R | ATGCAAAAATCGGTCTCCCC | SEQ ID NO: 792 |
| TPS4-likeJL | 2a | TPS4-likeJL-2aF | TTCTTATGTGATGATTGGAGTAATCG | SEQ ID NO: 793 |
| TPS4-likeJL | 2a | TPS4-likeJL-2aR | GCTTATTTTGTTGTAAATGTGTTGAAGC | SEQ ID NO: 794 |
| TPS4-likeJL | 2b | TPS4-likeJL-2bF | CGATTGAAGTTAATTGATGTGGTGC | SEQ ID NO: 795 |
| TPS4-likeJL | 2b | TPS4-likeJL-2bR | AAAGAAACCCTATATCCATGTTGTCG | SEQ ID NO: 796 |
| TPS4-likeJL | 3a | TPS4-likeJL-3aF | GAATGTTTGGCGAGTGACACC | SEQ ID NO: 797 |
| TPS4-likeJL | 3a | TPS4-likeJL-3aR | AAGGGCCTCTCTAGGGCTCG | SEQ ID NO: 798 |
| TPS4-likeJL | 3b | TPS4-likeJL-3bF | AAGAACACCCCAATGATGATCC | SEQ ID NO: 799 |
| TPS4-likeJL | 3b | TPS4-likeJL-3bR | ACTAAGCTCCTTTTTGTGCATGG | SEQ ID NO: 800 |
| TPS4-likeJL | 4 | TPS4-likeJL-4F | GGTGGAAGGAATTAGACAGTGC | SEQ ID NO: 801 |
| TPS4-likeJL | 4 | TPS4-likeJL-4R | TCAGCGATTGAGGAAAGTGC | SEQ ID NO: 802 |
| TPS4-likeJL | 5 | TPS4-likeJL-5F | GGGACAAAAATTGTATGGATAAACTCC | SEQ ID NO: 803 |
| TPS4-likeJL | 5 | TPS4-likeJL-5R | TCCTTTTCAAACTCTTGTTCAAATTCC | SEQ ID NO: 804 |
| TPS4-likeJL | 6a | TPS4-likeJL-6aF | AGCTCGATGGTTGAATGAAGG | SEQ ID NO: 805 |
| TPS4-likeJL | 6a | TPS4-likeJL-6aR | GGTCTTTGGAGAGCCACTCG | SEQ ID NO: 806 |
| TPS4-likeJL | 6b | TPS4-likeJL-6bF | TGATGGCTTGCTCTTTAGTTGG | SEQ ID NO: 807 |
| TPS4-likeJL | 6b | TPS4-likeJL-6bR | AGCCACGTCATCCATGTACC | SEQ ID NO: 808 |
| TPS4-likeJL | 7a | TPS4-likeJL-7aF | AGAATGAGCAAGAGAGAAATCATATACC | SEQ ID NO: 809 |
| TPS4-likeJL | 7a | TPS4-likeJL-7aR | CCATGCAATAACCACTCGCC | SEQ ID NO: 810 |
| TPS4-likeJL | 7b | TPS4-likeJL-7bF | TCTCAAACCAACTGAAGCAGC | SEQ ID NO: 811 |
| TPS4-likeJL | 7b | TPS4-likeJL-7bR | TTGGATCAATGAGCAAGACAGC | SEQ ID NO: 812 |
| TPS50JL | 1 | TPS50JL-1F | GGTCTTCATCCTTTGGAAAATCC | SEQ ID NO: 813 |
| TPS50JL | 1 | TPS50JL-1R | AAGCAACCGAAATCGAAGAGC | SEQ ID NO: 814 |
| TPS50JL | 2 | TPS50JL-2F | AGCTTCACAAATGAGAGTTCGC | SEQ ID NO: 815 |
| TPS50JL | 2 | TPS50JL-2R | TCGTGAGAAGGTAGTTGATGG | SEQ ID NO: 816 |
| TPS50JL | 3 | TPS50JL-3F | ACAGAATAGTGGAGTGTTACATTTGG | SEQ ID NO: 817 |
| TPS50JL | 3 | TPS50JL-3R | ATAATAGTAAGCAAAGAAATCAACTTGG | SEQ ID NO: 818 |
| TPS50JL | 4 | TPS50JL-4F | ACAACTTGCCAGAGTACATGAAAG | SEQ ID NO: 819 |
| TPS50JL | 4 | TPS50JL-4R | CGCGCAGTATGAATTTTCCTTTG | SEQ ID NO: 820 |
| TPS50JL | 5 | TPS50JL-5F | GGATCTGAAGAGCTAATATCAATGGC | SEQ ID NO: 821 |
| TPS50JL | 5 | TPS50JL-5R | AGTTCGTATAATCTTAGGTTGGGG | SEQ ID NO: 822 |
| TPS50JL | 6a | TPS50JL-6aF | TCAAAAGATGTAGTGGCTTCTGC | SEQ ID NO: 823 |
| TPS50JL | 6a | TPS50JL-6aR | ATTAAGAGTGGTCTAGGAATAGCG | SEQ ID NO: 824 |
| TPS50JL | 6b | TPS50JL-6bF | CAATATGGTGTAACAGATGAAGAAGC | SEQ ID NO: 825 |
| TPS50JL | 6b | TPS50JL-6bR | TGAGAGTATCAATCAAATTCTTGAGC | SEQ ID NO: 826 |
| TPS51JL | 1 | TPS51JL-1F | AGCCAACTTTGAACCATCCA | SEQ ID NO: 827 |
| TPS51JL | 1 | TPS51JL-1R | AAAGAGACTGAATGAAATCAAAAGACCA | SEQ ID NO: 828 |
| TPS51JL | 2 | TPS51JL-2F | GAGGAAGATGTGAAAAGGATGC | SEQ ID NO: 829 |
| TPS51JL | 2 | TPS51JL-2R | GCCTAAATTCAAGAGCAGTGGC | SEQ ID NO: 830 |
| TPS51JL | 3a | TPS51JL-3aF | CAGGAAAATTCAAAACAAACATAAGTGG | SEQ ID NO: 831 |
| TPS51JL | 3a | TPS51JL-3aR | AGCTTCCTCTAAAATGCTTTCGC | SEQ ID NO: 832 |
| TPS51JL | 3b | TPS51JL-3bF | AGGCGAAAGCATTTTAGAGGAAGC | SEQ ID NO: 833 |
| TPS51JL | 3b | TPS51JL-3bR | AGCTTCTATTCTTGTGGTCCTTCG | SEQ ID NO: 834 |
| TPS51JL | 4 | TPS51JL-4F | GGTGGAGGCATACTAAACTTGG | SEQ ID NO: 835 |
| TPS51JL | 4 | TPS51JL-4R | TGAAAAGCTCTAATTCATCTAATGTTCC | SEQ ID NO: 836 |
| TPS51JL | 5 | TPS51JL-5F | GATGGGATGTGGAAATGATAAATGA | SEQ ID NO: 837 |
| TPS51JL | 5 | TPS51JL-5R | ATTTTGATGGTGATGTGTTGATCT | SEQ ID NO: 838 |
| TPS51JL | 6a | TPS51JL-6aF | CTACAAGAAGCAAAATGGTATTACAGTGG | SEQ ID NO: 839 |
| TPS51JL | 6a | TPS51JL-6aR | GCACAATAAGAACTGGTGCTCCC | SEQ ID NO: 840 |
| TPS51JL | 6b | TPS51JL-6bF | GGAGCACCAGTTCTTATTGTGC | SEQ ID NO: 841 |
| TPS51JL | 6b | TPS51JL-6bR | GCACTGTGACGAATTATGGTAGG | SEQ ID NO: 842 |
| TPS51JL | 7 | TPS51JL-7F | AAAGAGGTGATGCTCCGACG | SEQ ID NO: 843 |
| TPS51JL | 7 | TPS51JL-7R | AGATTATCCTGAGAACTGTGACC | SEQ ID NO: 844 |
| TPS52JL | 1 | TPS52JL-1F | TCAGAAGAGATCAGCAAACTATCAAC | SEQ ID NO: 845 |
| TPS52JL | 1 | TPS52JL-1R | CTTGAAAGGAGTAGAAAGTGATTGA | SEQ ID NO: 846 |
| TPS52JL | 2 | TPS52JL-2F | TGCAAAGACTTGGAATCTCTTACC | SEQ ID NO: 847 |
| TPS52JL | 2 | TPS52JL-2R | CATACACATTTTTGTTGGTGTTGC | SEQ ID NO: 848 |
| TPS52JL | 3 | TPS52JL-3F | GAGGCAATTTTATGGTGTCTTCC | SEQ ID NO: 849 |
| TPS52JL | 3 | TPS52JL-3R | TTGTTTTGTGTCTTGTCTCTTCC | SEQ ID NO: 850 |
| TPS52JL | 4 | TPS52JL-4F | GGACAATAGGAGTTTCATTTGAGCC | SEQ ID NO: 851 |
| TPS52JL | 4 | TPS52JL-4R | TCTCAACCACATTAGTGAAGAGC | SEQ ID NO: 852 |
| TPS52JL | 5 | TPS52JL-5F | TGGGATGTTAGTGCTATGAATGGG | SEQ ID NO: 853 |
| TPS52JL | 5 | TPS52JL-5R | GAGGCTTTTTCCTTTTAACACATCAAATGC | SEQ ID NO: 854 |
| TPS52JL | 6 | TPS52JL-6F | TAAGAGAAGCAAGATGGTATTATGATGG | SEQ ID NO: 855 |
| TPS52JL | 6 | TPS52JL-6R | GTATTATGGTAGGGTATCCATTAAAGC | SEQ ID NO: 856 |
| TPS52JL | 7 | TPS52JL-7F | CGTGTATGTGAAGAGAAAGCTCG | SEQ ID NO: 857 |
| TPS52JL | 7 | TPS52JL-7R | AGTACATGATCTTTTGTTTGGCG | SEQ ID NO: 858 |
| TPS53JL | 1 | TPS53JL-1F | CAGCCAACTTTGAACCATCCA | SEQ ID NO: 859 |
| TPS53JL | 1 | TPS53JL-1R | GCTTGAAAGAGACTGAACGAAA | SEQ ID NO: 860 |
| TPS53JL | 2 | TPS53JL-2F | CGACGAAGATGTGAAAAGGATGC | SEQ ID NO: 861 |
| TPS53JL | 2 | TPS53JL-2R | ACCATGTTGACGTAGAAGCC | SEQ ID NO: 862 |
| TPS53JL | 3a | TPS53JL-3aF | AGCTAGAATTTTCACAACTGAACG | SEQ ID NO: 863 |
| TPS53JL | 3a | TPS53JL-3aR | GCTTCTATTCTTGTGGTCCTTCG | SEQ ID NO: 864 |
| TPS53JL | 3b | TPS53JL-3bF | TACGATATTGAAGTAGTGAATCATGC | SEQ ID NO: 865 |
| TPS53JL | 3b | TPS53JL-3bR | TTGGCAAACTCAAGCAAAATAGG | SEQ ID NO: 866 |
| TPS53JL | 4 | TPS53JL-4F | TGGTGGAGGCATACTAAACTTGG | SEQ ID NO: 867 |
| TPS53JL | 4 | TPS53JL-4R | GTGAAAAGCTCTAATTCATCTAATGTTCC | SEQ ID NO: 868 |
| TPS53JL | 5 | TPS53JL-5F | GATGGGATGTGGAAATGATAAATGAA | SEQ ID NO: 869 |
| TPS53JL | 5 | TPS53JL-5R | ATTTTGATGGTGATTTGTTGATCTCT | SEQ ID NO: 870 |
| TPS53JL | 6a | TPS53JL-6aF | CTACAAGAAGCAAAATGGTACTACAGTGG | SEQ ID NO: 871 |
| TPS53JL | 6a | TPS53JL-6aR | GCACAATAAGAACTGGTGCTCCC | SEQ ID NO: 872 |
| TPS53JL | 6b | TPS53JL-6bF | GGAGCACCAGTTCTTATTGTGC | SEQ ID NO: 873 |
| TPS53JL | 6b | TPS53JL-6bR | GCACTGTGACGAATTATGGTAGG | SEQ ID NO: 874 |
| TPS53JL | 7 | TPS53JL-7F | AAGAGGTGATGCTCCGACG | SEQ ID NO: 875 |
| TPS53JL | 7 | TPS53JL-7R | TAGATTATCCTGAGAACTGTGACC | SEQ ID NO: 876 |
| TPS54JL | 1a | TPS54JL-1aF | ACAAAGAGAGTTATGTGAAGATTATTGAGC | SEQ ID NO: 877 |
| TPS54JL | 1a | TPS54JL-1aR | CCGAAAGCGAAGAGCATCAGC | SEQ ID NO: 878 |
| TPS54JL | 1b | TPS54JL-1bF | AATTACTTCATGGTCTTCAACCTTTGG | SEQ ID NO: 879 |
| TPS54JL | 1b | TPS54JL-1bR | CTATAAAATAGCCTTGTTCCCTAAGC | SEQ ID NO: 880 |
| TPS54JL | 2a | TPS54JL-2aF | AGCTTCACAAATGAGAGTTCGC | SEQ ID NO: 881 |
| TPS54JL | 2a | TPS54JL-2aR | CGGATAGGGTGCATTAGGGC | SEQ ID NO: 882 |
| TPS54JL | 2b | TPS54JL-2bF | ATGCACCCTATCCGAAAGAGC | SEQ ID NO: 883 |
| TPS54JL | 2b | TPS54JL-2bR | TTCCTTTTGATGTAGTTGTTGAAGC | SEQ ID NO: 884 |
| TPS54JL | 3 | TPS54JL-3F | ACAGAATAGTGGAGTGTTACATTTGG | SEQ ID NO: 885 |
| TPS54JL | 3 | TPS54JL-3R | ATAATAGTAAGCAAAGAAATCAACTTGG | SEQ ID NO: 886 |
| TPS54JL | 4 | TPS54JL-4F | TCATCAGGTGGGATATTTTTGCT | SEQ ID NO: 887 |
| TPS54JL | 4 | TPS54JL-4R | ACATAACTCGCGCAGAATGAA | SEQ ID NO: 888 |
| TPS54JL | 5 | TPS54JL-5F | GGATCTGAAGAGCTAATATCAATGGC | SEQ ID NO: 889 |
| TPS54JL | 5 | TPS54JL-5R | TCGTATAATGTTAGGTTGGGGC | SEQ ID NO: 890 |
| TPS54JL | 6 | TPS54JL-6F | TCAAAAGATGTAGTGGCTTCTGC | SEQ ID NO: 891 |
| TPS54JL | 6 | TPS54JL-6R | ATTAAGAGTGGTCTAGGAATAGCG | SEQ ID NO: 892 |
| TPS55JL | 1 | TPS55JL-1F | ACAAAAGGTTGAGGAATTAAAAGAGG | SEQ ID NO: 893 |
| TPS55JL | 1 | TPS55JL-1R | CCCACAAGGAAACATCATGC | SEQ ID NO: 894 |
| TPS55JL | 2 | TPS55JL-2F | CACACCTAAATGAGTTTTTGGCG | SEQ ID NO: 895 |
| TPS55JL | 2 | TPS55JL-2R | CCTGCTGATTTCACTAAGCTCC | SEQ ID NO: 896 |
| TPS55JL | 3 | TPS55JL-3F | TGTGGATGTTAGGAGTCTATTATGAACC | SEQ ID NO: 897 |
| TPS55JL | 3 | TPS55JL-3R | ATTTCTTGCCAAAGAGTATTGGGG | SEQ ID NO: 898 |
| TPS55JL | 4 | TPS55JL-4F | TTGTTATGAAGAGTTTGAGCAAGTG | SEQ ID NO: 899 |
| TPS55JL | 4 | TPS55JL-4R | CTCTATAAGTTTCTTCTTTTGTAAGCAC | SEQ ID NO: 900 |
| TPS55JL | 5 | TPS55JL-5F | GAAGCTCGATGGTTGAATAGTGG | SEQ ID NO: 901 |
| TPS55JL | 5 | TPS55JL-5R | CATGAGCCTACAAATAATAACAGATGC | SEQ ID NO: 902 |
| TPS55JL | 6 | TPS55JL-6F | AAATTAGCTTCACCCATATTACTTAGG | SEQ ID NO: 903 |
| TPS55JL | 6 | TPS55JL-6R | TGGGATCAATTAGCAAAGCAGC | SEQ ID NO: 904 |
| TPS56JL | 1 | TPS56JL-1F | TCCACTCAAATCTTAGCATCATCA | SEQ ID NO: 905 |
| TPS56JL | 1 | TPS56JL-1R | TGGATGAAATGTTTTTGTAGGACGA | SEQ ID NO: 906 |
| TPS56JL | 2 | TPS56JL-2F | GAAAAGGTTGAGGAATTAAAAGAAGTGG | SEQ ID NO: 907 |
| TPS56JL | 2 | TPS56JL-2R | GTTTCAAAATGATAAGACAATGCCAAACG | SEQ ID NO: 908 |
| TPS56JL | 3 | TPS56JL-3F | AAAATGTTTGGCAAGTGATACCC | SEQ ID NO: 909 |
| TPS56JL | 3 | TPS56JL-3R | GGGCCTCTCTAGGGCTCG | SEQ ID NO: 910 |
| TPS56JL | 4 | TPS56JL-4F | TAGGATTGTGGAATTGTACCTTTGG | SEQ ID NO: 911 |
| TPS56JL | 4 | TPS56JL-4R | TGCATCATAATCAGTAATTGAGGC | SEQ ID NO: 912 |
| TPS56JL | 5a | TPS56JL-5aF | AGGACGTTTTGAATTGTTATGAAGAGT | SEQ ID NO: 913 |
| TPS56JL | 5a | TPS56JL-5aR | TCTTTATGTTACTTCTTTTTCTAGCACT | SEQ ID NO: 914 |
| TPS56JL | 5b | TPS56JL-5bF | ACTCAATCAAGAATACATGCAAACAT | SEQ ID NO: 915 |
| TPS56JL | 5b | TPS56JL-5bR | GCACTTGCTCAAACTCTTCATAAC | SEQ ID NO: 916 |
| TPS56JL | 6a | TPS56JL-6aF | TGTTTGCATAGAGGACTCATCCC | SEQ ID NO: 917 |
| TPS56JL | 6a | TPS56JL-6aR | TCTTTCGATCTTTGGAGAGCC | SEQ ID NO: 918 |
| TPS56JL | 6b | TPS56JL-6bF | GTTTGCATAGAGGACTCATCCC | SEQ ID NO: 919 |
| TPS56JL | 6b | TPS56JL-6bR | AGTTTTCATTCCAACCAAAGAGC | SEQ ID NO: 920 |
| TPS56JL | 7a | TPS56JL-7aF | TGGTGTTTGTGAAGAAGAAGCC | SEQ ID NO: 921 |
| TPS56JL | 7a | TPS56JL-7aR | GGTGAAGCTACTTGAGTTGGC | SEQ ID NO: 922 |
| TPS56JL | 7b | TPS56JL-7bF | TTTGTGAAGCCAACTCAAGTAGC | SEQ ID NO: 923 |
| TPS56JL | 7b | TPS56JL-7bR | ATTGGATGAATAAGCAAAGCAGC | SEQ ID NO: 924 |
| TPS57JL | 1 | TPS57JL-1F | TCATCCTTTGGAAAACCCTTTGG | SEQ ID NO: 925 |
| TPS57JL | 1 | TPS57JL-1R | AAGCAACCGAAAGCAAAGAGC | SEQ ID NO: 926 |
| TPS57JL | 2 | TPS57JL-2F | CATATTCGACAAGTACAAGAATGAAAAAGG | SEQ ID NO: 927 |
| TPS57JL | 2 | TPS57JL-2R | CTCCATGAACTCTCATTTGTGC | SEQ ID NO: 928 |
| TPS57JL | 3 | TPS57JL-3F | GGAGTGCTATTTCTGGGTTTATGG | SEQ ID NO: 929 |
| TPS57JL | 3 | TPS57JL-3R | AGGCTCTAGTTCTTCAAGTGTACC | SEQ ID NO: 930 |
| TPS57JL | 5 | TPS57JL-5F | GTTGGGGTTGATAGTGCAGG | SEQ ID NO: 931 |
| TPS57JL | 5 | TPS57JL-5R | TCATTCATAACTCTTCCAACTATTGCC | SEQ ID NO: 932 |
| TPS57JL | 6 | TPS57JL-6F | GGAGCGGAAGAAATCATCAGG | SEQ ID NO: 933 |
| TPS57JL | 6 | TPS57JL-6R | TGGGTTGTGTAAAGCCATCTCC | SEQ ID NO: 934 |
| TPS58JL | 1 | TPS58JL-1F | ATTCAACGATTGGGGTTGTCTT | SEQ ID NO: 935 |
| TPS58JL | 1 | TPS58JL-1R | CCGAAAGCAAAGAGCATCAGC | SEQ ID NO: 936 |
| TPS58JL | 2 | TPS58JL-2F | CATATTCGACAAGTACAAGAATGAAAAAGG | SEQ ID NO: 937 |
| TPS58JL | 2 | TPS58JL-2R | CTCCATGAACTCTCATTTGTGC | SEQ ID NO: 938 |
| TPS58JL | 3a | TPS58JL-3aF | AGAATAGTGGAGTGCTATTTCTGG | SEQ ID NO: 939 |
| TPS58JL | 3a | TPS58JL-3aR | ATTATTAGTCTGATTTGGGAAGTTTCTGC | SEQ ID NO: 940 |
| TPS58JL | 3b | TPS58JL-3bF | TGGAGTGCTATTTCTGGGTTTATGG | SEQ ID NO: 941 |
| TPS58JL | 3b | TPS58JL-3bR | ATTTGGTGATTATTAGTCTGATTTGGG | SEQ ID NO: 942 |
| TPS58JL | 5 | TPS58JL-5F | GTTGGGGTTGATAGTGCAGG | SEQ ID NO: 943 |
| TPS58JL | 5 | TPS58JL-5R | TCATTCATAACTCTTCCAACTATTGCC | SEQ ID NO: 944 |
| TPS58JL | 6a | TPS58JL-6aF | GAAATTGTGGCTTCAACTGTGG | SEQ ID NO: 945 |
| TPS58JL | 6a | TPS58JL-6aR | GAGCAAAGTGGGTTGTGTAAAGC | SEQ ID NO: 946 |
| TPS58JL | 6b | TPS58JL-6bF | AGAAATCATCAGGAGAAATTGTGGC | SEQ ID NO: 947 |
| TPS58JL | 6b | TPS58JL-6bR | TGGGTTGTGTAAAGCCATCTCC | SEQ ID NO: 948 |
| TPS59JL | 1 | TPS59JL-1F | CACACAAATCTTAGTATCTTCAAATGACA | SEQ ID NO: 949 |
| TPS59JL | 1 | TPS59JL-1R | CAAATCTTGTTGTGAAATGTTGTAATG | SEQ ID NO: 950 |
| TPS59JL | 2 | TPS59JL-2F | AAAAGGTTGAGGAATTAAAAGAAGTGG | SEQ ID NO: 951 |
| TPS59JL | 2 | TPS59JL-2R | AGTTTCAAAATGATAAGACAATCCCAAACG | SEQ ID NO: 952 |
| TPS59JL | 3a | TPS59JL-3aF | TGAAAAATTCAAAGACGAGGATGGG | SEQ ID NO: 953 |
| TPS59JL | 3a | TPS59JL-3aR | CATTTAGGTGTGTTGTTGTGAAAGC | SEQ ID NO: 954 |
| TPS59JL | 3b | TPS59JL-3bF | ACTTGCTTTCACAACAACACACC | SEQ ID NO: 955 |
| TPS59JL | 3b | TPS59JL-3bR | AAGGGCCTCTCTAGGGCTCG | SEQ ID NO: 956 |
| TPS59JL | 3c | TPS59JL-3cF | ACACCATGAAGACGATGATCC | SEQ ID NO: 957 |
| TPS59JL | 3c | TPS59JL-3cR | ACCTGCTGACTTCACTAAGTTCC | SEQ ID NO: 958 |
| TPS59JL | 4 | TPS59JL-4F | AGATAGGATTGTGGAATTATACCTTTGG | SEQ ID NO: 959 |
| TPS59JL | 4 | TPS59JL-4R | AGCTCAAGTTCTTCAAGTGTACC | SEQ ID NO: 960 |
| TPS59JL | 5 | TPS59JL-5F | TTTGAATTGTTATGAAGAGTTTGAGC | SEQ ID NO: 961 |
| TPS59JL | 5 | TPS59JL-5R | TGTATTGTTCTTCTTTTTCTAGCACTT | SEQ ID NO: 962 |
| TPS59JL | 6 | TPS59JL-6F | ATTTGGATGAAGCTCGATGTTTGC | SEQ ID NO: 963 |
| TPS59JL | 6 | TPS59JL-6R | CCATGAGCCTACAAATAGTAACAGATGC | SEQ ID NO: 964 |
| TPS59JL | 7a | TPS59JL-7aF | TGAGGAACATTCAGCAGTGG | SEQ ID NO: 965 |
| TPS59JL | 7a | TPS59JL-7aR | GCTTCAAAAACTCTTCATTTATTTCTTTCC | SEQ ID NO: 966 |
| TPS59JL | 7b | TPS59JL-7bF | AAGAAATAAATGAAGAGTTTTTGAAGCC | SEQ ID NO: 967 |
| TPS59JL | 7b | TPS59JL-7bR | CCAACGTGTGTATAACCATCTCC | SEQ ID NO: 968 |
| TPS5FN | 1 | TPS5FN-1F | ATGTCACTATCAGGACTAATCTCC | SEQ ID NO: 969 |
| TPS5FN | 1 | TPS5FN-1R | AAAATGAGCATCCCACAATGG | SEQ ID NO: 970 |
| TPS5FN | 2 | TPS5FN-2F | AGCTATGGGAAAAGAATCAATGAGC | SEQ ID NO: 971 |
| TPS5FN | 2 | TPS5FN-2R | GAGTTTGAAATGAAGAGCAGTGG | SEQ ID NO: 972 |
| TPS5FN | 3 | TPS5FN-3F | GACGAGAAAAAGGAGTTCAAGG | SEQ ID NO: 973 |
| TPS5FN | 3 | TPS5FN-3R | TCATCAAGTATTGTTTGAGATGTTTGG | SEQ ID NO: 974 |
| TPS5FN | 4 | TPS5FN-4F | GCCGAAAAATGCTCACAAAGATTGG | SEQ ID NO: 975 |
| TPS5FN | 4 | TPS5FN-4R | CTTCCCAATGCGTGTTGAAAGAGG | SEQ ID NO: 976 |
| TPS5FN | 5 | TPS5FN-5F | ACCAACTTCCAGATTACATGAAGATA | SEQ ID NO: 977 |
| TPS5FN | 5 | TPS5FN-5R | TAGCACGTCATACGCCATTT | SEQ ID NO: 978 |
| TPS5FN | 6 | TPS5FN-6F | GAACCCTTAATTCTAGTCAATCTTTATTGC | SEQ ID NO: 979 |
| TPS5FN | 6 | TPS5FN-6R | TTCCTAAATCATCAACAAGTCGAGC | SEQ ID NO: 980 |
| TPS5FN | 7a | TPS5FN-7aF | TGAACTGAAAAGAGGAGACAATCC | SEQ ID NO: 981 |
| TPS5FN | 7a | TPS5FN-7aR | AATGGAGACTCACCCACTCG | SEQ ID NO: 982 |
| TPS5FN | 7b | TPS5FN-7bF | GCCATAGATTTCGTTAGGACAGC | SEQ ID NO: 983 |
| TPS5FN | 7b | TPS5FN-7bR | GGGAATGGAAGTGAAGAACAAGG | SEQ ID NO: 984 |
| TPS5JL | 1 | TPS5JL-1F | TCAAAGAACAACCAGCAATAGTCC | SEQ ID NO: 985 |
| TPS5JL | 1 | TPS5JL-1R | TTGGAGTGATTGGATAAAATGAGC | SEQ ID NO: 986 |
| TPS5JL | 2a | TPS5JL-2aF | GCTATGGGAAAAGAATCAATGAGC | SEQ ID NO: 987 |
| TPS5JL | 2a | TPS5JL-2aR | AGTTTGAAATGAAGAGCAGTGG | SEQ ID NO: 988 |
| TPS5JL | 2b | TPS5JL-2bF | TCTTGAGAAGGAGGCTGAAAATCC | SEQ ID NO: 989 |
| TPS5JL | 2b | TPS5JL-2bR | GGAGTTTGAAATGAAGAGCAGTGG | SEQ ID NO: 990 |
| TPS5JL | 3a | TPS5JL-3aF | GGAGAGTTTGAGTAAAGATGTGAAAGG | SEQ ID NO: 991 |
| TPS5JL | 3a | TPS5JL-3aR | TCCAACCTTTTCATTCTCCAATGC | SEQ ID NO: 992 |
| TPS5JL | 3b | TPS5JL-3bF | TTTGAGTAAAGATGTGAAAGGAATGG | SEQ ID NO: 993 |
| TPS5JL | 3b | TPS5JL-3bR | GATGTTTGGTTGTGAAATCTCTTGC | SEQ ID NO: 994 |
| TPS5JL | 4 | TPS5JL-4F | GAGCCACAGTTTAGATATTGCCG | SEQ ID NO: 995 |
| TPS5JL | 4 | TPS5JL-4R | AGAGGCTTAGCTCATCAAGCG | SEQ ID NO: 996 |
| TPS5JL | 5 | TPS5JL-5F | CGACCAACTTCCAGATTACATGAAG | SEQ ID NO: 997 |
| TPS5JL | 5 | TPS5JL-5R | AGCACGTCATACGCCATTTCA | SEQ ID NO: 998 |
| TPS5JL | 6a | TPS5JL-6aF | TGGAGGCAAATTGGTATCATAGTGG | SEQ ID NO: 999 |
| TPS5JL | 6a | TPS5JL-6aR | AAAGGTGGGATATTGAAGCAAGC | SEQ ID NO: 1000 |
| TPS5JL | 6b | TPS5JL-6bF | GAACCCTTAATTCTAGTCAATCTTTATTGC | SEQ ID NO: 1001 |
| TPS5JL | 6b | TPS5JL-6bR | TTCCTAAATCATCAACAAGTCGAGC | SEQ ID NO: 1002 |
| TPS5JL | 7a | TPS5JL-7aF | TGAACTGAAAAGAGGAGACAATCC | SEQ ID NO: 1003 |
| TPS5JL | 7a | TPS5JL-7aR | AATGGAGACTCACCCACTCG | SEQ ID NO: 1004 |
| TPS5JL | 7b | TPS5JL-7bF | TGGAAGGAAATGAATGAAGCTCG | SEQ ID NO: 1005 |
| TPS5JL | 7b | TPS5JL-7bR | ACCCCATCTTGCTCCTTTTGG | SEQ ID NO: 1006 |
| TPS60JL | 1 | TPS60JL-1F | GAGCTTGACTATGTTGAAAACCC | SEQ ID NO: 1007 |
| TPS60JL | 1 | TPS60JL-1R | ATAACCTTGTTGTCTAACCAATCG | SEQ ID NO: 1008 |
| TPS60JL | 2a | TPS60JL-2aF | GCTTCACAACTAAGAGTGCATGG | SEQ ID NO: 1009 |
| TPS60JL | 2a | TPS60JL-2aR | TAATGTCTAGCCTGTCTCCTTTGC | SEQ ID NO: 1010 |
| TPS60JL | 2b | TPS60JL-2bF | AACGATATTCGAGGAATGTTAAGC | SEQ ID NO: 1011 |
| TPS60JL | 2b | TPS60JL-2bR | AGGTTTTAGCTCCAGATTCGAGG | SEQ ID NO: 1012 |
| TPS60JL | 3 | TPS60JL-3F | TAATGGATTGTTACTTTTGGACTTTTGG | SEQ ID NO: 1013 |
| TPS60JL | 3 | TPS60JL-3R | GTATTGCTTCAGTTAAGAGGTGTAGC | SEQ ID NO: 1014 |
| TPS60JL | 5 | TPS60JL-5F | TCAACTACCAGAGTACATTCAACC | SEQ ID NO: 1015 |
| TPS60JL | 5 | TPS60JL-5R | GCAATATGATTTGTCCTTAGTAAATTCTCC | SEQ ID NO: 1016 |
| TPS60JL | 6 | TPS60JL-6F | CACTTGTTACTGCTGCTTCTCC | SEQ ID NO: 1017 |
| TPS60JL | 6 | TPS60JL-6R | GCCAATGTCGTTAAGAACTCTACC | SEQ ID NO: 1018 |
| TPS60JL | 7 | TPS60JL-7F | TCTGAATGAAGAGTGTCTCTATCCC | SEQ ID NO: 1019 |
| TPS60JL | 7 | TPS60JL-7R | AGCTTCTCTTGTAAAACCATCTCC | SEQ ID NO: 1020 |
| TPS61JL | 1a | TPS61JL-1aF | ATCATCGAAATTGAAAGACACAGG | SEQ ID NO: 1021 |
| TPS61JL | 1a | TPS61JL-1aR | GGGCAAGATAGTTGCTCTGC | SEQ ID NO: 1022 |
| TPS61JL | 1b | TPS61JL-1bF | GAGCAACTATCTTGCCCAACC | SEQ ID NO: 1023 |
| TPS61JL | 1b | TPS61JL-1bR | TCATATTTGTTGTTAAGAGACTCCAGG | SEQ ID NO: 1024 |
| TPS61JL | 2 | TPS61JL-2F | AGCTTATCGAGGATGTGAGGC | SEQ ID NO: 1025 |
| TPS61JL | 2 | TPS61JL-2R | TCCATGAAGCCTAAGAAGCC | SEQ ID NO: 1026 |
| TPS61JL | 3 | TPS61JL-3F | TGTTTCACAAGGCATATTTGTTGG | SEQ ID NO: 1027 |
| TPS61JL | 3 | TPS61JL-3R | AGTTCCAAAGCATGAACCACC | SEQ ID NO: 1028 |
| TPS61JL | 4 | TPS61JL-4F | GTTGGTGGAAGAATGTGGGC | SEQ ID NO: 1029 |
| TPS61JL | 4 | TPS61JL-4R | GGCATTGGTGAAGTGTCTGAGC | SEQ ID NO: 1030 |
| TPS61JL | 5 | TPS61JL-5F | TGTACGGGAAACTGAAAAACTTCC | SEQ ID NO: 1031 |
| TPS61JL | 5 | TPS61JL-5R | CCCTTTAGATGAGGTAAAACTAACTTGC | SEQ ID NO: 1032 |
| TPS61JL | 6 | TPS61JL-6F | GCTTGGATTTCATCTTCGGGC | SEQ ID NO: 1033 |
| TPS61JL | 6 | TPS61JL-6R | CGCTGAAGTTCCTAAATCGTTGC | SEQ ID NO: 1034 |
| TPS61JL | 7 | TPS61JL-7F | CCTCTCATTCCCAATAAGTAAACTAGC | SEQ ID NO: 1035 |
| TPS61JL | 7 | TPS61JL-7R | TCCTCAAAGTACAGGAGACATCG | SEQ ID NO: 1036 |
| TPS62JL | 1 | TPS62JL-1F | TTGATGCTATTCAACGGCTAGG | SEQ ID NO: 1037 |
| TPS62JL | 1 | TPS62JL-1R | CAGCAGGAACGAAGTGACCG | SEQ ID NO: 1038 |
| TPS62JL | 2 | TPS62JL-2F | ATATGAAGCCTCCCATCTATGC | SEQ ID NO: 1039 |
| TPS62JL | 2 | TPS62JL-2R | TGGGATAGACACCAAAAAGGTCC | SEQ ID NO: 1040 |
| TPS62JL | 3 | TPS62JL-3F | GGTGGCGAGACATTGGTTTAGC | SEQ ID NO: 1041 |
| TPS62JL | 3 | TPS62JL-3R | ATGGATTTTGTAAGCGCAACCC | SEQ ID NO: 1042 |
| TPS62JL | 4 | TPS62JL-4F | TATAGAAAAACTTCCAGACTCCATGA | SEQ ID NO: 1043 |
| TPS62JL | 4 | TPS62JL-4R | TAAAGGGCTCCATCCACGC | SEQ ID NO: 1044 |
| TPS62JL | 5 | TPS62JL-5F | GGGCAAGTTTGTGCGAAGC | SEQ ID NO: 1045 |
| TPS62JL | 5 | TPS62JL-5R | TGGCACTACCAAAGTCATCCC | SEQ ID NO: 1046 |
| TPS62JL | 6a | TPS62JL-6aF | AGGACATGACGGATCTTATGTGG | SEQ ID NO: 1047 |
| TPS62JL | 6a | TPS62J L -6aR | AGGAATGGTGGTGGAAACGC | SEQ ID NO: 1048 |
| TPS62JL | 6b | TPS62JL-6bF | TCCACCACCATTCCTCAAAGC | SEQ ID NO: 1049 |
| TPS62JL | 6b | TPS62JL-6bR | ATGTTCTTCCAAGTGGGGTAGG | SEQ ID NO: 1050 |
| TPS63JL | 1 | TPS63JL-1F | GAACTTGGTCAACGCTGTGC | SEQ ID NO: 1051 |
| TPS63JL | 1 | TPS63JL-1R | ACCTCCTTGTCTCAGTAATCGG | SEQ ID NO: 1052 |
| TPS63JL | 2a | TPS63JL-2aF | TAGTGGAAGAAGACAGAGAGGG | SEQ ID NO: 1053 |
| TPS63JL | 2a | TPS63JL-2aR | AGTGTTGGCTGAGAAACTGTCC | SEQ ID NO: 1054 |
| TPS63JL | 2b | TPS63JL-2bF | TCTCAGCCAACACTGCTACC | SEQ ID NO: 1055 |
| TPS63JL | 2b | TPS63JL-2bR | GAGACTTGAACAATTTCCCTTTGG | SEQ ID NO: 1056 |
| TPS63JL | 3 | TPS63JL-3F | GGTGGAAAGAGCTTGGTTTGG | SEQ ID NO: 1057 |
| TPS63JL | 3 | TPS63JL-3R | GAGTGAGTTCGTCTAGTGTCC | SEQ ID NO: 1058 |
| TPS63JL | 4 | TPS63JL-4F | GGGAAATTAAAGAGCAGTTACCCG | SEQ ID NO: 1059 |
| TPS63JL | 4 | TPS63JL-4R | GGGTTCCATCCATGTTTTCTGT | SEQ ID NO: 1060 |
| TPS63JL | 5a | TPS63JL-5aF | GGTTTGGTTGTGGGAAGTTGC | SEQ ID NO: 1061 |
| TPS63JL | 5a | TPS63JL-5aR | CTGCTGTAGAAGACACAAGGC | SEQ ID NO: 1062 |
| TPS63JL | 5b | TPS63JL-5bF | GAACGCGATTGTGAGTTCTGG | SEQ ID NO: 1063 |
| TPS63JL | 5b | TPS63JL-5bR | GGCACTTCCTAAGTCATCCC | SEQ ID NO: 1064 |
| TPS63JL | 6a | TPS63JL-6aF | ATGGGCATGATGGGTCTTACG | SEQ ID NO: 1065 |
| TPS63JL | 6a | TPS63JL-6aR | CTCCTTATTGAGGCGTTCCC | SEQ ID NO: 1066 |
| TPS63JL | 6b | TPS63JL-6bF | GCAAGGGAGCAAGTGATTCG | SEQ ID NO: 1067 |
| TPS63JL | 6b | TPS63JL-6bR | CTGGAGGCTTGGAAGGCG | SEQ ID NO: 1068 |
| TPS64JL | 1 | TPS64JL-1F | ACTCGGCTACTTTCTCAAAGAGG | SEQ ID NO: 1069 |
| TPS64JL | 1 | TPS64JL-1R | ATGGATTACCAAAACGAGGAAGC | SEQ ID NO: 1070 |
| TPS64JL | 2a | TPS64JL-2aF | AGGCTCATAGATAGCATCCAACGG | SEQ ID NO: 1071 |
| TPS64JL | 2a | TPS64JL-2aR | GTAGCCGAAAACGAAGCCCC | SEQ ID NO: 1072 |
| TPS64JL | 2b | TPS64JL-2bF | TCCTTCAGATGTTGTCAGATTTCAATTCC | SEQ ID NO: 1073 |
| TPS64JL | 2b | TPS64JL-2bR | GAGGTAGTTGGAAAGCCATTATGC | SEQ ID NO: 1074 |
| TPS64JL | 3a | TPS64JL-3aF | ATGCTGAGCTTGTATGAGGC | SEQ ID NO: 1075 |
| TPS64JL | 3a | TPS64JL-3aR | CAAGAAGAGCAGGGCAGTGG | SEQ ID NO: 1076 |
| TPS64JL | 3b | TPS64JL-3bF | GACACCTGAGGATGGCACC | SEQ ID NO: 1077 |
| TPS64JL | 3b | TPS64JL-3bR | TTGTGGAGTGACTGAAGCTCG | SEQ ID NO: 1078 |
| TPS64JL | 4 | TPS64JL-4F | GTGGAAACAGTTGGGTCTGG | SEQ ID NO: 1079 |
| TPS64JL | 4 | TPS64JL-4R | GTGGAGTTCATCCAAAGATCCG | SEQ ID NO: 1080 |
| TPS64JL | 5 | TPS64JL-5F | GATGGGATCTTGGTGCAATGG | SEQ ID NO: 1081 |
| TPS64JL | 5 | TPS64JL-5R | ACATATTTTCATGTACTCAGGAAGC | SEQ ID NO: 1082 |
| TPS64JL | 6 | TPS64JL-6F | TGAAATTGGCTACAGAGTTCTCA | SEQ ID NO: 1083 |
| TPS64JL | 6 | TPS64JL-6R | AGTGTTGTGTAACGCATAATCCA | SEQ ID NO: 1084 |
| TPS64JL | 7a | TPS64JL-7aF | GCATTTCTAACCGAAGCAGAATGG | SEQ ID NO: 1085 |
| TPS64JL | 7a | TPS64JL-7aR | TGGCTGTTCCCAAATCATCCC | SEQ ID NO: 1086 |
| TPS64JL | 7b | TPS64JL-7bF | CACTCATTTTTCCTCATAGGTCATGG | SEQ ID NO: 1087 |
| TPS64JL | 7b | TPS64JL-7bR | TTGGCTGTTCCCAAATCATCCC | SEQ ID NO: 1088 |
| TPS64JL | 8a | TPS64JL-8aF | AGAGAGGAGATGTTGCTTCTAGC | SEQ ID NO: 1089 |
| TPS64JL | 8a | TPS64JL-8aR | AAGGTCAAAACAGGCCGTGG | SEQ ID NO: 1090 |
| TPS64JL | 8b | TPS64JL-8bF | AGCTCGTGGATAGAGCTAAACG | SEQ ID NO: 1091 |
| TPS64JL | 8b | TPS64JL-8bR | CCATGTTGATAGATGACTTGGGC | SEQ ID NO: 1092 |
| TPS6FN | 1 | TPS6FN-1F | ATGCTACCCCATCCAATGTGC | SEQ ID NO: 1093 |
| TPS6FN | 1 | TPS6FN-1R | AATATAATCGAAAGACCAAATGGAGGGC | SEQ ID NO: 1094 |
| TPS6FN | 2 | TPS6FN-2F | TTAGTTGAGATGGAAAACTCTTTAGC | SEQ ID NO: 1095 |
| TPS6FN | 2 | TPS6FN-2R | ATAGCCATGTTGACGTAGAAGC | SEQ ID NO: 1096 |
| TPS6FN | 3 | TPS6FN-3F | TCCCACTTCATCGGAGGACC | SEQ ID NO: 1097 |
| TPS6FN | 3 | TPS6FN-3R | TTGGCTAACTCAAGCAACATAGG | SEQ ID NO: 1098 |
| TPS6FN | 4 | TPS6FN-4F | GGTGGAGACATACTAAACTTGGAGA | SEQ ID NO: 1099 |
| TPS6FN | 4 | TPS6FN-4R | GCTTTGGTGAAAAGCTCTAATTCAT | SEQ ID NO: 1100 |
| TPS6FN | 5 | TPS6FN-5F | ATGAGTTACCAGAATACATGAAGATGC | SEQ ID NO: 1101 |
| TPS6FN | 5 | TPS6FN-5R | GGTATTGAATGTTGATGGAGATTTCTTGG | SEQ ID NO: 1102 |
| TPS6FN | 6a | TPS6FN-6aF | GGTCGATATGTGTAAAAGTTTCTTGC | SEQ ID NO: 1103 |
| TPS6FN | 6a | TPS6FN-6aR | ACTGGTGCTCCTACTGAAATCC | SEQ ID NO: 1104 |
| TPS6FN | 6b | TPS6FN-6bF | AATGGTTGGATTTCAGTAGGAGC | SEQ ID NO: 1105 |
| TPS6FN | 6b | TPS6FN-6bR | TGGCACTGTGACGAATAATGG | SEQ ID NO: 1106 |
| TPS6FN | 7a | TPS6FN-7aF | ATTGAAAAGAGGTGATGCTCCG | SEQ ID NO: 1107 |
| TPS6FN | 7a | TPS6FN-7aR | TTGGATTGATTATCTTGAGAACTATGACC | SEQ ID NO: 1108 |
| TPS6FN | 7b | TPS6FN-7bF | ATTGTATCTGAAGAGGAAGCTCG | SEQ ID NO: 1109 |
| TPS6FN | 7b | TPS6FN-7bR | TATAAGGGAATAGGTTCAATAATCAAGG | SEQ ID NO: 1110 |
| TPS6JL | 1 | TPS6JL-1F | ATGTGCTGTGGTCAATAGTTCT | SEQ ID NO: 1111 |
| TPS6JL | 1 | TPS6JL-1R | AAAGACCAAATGGAGGGCTCA | SEQ ID NO: 1112 |
| TPS6JL | 2 | TPS6JL-2F | ACAGGTCGAGTCAAAGAATTGG | SEQ ID NO: 1113 |
| TPS6JL | 2 | TPS6JL-2R | GCCATGTTGACGTAGAAGCC | SEQ ID NO: 1114 |
| TPS6JL | 3a | TPS6JL-3aF | CTCCCACTTCATTGGAGGACT | SEQ ID NO: 1115 |
| TPS6JL | 3a | TPS6JL-3aR | AGCAAACTCAAGCAAAATAGGATT | SEQ ID NO: 1116 |
| TPS6JL | 3b | TPS6JL-3bF | GCTAAGTGGTTCATCGACGC | SEQ ID NO: 1117 |
| TPS6JL | 3b | TPS6JL-3bR | TGATGTGTTGATTGTATCATGTTGA | SEQ ID NO: 1118 |
| TPS6JL | 4 | TPS6JL-4F | GGTGGAGGCATACTAAACTTGG | SEQ ID NO: 1119 |
| TPS6JL | 4 | TPS6JL-4R | GAAAAGCTCTAATTCATCCAATGTTCC | SEQ ID NO: 1120 |
| TPS6JL | 5 | TPS6JL-5F | GATGGGATATGGAAATGATAAATGAGT | SEQ ID NO: 1121 |
| TPS6JL | 5 | TPS6JL-5R | TGTTGATGGAGATGTGTTGGTCT | SEQ ID NO: 1122 |
| TPS6JL | 6 | TPS6JL-6F | GATTTCAGTGGGAGCACCG | SEQ ID NO: 1123 |
| TPS6JL | 6 | TPS6JL-6R | ACTATGACGAATTATGGCGGG | SEQ ID NO: 1124 |
| TPS6JL | 7 | TPS6JL-7F | GAACTGAAAAGAGGTGATGCTCC | SEQ ID NO: 1125 |
| TPS6JL | 7 | TPS6JL-7R | TTTGGATTGATTATCTTGAGAACTATGACC | SEQ ID NO: 1126 |
| TPS6-likeJL | 1 | TPS6-likeJL-1F | ATGCTACCCCATCCAATGTGC | SEQ ID NO: 1127 |
| TPS6-likeJL | 1 | TPS6-likeJL-1R | AATATAATCAAAAGACCAAATGGAGGGC | SEQ ID NO: 1128 |
| TPS6-likeJL | 2 | TPS6-likeJL-2F | CAACTTGAACTCATTGATACATTGC | SEQ ID NO: 1129 |
| TPS6-likeJL | 2 | TPS6-likeJL-2R | TAGCCATGTTGACGTAGAAGCC | SEQ ID NO: 1130 |
| TPS6-likeJL | 3 | TPS6-likeJL-3F | AGCTCCCACTTCATCGGAGG | SEQ ID NO: 1131 |
| TPS6-likeJL | 3 | TPS6-likeJL-3R | TTTGGCTAACTCAAGCAACATAGG | SEQ ID NO: 1132 |
| TPS6-likeJL | 4 | TPS6-likeJL-4F | GGTGGAGACATACTAAACTTGGAGA | SEQ ID NO: 1133 |
| TPS6-likeJL | 4 | TPS6-likeJL-4R | GCTTTGGTGAAAAGCTCTAATTCAT | SEQ ID NO: 1134 |
| TPS6-likeJL | 5 | TPS6-likeJL-5F | AGTTACCAGAATACATGAAGATGCC | SEQ ID NO: 1135 |
| TPS6-likeJL | 5 | TPS6-likeJL-5R | TTCTTGGTCTCTTAACACCTCAAA | SEQ ID NO: 1136 |
| TPS6-likeJL | 6a | TPS6-likeJL-6aF | GGTCGATATGTGTAAAAGTTTCTTGC | SEQ ID NO: 1137 |
| TPS6-likeJL | 6a | TPS6-likeJL-6aR | ACTGGTGCTCCTACTGAAATCC | SEQ ID NO: 1138 |
| TPS6-likeJL | 6b | TPS6-likeJL-6bF | GAAAATGGTTGGATTTCAGTAGGAGC | SEQ ID NO: 1139 |
| TPS6-likeJL | 6b | TPS6-likeJL-6bR | TGGCACTGTGACGAATAATGGC | SEQ ID NO: 1140 |
| TPS6-likeJL | 7 | TPS6-likeJL-7F | AATTGAAAAGAGGTGATGCTCCG | SEQ ID NO: 1141 |
| TPS6-likeJL | 7 | TPS6-likeJL-7R | TTTGGATTGATTATCTTGAGAACTATGACC | SEQ ID NO: 1142 |
| TPS7FN | 1 | TPS7FN-1F | AGTCAAGTGTTAGCTTCATCTCA | SEQ ID NO: 1143 |
| TPS7FN | 1 | TPS7FN-1R | CGCCCCAAATAGAAGGGTGA | SEQ ID NO: 1144 |
| TPS7FN | 2 | TPS7FN-2F | GAGAGTGAAATTGAGAAATTGTTGG | SEQ ID NO: 1145 |
| TPS7FN | 2 | TPS7FN-2R | TGTCTTAATAATCTAAACCAAAGAGAAGC | SEQ ID NO: 1146 |
| TPS7FN | 3a | TPS7FN-3aF | TGATAACCGACGTTTCGGG | SEQ ID NO: 1147 |
| TPS7FN | 3a | TPS7FN-3aR | CTAGCATGAAGCCTCTCTAAGG | SEQ ID NO: 1148 |
| TPS7FN | 3b | TPS7FN-3bF | GGAAAGGCCACTAAGAATGACC | SEQ ID NO: 1149 |
| TPS7FN | 3b | TPS7FN-3bR | ACCTCACAATTTCACTAAGCTCC | SEQ ID NO: 1150 |
| TPS7FN | 4 | TPS7FN-4F | GTGGAAGGAGCATGAGTTTGC | SEQ ID NO: 1151 |
| TPS7FN | 4 | TPS7FN-4R | TGACTTTGGTTAGAAGTTTTCTTGC | SEQ ID NO: 1152 |
| TPS7FN | 6a | TPS7FN-6aF | GAAGAAGCTCGATGGTTAAATGAAGG | SEQ ID NO: 1153 |
| TPS7FN | 6a | TPS7FN-6aR | AGTAGAAGCTGAAACAATCTTAGGG | SEQ ID NO: 1154 |
| TPS7FN | 6b | TPS7FN-6bF | TCTGGTTACGTTTTGTTGATAGC | SEQ ID NO: 1155 |
| TPS7FN | 6b | TPS7FN-6bR | TGAATCTTGAGAGGAGAGTAGAAGC | SEQ ID NO: 1156 |
| TPS7FN | 7 | TPS7FN-7F | TGAAGCAATATGAGGTTTCAGAGG | SEQ ID NO: 1157 |
| TPS7FN | 7 | TPS7FN-7R | ATGGGATGGGATCTATAAGTAAAGC | SEQ ID NO: 1158 |
| TPS8FN | 1 | TPS8FN-1F | AAGTCTTAGCTTCATCTCAATTATGTGAC | SEQ ID NO: 1159 |
| TPS8FN | 1 | TPS8FN-1R | TCGATCACCCCAAATAGAAGGG | SEQ ID NO: 1160 |
| TPS8FN | 2 | TPS8FN-2F | CGAGAGTGAAATCGAGAAATTATTGG | SEQ ID NO: 1161 |
| TPS8FN | 2 | TPS8FN-2R | CACTTTGTCTTAATAGTCTGAACCG | SEQ ID NO: 1162 |
| TPS8FN | 3 | TPS8FN-3F | AGCTTGTATGAGGCTTCGC | SEQ ID NO: 1163 |
| TPS8FN | 3 | TPS8FN-3R | TCTCTATGGTCTTTCTTAATGGCG | SEQ ID NO: 1164 |
| TPS8FN | 4 | TPS8FN-4F | GGTGGAACTGTATTTTTGGATATTGGG | SEQ ID NO: 1165 |
| TPS8FN | 4 | TPS8FN-4R | ACCTTTGAATTGCTTTGGTAAGAAGC | SEQ ID NO: 1166 |
| TPS8FN | 6a | TPS8FN-6aF | GAAGCTCGATGGTTGAATGAAGG | SEQ ID NO: 1167 |
| TPS8FN | 6a | TPS8FN-6aR | GCGAGAGCCTATGTCATCC | SEQ ID NO: 1168 |
| TPS8FN | 6b | TPS8FN-6bF | TGTGGTTACGTTATGTTGATAGCC | SEQ ID NO: 1169 |
| TPS8FN | 6b | TPS8FN-6bR | AATCTTGATAGGAGAGTGGAAGC | SEQ ID NO: 1170 |
| TPS8FN | 7 | TPS8FN-7F | GTTTGAGCAAGAGAGAAATCACATACC | SEQ ID NO: 1171 |
| TPS8FN | 7 | TPS8FN-7R | TGTAAAATTAAGAACACGAACTAAGATAGG | SEQ ID NO: 1172 |
| TPS8JL | 1 | TPS8JL-1F | TCAAGTCTTAGCTTCATCTCAATTATGT | SEQ ID NO: 1173 |
| TPS8JL | 1 | TPS8JL-1R | TCGATCACCCCAAATAGAAGG | SEQ ID NO: 1174 |
| TPS8JL | 2 | TPS8JL-2F | TTTGAGAGTGAAATTGAGAAATTGTTGG | SEQ ID NO: 1175 |
| TPS8JL | 2 | TPS8JL-2R | TAAATCCATGTTGTCTTAATAATCTAAACC | SEQ ID NO: 1176 |
| TPS8JL | 3a | TPS8JL-3aF | GACGCTGAAGGTAATTTTAAGAAAAGC | SEQ ID NO: 1177 |
| TPS8JL | 3a | TPS8JL-3aR | AAGGTGAGTGGTTGTGAAAGC | SEQ ID NO: 1178 |
| TPS8JL | 3b | TPS8JL-3bF | GCTTCACACTTGAGTTATGTTGG | SEQ ID NO: 1179 |
| TPS8JL | 3b | TPS8JL-3bR | CTCTCTATGGTCTTTCTTAGAGGC | SEQ ID NO: 1180 |
| TPS8JL | 4 | TPS8JL-4F | AGGATGGTGGAACTGTATTTTITGG | SEQ ID NO: 1181 |
| TPS8JL | 4 | TPS8JL-4R | CCTTTGAATTGCTTTGGTAAGAAGC | SEQ ID NO: 1182 |
| TPS8JL | 6 | TPS8JL-6F | GAAGAAGCTCGATGGTTGAATGAAGG | SEQ ID NO: 1183 |
| TPS8JL | 6 | TPS8JL-6R | AGAGTGGAAGCTGAAACAATCTTAGG | SEQ ID NO: 1184 |
| TPS8JL | 7a | TPS8JL-7aF | TGAGCAACAGAGAAATCACATACC | SEQ ID NO: 1185 |
| TPS8JL | 7a | TPS8JL-7aR | GAACACGAAGTAAGATAGGAAAAGGC | SEQ ID NO: 1186 |
| TPS8JL | 7b | TPS8JL-7bF | GTGCCTTTTCCTATCTTACTTCG | SEQ ID NO: 1187 |
| TPS8JL | 7b | TPS8JL-7bR | CAATGCTTTCTTTGAGCACTTTTCC | SEQ ID NO: 1188 |
| TPS8-likeJL | 2 | TPS8-likeJL-2F | TTTGAGAGTGAAATTGAGAAATTGTTGG | SEQ ID NO: 1189 |
| TPS8-likeJL | 2 | TPS8-likeJL-2R | TAAATCCATGTTGTCTTAATAATCTAAACC | SEQ ID NO: 1190 |
| TPS8-likeJL | 3a | TPS8-likeJL-3aF | GCTTGATAACCGATGTTTCGGG | SEQ ID NO: 1191 |
| TPS8-likeJL | 3a | TPS8-likeJL-3aR | GAGGTGAGTGGTTGTGAAAGC | SEQ ID NO: 1192 |
| TPS8-likeJL | 3b | TPS8-likeJL-3bF | TCACCTCAAGGCTATTGTGGC | SEQ ID NO: 1193 |
| TPS8-likeJL | 3b | TPS8-likeJL-3bR | AGATGTAAAACCTAGCATGAAGCC | SEQ ID NO: 1194 |
| TPS8-likeJL | 4 | TPS8-likeJL-4F | GGTGGAAGGAGCATGAGTTTGC | SEQ ID NO: 1195 |
| TPS8-likeJL | 4 | TPS8-likeJL-4R | TTGAGGCTAATGCAATGACTTTGG | SEQ ID NO: 1196 |
| TPS8-likeJL | 5 | TPS8-likeJL-5F | GGGACATAAATTGTCTGGATAAACTTGA | SEQ ID NO: 1197 |
| TPS8-likeJL | 5 | TPS8-likeJL-5R | TTTTTAAGCTCCTTTTCAAATTCTTCATAA | SEQ ID NO: 1198 |
| TPS8-likeJL | 6 | TPS8-likeJL-6F | AGCTCGATGGTTGAGTGAAGG | SEQ ID NO: 1199 |
| TPS8-likeJL | 6 | TPS8-likeJL-6R | ACCTAGCAAGTAGAGTGGAAGC | SEQ ID NO: 1200 |
| TPS8-likeJL | 7a | TPS8-likeJL-7aF | TGAGCAAAAGAGAAATCACATACC | SEQ ID NO: 1201 |
| TPS8-likeJL | 7a | TPS8-likeJL-7aR | TTTCTTTCCAGTGGGTGTCC | SEQ ID NO: 1202 |
| TPS8-likeJL | 7b | TPS8-likeJL-7bF | TATGGGGTATCAGAGAAAGAGGC | SEQ ID NO: 1203 |
| TPS8-likeJL | 7b | TPS8-likeJL-7bR | GAACACGAACTAAGATAGGAAAAGGC | SEQ ID NO: 1204 |
| TPS8-likeJL | 7c | TPS8-likeJL-7cF | GTGCCTTTTCCTATCTTAGTTCG | SEQ ID NO: 1205 |
| TPS8-likeJL | 7c | TPS8-likeJL-7cR | CAATGCTTTCTTTGAGCACTTTTCC | SEQ ID NO: 1206 |
| TPS9FN | 1 | TPS9FN-1F | TCCACTCAAATCTTAGCAACCTC | SEQ ID NO: 1207 |
| TPS9FN | 1 | TPS9FN-1R | TGCAAAAATCGGTCTCCCCA | SEQ ID NO: 1208 |
| TPS9FN | 2a | TPS9FN-2aF | TGAAGTTAATTGATGTGGTAGAACG | SEQ ID NO: 1209 |
| TPS9FN | 2a | TPS9FN-2aR | AGAAACCCTATATCCATGTTGTCG | SEQ ID NO: 1210 |
| TPS9FN | 2b | TPS9FN-2bF | CAAGTTGAAGAATTGAAAGAAGTGG | SEQ ID NO: 1211 |
| TPS9FN | 2b | TPS9FN-2bR | AAATGATAGGACAATCCCAAACG | SEQ ID NO: 1212 |
| TPS9FN | 3a | TPS9FN-3aF | GCGAGTGACACCGTTGGTT | SEQ ID NO: 1213 |
| TPS9FN | 3a | TPS9FN-3aR | TGGATCATCATTGGGGTGTTCTTT | SEQ ID NO: 1214 |
| TPS9FN | 3b | TPS9FN-3bF | AAAGAACACCCCAATGATGATCC | SEQ ID NO: 1215 |
| TPS9FN | 3b | TPS9FN-3bR | GGGTCTTTCTTAAGGGCCTC | SEQ ID NO: 1216 |
| TPS9FN | 4 | TPS9FN-4F | TGGTGGAAGGAATTAGACAGTGC | SEQ ID NO: 1217 |
| TPS9FN | 4 | TPS9FN-4R | ATCAGCGATTGAGGAAAGTGC | SEQ ID NO: 1218 |
| TPS9FN | 5 | TPS9FN-5F | GATAAACTCCATCCAGAATACTTGC | SEQ ID NO: 1219 |
| TPS9FN | 5 | TPS9FN-5R | ACTTTGTAAGTTTCCTCCTTTTCAA | SEQ ID NO: 1220 |
| TPS9FN | 6a | TPS9FN-6aF | GAAGCTCGATGGTTGAATGAAGG | SEQ ID NO: 1221 |
| TPS9FN | 6a | TPS9FN-6aR | CGCTTGCCCTAACAATCTTGG | SEQ ID NO: 1222 |
| TPS9FN | 6b | TPS9FN-6bF | GTTGATGGCTTGCTCTTTAGTTGG | SEQ ID NO: 1223 |
| TPS9FN | 6b | TPS9FN-6bR | TGTGACCAGCCACGTCATCC | SEQ ID NO: 1224 |
| TPS9FN | 7 | TPS9FN-7F | TGTGATGAAATGAATAGGCGAGTGG | SEQ ID NO: 1225 |
| TPS9FN | 7 | TPS9FN-7R | CCATAACCCTAGCAAGATTCAGAGC | SEQ ID NO: 1226 |
| TPS9JL | 1 | TPS9JL-1F | TCAAGTTTTAGCCTCATCACAAAA | SEQ ID NO: 1227 |
| TPS9JL | 1 | TPS9JL-1R | CCCAAATAGAAGGTTGATAAGTTGT | SEQ ID NO: 1228 |
| TPS9JL | 2 | TPS9JL-2F | GCAGCGAGTTGACGAATTAAAGG | SEQ ID NO: 1229 |
| TPS9JL | 2 | TPS9JL-2R | TGAAACCATAAATCCATGTTGGC | SEQ ID NO: 1230 |
| TPS9JL | 3a | TPS9JL-3aF | TGCTTGATAACTGACATTCCCG | SEQ ID NO: 1231 |
| TPS9JL | 3a | TPS9JL-3aR | AAGGTGAGTGGTGGTGAAAGC | SEQ ID NO: 1232 |
| TPS9JL | 3b | TPS9JL-3bF | AGTGCTTGATAACTGACATTCCC | SEQ ID NO: 1233 |
| TPS9JL | 3b | TPS9JL-3bR | ATGAAATGTAATGCCTAGCGTGG | SEQ ID NO: 1234 |
| TPS9JL | 4 | TPS9JL-4F | TGCAAGAGATAGGATTGTGGAGC | SEQ ID NO: 1235 |
| TPS9JL | 4 | TPS9JL-4R | TCTGCAACTGAGGCCAATGC | SEQ ID NO: 1236 |
| TPS9JL | 5 | TPS9JL-5F | ACTTAAATTGTGCGGATCAACTACG | SEQ ID NO: 1237 |
| TPS9JL | 5 | TPS9JL-5R | GTAAACTTTGTAACTTTCCTCCTTTCC | SEQ ID NO: 1238 |
| TPS9JL | 6 | TPS9JL-6F | TCAGTGAAGCTCGATGGTTGC | SEQ ID NO: 1239 |
| TPS9JL | 6 | TPS9JL-6R | TGCAATCTCTCATTATCTTGGGG | SEQ ID NO: 1240 |
| TPS9JL | 7 | TPS9JL-7F | GAGATCATTCACCGTCTACCG | SEQ ID NO: 1241 |
| TPS9JL | 7 | TPS9JL-7R | TTTTTGTCTCTTTTCCAACATGCG | SEQ ID NO: 1242 |
| TPS9-like2JL | 1 | TPS9-like2JL-1F | TGTCGTCTCAAATCTTAGCAACC | SEQ ID NO: 1243 |
| TPS9-like2JL | 1 | TPS9-like2JL-1R | ATGCAAAAATCGGTCTCCCC | SEQ ID NO: 1244 |
| TPS9-like2JL | 2a | TPS9-like2JL-2aF | GCCAAGTTGAAGAATTGAAAGAAGTGG | SEQ ID NO: 1245 |
| TPS9-like2JL | 2a | TPS9-like2JL-2aR | ACTCTCAAAATGATAGGACAATCCC | SEQ ID NO: 1246 |
| TPS9-like2JL | 2b | TPS9-like2JL-2bF | AGAAGTGGTAAGAAAGGAGATATTTGG | SEQ ID NO: 1247 |
| TPS9-like2JL | 2b | TPS9-like2JL-2bR | AAGAAACCCTATATCCATGTTGTCG | SEQ ID NO: 1248 |
| TPS9-like2JL | 3a | TPS9-like2JL-3aF | GAATGTTTGGCGAGTGACACC | SEQ ID NO: 1249 |
| TPS9-like2JL | 3a | TPS9-like2JL-3aR | AAGGGCCTCTCTAGGGCTCG | SEQ ID NO: 1250 |
| TPS9-like2JL | 3b | TPS9-like2JL-3bF | AAGAACACCCCAATGATGATCC | SEQ ID NO: 1251 |
| TPS9-like2JL | 3b | TPS9-like2JL-3bR | ACTAAGCTCCTTTTTGTGCATGG | SEQ ID NO: 1252 |
| TPS9-like2JL | 4a | TPS9-like2JL-4aF | TGGTGGAAGGAATTAGACAGTGC | SEQ ID NO: 1253 |
| TPS9-like2JL | 4a | TPS9-like2JL-4aR | ATCAGCGATTGAGGAAAGTGC | SEQ ID NO: 1254 |
| TPS9-like2JL | 4b | TPS9-like2JL-4bF | TTATGAACCCCAATACTCTTTTGC | SEQ ID NO: 1255 |
| TPS9-like2JL | 4b | TPS9-like2JL-4bR | AGGAGCTTATGTTCTTCAAATATACC | SEQ ID NO: 1256 |
| TPS9-like2JL | 5 | TPS9-like2JL-5F | GGATAAACTCCATCCAGAATACTTGC | SEQ ID NO: 1257 |
| TPS9-like2JL | 5 | TPS9-like2JL-5R | TCTTGTTCAAATTCCTCAAAAGATTGC | SEQ ID NO: 1258 |
| TPS9-like2JL | 6a | TPS9-like2JL-6aF | GAAGCTCGATGGTTGAATGAAGG | SEQ ID NO: 1259 |
| TPS9-like2JL | 6a | TPS9-like2JL-6aR | CGCTTGCCCTAACAATCTTGG | SEQ ID NO: 1260 |
| TPS9-like2JL | 6b | TPS9-like2JL-6bF | TGATGGCTTGCTCTTTAGTTGG | SEQ ID NO: 1261 |
| TPS9-like2JL | 6b | TPS9-like2JL-6bR | AGCCACGTCATCCATGTACC | SEQ ID NO: 1262 |
| TPS9-like2JL | 7a | TPS9-like2JL-7aF | AGAATGAGCAAGAGAGAAATCATATACC | SEQ ID NO: 1263 |
| TPS9-like2JL | 7a | TPS9-like2JL-7aR | CCATGCAATAACCACTCGCC | SEQ ID NO: 1264 |
| TPS9-like2JL | 7b | TPS9-like2JL-7bF | TAGGCGAGTGGTTATTGCATGG | SEQ ID NO: 1265 |
| TPS9-like2JL | 7b | TPS9-like2JL-7bR | CCTAGCAAGATTCAGAGCACG | SEQ ID NO: 1266 |
| TPS9-likeJL | 1 | TPS9-likeJL-1F | AAAATGAGAAAATACATAAAATTGTTCGAC | SEQ ID NO: 1267 |
| TPS9-likeJL | 1 | TPS9-likeJL-1R | TCGATCTCCCCAAATAGATGGA | SEQ ID NO: 1268 |
| TPS9-likeJL | 2 | TPS9-likeJL-2F | TTGAAGAATTGAAAGAAGTAGTAAGAAAGG | SEQ ID NO: 1269 |
| TPS9-likeJL | 2 | TPS9-likeJL-2R | TCAAAATGATAGGACAATCCCAAACG | SEQ ID NO: 1270 |
| TPS9-likeJL | 3a | TPS9-likeJL-3aF | TGACAAGTTCAAAGATGAGAATGGC | SEQ ID NO: 1271 |
| TPS9-likeJL | 3a | TPS9-likeJL-3aR | TAGTAAATCTTCCCCGACGC | SEQ ID NO: 1272 |
| TPS9-likeJL | 3b | TPS9-likeJL-3bF | ACATTTGAGTTGCGTCGGGG | SEQ ID NO: 1273 |
| TPS9-likeJL | 3b | TPS9-likeJL-3bR | TAGCTTGGAGCCTGTTTAGGG | SEQ ID NO: 1274 |
| TPS9-likeJL | 3c | TPS9-likeJL-3cF | AAGAAAAACCCTAAACAGGCTCC | SEQ ID NO: 1275 |
| TPS9-likeJL | 3c | TPS9-likeJL-3cR | CTAAGCTCCTTTTTGTGCATGG | SEQ ID NO: 1276 |
| TPS9-likeJL | 4 | TPS9-likeJL-4F | AGAGACAGGAGTGTGGAACTATACC | SEQ ID NO: 1277 |
| TPS9-likeJL | 4 | TPS9-likeJL-4R | TGTCATCAGCTATTGTGGCAAAGG | SEQ ID NO: 1278 |
| TPS9-likeJL | 5 | TPS9-likeJL-5F | TGAAGAATTTGAGCAAGGGCTTA | SEQ ID NO: 1279 |
| TPS9-likeJL | 5 | TPS9-likeJL-5R | GCGTAATGAACTCTGTAAGTTTCT | SEQ ID NO: 1280 |
| TPS9-likeJL | 6 | TPS9-likeJL-6F | GTGGTTAAAGAAAGCTGAACGC | SEQ ID NO: 1281 |
| TPS9-likeJL | 6 | TPS9-likeJL-6R | GCCGACAAATAGTAGTGGATGC | SEQ ID NO: 1282 |
| TPS9-likeJL | 7 | TPS9-likeJL-7F | TGAATAGGCGAGTGGTTATTGC | SEQ ID NO: 1283 |
| TPS9-likeJL | 7 | TPS9-likeJL-7R | TGCAAGATTCAGGGCACG | SEQ ID NO: 1284 |

One or more of the polynucleotide primer pairs listed above in Table B, as well as additional polynucleotide primer pairs and/or groups of polynucleotide primer pairs as provided below, including B3/F3 polynucleotide primer pairs from the Tables depicting primers selected for LAMP assays (Tables 17-35), can be used to identify/select/breed plant cultivars of interest based on their energetic, anti-nociceptive and/or insecticidal terpene profiles. These polynucleotide primers/groups of polynucleotide primers, along with their expected terpene profiles (main product(s) and side product(s)), are set forth in Tables 1-16 below. Provided herein are plant cultivars identified and/or selected as expressing, or not expressing, at least one terpene synthase allele that is amplified, or a portion of which is amplified, using polynucleotide primer pairs (forward “F” and reverse “R” primers, or B3 and F3 LAMP primers, for HRM analysis), or is analyzed using sets of LAMP primers, for LAMP analysis. In aspects, the plant cultivars so selected have a terpene chemical phenotype that includes at least one of the main or side products generated by one or more of the terpene synthase profiles analyzed by amplification using primer pairs as depicted in Tables 1-15 (terpene chemical phenotype corresponding to each Table is provided immediately below the Table, i.e., the terpene chemical phenotype corresponding to each terpene synthase profile depicted in Tables 1-15 is set forth directly beneath each of Tables 1-15, respectively), and/or one or more of alpha-terpinene and gamma-terpinene, as identified, for example, by amplification using HRM primers and/or LAMP primer sets corresponding to primer group 6 or primer group 19. In aspects, the plant cultivars identified and/or so selected contain at least one of the main products of the terpene chemical phenotype corresponding to one or more of Tables 1-15, and/or one or more of alpha-terpinene and gamma-terpinene, as identified, for example, by amplification using HRM primers and/or LAMP primer sets corresponding to primer group 6 or primer group 19. In certain aspects, the plant cultivars identified and/or so selected contain a fraction of the products of the terpene chemical phenotype corresponding to one or more of Tables 1-15, and/or one or more of alpha-terpinene and gamma-terpinene, as identified, for example, by amplification using HRM primers and/or LAMP primer sets corresponding to primer group 6 or primer group 19. In aspects, the plant cultivars identified and/or so selected contain all of the products of the terpene chemical phenotype corresponding to one or more of Tables 1-15, and/or one or more of alpha-terpinene and gamma-terpinene, as identified, for example, by amplification using HRM primers and/or LAMP primer sets corresponding to primer group 6 or primer group 19.

The plant cultivars identified by any of the methods provided herein (e.g., using any of the primer pairs and/or primer sets as provided herein), and/or samples or extracts thereof, can be used for breeding methods as provided herein, for methods of treatment, e.g., by administering a sample containing one or more terpenes having an energetic profile, one or more terpenes having an anti-nociceptive profile, one or more terpenes having a sedative profile, or any combination thereof, or for methods of imparting insect resistance.

TABLE 1

Primer Group-Target : Primer Group 1 - *CsTPS2SK
Characterized Seq: **DQ839405.1
Assessment: Primers TPS2FN-1aF/R*** (SEQ ID NOs 385 (F) and 386 (R)), 1bF/R (SEQ ID NOs 387 (F) and 388 (R)), 3aF/R (SEQ ID NOs 391 (F) and 392 (R)), 3bF/R (SEQ ID NOs 394 (F) and 393 (R)), and 4aF/R (SEQ ID NOs 395 (F) and 396 (R)) are specific to DQ839405.1

| Primer Set | Gene | Gene Position Start | Amplicon Length |
|---|---|---|---|
| TPS2FN-1aF/R_amp_1 | TPS2 | 51 | 59 |
| TPS2FN-1aF/R_amp_2 | TPS2JL | 51 | 59 |
| TPS2FN-1bF/R_amp_1 | TPS2 | 47 | 51 |
| TPS2FN-1bF/R_amp_2 | TPS2JL | 47 | 51 |
| TPS2FN-3aF/R_amp_1 | TPS2 | 409 | 224 |
| TPS2FN-3aF/R_amp_2 | TPS2JL | 409 | 224 |
| TPS2FN-3bF/R_amp_1 | TPS2 | 475 | 246 |
| TPS2FN-3bF/R_amp_2 | TPS2JL | 475 | 246 |
| TPS2FN-4aF/R_amp_1 | TPS2 | 812 | 100 |
| TPS2FN-4aF/R_amp_2 | TPS2JL | 812 | 100 |

| | |
|---|---|
| Main Product(s) | Side Product(s) |
| (+)-α-pinene | (+)-β-pinene, myrcene, (-)-limonene, β-phellandrene and a monoterpene tentatively identified as isoterpinolene |

*Indicates names of terpene synthase (TPS) genes where mentioned in Tables 1-35 provided herein **Indicates Accession Nos. where mentioned in Tables 1-35 provided herein ***F or For indicates forward primer; R or Rev indicates reverse primer in the forward/reverse primer sets (F/R or For/Rev) listed in Tables 1-16 provided herein

TABLE 2

| | |
|---|---|
| Primer Group-Target : | Primer Group 2 - *CsTPS5FN |
| Characterized Seq: | **KY014560 |

Assessment: Primers TPS5FN-3F/R (SEQ ID NOs 973 (F) and 974 (R)) and 4F/R (SEQ ID NOs 975 (F) and 976 (R)) are specific to KY014560

| | | | |
|---|---|---|---|
| Primer Set | Gene | Gene Position Start | Amplicon Length |
| TPS5FN-3F/R_amp_1 | MSTRG.6589.1 | 653 | 151 |
| TPS5FN-3F/R_amp_2 | TPS5JL | 412 | 151 |
| TPS5FN-3F/R_amp_3 | Icl\|KY014560.1_cds_ARE72256.1_1 | 418 | 151 |
| TPS5FN-4F/R_amp_1 | MSTRG.6589.1 | 1158 | 109 |
| TPS5FN-4F/R_amp_2 | TPS5JL | 917 | 109 |
| TPS5FN-4F/R_amp_3 | Icl\|KY014560.1_cds_ARE72256.1_1 | 923 | 109 |

| | |
|---|---|
| Main Product(s) | Side Product(s) |
| Myrcene | (-)-a-pinene (24%), (-)-limonene (17%), (-)-β-pinene (15%), and sabinene (7%). |

TABLE 3

Primer Group-Target: Primer Group 3 - *CsTPS30PK
Characterized Seq: **KY624367
Assessment: Since TPS30-likeJL and TPS13-like1JL = TPS30PK/KY624367, specific Primers are TPS30-likeJL-6F/R (SEQ ID NOs 439 (F) and 440 (R)), 7F/R (SEQ ID NOs 441 (F) and 442 (R)), 8aF/R (SEQ ID NOs 443 (F) and 444 (R)), 8bF/R (SEQ ID NOs 445 (F) and 446 (R)) and TPS13-like1JL 1F/R (SEQ ID NOs 91 (F) and 92 (R)), 3F/R (SEQ ID NOs 95 (F) and 96 (R)), 6F/R (SEQ ID NOs 103 (F) and 104 (R)), and 7F/R (SEQ ID NOs 105 (F) and 106 (R)).

| Primer Set | Gene | Gene Position Start | Amplicon Length |
|---|---|---|---|
| TPS30-likeJL-6F/R_amp_1 | TPS30-like | 1184 | 134 |
| TPS30-likeJL-6F/R_amp_2 | TPS30-likeJL | 1184 | 134 |
| TPS30-likeJL-7F/R_amp_1 | TPS30-like | 1345 | 189 |
| TPS30-likeJL-7F/R_amp_2 | TPS30-likeJL | 1345 | 189 |
| TPS30-likeJL-8aF/R_amp_1 | TPS30-like | 1629 | 126 |
| TPS30-likeJL-8aF/R_amp_2 | TPS30-likeJL | 1629 | 126 |
| TPS30-likeJL-8bF/R_amp_1 | TPS30-like | 1726 | 158 |
| TPS30-likeJL-8bF/R_amp_2 | TPS30-likeJL | 1726 | 158 |
| TPS13-like1JL-1F/R_amp_1 | TPS13-like1JL | 150 | 136 |
| TPS13-like1JL-3F/R_amp_1 | TPS13-like1JL | 509 | 120 |
| TPS13-like1JL-6F/R_amp_1 | TPS13-like1JL | 1403 | 100 |
| TPS13-like1JL-6F/R_amp_2 | TPS30-like | 1457 | 100 |
| TPS13-like1JL-6F/R_amp_3 | TPS30-likeJL | 1457 | 100 |
| TPS13-like1JL-7F/R_amp_1 | TPS13-like1JL | 1573 | 261 |

| | |
|---|---|
| Main Product(s) | Side Product(s) |
| Myrcene | (-)-a-pinene (24%), (-)-limonene (17%), (-)-β-pinene (15%), and sabinene (7%), or (-)-a-pinene (23%), (-)-limonene (17%), sabinene (15%), and (-)-β-pinene (8%) |

TABLE 4

Primer Group-Target: Primer Group 4 - *CsTPS32PK
Characterized Seq: **MN967484
Assessment: Primers TPS5FN-3F/R (SEQ ID NOs 973 (F) and 974 (R)) and 4F/R (SEQ ID NOs 975 (F) and 976 (R)) and TPS5JL-3aF/R (SEQ ID NOs 991 (F) and 992 (R)), 3bF/R (SEQ ID NOs 993 (F) and 994 (R)), and 5F/R (SEQ ID NOs 997 (F) and 998 (R)) are specific to KY014560, but none specific to MN967484

| Primer Set | Gene | Gene Position Start | Amplicon Length |
|---|---|---|---|
| TPS5FN-3F/R_amp_1 | MSTRG.6589.1 | 653 | 151 |
| TPS5FN-3F/R_amp_2 | TPS5JL | 412 | 151 |
| TPS5FN-3F/R_amp_3 | Icl\|KY014560.1_cds_ARE72256.1_1 | 418 | 151 |
| TPS5FN-4F/R_amp_1 | MSTRG.6589.1 | 1158 | 109 |
| TPS5FN-4F/R_amp_2 | TPS5JL | 917 | 109 |
| TPS5FN-4F/R_amp_3 | Icl\|KY014560.1_cds_ARE72256.1_1 | 923 | 109 |
| TPS5JL-3aF/R_amp_1 | MSTRG.6589.1 | 673 | 240 |
| TPS5JL-3aF/R_amp_2 | TPS5JL | 432 | 240 |
| TPS5JL-3aF/R_amp_3 | Icl\|KY014560.1_cds_ARE72256.1_1 | 438 | 240 |
| TPS5JL-3bF/R_amp_1 | MSTRG.6589.1 | 679 | 107 |
| TPS5JL-3bF/R_amp_2 | TPS5JL | 438 | 107 |
| TPS5JL-3bF/R_amp_3 | Icl\|KY014560.1_cds_ARE72256.1_1 | 444 | 107 |
| TPS5JL-5F/R_amp_1 | MSTRG.6589.1 | 1285 | 78 |
| TPS5JL-5F/R_amp_2 | TPS5JL | 1044 | 78 |
| TPS5JL-5F/R_amp_3 | Icl\|KY014560.1_cds_ARE72256.1_1 | 1050 | 78 |

| | |
|---|---|
| Main Product(s) | Side Product(s) |
| Geraniol/Himachalane | himachalane (32%), bisabolol (31%), (E)-β-farnesene (14%), β-bisabolene (12%), α-bergamotene (10%), and nerolidol (2%). geraniol (23%), α-pinene (20%), myrcene (16%), limonene (13%), β-phellandrene (10%), terpinolene (5%), α-terpineol (13%), and camphene (1%) |

TABLE 5

Primer Group-Target : Primer Group 5 - *CsTPS31 PK
Characterized Seq: **MN967474
Assessment: Primers TPS11JL-1F/R (SEQ ID NOs 1 (F) and 2 (R)), 2bF/R (SEQ ID NOs 5 (F) and 6 (R)), 3aF/R (SEQ ID NOs 7 (F) and 8 (R)), 6F/R (SEQ ID NOs 15 (F) and 16 (R)) are specific to MN967474

| Primer Set | Gene | Gene Position Start | Amplicon Length |
|---|---|---|---|
| TPS11JL-1F/R_amp_1 | MSTRG.6585.1 | 30 | 57 |
| TPS11JL-1F/R_amp_2 | TPS11JL | 10 | 57 |
| TPS11JL-1F/R_amp_3 | IclIMN967474.1_cds_QLC36839.1_1 | 58 | 57 |
| TPS11JL-1F/R_amp_4 | Icl\|NC_044376.1_cds_XP_030508374.1_27714 | 40 | 57 |
| TPS11JL-2bF/R_amp_1 | MSTRG.6585.1 | 195 | 197 |
| TPS11JL-2bF/R_amp_2 | TPS11JL | 175 | 197 |
| TPS11JL-2bF/R_amp_3 | Icl\|MN967474.1_cds_QLC36839.1_1 | 223 | 197 |
| TPS11JL-3aF/R_amp_1 | MSTRG.6585.1 | 521 | 140 |
| TPS11JL-3aF/R_amp_2 | TPS11JL | 501 | 140 |
| TPS11JL-3aF/R_amp_3 | Icl\|MN967474.1_cds_QLC36839.1_1 | 549 | 140 |
| TPS11JL-6F/R_amp_1 | MSTRG.6585.1 | 1166 | 209 |
| TPS11JL-6F/R_amp_2 | TPS11JL | 1146 | 209 |
| TPS11JL-6F/R_amp_3 | Icl\|MN967474.1_cds_QLC36839.1_1 | 1194 | 209 |

| | |
|---|---|
| Main Product(s) | Side Product(s) |
| Terpinolene | α-terpineol (19%), linalool (14%), β-pinene (6%) and terpinen-4-ol (4%). 6% bulnesol, 2% bisabolol, and trace amounts of α-bergamotene and a cadinane type sesquiterpene with RI 1494.18 |

TABLE 6

Primer Group-Target: Primer Group 6 - CsTPS37FN and TPS37LPA5
Characterized Seq: MK614216
Assessment: Primers TPS37FN-2F/R (SEQ ID NOs 487 (F) and 488 (R)), 3F/R (SEQ ID NOs 489 (F) and 490 (R)), 4F/R (SEQ ID NOs 491 (F) and 492 (R)), 5F/R (SEQ ID NOs 493 (F) and 494 (R)), 7F/R (SEQ ID NOs 495 (F) and 496 (R)), 8aF/R (SEQ ID NOs 497 (F) and 498 (R)), and 8bF/R (SEQ ID NOs 499 (F) and 500 (R)) are specific to MK614216

| Primer Set | Gene | Gene Position Start | Amplicon Length |
|---|---|---|---|
| TPS37FN-2F/R_amp_1 | MK614216.1 | 177 | 100 |
| TPS37FN-2F/R_amp_2 | Icl\|MK614216.1_cds_QEM23725.1_1 | 177 | 100 |
| TPS37FN-3F/R_amp_1 | MK614216.1 | 334 | 215 |
| TPS37FN-3F/R_amp_2 | Icl\|MK614216.1_cds_QEM23725.1_1 | 334 | 215 |
| TPS37FN-4F/R_amp_1 | MK614216.1 | 648 | 194 |
| TPS37FN-4F/R_amp_2 | Icl\|MK614216.1_cds_QEM23725.1_1 | 648 | 194 |
| TPS37FN-5F/R_amp_1 | MK614216.1 | 963 | 204 |
| TPS37FN-5F/R_amp_2 | Icl\|MK614216.1_cds_QEM23725.1_1 | 963 | 204 |
| TPS37FN-7/RF_amp_1 | MK614216.1 | 1441 | 174 |
| TPS37FN-7F/R_amp_2 | Icl\|MK614216.1_cds_QEM23725.1_1 | 1441 | 174 |
| TPS37FN-8AF/2_amp_1 | MK614216.1 | 1630 | 229 |
| TPS37FN-8AF/R_amp_2 | Icl\|MK614216.1_cds_QEM23725.1_1 | 1630 | 229 |
| TPS37FN-8bF/R_amp_1 | MK614216.1 | 1733 | 129 |
| TPS37FN-8bF/R_amp_2 | Icl\|MK614216.1_cds_QEM23725.1_1 | 1733 | 129 |

| | |
|---|---|
| Main Product(s) | Side Product(s) |
| Terpinolene | 2.5% 3-Carene, 5.7% Alpha-phellandrene, 3.9% alpha-pinene, 2.7% alpha-terpineol, 3% Myrcene, 5.4% beta-pinene, 5.4% ocimene, 0.9% gamma-terpineol |

In Table 6, the gene designated as MK614216.1 is a terpinolene synthase (terpinolene being the main product) encoded by the nucleic acid sequence set forth in SEQ ID NO:1654 (see, e.g., FIG. 1) and having the peptide sequence set forth below:

MQCMAFHQFAPSSSLPIWSSINNRFTPKTSITSISKPKPKLKPKSNLKSRSRSSTCYPIQCTVVDNP
SSTITNNSDRRSANYGPPIWSFDFIQSLSTQYKGELYTSRLNKLEKDVKRILVGEENCLAQLELIDTI
QRLGLSYRFENEIISILKEKFTNNNNNPNYDLYATALQFRLLRQYGFEVPQEIFNNFKDQKTGEFKA
NISNDIMGALGLYEASFYGKKGESILDEARIFTTKCLKNYIEKNKLLDDDNNIIALFVNHALETPLHWR
INRLEARWFIEMYQKKKDMNFTLLEFAKLDFNMLQSIHQEDLKHLSRWWEQSKLGEKKMENYVRD
RLVEAFLWQIGVKFEPQFSQFRRISARLYVLITVIDDIYDVYGTLEELELFTKAIERWDVKAINELPEY
MRMPFFFLFNTVNEMGYDTLTDKNFINIEYLKKSWVVWSKYQLEEAKWFYSGYKPTLEEYMKNT
WISVGGPIILLHAYFAFTNPLEKASIKFLEEGYDDPSINIHEGSLEISNDGYPTIFHLGSILLRLEDDLG
TSSDEMKRGDVPKSIQCYMSDTGVSEDEAREHIKFLIMETWKEMNKEMDFNNYFSKEVVHVCKN
LGRTAKFIYLYGDGHASQNNLSKGHISDLIINPIPM (SEQ ID NO:1669)

A CsTPS37FN enzyme described in Livingston et al., The Plant Journal, 101:36-56 (2019), identifies the following terpene products produced by this enzyme: terpinolene (major product), α-Pinene, β-Pinene and limonene. The csTPS37FN from LPA5 cDNA, as described herein and as recited in Table 6 (CsTPS37LPA5 gene, used interchangeably herein with csTPS37LPA5 gene or TPS37LPA5 gene), on the other hand, was found to produce the following nine terpenes as identified by GC-MS (detection times indicated in parentheses): terpinolene (major product; 6.4 minutes), α-phellandrene (5.05 minutes), α-terpinene (5.25 minutes), α-pinene (4.65 minutes), β-pinene (4.7 minutes), β-myrcene (4.77 minutes), 3-carene (5.1 minutes), γ-Terpinene (5.88 minutes) and an unknown monoterpene (4.55 minutes). The sequence of the mRNA encoding the TPS37LPA5 mature peptide is shown below:

ACTGTGGTCGATAACCCTAGTTCTACGATTATTAATAATAGTGATCGAAGATCGGCCAAC
TATGGACCTCCCATTTGGTCTTTTGATTTCATTCAATCTCTTTCAACTCAATATAAGGGT
GAACTTTATACAAGTCGATTAAATAAGCTGGAGAAAGACGTGAAAAGGATACTGGTTGGA
GAGGAAAATTGTTTAGCTCAACTTGAGCTAATTGATACAATACAAAGACTTGGATTATCT
TATCGTTTTGAGAATGAAATCATTTCTATTTTGAAAGAAAAATTCACCAATAATAATAAC
AACCCTAATTATGATTTATATGCTACTGCTCTCAAATTTAGGCTTCTACGTCAATATGGA
TTTGAAGTATCTCAAGAAATTTTCAATAATTTCAAAGATCAGAAGACAGGAGAGTTCAAG
GCAAATATAAGTAATGATATTATGGGAGCATTAGGCTTATATGAAGCTTCATTCTATGGG
AAAAAGGGTGAAAGTATTTTGGATGAAGCAAGAATTTTCACAACAAAATGTCTCAAAAAT
TACATAGAGAAAAACAAATTATTAGATGATGATAATAATATTATTGCATTATTTGTTAAC
CATGCTTTGGAGACTCCACTTCATTGGAGAATAAATAGGTTGGAAGCTAGGTGGTTCATT
GAGATGTACCAGAAGAAACATGACATGAATTTCACATTACTTGAATTTGCCAAATTGGAT
TTTAACATGCTCCAATCAATACACCAAGAAGATCTAAAACATCTATCAAGATGGTGGGAG
CAATCTAAACTTGGAGAAAAGAAAATGGAAAATTATGTTAGAGATAGATTGGTGGAGGCT
TTTTTATGGCAGATTGGAGTAAAATTTGAGCCACAATTCAGTCAATTTAGAAGAATATCT
GCAAGATTATATGTTCTAATTACAGTAATTGATGACATATATGATGTGTATGGAACATTG
GAGGAATTAGAGCTTTTCACAAAAGCTATTGAAAGATGGGATGTGAAAGCCATAAATGAG
TTACCAGAATATATGAGAATGCCTTTCTTTTTCTTATTCAATACTGTGAATGAAATGGGG
TATGATACCTTAGCAGACAAAAATTTCATCAACATTGAATACCTCAAGAAATCGTGGGTG
GTTTGGTCTAAATATCAATTAGAAGAGGCAAAATGGTTCTATAGTGGATACAAACCAACA
TTAGAAGAATATATGAAGAATACATGGATTTCAGTTGGGGGACCAATTATTCTTTTGCAT
GCTTATTTTGCTTTCACAAATCCCTTAGAAAAAGCTTCCATAAAATTCTTGGAAGAAGGT
TATGATGATCCTTCCATAAATATTCATGAAGGATCCCTGGAAATATCAAATGATGGTTAC
CCTACCATATTTCATCTTGGATCCATACTTTTACGACTTGAAGATGACCTAGGAACATCG
TCGGATGAGATGAAAAGAGGAGATGTTCCGAAATCAATTCAATGTTACATGAACGATACA
GGTGTTTCTGAAGATGAAGCTCGCGAGCACATCAAATTTTTGATAATGGAAACATGGAAA
GAGATGAATAAAGAAATGGACTTCAATAATTATTTCTCGAAAGAAGTTGTTCATGTTTGC
AAAAATCTTGGTAGAACAGCCAAATTTATATACCTTTATGGTGATGGACATGCTTCTCAG
AATAATTTATCAAAGGGACATATTTCAGATTTGATTATTAACCCTATTCCCATGTAA (SEQ ID
NO:1670).

The mature peptide encoded by the TPS37LPA5 gene has the sequence shown below:

TVVDNPSSTIINNSDRRSANYGPPIWSFDFlQSLSTQYKGELYTSRLNKLEKDVKRILVG
EENCLAQLELIDTIQRLGLSYRFENEIISILKEKFTNNNNNPNYDLYATALKFRLLRQYG
FEVSQEIFNNFKDQKTGEFKANISNDIMGALGLYEASFYGKKGESILDEARIFTTKCLKN
YIEKNKLLDDDNNIIALFVNHALETPLHWRINRLEARWFIEMYQKKHDMNFTLLEFAKLD
FNMLQSIHQEDLKHLSRWWEQSKLGEKKMENYVRDRLVEAFLWQIGVKFEPQFSQFRRIS
ARLYVLITVIDDIYDVYGTLEELELFTKAIERWDVKAINELPEYMRMPFFFLFNTVNEMG
YDTLADKNFINIEYLKKSWVVWSKYQLEEAKWFYSGYKPTLEEYMKNTWISVGGPIILLH
AYFAFTNPLEKASIKFLEEGYDDPSINIHEGSLEISNDGYPTIFHLGSILLRLEDDLGTS
SDEMKRGDVPKSIQCYMNDTGVSEDEAREHIKFLIMETWKEMNKEMDFNNYFSKEVVHVC
KNLGRTAKFIYLYGDGHASQNNLSKGHISDLIINPIPM* (SEQ ID NO: 1671).

Thus, provided herein is a terpene synthase gene that facilitates the synthesis of terpinolene and at least two other terpenes selected from among α-phellandrene, α-teminene, 3-carene and γ-Terpinene. In certain aspects, the terpene synthase gene facilitates the synthesis of 3-carene and γ-Terpinene. In aspects, the terpene synthase gene facilitates the synthesis of α-phellandrene, 3-carene and γ-Terpinene.

TABLE 7

Primer Group-Target: Primer Group 7 - CsTPS1 8Choc Characterized Seq: MN967473/MK801763/MK801764
Assessment: Primers TPS18VF-2F/R (SEQ ID NOs 257 (F) and 258 (R)), 5F/R (SEQ ID NOs 265 (F) and 266 (R)), and TPS19BL-3F/R (SEQ ID NOs 281 (F) and 282 (R)), 5aF/R (SEQ ID NOs 285 (F) and 286 (R)), and TPS62JL-4F/R (SEQ ID NOs 1043 (F) and 1044 (R)) are best specific to MN967473/MK801763/MK801764, but really most of this panel would likely pick up the linalool/neriodiol subclade

| Primer Set | Gene | Gene Position Start | Amplicon Length |
|---|---|---|---|
| TPS18VF-2F/R_amp_1 | Icl\|MK801764.1_cds_QCY41292.1_1 | 180 | 218 |
| TPS18VF-5F/R_amp_1 | Icl\|MK801764.1_cds_QCY41292.1_1 | 1014 | 111 |
| TPS18VF-5F/R_amp_2 | Icl\|MN967473.1_cds_QLC36838.1_1 | 1014 | 111 |
| TPS19BL-3F/R_amp_1 | Icl\|MK801763.1_cds_QCY41291.1_1 | 480 | 216 |
| TPS19BL-3F/R_amp_2 | Icl\|MK801764.1_cds_QCY41292.1_1 | 480 | 216 |
| TPS19BL-3F/R_amp_3 | Icl\|NC_044371.1_cds_XP_030491262.1_4287 | 273 | 216 |
| TPS19BL-5aF/R_amp_1 | MSTRG.5536.5 | 1229 | 97 |
| TPS19BL-5aF/R_amp_2 | TPS62JL | 873 | 97 |
| TPS19BL-5aF/R_amp_3 | Icl\|MK801763.1_cds_QCY41291.1_1 | 1020 | 97 |
| TPS62JL-4F/R_amp_1 | MSTRG.5536.5 | 1226 | 106 |
| TPS62JL-4F/R_amp_2 | TPS62JL | 870 | 106 |
| TPS62JL-4F/R_amp_3 | Icl\|MK801763.1_cds_QCY41291.1_1 | 1017 | 106 |

| | |
|---|---|
| Main Product(s) | Side Product(s) |
| (R)-Linalool | |

TABLE 8

| Primer Group-Target : | Primer Group 8 - CsTPS5PK |
|---|---|
| Characterized Seq: | MN967481 |

Redundant from Primer Group 2, Primers TPS5FN-3F/R (SEQ ID NOs 973 (F) and 974 (R)) and 4F/R (SEQ ID NOs 975 (F) and 976 (R))
CsTPS5(PK) has 96% amino acid identity to CsTPS5(FN) (Primer Group 2) and these primers amplify both terpene synthases

| | |
|---|---|
| Main Product(s) | Side Product(s) |
| α-pinene (33%) (GPP as substrate) α-bisabolol (46%) (FPP assubstrate) | myrcene (18%), α-terpineol (18%), limonene (17%), and β-pinene (14%) (GPP as substrate) himachalane (27%), (E)-β-farnesene (11%), α-bergamotene (7%), and a compound tentatively identified as a cyclounitriene (9%) (FPP as substrate). |

TABLE 9

Primer Group-Target : Primer Group 9 - CsTPS18VF/R
Characterized Seq: MK801764
Assessment: Redundant from Primer Group 7, Primers TPS18VF-2F/R (SEQ ID NOs 257 (F) and 258 (R)) and 5F/R (SEQ ID NOs 265 (F) and 266 (R)), and TPS19BL-3F/R (SEQ ID NOs 281 (F) and 282 (R)), 5aF/R (SEQ ID NOs 285 (F) and 286 (R)), and TPS62JL-4F/R (SEQ ID NOs 1043 (F) and 1044 (R)) are best specific to MN967473/MK801763/MK801764, but TPS18VF-2F/R (SEQ ID NOs 257 (F) and 258 (R)) would pick up the specific MK801764 from the linalool/neriodiol subclade

| Primer Set | Gene | Gene Position Start | Amplicon Length |
|---|---|---|---|
| TPS18VF-2F/R_amp_1 | Icl\|MK801764.1_cds_QCY41292.1_1 | 180 | 218 |

| | |
|---|---|
| Main Product(s) | Side Product(s) |
| Linalool | |

TABLE 10

Primer Group-Target : Primer Group 10 - CsTPS19BL
Characterized Seq: MK801763
Assessment: Redundant from Primer Group 7, Primers TPS18VF-2F/R (SEQ ID NOs 257 (F) and 258 (R)) and 5F/R (SEQ ID NOs 265 (F) and 266 (R)), and TPS19BL-3F/R (SEQ ID NOs 281 (F) and 282 (R)), 5aF/R (SEQ ID NOs 285 (F) and 286 (R)), and TPS62JL-4F/R (SEQ ID NOs 1043 (F) and 1044 (R)) are best specific to MN967473/MK801763/MK801763, but TPS19BL-5aF/R (SEQ ID NOs 285 (F) and 286 (R)) and TPS62JL-4F/R (SEQ ID NOs 1043 (F) and 1044 (R)) would pick up the specific MK801763 from the linalool/neriodiol subclade

| Primer Set | Gene | Gene Position Start | Amplicon Length |
|---|---|---|---|
| TPS19BL-5aF/R_amp_1 | MSTRG.5536.5 | 1229 | 97 |
| TPS19BL-5aF/R_amp_2 | TPS62JL | 873 | 97 |
| TPS19BL-5aF/R_amp_3 | Icl\|MK801763.1_cds_QCY41291.1_1 | 1020 | 97 |
| TPS62JL-4F/R_amp_1 | MSTRG.5536.5 | 1226 | 106 |
| TPS62JL-4F/R_amp_2 | TPS62JL | 870 | 106 |
| TPS62JL-4F/R_amp_3 | Icl\|MK801763.1_cds_Q CY41291.1_1 | 1017 | 106 |

| | |
|---|---|
| Main Product(s) | Side Product(s) |
| Linalool/Nerolidol | |

TABLE 11

| Primer Group-Target : | Primer Group 11 - CsTPS35LS | | |
|---|---|---|---|
| Characterized Seq: | MN967475 | | |
| Assessment: Primers TPS63JL-2aF/R (SEQ ID NOs 1053 (F) and 1054 (R)), 2bF/R (SEQ ID NOs 1055 (F) and 1056 (R)), and TPS63JL-6bF/R (SEQ ID NOs 1067 (F) and 1068 (R)) are specific to MN967475 | | | |
| Primer Set | Gene | Gene Position Start | Amplicon Length |
| TPS63JL-2aF/R_amp_1 | MSTRG.5531.1 | 560 | 242 |
| TPS63JL-2aF/R_amp_2 | TPS63JL | 269 | 242 |
| TPS63JL-2aF/R_amp_3 | IcIMN967475.1_cds_QLC36840.1_1 | 416 | 242 |
| TPS63JL-2bF/R_amp_1 | MSTRG.5531.1 | 788 | 121 |
| TPS63JL-2bF/R_amp_2 | TPS63JL | 497 | 121 |
| TPS63JL-2bF/R_amp_3 | Icl\|MN967475.1_cds_QLC36840.1_1 | 644 | 121 |
| TPS63JL-6bF/R_amp_1 | MSTRG.5531.1 | 1591 | 171 |
| TPS63JL-6bF/R_amp_2 | TPS63JL | 1300 | 171 |

| | |
|---|---|
| Main Product(s) | Side Product(s) |
| Linalool/Nerolidol | citronellol (5%) and myrcene (2%) |

TABLE 12

Primer Group-Target : Primer Group 15 - CsTPS25LS
Characterized Seq: MN967472
Assessment: Primers TPS14JL-1F/R (SEQ ID NOs 163 (F) and 164 (R)), 4F/R (SEQ ID NOs 169 (F) and 170 (R)), and 6F/R (SEQ ID NOs 171 (F) and 172 (R)) are specific to MN967472

| Primer Set | Gene | Gene Position Start | Amplicon Length |
|---|---|---|---|
| TPS14JL-1F/R_amp_1 | MN967472.1 | 8 | 86 |
| TPS14JL-1F/R_amp_2 | MSTRG.12576.1 | 420 | 86 |
| TPS14JL-1F/R_amp_3 | TPS14JL | 8 | 86 |
| TPS14JL-1F/R_amp_4 | IcI\|MN967472.1_cds_QLC36837.1_1 | 8 | 86 |
| TPS14JL-1F/R_amp_5 | IcI\|MN967477.1_cds_QLC36842.1_1 | 8 | 86 |
| TPS14JL-4F/R_amp_1 | MSTRG.12576.1 | 1169 | 149 |
| TPS14JL-4F/R_amp_2 | TPS14JL | 757 | 149 |
| TPS14JL-6F/R_amp_1 | MSTRG.12576.1 | 1558 | 189 |
| TPS14JL-6F/R_amp_2 | TPS14JL | 1146 | 189 |

| | |
|---|---|
| Main Product(s) | Side Product(s) |
| (E)-β-farnesene | cadinane type compound (22%) with RI 1494.76, (Z, E)-α-famesene (15%), and nerolidol (7%) |

TABLE 13

Primer Group 16 includes polynucleotide primer pairs for selection of terpene synthases (e.g., TPS20_LPA5, used synonymously with TPS20_LPA005) producing the contact insecticide guaiol (QTL - quantitative trait loci), e.g., using HRM to detect the presence or absence (PAV) of a terpene synthase

| | Sequenc e (5′->3′) | Template strand | Length | Start | Stop | Tm | GC% | Self complem entarity | Self 3′ complem entarity |
|---|---|---|---|---|---|---|---|---|---|
| Forward primer | TGTTTA GAGTTT GCTGTT AGGCA (SEQ ID NO: 1401) | Plus | 23 | 76790560 | 76790582 | 58.54 | 39.13 | 3.00 | 1.00 |
| Reverse primer | GGTGAG ACCGCC GTATAT CTT (SEQ ID NO: 1402) | Minus | 21 | 76790661 | 76790641 | 59.39 | 52.38 | 4.00 | 0.00 |
| Product length | 102 | | | | | | | | |

Primer Group 16 - TPS20-LPA004/LPA005- HRM Discrimination Set that discriminates between the LPA004 and LPA005 alleles - Reverse primer is single loci specific to TPS20

| | | | | | | | | | |
|---|---|---|---|---|---|---|---|---|---|
| Sequenc e(5′->3′) | Sequenc e (5′->3′ | Template strand | Length | Start | Stop | Tm | GC% | Self complem entarity | Self 3′ complem entarity |
| Forward primer | TGAGGC TTCGCA CTTGAG TT (SEQ ID NO: 1403) | Plus | 20 | 459 | 478 | 59.89 | 50.00 | 4.00 | 2.00 |
| Reverse primer | GGCCTA TGCAAG GCTATG GA (SEQ ID NO: 1404) | Minus | 20 | 599 | 580 | 59.60 | 55.00 | 8.00 | 3.00 |
| Product length | | | | | | | | | |

Primer Group 16 - TPS20LPA005 --Specific PAV set - will only amplify TPS20LPA5 - Reverse primer Specific by 1 SNP

| Sequence (5′->3′) | Template strand | Length | Start | Stop | Tm | GC% | Self complem entarity | Selt 3′ complem entarity | |
|---|---|---|---|---|---|---|---|---|---|
| Forward primer | GAAGCA CTTGCT TTCACA AA (SEQ ID NO: 1405) | Plus | 20 | 502 | 521 | 55.31 | 40.00 | 8.00 | 3.00 |
| Reverse primer | GGCCTA TGCAAG GCTATG G (SEQ ID NO: 1406) | Minus | 19 | 599 | 581 | 58.05 | 57.89 | 8.00 | 2.00 |
| Product length | 98 | | | | | | | | |

The TPS20LPA4 (non-guaiol synthase) and TPS20LPA5 (guaiol synthase) alleles share 99.46% nucleotide similarity and 98.91 % amino acid similarity. This represents 6 amino acid changes between the LPA004 and LPA005 versions of TPS20. The mRNA coding sequences for each are shown below:

### TPS20 Sequences For LPA005 and LPA004

TPS20LPA4_mRNA:1-1656

ATGTCAAATATTCAAGTCTTAGCTTCATCTCAATTAAGTGACAAAATTGTTGCTCGYCCA
ACAACAAAATTTCACCCTTCTATTTGGGGCGATCGATTCCTCCATTACAATATTTCAGAA
CAAGACTTGGTTTGCAAACAAGAAARAATTGAAGAATTAATACAAGTTGTAAAGAAAGAG
ATATTATCTTCAAATCATGATCAATTGAAGTTGATTGACAATCTCCAACGTTTGGGATTA
TCATATCATTTTGAGAGTGAAATTGAGAAATTGTTGGAACAATTAAGTACCACTCATCAT
CAAAATCATCAAGATCTACATGATGCTTCTCTTTGGTTTAGATTATTAAGACAACATGGA
TTTAATGTTTCATCAAGTATATTTGAAAAATTCAAAGACGAGGAAGGTAACTTTAAAGAA
AGCCTAATAACYGATGTTCCAGGTTTGCTTAGCTTGTATGAGGCTTCGCACTTGAGTTAT
GTTGGAGAAAGCATACTAGATGAAGCACTTGCTTTCACAACCACTCACCTTAAGGCTATT
GTGGCAAATAGTAAAGATCATCCATTATCACATCAAATATCCATAGCCTTGCATAGGCCT
CTAAGAAAGACCATAGAGAGGCTTCATGCTAGGTTTTACATCTCAATCTATGAAAAGGAT
GCCTCTCATAACAAACTATTGCTAGAGCTTGCAAAGTTAGACTTCAATCTACTTCAATGT
TTCCACAAAAAGGAACTTAGTGAAATTACGAGGTGGTGGAAGGAGCATGAGTTTGCAAAG
AAATTCCCTTTTGCAAGAGATAGGATGGTGGAACTGTATTTTTGGATATTGGGTGTATAT
TATGAACCCAAATACTCTCGAGCAAGGAAGCTTTTAACCAAAGTCATTGCATTGACCTCA
ATCACTGATGATATTTATGATGCATATGGTACTATTGATGAGCTTCAGCTTCTTACCAAA
GCAATTCAAAGGTGGGACATAAATTGTATGGATAAACTTAAGCAAGAATATTTAAAGACA
TATTATAAGGTAATGTTGGATTCTTATGAAGAATTTGAAAAGGAGCTTAAAAAGGAAGAA
TTATACAAACTTGAGTATGCAAAAGAAGAGATGAAAAGAATTATTGGAGGTTATTTTGAA
GAAGCTCGATGGTTGAATGAAGGATATTTCCCAAGCTTCGATGAGCATTTGAGAGTCTCT
TATGTTTCTTCTGGTAACGTTTTGTTGATAGCCACAAGTTTTGTAGGGATGCATGATGTT
GTAACACATGAAACTCTAGATTGGCTCTCCAAAGACCCTAAAATTGTTTCAGCTTCCACT
CTCCTTKCTAGGTTCATGGATGATATTGGTTCTCGCAAGTTTGAGCAAAAGAGAAATCAC
ATACCATCTACAGTGGATTGTTACATGAAACAATATGGGGTATCAGAGGAAGAGGCAATT
AAAGAACTTAATAAAAGAGTGGACACCCACTGGAAAGAAATTAATGAAGACTTTATTAGG
CCAGCAGTTGTGCCCTTTCCTATCTTAGTTCGTGTTCTTAATTTTACAAAAATAGTAGAT
CTTCTTTACAAAGAGGGCGATGATCAATACACAAATGTTGGAAAAGTGCTCAAAGAAAGC
ATTGCTGCTTTGCTTATAGATTCAATCCCATTATAA (SEQ ID NO: 1407)

TPS20_LPA5_mRNA:1-1656

ATGTCAAATATTCAAGTCTTAGCTTCATCTCAATTAAGTGACAAAATTATTGCTCGTCCA
ACAACAAAATTTCACCCTTCTATTTGGGGTGATCGATTCCTCCATTACAATATTTCAGAA
CAAGACTTGGTTTGCAAACAAGAAAAAGTTGAAGAATTAATACAAGTTGTAAAGAAAGAG
ATATTATCTTCAAATCATGATCAATTGAAGTTGATTGACAATCTCCAACGTTTGGGATTA
TCATATCATTTTGAGAGTGAAATTGAGAAATTGTTGGAACAATTAAGTACCACTCATCAT
CAAAATCATCAAGATCTACATGATGCTTCTCTTTGGTTTAGATTATTAAGACAACATGGA
TTTAATGTTTCATCAAGTATATTTGAAAAATTCAAAGACGAGGAAGGTAACTTTAAAGAA
AGCCTAATAACYGATGTTCCAGGTTTGCTTAGCTTGTATGAGGCTTCGCACTTGAGTTAT
GTTGGAGAAAGCATACTAGATGAAGCACTTGCTTTCACAAACACTCACCTTAAGGCTATT
GTGGCAAATAGTAAAGATCATCCATTATCACATCAAATATCCATAGCCTTGCATAGGCCT
CTAAGAAAGACCATAGAGAGGCTTCATGCTAGGTTTTACATCTCAATCTATGAAAAGGAT
GCCTCTCATAACAAACTATTGCTAGAGCTTGCAAAGTTAGACTTCAATCTACTTCAATGT
TTCCACAAAAAGGAACTTAGTGAAATTACGAGGTGGTGGAAGGAGCATGAGTTTGCAAAG
AAATTCCCTTTTGCAAGAGATAGGATGGTGGAACTGTATTTTTGGATATTGGGTGTATAT
TATGAACCCAAATACTCTCGAGCAAGGAAGCTTTTAACCAAAGTCATTGCATTGACCTCA
ATCACTGATGATATTTATGATGCATATGGTACTATTGATGAGCTTCAGCTTCTTACCAAA
GCAATTCAAAGGTGGGACATAAATTGTATGGATAAACTTAAGCAAGAATATTTAAAGACA
TATTATAAGGTAATGTTGGATTCTTATGAAGAATTTGAAAAGGAGCTTAAAAAGGAAGAA
TTATACAAACTTGAGTATGCAAAAGAAGAGATGAAAAGAATTATTGGAGGTTATTTTGAA
GAAGCTCGATGGTTGAATGAAGGATATTTCCCAAGCTTCGATGAGCATTTGAGAGTCTCT
TATGTTTCTTCTGGTAACGTTTTGTTGATAGCCACAAGTTTTGTAGGGATGCATGATGTT
GTAACACATGAAACTCTAGATTGGCTCTCGAAAGACCCTAAAATTGTTTCAGCTTCTACT
CTCCTTTCTAGGTTCATGGATGATATTGGTTCTCGCAAGTTTGAGCAAAAGAGAAATCAC
ATACCATCTACAGTGGATTGTTACATGAAACAATATGGGGTATCAGAGGAAGAGGCAATT
AAAGAACTTAATAAAAGAGTGGACACCCACTGGAAAGAAATTAATGAAGACTTTATTAGG
CCAGCAGTTGTGCCCTTTCCTATCTTAGTTCGTGTTCTTAATTTTACAAAAATAGTAGAT
CTTCTTTACAAAGAGGGCGATGATCAATACACAAATGTTGGAAAAGTGCTCAAAGAAAGC
ATTGCTGCTTTGCTTATAGATTCAATCCCATTATAA (SEQ ID NO: 1408)

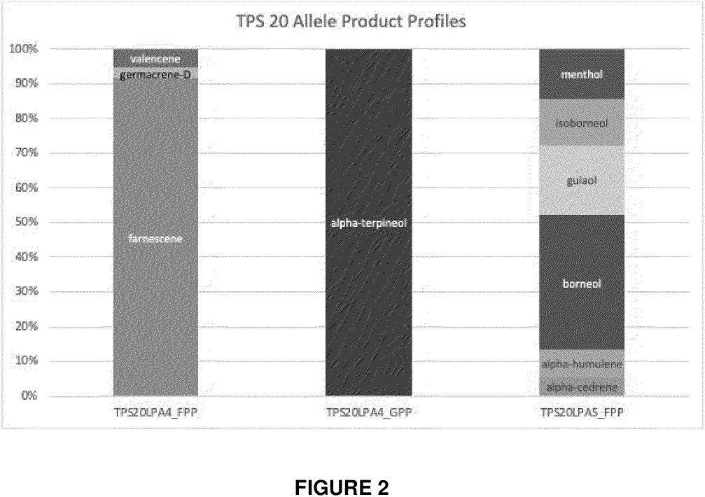

Despite the close similarity in sequences, the terpene product profiles produced by each of these alleles are different, as shown in FIG. 2 (FPP = farnesyl pyrophosphate as precursor; GPP =geranyl pyrophosphate as precursor). Only one allele, TPS20_LPA5 (LPA005) produces the insecticidal terpene, guaiol. The methods provided herein permit specific identification/detection of the guaiol producing allele in a plant, thereby permitting selection of a plant cultivar having a desired insecticidal profile. It may be possible, in embodiments, to modify a plant cultivar containing the LPA004 allele using genetic engineering, e.g., by swapping exons 1, 2, 3, and 6 of the LPA004 allele with the LPA005 versions of these exons to, e.g., produce guaiol, menthol, isoborneol, borneol, alpha-humulene, and alpha-cedrene instead of valencene, germacrene-B and farnescene.

TABLE 14

Primer Group-Target: Primer Group 17 - CsTPS29BC
Characterized Seq:MN967468
Assessment: Primers TPS43JL-1 F/R (SEQ ID NOs 631 (F) and 632 (R)), 2aF/R (SEQ ID NOs 633 (F) and 634 (R)), 2bF/R (SEQ ID NOs 635 (F) and 636 (R)), 3F/R (SEQ ID NOs 637 (F) and 638 (R)), and 4F/R (SEQ ID NOs 639 (F) and 640 (R)) are specific to MN967468

| Primer Set | Gene | Gene Position Start | Amplicon Length |
|---|---|---|---|
| TPS43JL-1F/R_amp_1 | TPS43JL | 28 | 204 |
| TPS43JL-1F/R_amp_2 | XM_030628902.1 | 265 | 204 |
| TPS43JL-1F/R_amp_3 | IcI\|MN967468.1_cds_QLC36833.1_1 | 265 | 204 |
| TPS43JL-1F/R_amp_4 | IcI\|NC_044379.1_cds_XP_030484762.1 25100 | 265 | 204 |
| TPS43JL-2aF/R_amp_1 | TPS43JL | 245 | 136 |
| TPS43JL-2aF/R_amp_2 | XM_030628902.1 | 482 | 136 |
| TPS43JL-2aF/R_amp_3 | IcI\|MN967468.1_cds_QLC36833.1_1 | 482 | 136 |
| TPS43JL-2aF/R_amp_4 | IcI\|NC_044379.1_cds_XP_030484762.1_25100 | 482 | 136 |
| TPS43JL-2bF/R_amp_1 | TPS43JL | 321 | 244 |
| TPS43JL-3F/R_amp_1 | TPS43JL | 654 | 174 |
| TPS43JL-3F/R_amp_2 | XM_030628902.1 | 891 | 174 |
| TPS43JL-3F/R_amp_3 | IcI\|MN967468.1_cds_QLC36833.1_1 | 891 | 174 |
| TPS43JL-3F/R_amp_4 | IcI\|NC_044379.1_cds_XP_030484762.1_25100 | 891 | 174 |
| TPS43JL-4F/R_amp_1 | TPS43JL | 835 | 88 |
| TPS43JL-4F/R_amp_2 | XM_030628902.1 | 1072 | 88 |
| TPS43JL-4F/R_amp_3 | IcI\|MN967468.1_cds_QLC36833.1_1 | 1072 | 88 |
| TPS43JL-4F/R_amp_4 | IcI\|NC_044379.1_cds_XP_030484762.1_25100 | 1072 | 88 |
| TPS43JL-5F/R_amp_1 | TPS43JL | 1072 | 159 |
| TPS43JL-5F/R_amp_2 | XM_030628902.1 | 1309 | 159 |
| TPS43JL-5F/R_amp_3 | IcI\|MN967468.1_cds_QLC36833.1 1 | 1309 | 159 |
| TPS43JL-5F/R_amp_4 | IcI\|NC_044379.1_cds_XP_030484762.1_25100 | 1309 | 159 |
| TPS43JL-6F/R_amp_1 | TPS43JL | 1264 | 145 |

| | |
|---|---|
| Main Priduct(s) | Side Product(s) |
| Linalool/Nerolidol | |

TABLE 15

Primer Group-Target: Primer Group 18 - CsTPS1 7AK
Characterized Seq: MN967470
Assessment: Primers TPS51JL-1F/R (SEQ ID NOs 827 (F) and 828 (R)), 2F/R (SEQ ID NOs 829 (F) and 830 (R)), 3aF/R (SEQ ID NOs 831 (F) and 832 (R)), and 3bF/R (SEQ ID NOs 833 (F) and 834 (R)) are specific to MN967470

| Primer Set | Gene | Gene Position Start | Amplicon Length |
|---|---|---|---|
| TPS51JL-1F/R_amp_1 | MSTRG.26703.2 | 91 | 50 |
| TPS51JL-1F/R_amp_2 | TPS51JL | 33 | 50 |
| TPS51JL-1F/R_amp_3 | IcI\|MN967470.1_cds_QLC36835.1_1 | 63 | 50 |
| TPS51JL-2F/R_amp_1 | MSTRG.26703.2 | 188 | 202 |
| TPS51JL-2F/R_amp_2 | TPS51JL | 130 | 202 |
| TPS51JL-2F/R_amp_3 | IcI\|MN967470.1_cds_QLC36835.1_1 | 160 | 202 |
| TPS51JL-2F/R_amp_4 | IcI\|NW_022060378.1_cds_XP 030485348.1_33426 | 160 | 202 |
| TPS51JL-2F/R_amp_5 | IcI\|NW_022060378.1_cds_XP_030485349.1_33424 | 160 | 202 |
| TPS51JL-2F/R_amp_6 | IcI\|NW_022060378.1_cds_XP_030485350.1_33425 | 160 | 202 |
| TPS51JL-3aF/R_amp_1 | MSTRG.26703.2 | 450 | 104 |
| TPS51JL-3aF/R_amp_2 | TPS51JL | 392 | 104 |
| TPS51JL-3aF/R_amp_3 | IcI\|MN967470.1_cds_QLC36835.1_1 | 422 | 104 |
| TPS51JL-3aF/R_amp_4 | IcI\|NW_022060378.1_cds_XP_030485348.1_33426 | 425 | 104 |
| TPS51JL-3aF/R_amp_5 | IcI\|NW_022060378.1_cds_XP_030485349.1_33424 | 422 | 104 |
| TPS51JL-3aF/R_amp_6 | IcI\|NW_022060378.1_cds_XP_030485350.1_33425 | 425 | 104 |
| TPS51JL-3bF/R_amp_1 | MSTRG.26703.2 | 529 | 184 |
| TPS51JL-3bF/R_amp_2 | TPS51JL | 471 | 184 |
| TPS51JL-3bF/R_amp_3 | IcI\|MN967470.1_cds_QLC36835.1_1 | 501 | 184 |
| TPS51JL-3bF/R_amp_4 | IcI\|NW_022060378.1_cds XP_030485348.1_33426 | 504 | 184 |
| TPS51JL-3bF/R_amp_5 | IcI\|NW_022060378.1_cds XP_030485349.1_33424 | 501 | 184 |
| TPS51JL-3bF/R_amp_6 | IcI\|NW_022060378.1_cds XP_030485350.1_33425 | 504 | 184 |
| TPS51JL-5F/R_amp_1 | MSTRG.26703.2 | 1053 | 124 |
| TPS51JL-5F/R_amp_2 | TPS51JL | 995 | 124 |
| TPS51JL-6aF/R_amp_1 | MSTRG.26703.2 | 1220 | 100 |
| TPS51JL-6aF/R_amp_2 | TPS51JL | 1162 | 100 |

| | |
|---|---|
| Main Product(s) | Side Product(s) |
| Myrcene & Linalool | geraniol (16%), (E)-β-ocimene (8%) and α-terpineol (9%) |

TABLE 16

Primer Group 19: TPS33PK (KY624371) - PAV/HRM Primers - gDNA/cDNA HRM/PAV Primer Set

| | Sequence (5′->3′) | Temple te strand | Length | Start | Stop | Tm | GC% | Self complem entarity | Self 3′ complem entarity |
|---|---|---|---|---|---|---|---|---|---|
| Forward primer | TGGATTTCAGT AGGAGCACCA (SEQ ID NO: 1409) | Plus | 21 | 92 | 112 | 58.73 | 47.62 | 4.00 | 2.00 |
| Reverse primer | ATTCCAAGCAT TCGAAAATCTC TT (SEQ ID NO: 1410) | Minus | 24 | 197 | 174 | 57.48 | 33.33 | 6.00 | 2.00 |
| Product length | 106 | | | | | | | | |

Tables 17-35 provide sets of primers for identifying/selecting plant cultivars having desired energetic, anti-nociceptive and/or insecticidal profiles by LAMP assay. The B3 and F3 primers of the LAMP assay also can be used as polynucleotide primer pairs, e.g., in PCR, qPCR or HRM assays to detect the presence of a desired terpene synthase gene or allele.

LAMP primers designated as “common” amplify a gene rather than a particular characterized allele. For example, “Primer Group 5: TPS31 PK-common exon 3 LAMP primers” can amplify TPS31Cs10, TPS31 PK, and other very similar TPS31 alleles, whereas “Primer Group 5: TPS31 PK-specific exon 3 LAMP primers” only amplifies the PK-type allele of TPS31 (denoted as specific or unique). The goal of this is to cover both possibilities of (1) whether all TPS31 genes made the same product and selecting for any allele of TPS31 would allow you to select for a cultivars producing a desired terpene or set of terpenes (e.g., terpinolene in this case), or (2) if only the PK-type allele of TPS31 (allele that’s been functionally characterized) makes terpinolene and then other alleles, like TPS31Cs10, make different product profiles. The FIP and BIP primers can, in embodiments, include a poly T linker linking the F2 and F1c primers (FIP) and/or the B2 and B1c primers (BIP) (i.e., the hyphens shown in FIP and BIP can be a poly T linker or can directly connect the two primers). The length of the poly T linker, when present, can be from about 3 Ts to about 15 Ts, generally between about 4 Ts to about 6 Ts, or about 3, 4, 5, 6, 7, 8, 9, 10, 11, 12, 13, 14 or 15 Ts.

TABLE 17

LAMP Assay Designs for Primer Group 6 (Terpinolene)
Primer Group 6: TPS37FN - 1638-1846 - Exon 7 - gDNA/cDNA LAMP Design:

| 1 | ID:1 | | dimer(minimum)dG=-1.67 | | | | | |
|---|---|---|---|---|---|---|---|---|
| label | 5′pos | 3′pos | len | Tm | 5′dG | 3′dG | GCrate | Sequence |
| F3 | 13 | 32 | 20 | 55.81 | -4.69 | -4.34 | 0.40 | AAGAGGAGATGTTCCGAAAT (SEQ ID NO: 1411) |
| B3 | 221 | 239 | 19 | 55.87 | -4.60 | -4.76 | 0.47 | TCTGAGAAGCATGTCCATC (SEQ ID NO: 1412) |
| FIP | | | 45 | | | | | TCAAAAATTTGATGTGCTCGCGA-AATGTTACATGAACGATACAGG (SEQ ID NO: 1413) |
| BIP | | | 50 | | | | | CTCGAAAGAAGTTGTTCATGTTTGC-CCATAAAGGTATATAAATTTGGCTG (SEQ ID NO: 1414) |
| F2 | 39 | 60 | 22 | 55.81 | -3.71 | -4.58 | 0.36 | AATGTTACATGAACGATACAGG (SEQ ID NO: 1415) |
| F1c | 79 | 101 | 23 | 61.84 | -3.69 | -6.69 | 0.39 | TCAAAAATTTGATGTGCTCGCGA (SEQ ID NO: 1416) |
| B2 | 195 | 219 | 25 | 55.95 | -3.74 | -6.25 | 0.32 | CCATAAAGGTATATAAATTTGGCTG (SEQ ID NO: 1417) |
| B1c | 154 | 178 | 25 | 61.22 | -5.04 | -5.17 | 0.40 | CTCGAAAGAAGTTGTTCATGTTTGC (SEQ ID NO: 1418) |

Primer Group 6: TPS37FN - 1400-1626 - Exon 6 - gDNA/cDNA LAMP Design:

| ID:25 | | dimer(minimum)dG:::-2.28 | | | | | | |
|---|---|---|---|---|---|---|---|---|
| label | 5′pos | 3′pos | len | Tm | 5′dG | 3′dG | GCrate | Sequence |
| F3 | 84 | 105 | 22 | 55.82 | -2.98 | -5.00 | 0.36 | AGAATACATGGATTTCAGTTGG (SEQ ID NO: 1419) |
| B3 | 292 | 310 | 19 | 57.80 | -6.37 | -5.25 | 0.53 | CGACGATGTTCCTAGGTCA (SEQ ID NO: 1326) |
| FIP | | | 47 | | | | | GGATCATCATAACCTTCTTCCAAGATCTTTTGCATGCTTATTTTGCT (SEQ ID NO: 1287) |
| BIP | | | 47 | | | | | TGAAGGATCCCTGGAAATATCAAATTCTTCAAGTCGTAAAAGTATGG (SEQ ID NO: 1288) |
| F2 | 118 | 139 | 22 | 57.40 | -3.52 | -4.91 | 0.32 | TCTTTTGCATGCTTATTTTGCT (SEQ ID NO: 1289) |
| F1c | 174 | 198 | 25 | 60.23 | -4.76 | -4.86 | 0.40 | GGATCATCATAACCTTCTTCCAAGA (SEQ ID NO: 1290) |
| B2 | 270 | 291 | 22 | 55.41 | 4.27 | -4.23 | 0.36 | TCTTCAAGTCGTAAAAGTATGG (SEQ ID NO: 1291) |
| B1c | 214 | 238 | 25 | 60.09 | -4.86 | -3.57 | 0.36 | TGAAGGATCCCTGGAAATATCAAAT (SEQ ID NO: 1292) |
| LF | 142 | 166 | 25 | 60.48 | -5.70 | -4.33 | 0.40 | GGAAGCTTTTTCTAAGGGATTTGTG (SEQ ID NO: 1293) |

TABLE 18

LAMP Assay Designs for Primer Group 1 (a-pinene > β-pinene)
Primer Group 1: TPS2SK - 384-781 - Exon 3 - gDNA/cDNA LAMP Design - degenerate; common to SK and FN

| | dimer(minimum)dG=-2.16 | | | | | | | |
|---|---|---|---|---|---|---|---|---|
| label | 5′pos | 3′pos | len | Tm | 5′dG | 3′dG | GCrate | Sequence |
| F3 | 62 | 85 | 24 | 55.69 | -2.63 | -4.09 | 0.33 | GATATTAAGGGAGTATTGGCTTTA (SEQ ID NO: 1420) |
| B3 | 275 | 297 | 23 | 55.73 | -3.03 | -5.00 | 0.30 | TCATATTCTTCAATGAACCACTT (SEQ ID NO: 1421) |
| FIP | | | 48 | | | | | TGAGATATTCTGTTGTGAAAACCCTTTCATTCTATGTGAAAAATGGYG (SEQ ID NO: 1422) |
| BIP | | | 46 | | | | | TATGGCAATATTAGTGAGACATGCCGCTTCTGCTCTTATAGTCCTC (SEQ ID NO: 1423) |
| F2 | 94 | 116 | 23 | 57.66 | -3.57 | -6.57 | 0.35 | TTCATTCTATGTGAAAAATGGYG (SEQ ID NO: 1424) |
| Flc | 137 | 161 | 25 | 60.32 | -4.15 | -5.34 | 0.36 | TGAGATATTCTGTTGTGAAAACCCT (SEQ ID NO: 1425) |
| B2 | 253 | 273 | 21 | 56.84 | --5.09 | --5.20 | 0.48 | GCTTCTGCTCTTATAGTCCTC (SEQ ID NO: 1426) |
| B1c | 208 | 232 | 25 | 60.81 | -4.98 | -5.90 | 0.40 | TATGGCAATATTAGTGAGACATGCC (SEQ ID NO: 1427) |

Primer Group 1: TPS2SK - 1302-1549 - Exon 7 - gDNA/cDNA LAMP Design - degenerate; common to SK and FN

| | | | | | | | | |
|---|---|---|---|---|---|---|---|---|
| 1 | ID:9 | | dimer(minimum)dG=-2.38 | | | | | |
| label | 5′pos | 3′pos | len | Tm | 5′dG | 3′dG | GCrate | Sequence |
| F3 | 25 | 43 | 19 | 55.35 | -4.53 | -4.16 | 0.42 | TTTGGAAGAGGCAAAATGG (SEQ ID NO: 1428) |
| B3 | 220 | 239 | 20 | 55.53 | -3.80 | -4.67 | 0.45 | GATCATCTGCAAGTCGTAAC (SEQ ID NO: 1429) |
| FIP | | | 41 | | | | | CCTATTGAAAGCGAGGCGTT-YTATAGCGGATACAAACCAAC (SEQ ID NO: 1430) |
| BIP | | | 42 | | | | | ACAAAGTCCATAACAAAYGAGGCCAAGGATCCTTGGCGACAT (SEQ ID NO: 1431) |
| F2 | 46 | 66 | 21 | 55.24 | -2.31 | -5.16 | 0.38 | YTATAGCGGATACAAACCAAC (SEQ ID NO: 1432) |
| F1c | 86 | 105 | 20 | 60.15 | -3.57 | --6.68 | 0.50 | CCTATTGAAAGCGAGGCGTT (SEQ ID NO: 1433) |
| B2 | 199 | 216 | 18 | 57.68 | -4.24 | -5.23 | 0.50 | AAGGATCCTTGGCGACAT (SEQ ID NO: 1434) |
| B1c | 143 | 166 | 24 | 61.55 | -4.16 | -6.54 | 0.42 | ACAAAGTCCATAACAAAYGAGGCC (SEQ ID NO: 1435) |
| LB | 176 | 195 | 20 | 60.12 | -4.65 | -5.42 | 0.50 | TTGCAAGAGGGTCATYACGC (SEQ ID NO: 1436) |

TABLE 19

LAMP Assay Designs for Primer Group 3 (a-pinene > β-pinene)
Primer Group 3: TPS30PK - Exon 2 - - gDNA/cDNA LAMP Design

| 2 | ID:1 | | dimer(minimum)dG=-1.74 | | | | | |
|---|---|---|---|---|---|---|---|---|
| label | 5′pos | 3′pos | len | Tm | 5′dG | 3′dG | GCrate | Sequence |
| F3 | 2 | 19 | 18 | 55.97 | -5.30 | -5.92 | 0.50 | GGAAGGAGCATATGCCAA (SEQ ID NO: 1437) |
| B3 | 219 | 241 | 23 | 56.58 | -3.82 | -5.40 | 0.35 | AGTCTAAATTCAAGAGAATTGGC (SEQ ID NO: 1438) |
| FIP | | | 45 | | | | | GCTTCTCCTCCTCTTTTGCTCTCGAGTTGAGAAAGTAAAGGAAGA (SEQ ID NO: 1439) |
| BIP | | | 48 | | | | | AAGACTTGGAATCTCTTACCACTTT-GTGTTGTACACATTATTGTTGTT (SEQ ID NO: 1440) |
| F2 | 22 | 43 | 22 | 55.05 | -4.51 | -4.71 | 0.36 | GAGTTGAGAAAGTAAAGGAAGA (SEQ ID NO: 1441) |
| F1c | 62 | 84 | 23 | 62.60 | -5.09 | -5.44 | 0.52 | GCTTCTCCTCCTCTTTTGCTCTC (SEQ ID NO: 1442) |
| B2 | 180 | 202 | 23 | 55.55 | -4.82 | -4.32 | 0.30 | GTGTTGTACACATTATTGTTGTT (SEQ ID NO: 1443) |
| B1c | 119 | 143 | 25 | 60.05 | -4.24 | -4.16 | 0.36 | AAGACTTGGAATCTCTTACCACTTT (SEQ ID NO: 1444) |

Primer Group 3: TPS30PK - Exon 2 - - gDNA/cDNA LAMP Design

| ID:5 | | dimer(minimum)dG=-1.87 | | | | | | |
|---|---|---|---|---|---|---|---|---|
| label | 5′pos | 3′pos | len | Tm | 5′dG | 3′dG | GCrate | Sequence |
| F3 | 10 | 30 | 21 | 55.48 | -2.85 | -4.27 | 0.38 | CATATGCCAAAAGAGTTGAGA (SEQ ID NO: 1445) |
| B3 | 203 | 225 | 23 | 55.78 | -4.16 | -4.33 | 0.30 | AATTGGCATACACATTATTGTTG (SEQ ID NO: 1446) |
| FIP | | | 46 | | | | | TCAAGTTGAGATAAAGGCTTCTCC-AGGAAGAGGTAAGAGTAATGGT (SEQ ID NO: 144′7) |
| BIP | | | 48 | | | | | AAGACTTGGAATCTCTTACCACTTT-GTGTTGTACACATTATTGTTGTT (SEQ ID NO: 1440) |
| F2 | 37 | 58 | 22 | 57.59 | -4.69 | -4.55 | 0.41 | AGGAAGAGGTAAGAGTAATGGT (SEQ ID NO: 1448) |
| F1c | 77 | 100 | 24 | 60.26 | -4.41 | -4.71 | 0.42 | TCAAGTTGAGATAAAGGCTTCTCC (SEQ ID NO: 1449) |
| B2 | 180 | 202 | 23 | 55.55 | -4.82 | -4.32 | 0.30 | GTGTTGTACACATTATTGTTGTT (SEQ ID NO: 1443) |
| B1c | 119 | 143 | 25 | 60.05 | -4.24 | -4.16 | 0.36 | AAGACTTGGAATCTCTTACCACTTT (SEQ ID NO: 1444) |

TABLE 20

LAMP Assay Designs for Primer Group 12 (β-ocimene producing; Accession No. KY014563)
Primer Group 12: TPS6FN - 1224-1495 - Exon 6 - gDNA/cDNA LAMP Design

| | dimer(minimum)dG=-2.46 | | | | | | | |
|---|---|---|---|---|---|---|---|---|
| label | 5′pos | 3′pos | len | Tm | 5′dG | 3′dG | GCrate | Sequence |
| F3 | 4 | 26 | 23 | 56.28 | -6.04 | -4.01 | 0.35 | GGTCGATATGTGTAAAAGTTTCT (SEQ ID NO: 1450) |
| B3 | 218 | 235 | 18 | 55.12 | -4.98 | -4.73 | 0.44 | TATGGCACTGTGACGAAT (SEQ ID NO: 1451) |
| FIP | | | 45 | | | | | TCCTACTGAAATCCAACCATTTT CA---GCAAGAGGCAAAATGGTACT (SEQ ID NO: 1452) |
| BIP | | | 45 | | | | | CTTGTGCATGCTTATTTTTTTCA CG-AACCATATTCCAAGCATTCG (SEQ ID NO: 1453) |
| F2 | 28 | 47 | 20 | 57.95 | -5.26 | -4.57 | 0.45 | GCAAGAGGCAAAATGGTACT (SEQ ID NO: 1454) |
| F1c | 82 | 106 | 25 | 60.59 | -4.43 | -3.69 | 0.36 | TCCTACTGAAATCCAACCATTTT CA (SEQ ID NO: 1455) |
| B2 | 184 | 203 | 20 | 55.57 | -4.55 | -4.84 | 0.40 | AACCATATTCCAAGCATTCG (SEQ ID NO: 1456) |
| B1c | 119 | 143 | 25 | 60.29 | -4.66 | -5.35 | 0.36 | CTTGTGCATGCTTATTTTTTTCA CG (SEQ ID NO: 1457) |
| LF | 51 | 73 | 23 | 60.92 | -4.53 | -4.90 | 0.43 | TTCCAATGTTGGTGTGTATCCAC (SEQ ID NO: 1458) |

Primer Group 12: TPS6FN - 1224-1495 - Exon 6 - gDNA/cDNA LAMP Design

| ID:5 | | dimer(minimum)dG=-2.33 | | | | | | |
|---|---|---|---|---|---|---|---|---|
| label | 5′pos | 3′pos | len | Tm | 5′dG | 3′dG | GCrate | Sequence |
| F3 | 20 | 37 | 18 | 55.97 | -4.01 | -6.10 | 0.44 | AGTTTCTTGCAAGAGGCA (SEQ ID NO: 1459) |
| B3 | 219 | 236 | 18 | 55.35 | -3.74 | -5.20 | 0.44 | TTATGGCACTGTGACGAA (SEQ ID NO: 1460) |
| FIP | | | 50 | | | | | TCCTACTGAAATCCAACCATTTTCAAAATGGTACTATAGTGGATACACAC (SEQ ID NO: 1461) |
| BIP | | | 45 | | | | | CTTGTGCATGCTTATTTTTTTCACG-ACCATATTCCAAGCATTCGA (SEQ ID NO: 1462) |
| F2 | 38 | 62 | 25 | 57.91 | -4.16 | -5.21 | 0.36 | AAATGGTACTATAGTGGATACACAC (SEQ ID NO: 1463) |
| F1c | 82 | 106 | 25 | 60.59 | -4.43 | -3.69 | 0.36 | TCCTACTGAAATCCAACCATTTTCA (SEQ ID NO: 1455) |
| B2 | 183 | 202 | 20 | 56.53 | -4.13 | -4.59 | 0.40 | ACCATATTCCAAGCATTCGA (SEQ ID NO: 1464) |
| B1c | 119 | 143 | 25 | 60.29 | -4.66 | -5.35 | 0.36 | CTTGTGCATGCTTATTTTTTTCACG (SEQ ID NO: 1457) |

TABLE 21

LAMP Assay Design for Primer Group 13 (Accession No. KY014558)
Primer Group 13: TPS13PK - Exon 6 - Degenerate - gDNA/cDNA LAMP Design

| label | 5′pos | 3′pos | len | Tm | 5′dG | 3′dG | GCrate | Sequence |
|---|---|---|---|---|---|---|---|---|
| F3 | 15 | 39 | 25 | 56.76 | -4.56 | -5.24 | 0.32 | GCAAATCTTATTTAAGAGAAGCAAG (SEQ ID NO: 1465) |
| B3 | 207 | 228 | 22 | 55.03 | -4.18 | -4.06 | 0.36 | CGTAAAATCATRGATGAATGTC (SEQ ID NO: 1466) |
| FIP | | | 49 | | | | | GGYCCTGATATTGATATCCATGCATATGGTATTATAATGGATACACACC (SEQ ID NO: 1467) |
| BIP | | | 48 | | | | | TTGTAGTGAATCCAAWCAAGGAARAGTATTATGGTAGGGTATCCATCA (SEQ ID NO: 1468) |
| F2 | 40 | 63 | 24 | 55.76 | -4.13 | -5.66 | 0.33 | ATGGTATTATAATGGATACACACC (SEQ ID NO: 1469) |
| F1c | 87 | 111 | 25 | 60.49 | -5.69 | -4.41 | 0.40 | GGYCCTGATATTGATATCCATGCAT (SEQ ID NO: 1470) |
| B2 | 184 | 206 | 23 | 56.36 | -2.47 | -4.91 | 0.39 | GTATTATGGTAGGGTATCCATCA (SEQ ID NO: 1471) |
| B1c | 138 | 162 | 25 | 60.09 | -3.74 | -4.71 | 0.36 | TTGTAGTGAATCCAAWCAAGGAARA (SEQ ID NO: 1472) |

TABLE 22

LAMP Assay Designs for Primer Group 2
Primer Group 2: TPS5FN - Specific from TPS5PK - - gDNA/cDNA LAMP Design

| | | | dimer(minimum)dG=-2.18 | | | | | |
|---|---|---|---|---|---|---|---|---|
| label | 5′pos | 3′pos | len | Tm | 5′dG | 3′dG | GCrate | Sequence |
| F3 | 206 | 227 | 22 | 55.03 | -2.63 | -4.58 | 0.32 | ATTATCATCATCATGGTCAACA (SEQ ID NO: 1473) |
| B3 | 378 | 398 | 21 | 56.77 | -5.43 | -4.18 | 0.38 | AGACGAGAAAAAGGAGTTCAA (SEQ ID NO: 1474) |
| FIP | | | 43 | | | | | GATGAAGCAAGAGATTTCACAACCATTGACCTTGCCTCGTCAT (SEQ ID NO: 1475) |
| BIP | | | 47 | | | | | TATGGGTTCACCCCTAAATGAATAGGGAGAGTTTGAGTAAAGATGTG (SEQ ID NO: 1476) |
| F2 | 230 | 247 | 18 | 57.86 | -5.02 | -5.23 | 0.50 | TTGACCTTGCCTCGTCAT (SEQ ID NO: 1477) |
| F1c | 271 | 295 | 25 | 61.73 | -3.92 | -5.17 | 0.40 | GATGAAGCAAGAGATTTCACAACCA (SEQ ID NO: 1478) |
| B2 | 356 | 377 | 22 | 56.00 | -5.04 | -4.56 | 0.41 | GGAGAGTTTGAGTAAAGATGTG (SEQ ID NO: 1479) |
| B1c | 299 | 323 | 25 | 60.07 | -4.58 | -3.08 | 0.40 | TATGGGTTCACCCCTAAATGAATAG (SEQ ID NO: 1480) |
| LB | 329 | 353 | 25 | 60.75 | -5.93 | -4.24 | 0.40 | AGCCTCATATAGACATACCATTCCT (SEQ ID NO: 1.481) |

Primer Group 2: TPSSFN — Specific from TPS5PK - - gDNA/cDNA LAMP Design

| | | | dimer(minimum)dG=-2.02 | | | | | |
|---|---|---|---|---|---|---|---|---|
| label | 5′pos | 3′pos | len | Tm | 5′dG | 3′dG | GCrate | Sequence |
| F3 | 224 | 242 | 19 | 55.32 | -3.71 | -6.10 | 0.42 | AACATTTTGACCTTGCCTC (SEQ ID NO: 1.482) |
| B3 | 394 | 414 | 21 | 55.25 | -3.69 | -5.18 | 0.33 | TTTTCAATGCTTTCAAAGACG (SEQ ID NO: 1483) |
| FIP | | | 49 | | | | | AGGGGTGAACCCATATTAGATGAAGGTCATCATCAAGTATTGTTTGAGA (SEQ ID NO: 1484) |
| BIP | | | 47 | | | | | AGCCTCATATAGACATACCATTCCTAGAAAAAGGAGTTCAAGGAGAG (SEQ ID NO: 1485) |
| F2 | 243 | 266 | 24 | 56.79 | -4.41 | -4.27 | 0.33 | GTCATCATCAAGTATTGTTTGAGA (SEQ ID NO: 1486) |
| F1c | 289 | 313 | 25 | 61.91 | -6.18 | -3.90 | 0.44 | AGGGGTGAACCCATATTAGATGAAG (SEQ ID NO: 1487) |
| B2 | 372 | 393 | 22 | 57.39 | -3.52 | -5.04 | 0.41 | AGAAAAAGGAGTTCAAGGAGAG (SEQ ID NO: 1488) |
| B1c | 329 | 353 | 25 | 60.75 | -5.93 | -4.24 | 0.40 | AGCCTCATATAGACATACCATTCCT (SEQ ID NO: 1481) |

TABLE 23

LAMP Assay Design for Primer Group 4 (Terpinolene and only α-pinene producing)
Primer Group 4: TPS32PK — Exon 7 — 1425-1728 — Unique to TPS32PK - - gDNA/cDNA LAMP Design

| 3 | ID:3 | | dimer(minimum)dG=-2.16 | | | | | |
|---|---|---|---|---|---|---|---|---|
| label | 5′pos | 3′pos | len | Tm | 5′dG | 3′dG | GCrate | Sequence |
| F3 | 22 | 43 | 22 | 56.09 | -4.18 | -4.74 | 0.36 | GAACAAGGAAATGATTCCATCT (SEQ ID NO: 1489) |
| B3 | 195 | 219 | 25 | 56.26 | -4.67 | -4.91 | 0.32 | GAACACATAAGGAATTTAATCAGTG (SEQ ID NO: 1490) |
| FIP | | | 50 | | | | | TTCGTTAGGACAGCAATGATGATAT-GTAATGATCAAAATTAGTACCAACC (SEQ ID NO: 1491) |
| BIP | | | 47 | | | | | ATGGCAGTTTCAATAAAAGCTTGAG-CATGGAAGGAAATGAATGAAGC (SEQ ID NO: 1492) |
| F2 | 49 | 73 | 25 | 56.04 | -3.39 | -5.61 | 0.32 | GTAATGATCAAAATTAGTACCAACC (SEQ ID NO: 1493) |
| F1c | 93 | 117 | 25 | 60.23 | -4.85 | -3.03 | 0.36 | TTCGTTAGGACAGCAATGATGATAT (SEQ ID NO: 1494) |
| B2 | 170 | 191 | 22 | 57.54 | -4.91 | -5.26 | 0.41 | CATGGAAGGAAATGAATGAAGC (SEQ ID NO: 1495) |
| B1c | 122 | 146 | 25 | 60.49 | -5.80 | -4.35 | 0.36 | ATGGCAGTTTCAATAAAAGCTTGAG (SEQ ID NO: 1496) |
| LB | 150 | 169 | 20 | 61.89 | -4.74 | -5.43 | 0.55 | ATGGAGACTCACCCACTCGA (SEQ ID NO: 1497 ) |

TABLE 24

LAMP Assay Designs for Primer Group 14 (Accession No. MK614217)
Primer Group 14: TPS38FN - Specific — Exon 3 — gDNA/cDNA LAMP Design

| 1 | ID:7 | | dimer(minimum)dG=1.85 | | | | | |
|---|---|---|---|---|---|---|---|---|
| label | 5′pos | 3′pos | len | Tm | 5′dG | 3′dG | GCrate | Sequence |
| F3 | 160 | 184 | 25 | 56.10 | -4.46 | -4.74 | 0.32 | CGAAAATTACATGATGATAACAGAG (SEQ ID NO: 1498) |
| B3 | 332 | 350 | 19 | 55.47 | -4.65 | -4.16 | 0.37 | TTGCAAACTCAAGCAAAGT (SEQ ID NO: 1499) |
| FIP | | | 50 | | | | | AAGCTCTAACGCATGTCTAATTACT-GAAAACAAATTAGATGATGACGATG (SEQ ID NO: 1500) |
| BIP | | | 45 | | | | | CACTTCATTGGAGGACTGCAA-GAATTCATATCTTGTCTTTTCTCG (SEQ ID NO: 1501) |
| F2 | 185 | 209 | 25 | 56.78 | -3.78 | -5.23 | 0.32 | GAAAACAAATTAGATGATGACGATG (SEQ ID NO: 1502) |
| F1c | 229 | 253 | 25 | 60.13 | -5.09 | -3.12 | 0.36 | AAGCTCTAACGCATGTCTAATTACT (SEQ ID NO: 1503) |
| B2 | 307 | 330 | 24 | 55.55 | -3.09 | -5.04 | 0.33 | GAATTCATATCTTGTCTTTTCTCG (SEQ ID NO: 1504) |
| B1c | 255 | 275 | 21 | 60.19 | -4.51 | -5.41 | 0.48 | CACTTCATTGGAGGACTGCAA (SEQ ID NO: 1505) |
| LB | 283 | 305 | 23 | 60.57 | -6.36 | -3.17 | 0.43 | GGCCAAGTGGTTTATCGATGTAT (SEQ ID NO: 1506) |

Primer Group 14: TPS38FN — Common- Exon 3 — gDNA/cDNA LAMP Design

| 1 | ID:7 | | dimer(minimnm)dG=-2.34 | | | | | |
|---|---|---|---|---|---|---|---|---|
| label | 5′pos | 3′pos | len | Tm | 5′dG | 3′dG | GCrate | Sequence |
| F3 | 184 | 208 | 25 | 57.82 | -4.13 | -5.08 | 0.32 | GGAAAACAAATTAGATGATGACGAT (SEQ ID NO: 1507) |
| B3 | 370 | 389 | 20 | 56.09 | -5.18 | -4.06 | 0.40 | CGTCTTGATGCATTGATTGT (SEQ ID NO: 1508) |
| FIP | | | 45 | | | | | CTCCGTTCTTGCAGTCCTCC- ATTGTAGTAATTAGACATGCGTTAG (SEQ ID NO: 1509) |
| BIP | | | 47 | | | | | GCCAAGTGGTTTATCGATGTATACGCCATGTTGAAATCTAGTTTTGC (SEQ ID NO: 1510) |
| F2 | 224 | 248 | 25 | 56.85 | -3.29 | -4.51 | 0.32 | ATTGTAGTAATTAGACATGCGTTAG (SEQ ID NO: 1511) |
| F1c | 264 | 283 | 20 | 61.97 | -6.02 | -5.55 | 0.60 | CTCCGTTCTTGCAGTCCTCC (SEQ ID NO: 1512 ) |
| B2 | 347 | 368 | 22 | 55.70 | -5.05 | -4.68 | 0.36 | CCATGTTGAAATCTAGTTTTGC (SEQ ID NO: 1513) |
| B1c | 284 | 308 | 25 | 61.58 | -5.85 | -3.64 | 0.44 | GCCAAGTGGTTTATCGATGTATACG (SEQ ID NO: 1514) |

TABLE 25

LAMP Assay Designs for Primer Group 7
Primer Group 7: TPS18Choc — Exon 3 - Specific - R-Linalool Nerolidol — LAMP Design

| 1 | ID:40 | | dimer(minimum)dG=-2.46 | | | | | |
|---|---|---|---|---|---|---|---|---|
| label | 5′pos | 3′pos | len | Tm | 5′dG | 3′dG | GCrate | Sequence |
| F3 | 55 | 72 | 18 | 55.84 | -4.43 | -5.34 | 0.50 | AGAGAGGACATCAAGGGT (SEQ ID NO: 1515) |
| B3 | 234 | 255 | 22 | 55.05 | -4.86 | -4.91 | 0.36 | CTTGGATAAACTTTTGTGAGTG (SEQ ID NO: 1516) |
| FIP | | | 46 | | | | | GCACTAAATAAAGCAGCTTCATCAA-GCTGAGCTTATATGAAGCCTC (SEQ ID NO: 1517) |
| BIP | | | 41 | | | | | CAACACTTGGAAGCGTCCAT-TTTGAAGTGTAGTTGCCACAT (SEQ ID NO: 1518) |
| F2 | 75 | 95 | 21 | 57.79 | -5.59 | -5.93 | 0.48 | GCTGAGCTTATATGAAGCCTC (SEQ ID NO: 1519) |
| F1c | 125 | 149 | 25 | 60.26 | -4.98 | -4.07 | 0.36 | GCACTAAATAAAGCAGCTTCATCAA (SEQ ID NO: 1520) |
| B2 | 209 | 229 | 21 | 57.70 | -3.69 | -5.05 | 0.38 | TTTGAAGTGTAGTTGCCACAT (SEQ ID NO: 1521) |
| B1c | 151 | 170 | 20 | 60.36 | -4.82 | -4.90 | 0.50 | CAACACTTGGAAGCGTCCAT (SEQ ID NO: 1522) |
| LF | 96 | 120 | 25 | 60.14 | -3.17 | -4.74 | 0.40 | ATTTTCTCCCTCTATGCATAGATGG (SEQ ID NO: 1523) |
| LB | 171 | 195 | 25 | 60.97 | -5.84 | -3.92 | 0.40 | GACACGTCTTCATCGTTATGATCAA (SEQ ID NO: 1524) |

### Anti-Nociceptive

Primer Group 4: TPS32PK is above in Table 23

TABLE 26

LAMP Assay Designs for Primer Group 8 (The B3 and F3 primers can be used to specifically Identify/select plant cultivars expressing TPSSPK and not TPS5FN)
Primer Group 8: TPS5PK - Specific against TPS5FN - gDNA/cDNA LAMP Design

| 1 | ID:53 | | dimer(minimum)dG=-1.69 | | | | | |
|---|---|---|---|---|---|---|---|---|
| label | 5′pos | 3′pos | len | Tm | 5′dG | 3′dG | GCrate | Sequence |
| F3 | 526 | 548 | 23 | 56.42 | -3.98 | -4.29 | 0.30 | AGAGATTTCACAACAAAACATCT (SEQ ID NO: 1525) |
| B3 | 744 | 766 | 23 | 55.39 | -2.98 | -4.27 | 0.30 | AATCTAGTTTAGCAAGTTGAAGA (SEQ ID NO: 1526) |
| FIP | | | 44 | | | | | CAAAGCATGCTCCACTAATTTCACC-ATGATGACGAGGCAAGGTA (SEQ ID NO: 1527) |
| BIP | | | 50 | | | | | AGTGCATTGGAGAATGAAAAGGTTG-TGAATTCATATGATGATGATGAGAC (SEQ ID NO: 1528) |
| F2 | 562 | 580 | 19 | 57.57 | -3.95 | -4.08 | 0.47 | ATGATGACGAGGCAAGGTA (SEQ ID NO: 1529) |
| F1c | 621 | 645 | 25 | 62.74 | -5.01 | -5.02 | 0.44 | CAAAGCATGCTCCACTAATTTCACC (SEQ ID NO: 1530) |
| B2 | 714 | 738 | 25 | 56.74 | -3.57 | -4.76 | 0.32 | TGAATTCATATGATGATGATGAGAC (SEQ ID NO: 1531) |
| B1c | 654 | 678 | 25 | 62.25 | -5.80 | -5.00 | 0.40 | AGTGCATTGGAGAATGAAAAGGTTG (SEQ ID NO: 1532) |
| LB | 679 | 703 | 25 | 61.20 | -6.10 | -3.17 | 0.40 | GAGGCAAGGTGGTTTATTGATATGT (SEQ ID NO: 1533) |

Primer Groups 2 & 8: TPS5PK/FN - Designed for PK but may work for FN - gDNA/cDNA LAMP Design

| 1 | ID:4 6 | | dimer(minimum)dG=-2.38 | | | | | |
|---|---|---|---|---|---|---|---|---|
| label | 5′pos | 3′pos | len | Tm | 5′dG | 3′dG | GCrate | Sequence |
| F3 | 550 | 571 | 22 | 55.44 | -3.83 | -5.60 | 0.32 | AAACAATACTTGATGATGACGA (SEQ ID NO: 1534) |
| B3 | 744 | 766 | 23 | 55.39 | -2.98 | -4.27 | 0.30 | AATCTAGTTTAGCAAGTTGAAGA (SEQ ID NO: 1526) |
| FIP | | | 49 | | | | | CAAAGCATGCTCCACTAATTTCACC-GGCAAGGTAAAACTATATCTGTTG (SEQ ID NO: 1535) |
| BIP | | | 50 | | | | | AGTGCATTGGAGAATGAAAAGGTTG-TGAATTCATATGATGATGATGAGAC (SEQ ID NO: 1528) |
| F2 | 572 | 595 | 24 | 57.38 | -5.85 | -4.66 | 0.38 | GGCAAGGTAAAACTATATCTGTTG (SEQ ID NO: 1536) |
| F1c | 621 | 645 | 25 | 62.74 | -5.01 | -5.02 | 0.44 | CAAAGCATGCTCCACTAATTTCACC (SEQ ID NO: 1530) |
| B2 | 714 | 738 | 25 | 56.74 | -3.57 | -4.76 | 0.32 | TGAATTCATATGATGATGATGAGAC (SEQ ID NO: 1531) |
| B1c | 654 | 678 | 25 | 62.25 | -5.80 | -5.00 | 0.40 | AGTGCATTGGAC4AATC;AAAAC3GTTG (SEQ ID NO: 1532) |
| LB | 679 | 703 | 25 | 61.20 | -6.10 | -3.17 | 0.40 | GAGGCAAGGTGGTTTATTGATATGT (SEQ ID NO: 1533) |

TABLE 27

LAMP Assay Designs for Primer Group 11
Primer Group 11: TPS35LS-Cs10 — Exon 2 - - gDNA/cDNA LAMP Design

| 1 | ID:1 | | dimer(minimum)dG=-2.28 | | | | | |
|---|---|---|---|---|---|---|---|---|
| label | 5′pos | 3′pos | len | Tm | 5dG | 3′dG | GCrate | Sequence |
| F3 | 90 | 112 | 23 | 56.61 | -4.25 | -4.41 | 0.35 | TGGTTACTAATATGGTCACTGAA (SEQ ID NO: 1537) |
| B3 | 262 | 284 | 23 | 55.93 | -4.57 | -4.27 | 0.35 | ACGTAATATACGAGAGATGAAGA (SEQ ID NO: 1538) |
| FIP | | | 46 | | | | | AGTCGGCATTGATTATCATGTCC-ATTATATGTTCCCTTTCAAGGAT (SEQ ID NO: 1539) |
| BIP | | | 45 | | | | | GCACAGCGTTGACCAAGTTCCGTAATGAACAGTATTATTTTGGCT (SEQ ID NO: 1540) |
| F2 | 114 | 136 | 23 | 55.07 | -1.86 | -4.24 | 0.30 | ATTATATGTTCCCTTTCAAGGAT (SEQ ID NO: 1541) |
| F1c | 154 | 176 | 23 | 60.60 | -6.02 | -4.90 | 0.43 | AGTCGGCATTGATTATCATGTCC (SEQ ID NO: 1542) |
| B2 | 236 | 260 | 25 | 57.81 | -4.06 | -5.75 | 0.32 | CGTAATGAACAGTATTATTTTGGCT (SEQ ID NO: 1543) |
| B1c | 181 | 200 | 20 | 61.67 | -5.90 | -4.01 | 0.55 | GCACAGCGTTGACCAAGTTC (SEQ ID NO: 1544) |

Primer Group 11: TPS35LS-Cs10 — Exon 7 - - gDNA/cDNA LAMP Design

| 1 | ID:1 5 | | dimer(minimum)dG=-2.35 | | | | | |
|---|---|---|---|---|---|---|---|---|
| label | 5′pos | 3′pos | len | Tm | 5′dG | 3′dG | GCrate | Sequence |
| F3 | 16 | 33 | 18 | 57.79 | -6.24 | -4.85 | 0.50 | ATGG(3CATGAT(3GGTCTT (SEQ ID NO: 1545) |
| B3 | 185 | 203 | 19 | 55.25 | -4.55 | -4.14 | 0.42 | AACCATCCTAGCAGCATTA (SEQ ID NO: 1546) |
| FIP | | | 42 | | | | | AATCACTTGCTCCCTTGCTTC-TTGAGTGCTACATGAAAAGAC (SEQ ID NO: 1547) |
| BIP | | | 43 | | | | | ATGATTAAAAATGAGTGGGAACGCC-TTTCTGAAGCACATGGGA (SEQ ID NO: 1548) |
| F2 | 37 | 57 | 21 | 55.65 | -4.41 | -4.01 | 0.38 | TTGAGTGCTACATGAAAAGAC (SEQ ID NO: 1549) |
| F1c | 78 | 98 | 21 | 60.55 | -4.06 | -5.26 | 0.48 | AATCACTTGCTCCCTTGCTTC (SEQ ID NO: 1550) |
| B2 | 158 | 175 | 18 | 55.34 | -4.02 | -5.25 | 0.44 | TTTCTGAAGCACATGGGA (SEQ ID NO: 1551) |
| B1c | 102 | 126 | 25 | 62.10 | -3.45 | -6.68 | 0.40 | ATGATTAAAAATGAGTGGGAACGCC (SEQ ID NO: 1552) |

TABLE 28

LAMP Assay Designs for Primer Group 5
Primer Group 5: TPS31PK - Common - Exon 3 - - gDNA/cDNA LAMP Design

| 1 | ID:24 | | dimer(minimum)dG=-2.47 | | | | | |
|---|---|---|---|---|---|---|---|---|
| label | 5′pos | 3′pos | len | Tm | 5′dG | 3′dG | GCrate | Sequence |
| F3 | 373 | 393 | 21 | 55.94 | -5.63 | -4.01 | 0.38 | ACTGCTCTTCATTTCAAACTC (SEQ ID NO: 1553) |
| B3 | 552 | 572 | 21 | 56.20 | -4.82 | -5.26 | 0.38 | GTTGTGAAATCTCTTGCTTCA (SEQ ID NO: 1554) |
| FIP | | | 48 | | | | | TCCTTGAACTCATTTTTCTCGTCTT-CTTAGACAATATGGATTCAGTCC (SEQ ID NO: 1555) |
| BIP | | | 45 | | | | | AAGTAAGGATGTGAAAGGAATGGT-CTAATATGGGTTCACCCCTAA (SEQ ID NO: 1556) |
| F2 | 394 | 416 | 23 | 55.92 | -3.43 | -5.35 | 0.39 | CTTAGACAATATGGATTCAGTCC (SEQ ID NO: 1557) |
| F1c | 446 | 470 | 25 | 60.68 | -4.86 | -5.18 | 0.36 | TCCTTGAACTCATTTTTCTCGTCTT (SEQ ID NO: 1558) |
| B2 | 530 | 550 | 21 | 55.95 | -2.31 | -4.53 | 0.43 | CTAATATGGGTTCACCCCTAA (SEQ ID NO: 1559) |
| B1c | 477 | 500 | 24 | 60.04 | -3.24 | -4.55 | 0.38 | AAGTAAGGATGTGAAAGGAATGGT (SEQ ID NO: 1560) |

Primer Group 5: TPS31PK - Specific- Exon 3 - - gDNA/cDNA LAMP Design

| | | | | | | | | |
|---|---|---|---|---|---|---|---|---|
| 2 | ID:7 8 | | dimer(minimum)dG=-2.13 | | | | | |
| label | 5′pos | 3′pos | len | Tm | 5′dG | 3′dG | GCrate | Sequence |
| F3 | 723 | 745 | 23 | 55.61 | -3.03 | -4.01 | 0.30 | TGATATGTATGCTGAAAGGAATT (SEQ ID NO: 1561) |
| B3 | 914 | 933 | 20 | 56.48 | -6.10 | -6.41 | 0.40 | TGCCTCATATTTTAGTGCCA (SEQ ID NO: 1562) |
| FIP | | | 47 | | | | | AGCTCTTTTTGGTATGCTGACTGAA-ATCCAACTTTTCTTGACTTTGC (SEQ ID NO: 1563) |
| BIP | | | 40 | | | | | CAAGGTGGTGGAGTGGTTCTTCCACTACTCTGTCTCTAGC (SEQ ID NO: 1564) |
| F2 | 755 | 776 | 22 | 57.30 | -4.41 | -5.01 | 0.36 | ATCCAACTTTTCTTGACTTTGC (SEQ ID NO: 1565) |
| F1c | 797 | 821 | 25 | 62.81 | -5.32 | -4..41 | 0.40 | AGCTCTTTTTGGTATGCTGACTGAA (SEQ ID NO: 1566) |
| B2 | 877 | 896 | 20 | 56.80 | -5.25 | -4.67 | 0.50 | TCCACTACTCTGTCTCTAGC (SEQ ID NO: 1567) |
| B1c | 833 | 852 | 20 | 61.40 | -5.00 | -4.85 | 0.55 | CAAGGTGGTGGAGTGGTTCT (SEQ ID NO: 1568) |

TABLE 29

LAMP Assay Designs for Primer Group 10
Primer Group 10: TPS19BL - Exon 7 - - gDNA/cDNA LAMP Design -

| 1 | ID:4 | | dimer(minimum)dG=-2.46 | | | | | |
|---|---|---|---|---|---|---|---|---|
| label | 5′pos | 3′pos | len | Tm | 5′dG | 3′dG | GCrate | Sequence |
| F3 | 37 | 54 | 18 | 56.12 | -6.10 | -4.41 | 0.50 | TAGGCGTTGGTTGTGATC (SEQ ID NO: 1569) |
| B3 | 211 | 230 | 20 | 56.55 | -4.46 | -6.54 | 0.45 | ACATGAAGGAATTAGGAGGC (SEQ ID NO: 1570) |
| FIP | | | 47 | | | | | GTTTCCACCACCATTCCTCAAA-TATACATCAAAGGAACTAATCTTGC (SEQ ID NO: 1571) |
| BIP | | | 43 | | | | | TCGAAGTATGCATTCTTTGTTGAGG-AGTGTTGAAGATGCACGT (SEQ ID NO: 1572) |
| F2 | 60 | 84 | 25 | 56.86 | -2.87 | -5.26 | 0.32 | TATACATCAAAGGAACTAATCTTGC (SEQ ID NO: 1573) |
| F1c | 100 | 121 | 22 | 60.84 | -4.62 | -4.02 | 0.45 | GTTTCCACCACCATTCCTCAAA (SEQ ID NO: 1574) |
| B2 | 193 | 210 | 18 | 55.98 | -4.55 | -6.73 | 0.44 | AGTGTTGAAGATGCACGT (SEQ ID NO: 1575) |
| B1c | 130 | 154 | 25 | 61.49 | -5.04 | -4.86 | 0.40 | TCGAAGTATGCATTCTTTGTTGAGG (SEQ ID NO: 1576) |
| LB | 157 | 181 | 25 | 60.57 | -4.86 | -4.13 | 0.40 | CTTCCATGCATCTGAAATCTTTTCC (SEQ ID NO: 1577) |

Primer Group 10: TPS19BL - Exon 7 - - gDNA/cDNA LAMP Design

| ID:1 | | dimer(minimum)dG=-2.44 | | | | | | |
|---|---|---|---|---|---|---|---|---|
| label | 5′pos | 3′pos | len | Tm | 5′dG | 3′dG | GCrate | Sequence |
| F3 | 14 | 34 | 21 | 55.15 | -3.15 | -5.00 | 0.38 | TTGATATGTTCTTCCAAGTGG (SEQ ID NO: 1578) |
| B3 | 185 | 202 | 18 | 55.46 | -5.14 | -4.01 | 0.44 | AGATGCACGTGAGGAAAT (SEQ ID NO: 1579) |
| FIP | | | 42 | | | | | GCTTCTCTCAATCTCGCAAGATTA-GTAGGCGTTGGTTGTGAT (SEQ ID NO: 1580) |
| BIP | | | 41 | | | | | GGAATGGTGGTGGAAACGCT-AAAAGATTTCAGATGCATGGA (SEQ ID NO: 1581) |
| F2 | 36 | 53 | 18 | 56.45 | -5.42 | -4.46 | 0.50 | GTAGGCGTTGGTTGTGAT (SEQ ID NO: 1582) |
| F1c | 76 | 99 | 24 | 60.53 | -5.09 | -2.98 | 0.42 | GCTTCTCTCAATCTCGCAAGATTA (SEQ ID NO: 1583) |
| B2 | 159 | 179 | 21 | 55.46 | -3.52 | -4.91 | 0.33 | AAAAGATTTCAGATGCATGGA (SEQ ID NO: 1584) |
| B1c | 105 | 124 | 20 | 62.64 | -4.51 | -6.07 | 0.55 | GGAATGGTGGTGGAAACGCT (SEQ ID NO: 1585) |
| LB | 130 | 154 | 25 | 61.49 | -5.04 | -4.86 | 0.40 | TCGAAGTATGCATTCTTTGTTGAGG (SEQ ID NO: 1576) |

TABLE 30

LAMP Assay Designs for Primer Groups 7 and 9
Primer Groups 7&9: TPS18Choc/VF - Common - Exon 3 - Linalool Synthase Marker -gDNA/cDNA LAMP Design

| ID:15 | | dimer(minimum)dG=-2.18 | | | | | | |
|---|---|---|---|---|---|---|---|---|
| label | 5′pos | 3′pos | len | Tm | 5′dG | 3′dG | GCrate | Sequence |
| F3 | 6 | 27 | 22 | 55.82 | -3.69 | -4.06 | 0.32 | TTTCAACAAGTACAAGGACAAT (SEQ ID NO: 1586) |
| B3 | 182 | 203 | 22 | 55.50 | -6.10 | -4.73 | 0.36 | GCCTGATATTGATCATAACGAT (SEQ ID NO: 1587) |
| FIP | | | 43 | | | | | TGGGAGGCTTCATATAAGCTCAG-TTGTTTCGACACAAGGCTAA (SEQ ID NO: 1588) |
| BIP | | | 43 | | | | | AGGGAGAAAATATCCTTGATGAAGC-GAAGACGTGTCATGGACG (SEQ ID NO: 1589) |
| F2 | 36 | 55 | 20 | 57.12 | -3.83 | -4.93 | 0.40 | TTGTTTCGACACAAGGCTAA (SEQ ID NO: 1590) |
| F1c | 76 | 98 | 23 | 61.53 | -5.70 | -5.59 | 0.48 | TGGGAGGCTTCATATAAGCTCAG (SEQ ID NO: 1591) |
| B2 | 164 | 181 | 18 | 56.77 | -4.36 | -6.19 | 0.56 | GAAGACGTGTCATGGACG (SEQ ID NO: 1592) |
| B1c | 110 | 134 | 25 | 60.77 | -5.53 | -5.26 | 0.40 | AGGGAGAAAATATCCTTGATGAAGC (SEQ ID NO: 1593) |
| LB | 136 | 160 | 25 | 60.66 | -4.09 | -5.00 | 0.40 | GCTTTATTTAGTGCTCAACACTTGG (SEQ ID NO: 1594) |

### Anti-Insecticidal

TABLE 31

LAMP Assay Design for Primer Group 15
Primer Group 15: B-Farnescene - CsTPS25LS - Exon 6 - gDNA/cDNA LAMP Design

| 1 | ID:1 | | dimer(minimum)dG=-2.28 | | | | | |
|---|---|---|---|---|---|---|---|---|
| label | 5′pos | 3′pos | len | Tm | 5′dG | 3′dG | GCrate | Sequence |
| F3 | 1 | 23 | 23 | 55.64 | -4.79 | -4.07 | 0.35 | CTTTCGAGAAGCTATATCATTCA (SEQ ID NO: 1595) |
| B3 | 192 | 214 | 23 | 55.88 | -4.18 | -5.37 | 0.30 | GTTGAACCAAAAATATTTCCCAA (SEQ ID NO: 1596) |
| FIP | | | 43 | | | | | CTCTTCAATGGCTCTCCAATGAC-TGTGTCTAGAAAGGAGAGTG (SEQ ID NO: 1597) |
| BIP | | | 44 | | | | | ATCCATTCCAACAAAACTTGTGGC-GCTTGGATGAGCATTTGAGA (SEQ ID NO: 1598) |
| F2 | 24 | 43 | 20 | 55.38 | -5.07 | -4.74 | 0.45 | TGTGTCTAGAAAGGAGAGTG (SEQ ID NO: 1599) |
| F1c | 64 | 86 | 23 | 61.01 | -4.20 | -4.06 | 0.48 | CTCTTCAATGGCTCTCCAATGAC (SEQ ID NO: 1600) |
| B2 | 172 | 191 | 20 | 57.71 | -5.85 | -4.27 | 0.45 | GCTTGGATGAGCATTTGAGA (SEQ ID NO: 1601) |
| B1c | 109 | 132 | 24 | 62.50 | -4.29 | -6.41 | 0.42 | ATCCATTCCAACAAAACTTGTGGC (SEQ ID NO: 1602) |

TABLE 32

LAMP Assay Designs for Primer Group 17
Primer Group 17 - TPS29BC - Linalool Synthase - Exon 3 - gDNA/cDNA LAMP Design

| 1 | ID:26 | | dimer(minimum)dG=-2.18 | | | | | |
|---|---|---|---|---|---|---|---|---|
| label | 5′pos | 3′pos | len | Tm | 5′dG | 3′dG | GCrate | Sequence |
| F3 | 54 | 73 | 20 | 56.08 | -4.98 | -4.01 | 0.40 | GCAGTAACAAGCATGGAATT (SEQ ID NO: 1603) |
| B3 | 220 | 239 | 20 | 56.38 | -4.41 | -4.85 | 0.45 | TCCAATACAGTGGAACTTCC (SEQ ID NO: 1604) |
| FIP | | | 46 | | | | | CTCTTGGCTTCCTCCAAAACATTT-TGAGTTTGTATGAAGCTTCACA (SEQ ID NO: 1605) |
| BIP | | | 41 | | | | | TTCACCACCAAAAGGCTGAG-TGATTGTTTCACTTGCTTACC (SEQ ID NO: 1606) |
| F2 | 75 | 96 | 22 | 57.62 | -4.41 | -4.58 | 0.36 | TGAGTTTGTATGAAGCTTCACA (SEQ ID NO: 1607) |
| F1c | 115 | 138 | 24 | 61.75 | -4.35 | -3.71 | 0.42 | CTCTTGGCTTCCTCCAAAACATTT (SEQ ID NO: 1608) |
| B2 | 197 | 217 | 21 | 56.22 | -4.07 | -4.18 | 0.38 | TGATTGTTTCACTTGCTTACC (SEQ ID NO: 1609) |
| B1c | 140 | 159 | 20 | 60.04 | -5.02 | -5.59 | 0.50 | TTCACCACCAAAAGGCTGAG (SEQ ID NO: 1610) |
| LB | 165 | 189 | 25 | 62.01 | -4.60 | -3.31 | 0.44 | TCTCAGCTGGGAAAATGGATACTAC (SEQ ID NO: 1611) |

Primer Group 17 - TPS29BC - Linalool Synthase - Exon 3 - gDNA/cDNA LAMP Design

| 3 | ID:68 | | dimer(minimum)dG=-2.28 | | | | | |
|---|---|---|---|---|---|---|---|---|
| label | 5′pos | 3′pos | len | Tm | 5′dG | 3′dG | GCrate | Sequence |
| F3 | 106 | 125 | 20 | 55.01 | -6.10 | -4.28 | 0.40 | GCCTGAAGAAAATGTTTTGG (SEQ ID NO: 1612) |
| B3 | 290 | 310 | 21 | 56.15 | -4.16 | -4.76 | 0.43 | CAAAGTCACACTCTTTGTCTC (SEQ ID NO: 1613) |
| FIP | | | 43 | | | | | AGTAGTATCCATTTTCCCAGCTGA-GGAAGCCAAGAGTTTCACC (SEQ ID NO: 1614) |
| BIP | | | 47 | | | | | GAAACAATCACTGGAAGTTCCACT-ATCCATTTGGTAGAGATCAATGA (SEQ ID NO: 1615) |
| F2 | 127 | 145 | 19 | 57.67 | -5.70 | -5.02 | 0.53 | GGAAGCCAAGAGTTTCACC (SEQ ID NO: 1616) |
| F1c | 167 | 190 | 24 | 61.25 | -3.96 | -5.49 | 0.42 | AGTAGTATCCATTTTCCCAGCTGA (SEQ ID NO: 1617) |
| B2 | 267 | 289 | 23 | 57.03 | -4.29 | -4.07 | 0.35 | ATCCATTTGGTAGAGATCAATGA (SEQ ID NO: 1618) |
| B1c | 208 | 231 | 24 | 61.15 | -4.18 | -5.25 | 0.42 | GAAACAATCACTGGAAGTTCCACT (SEQ ID NO: 1619) |
| LB | 237 | 259 | 23 | 64.24 | -5.55 | -5.09 | 0.52 | GGAGGATGCCAAGATCTGAAGCT (SEQ ID NO: 1620) |

TABLE 33

LAMP Assay Designs for Primer Group 18
Primer Group 18 - TPS17AK - Linalool Synthase - Exon 6 - gDNA/cDNA LAMP Design (These Two target the AK-specific Allele on an unplaced scaffold of Cs10, avoiding the 92% related gene on chromosome 5)

| ID:1 | | dimer(minimum)dG=-1.71 | | | | | | |
|---|---|---|---|---|---|---|---|---|
| label | 5′pos | 3′pos | len | Tm | 5′dG | 3′dG | GCrate | Sequence |
| F3 | 1 | 22 | 22 | 56.24 | -6.00 | -4.29 | 0.41 | GTGGGTAGATATGTGTAGATGT (SEQ ID NO: 1621) |
| B3 | 199 | 221 | 23 | 55.76 | -1.98 | -4.34 | 0.30 | TTATTATGGAACTGTGACGAATT (SEQ ID NO: 1622) |
| FIP | | | 47 | | | | | TCCCACTGAAATCCAACCATTTTC-TCTACAAGAAGCAAAATGGTACT (SEQ ID NO: 1623) |
| BIP | | | 48 | | | | | ACCAGTCCTTATTGTGCATGCTT-GATAATAACCATGTTCCAAACATTC (SEQ ID NO: 1624) |
| F2 | 25 | 47 | 23 | 57.62 | -3.99 | -4.57 | 0.35 | TCTACAAGAAGCAAAATGGTACT (SEQ ID NO: 1625) |
| F1c | 83 | 106 | 24 | 61.54 | -5.86 | -3.17 | 0.42 | TCCCACTGAAATCCAACCATTTTC (SEQ ID NO: 1626) |
| B2 | 167 | 191 | 25 | 56.57 | -2.63 | -4.06 | 0.32 | GATAATAACCATGTTCCAAACATTC (SEQ ID NO: 1627) |
| B1c | 109 | 131 | 23 | 62.70 | -5.39 | -4.79 | 0.43 | ACCAGTCCTTATTGTGCATGCTT (SEQ ID NO: 1628) |
| LF | 51 | 75 | 25 | 63.06 | -4.71 | -4.90 | 0.44 | TCTTCCAATGTTGGTGTGTATCCAC (SEQ ID NO: 1629) |

Primer Group 18- TPS17AK - Linalool Synthase - Exon 6 - gDNA/cDNA LAMP Design

| 1 | ID:6 | | dimer(minimum)dG=-2.01 | | | | | |
|---|---|---|---|---|---|---|---|---|
| label | 5′pos | 3′pos | len | Tm | 5′dG | 3′dG | GCrate | Sequence |
| F3 | 12 | 34 | 23 | 55.31 | -4.30 | -4.02 | 0.30 | TGTGTAGATGTTTTCTACAAGAA (SEQ ID NO: 1630) |
| B3 | 199 | 221 | 23 | 55.76 | -1.98 | -4.34 | 0.30 | TTATTATGGAACTGTGACGAATT (SEQ ID NO: 1622) |
| FIP | | | 47 | | | | | TCCCACTGAAATCCAACCATTTTC-CAAAATGGTACTACAGTGGATAC (SEQ ID NO: 1631) |
| BIP | | | 48 | | | | | ACCAGTCCTTATTGTGCATGCTT-GATAATAACCATGTTCCAAACATTC (SEQ ID NO: 1624) |
| F2 | 36 | 58 | 23 | 56.34 | -3.32 | -4.08 | 0.39 | CAAAATGGTACTACAGTGGATAC (SEQ ID NO: 1632) |
| F1c | 83 | 106 | 24 | 61.54 | -5.86 | -3.17 | 0.42 | TCCCACTGAAATCCAACCATTTTC (SEQ ID NO: 1626) |
| B2 | 167 | 191 | 25 | 56.57 | -2.63 | -4.06 | 0.32 | GATAATAACCATGTTCCAAACATTC (SEQ ID NO: 1627) |
| B1c | 109 | 131 | 23 | 62.70 | -5.39 | -4.79 | 0.43 | ACCAGTCCTTATTGTGCATGCTT (SEQ ID NO: 1628) |

TABLE 34

LAMP Assay Designs for Primer Group 19
Primer Group 19 - TPS33PK - Alpha/Gamma-Terpinene Synthase - Exon 7 - gDNA/cDNA LAMP Design

| ID:1 | | dimer(minimum)dG=-2.18 | | | | | | |
|---|---|---|---|---|---|---|---|---|
| label | 5′pos | 3′pos | len | Tm | 5′dG | 3′dG | GCrate | Sequence |
| F3 | 5 | 25 | 21 | 55.16 | -4.71 | -4.90 | 0.38 | GAGGAATTGAAAAGAGGTGAT (SEQ ID NO: 1633) |
| B3 | 170 | 191 | 22 | 57.16 | -4.74 | -4.98 | 0.41 | CCATCTTAGCAAAGTTTGTAGC (SEQ ID NO: 1634) |
| FIP | | | 43 | | | | | GTTCACGAGCTTCTTCTTCAGATCTCCGACATCAATTCAATGT (SEQ ID NO: 1635) |
| BIP | | | 45 | | | | | AATGAAGCATGGAAGGAGATGAAATCTTCAGTCAATGAGATTGGA (SEQ ID NO: 1636) |
| F2 | 27 | 46 | 20 | 55.19 | -5.88 | -4.21 | 0.40 | CTCCGACATCAATTCAATGT (SEQ ID NO: 1637) |
| F1c | 67 | 89 | 23 | 60.32 | -4.67 | -4.15 | 0.43 | GTTCACGAGCTTCTTCTTCAGAT (SEQ ID NO: 1638) |
| B2 | 148 | 169 | 22 | 56.69 | -3.75 | -4.41 | 0.36 | ATCTTCAGTCAATGAGATTGGA (SEQ ID NO: 1639) |
| B1c | 107 | 129 | 23 | 60.03 | -3.57 | -3.92 | 0.39 | AATGAAGCATGGAAGGAGATGAA (SEQ ID NO: 1640) |

TABLE 35

LAMP Assay Designs for Primer Group 16
Primer Group 16 - TPS20LPA005 — Guaiol Synthase - cDNA/gDNA Specific LAMP Primers

| | | | | | | | | |
|---|---|---|---|---|---|---|---|---|
| 1 | ID:10 0 | | dimer(minimum)dG=-2.20 | | | | | |
| label | 5′pos | 3′pos | len | Tm | 5′dG | 3′dG | GCrate | Sequence |
| F3 | 1125 | 1145 | 21 | 55.43 | -5.69 | -5.09 | 0.38 | GGAGGTTATTTTGAAGAAGCT (SEQ ID NO: 1641) |
| B3 | 1326 | 1348 | 23 | 57.12 | -3.74 | -4.27 | 0.39 | CCAATATCATCCATGAACCTAGA (SEQ I D NO: 1642) |
| FIP | | | 49 | | | | | TCAACAAAACGTTACCAGAAGAAACGATGGTTGAATGAAGGATATTTCC (SEQ ID NO: 1643) |
| BIP | | | 46 | | | | | TAGCCACAAGTTTTGTAGGGATGTGAAACAATTTTAGGGTCTTTCG (SEQ ID NO: 1644) |
| F2 | 1147 | 1170 | 24 | 57.49 | -4.90 | -4.01 | 0.38 | GATGGTTGAATGAAGGATATTTCC (SEQ ID NO: 1645) |
| F1c | 1203 | 1227 | 25 | 60.21 | -4.58 | -4.01 | 0.36 | TCAACAAAACGTTACCAGAAGAAAC (SEQ ID NO: 1646) |
| B2 | 1288 | 1310 | 23 | 57.24 | -4.18 | -4.79 | 0.35 | TGAAACAATTTTAGGGTCTTTCG (SEQ ID NO: 1647) |
| B1c | 1228 | 1250 | 23 | 60.43 | -5.33 | -5.35 | 0.43 | TAGCCACAAGTTTTGTAGGGATG (SEQ ID NO: 1648) |
| LF | 1178 | 1201 | 24 | 60.00 | -3.43 | -5.09 | 0.42 | TAAGAGACTCTCAAATGCTCATCG (SEQ ID NO: 1649) |
| LB | 1264 | 1287 | 24 | 60.29 | -4.71 | -5.93 | 0.42 | CACATGAAACTCTAGATTGGCTCT (SEQ ID NO: 1650) |

### Methods of Analyzing the TPS Gene Profile of a Plant Cultivar

Provided herein are methods and compositions for analyzing the TPS gene profile of a plant cultivar. The analyzing can include, for example, identifying and/or quantitating one or more TPS genes and/or paralogs thereof in a plant cultivar. The methods employ polymerase chain reaction (PCR) primers that are complementary to unique subsequences within each TPS gene that is in the genome of the plant cultivar, wherein hybridization of a subsequence of a TPS gene or paralog thereof to the primers uniquely identifies and/or quantitates the TPS gene or a paralog thereof. A unique subsequence of a TPS gene is a portion of the TPS gene that is different from other subsequences of the TPS gene and is different from subsequences of other TPS genes, thereby permitting identification of each TPS gene in the genome of the plant cultivar, such as a Cannabis genome. The subsequences can be an intron or a portion thereof, an exon or a portion thereof, or any region in-between that is identified as unique compared to other subsequences in the TPS gene and compared to the subsequences in other TPS genes. In embodiments, the subsequences to which the primers can be hybridized are exons, or portions thereof. In certain embodiments, more than one unique subsequence (e.g., exon) of a TPS gene can be analyzed, e.g., 2, 3, 4, 5, 6, 7, 8, 9, 10 or more unique subsequences (e.g., exons) of a TPS gene can be identified and/or quantitated, thereby increasing the accuracy of identifying a particular TPS gene in the genomic profile of a plant cultivar.

The primers provided herein can be used to amplify TPS genes and/or paralogs thereof prior to input in various common assays for variant identification, including high resolution melting (HRM), quantitative PCR (qPCR), loop-mediated isothermal amplification (LAMP), restriction endonuclease digestion, gel electrophoresis, and/or Sanger/Next-Generation sequencing. For each plant cultivar that is analyzed according to the methods provided herein, a barcode representing each TPS gene and/or paralog thereof that is identified and/or quantitated can be assigned, thereby providing an efficient way to visualize the TPS gene profile of a plant cultivar. The barcode for a given TPS gene can be based, for example, on the number and types of exons that are detected and/or quantified for that TPS gene — each detected exon can be assigned a number, and the total read of all detected exons can constitute a barcode.

### Detection of TPS Genes or Paralogs Thereof

Provided herein are methods for analyzing nucleic acid from a plant sample. Also provided herein are methods for generating nucleic acid amplification products from a plant sample. Also provided herein are methods for preparing a nucleic acid mixture. A method herein can include contacting nucleic acid of a plant sample with a polynucleotide primer pair under amplification conditions. In embodiments, a method herein includes contacting nucleic acid of a plant sample with one or more polynucleotide primer pairs under amplification conditions. In some embodiments, a method herein comprises contacting nucleic acid of a plant sample with a plurality of polynucleotide primer pairs under amplification conditions. A plurality of primer pairs can include two or more polynucleotide primer pairs, three or more polynucleotide primer pairs, four or more polynucleotide primer pairs, five or more polynucleotide primer pairs, six or more polynucleotide primer pairs, seven or more polynucleotide primer pairs, eight or more polynucleotide primer pairs, nine or more polynucleotide primer pairs, or ten or more polynucleotide primer pairs. Each of the plurality of primer pairs can be used to analyze a sample in a separate reaction container, such as a well. Alternately, if the amplicons expected to be obtained using the plurality of primer pairs are expected to be of different sizes and/or are otherwise distinguishable (e.g., using labeled primers), a plurality of primers can be used to analyze the sample in a single reaction container.

In certain embodiments, a method includes generating one or more amplification products. Amplification products can be generated by any suitable amplification method described herein or known in the art (e.g., polymerase chain reaction (PCR)). Suitable amplification conditions can include any conditions that can generate an amplification product, when a target nucleic acid, such as a unique subsequence (e.g., exon) of a TPS gene, is contacted with primers that are capable of hybridizing to the target nucleic acid. In embodiments, a method includes generating a mixture (e.g., a mixture of two or more amplification product species). A mixture of two or more amplification product species can be generated when two or more primer pairs hybridize to different regions of a target nucleic acid. Such amplification product species can have different lengths and/or different nucleotide sequences, which can include overlapping and/or non-overlapping sequences.

Generally, a primer pair includes a forward primer and a reverse primer. Examples of primer pairs that can be used to detect exons in 74 Cannabis sativa TPS genes are set forth in Table B (SEQ ID NOS: 1-1284; 1398 and 1399), in Primer Groups 1-19 as set forth in Tables 1-16 and in B3/F3 polynucleotide primer pairs set forth in Tables 17-35. Two primer pairs can include two different forward primer species (e.g., A-fwd and B-fwd) and two different reverse primer species (e.g., A-rev, B-rev); can include one forward primer species (e.g., A-fwd) and two different reverse primer species (e.g., A-rev, B-rev); or can include two different forward primer species (e.g., A-fwd and B-fwd) and one reverse primer species (e.g., A-rev), provided the combination of forward and reverse primer species is capable of generating two amplification product species. Further forward and reverse primer combinations are contemplated for additional primer pairs. Examples of forward and reverse primer pairing combinations, with the corresponding amplification product species, are provided in Table B and herein, e.g., in in Primer Groups 1-19 as set forth in Tables 1-16 and in B3/F3 polynucleotide primer pairs set forth in Tables 17-35. For example, the size of the product (amplicon) that is amplified by any of the pairs of primers provided herein, including primers prepared by any of the methods provided herein, can be, e.g., about 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290 or 295 base pairs, within +/- about 10% of each of the recited sizes of the amplicons. In examples, the size of the product (amplicon) that is amplified by any of the pairs of primers provided herein, including primers prepared by any of the methods provided herein, can be, e.g., about 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975 or 2000 or more base pairs, within +/- about 10%, 15%, 20%, 25% or 30% of each of the recited sizes of the amplicons. For example, the sizes of the primers used to generate the amplicons can be between about 10 bases to about 50 bases, generally between about 12, 13, 14 or 15 bases to about 20, 21, 22, 24, 25, 26, 27, 28, 29, 30, 31, 32, 33, 34 or 35 or more bases, for example between 15 to about 30 bases, such as 15, 16, 17, 18, 19, 20, 21, 22, 23, 24, 25, 26, 27, 28, 29 or 30 bases.

In certain embodiments, when a plurality of primer pairs is used, either in a single reaction container or in a separate reaction container for each primer pair, a majority of the polynucleotide primer pairs hybridize to subsequences of the TPS genes and/or paralogs thereof of the plant sample. A majority of the polynucleotide primer pairs can refer to greater than 50% of the primer pairs. For example, a majority of the polynucleotide primer pairs can refer to greater than 60% of the primer pairs, greater than 70% of the primer pairs, greater than 80% of the primer pairs, or greater than 90% of the primer pairs. In embodiments, all (e.g., 100%) of the polynucleotide primer pairs hybridize to subsequences of the TPS genes and/or paralogs thereof of a plant sample. In certain embodiments, the primer pairs are selected from among those set forth in Table B, in Primer Groups 1-19 as set forth in Tables 1-16 and in B3/F3 polynucleotide primer pairs set forth in Tables 17-35.

In certain embodiments, one or more of the unique subsequences to which the polynucleotide primers hybridize can contain one or more variant nucleotide position, such as a substitution, an insertion or a deletion i.e., the methods of analysis provided herein can detect a genetic modification in a TPS gene.

A unique subsequence of a TPS gene or paralog thereof, to which the primer pairs hybridize, can be referred to as a target sequence. A target sequence generally refers to a unique subsequence, such as an exon, of a TPS gene or paralog thereof, between the two hybridization sites of a corresponding primer pair, and generally does not include the primer hybridization sites themselves. In embodiments, the two primer hybridization sites are conserved sequence regions that flank a diverse sequence, i.e., a unique subsequence of a TPS gene or paralog thereof is diverse and can differ from other subsequences of the TPS gene and of other TPS genes by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 or more bases, such as 110, 120, 130, 140 or 150 or more bases. In embodiments, the variant positions described for a target sequence do not include positions in the primer hybridization sites. In certain embodiments, the TPS genes and/or paralogs thereof have an overall sequence identity of percentages from between about 40% to about 90, 91, 92, 93, 94, 95, 96, 97, 98, 99 or 100%, such as at least 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In embodiments, one or more plant cultivars are of the family Rosidae. In certain embodiments, the plant sample is from a Cannabis cultivar, and the TPS gene profile is of a Cannabis genome. In aspects, tthe Cannabis cultivar is selected from among one or more of Type 1, Type 2, Type 3, Type 4 and Type 5 cultivars. Examples of Cannabis genomes include, but are not limited to, a Cannabis sativa genome, Cannabis indica genome, or Cannabis ruderalis genome. Examples of Cannabis genomes include CS10 (GENBANK assembly accession: GCA_900626175.1; REFSEQ assembly accession: GCF_900626175.1), Arcata Trainwreck, Grape Stomper, Citrix, Black 84, Headcheese, Red Eye OG, Tahoe OG, Master Kush, Chem 91, Domnesia, Sour Tsunami, Sour Tsunami_x_CK, Tibor_1_2016, 80 E-1, 80 E-2, 80 E-3, Harlox, Saint Jack, Herijuana, Mothers Milk_5, Black Beauty, Sour Diesel, JL_1, JL_2, JL_3, JL_4, JL_5, JL_6, JL_father, BBCC_x_JL_father, JL_mother, JL_mother_p, IdaliaFT_1, Fedora17_6_1, Carmal_1_2016, CS_1_2016, ElCam_1_2016, C3/USO-1, Carmagnola_3, and Merino_S_1.

A subsequence (e.g., exon) that is non-identical to any subsequence, or complement thereof, in the TPS gene or paralog thereof of a Cannabis genome generally refers to a sequence containing one or more variant nucleotides when compared to any other subsequence, or complement thereof, in the same TPS gene or in other TPS genes of the Cannabis genome. The primers provided herein generally share a high degree of sequence identity to the regions of the subsequence to which they hybridize. In some embodiments, each polynucleotide in each primer pair contains a sequence that is at least about 95% identical to a subsequence, or complement thereof, of a TPS gene in the genome of the plant cultivar. In certain embodiments, each polynucleotide in each primer pair contains a sequence that is 100% identical to a subsequence, or complement thereof, of a TPS gene in the genome of the plant cultivar.

In some embodiments, a primer provided herein includes a polynucleotide where one or more nucleotide positions contain a nonstandard nucleotide and/or a degenerate nucleotide. A nonstandard nucleotide can be, for example, a non-natural base, a modified base, or a universal base. A universal base is a base capable of indiscriminately base pairing with each of the four standard nucleotide bases: A, C, G and T. Universal bases that may be incorporated into a primer herein include, but are not limited to, inosine, deoxyinosine, 2′-deoxyinosine (dl, dlnosine), nitroindole, 5-nitroindole, and 3-nitropyrrole (e.g., 5′ nitroindole, deoxyinosine, deoxynebularine). A degenerate nucleotide typically refers to a mixture of nucleotides at a given position and may be represented by a letter other than A, T, G or C. For example, a degenerate nucleotide may be represented by R (A or G), Y (C or T), S (G or C), W (A or T), K (G or T), M (A or C), B (C or G or T), D (A or G or T), H (A or C or T), V (A or C or G), or N (any base), for example. Such symbols for degenerate nucleotides are part of the International Union of Pure and Applied Chemistry (IUPAC) standard nomenclature for nucleotide base sequence names and represent degenerate or nonstandard nucleotides that can bind multiple nucleotides. For example, an “M” in a primer or probe would include a mixture of A and C at that position, and thus could bind to either T or G in a complementary DNA strand. An “N” in a primer or probe would include a mixture of A, T, G and C at that position, and thus could bind to any nucleotide at that position in the complementary DNA strand.

### Methods for Analyzing Nucleic Acids

Provided herein are methods for analyzing nucleic acids. In embodiments, methods herein include analyzing nucleic acid from a plant sample. In certain embodiments, methods provided herein include analyzing nucleic acid from a Cannabis plant sample. In certain embodiments, the methods provided herein include analyzing subsequences of TPS genes and/or paralogs thereof.

In embodiments, analyzing includes detecting the presence or absence of a TPS gene or a paralog thereof in the genome of a plant cultivar. In certain embodiments, analyzing includes determining the presence or absence of more than one TPS gene or a paralog thereof in the genome of a plant cultivar. In embodiments, analyzing includes determining all the TPS genes and/or paralogs thereof that are present in the genome of a plant cultivar. In certain embodiments (e.g., by analyzing cDNA from the plant sample to detect the presence or absence of TPS genes and/or paralogs thereof), the expression profile of TPS genes and/or paralogs thereof in a plant sample can be analyzed. In embodiments, analyzing includes determining the presence or absence of one or more TPS genes and/or paralogs thereof in genomic DNA from the plant cultivar sample. In embodiments, the plant sample is from a Cannabis plant cultivar. In certain embodiments, the presence or absence of a TPS gene or paralog thereof can be determined based on one or more amplification products generated using one or more primer pairs that specifically amplify unique subsequences of one or more TPS genes or paralogs thereof. In certain embodiments, the presence or absence of a TPS gene or paralog thereof can be determined based on two or more amplification products generated using one or more primer pairs that specifically amplify unique subsequences of one or more TPS genes or paralogs thereof. In embodiments, the presence or absence of a TPS gene or paralog thereof can be determined based on 3, 4, 5, 6, 7, 8, 9, 10, 20, 30, 35, 40, 45, 50, 55, 60, 70, 80, 90, 100, 150, 200, 250, 300, 350, 400, 450, 500, 550, 600 or 650 or more amplification products generated using one or more primer pairs that specifically amplify unique subsequences of one or more TPS genes or paralogs thereof. In certain embodiments, the number of TPS genes and/or paralogs thereof that are detected in the nucleic acid from the plant sample can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 or more genes and/or paralogs. In certain embodiments, the plant cultivar is a Cannabis cultivar.

In certain embodiments, analyzing includes detecting a variant of a TPS gene or a paralog thereof in the genome of a plant cultivar, when compared to a reference unmodified genome of the plant cultivar. In embodiments, one or more TPS genes and/or paralogs thereof in the TPS gene profile is modified by genetic modification methods to obtain desired terpene, cannabinoid and/or flavonoid production profiles, and the analyzing includes screening to identify whether the genetic modification is in fact present, when compared to a reference unmodified genome of the plant cultivar. For example, based on the analysis of a reference unmodified TPS gene profile, and the terpene (and/or flavonoid and/or cannabinoid) abundance profile that is expected or is obtained for the unmodified TPS gene profile, it may be desirable to genetically modify one or TPS genes and/or paralogs thereof to provide an improved terpene (and/or flavonoid and/or cannabinoid) abundance profile, e.g., to impart improved medicinal properties, or improved resistance to an organism or environment, or improved affinity for an organism or environment. The variant can include, e.g., one or more nucleotide substitutions, insertions, or deletions at one or more variant positions, thereby changing the terpene and/or cannabinoid and/or flavonoid profiles. Methods of genetically modifying nucleic acids are known to those of skill in the art and include, but are not limited to, ZFN (Zinc Finger Nuclease), TALEN (Transcription Activator-Like Effector Nucleases), CRISPR-cas (cas9, cas12, cas13), Cre-Lox, MiRNA, SiRNA, ShRNA or a combination thereof. In certain embodiments, analyzing includes determining a terpene abundance profile, a flavonoid abundance profile, or any combination thereof. Techniques for measuring terpenes include, but are not limited to, gas chromatography with a flame ionization detector (GC-FID), gas chromatography — mass spectrometry (GC-MS) and headspace solid-phase microextraction (HS-SPME) in conjunction with GC-MS. Techniques for measuring flavonoids include, but are not limited to, gas chromatography (GC), gas chromatography — mass spectrometry (GC-MS), HPLC, HPLC-UV and NIR (near infrared reflectance). Techniques for measuring cannabinoids include, but are not limited to, HPLC, ultra-HPLC, HPLC-UV, HPLC-MS, UHPLC-MS, time-of-flight mass spectrometry (TOF-MS), LC-TOF-MS and NIR (near infrared reflectance).

In embodiments, detecting one or more genetic variations in a TPS gene or paralog thereof includes contacting the nucleic acid of the plant sample with one or more primer pairs as provided herein, under conditions wherein the one or more primer pairs hybridize to the one or more unique subsequences of a TPS gene or paralog thereof, wherein the one or more unique subsequences contain one or more variant nucleotide positions relative to the corresponding wild-type or unmodified subsequence in the plant cultivar. Following hybridization, the amplification conditions can be the same amplification conditions as those used to amplify the corresponding wild-type or unmodified subsequence, or they can be a different set of amplification conditions. In embodiments, a set of primers can be designed to hybridize with greater specificity for the expected genetically modified variant sequence.

Any suitable method for genotype assessment may be used for detecting a genetic variation in a TPS gene and/or paralog thereof, such as, for example, nucleic acid sequencing (examples of which are described herein) and/or a high-resolution melting (HRM) assay provided herein. Generally, a sequencing process and/or an HRM assay are performed in conjunction with a nucleic acid amplification method described herein (e.g., using the amplification primers provided herein). In certain embodiments, one or more genetic variations can be determined based on the presence and/or absence of amplification products generated using certain amplification primers provided herein.

### Samples

Provided herein are methods and compositions for processing, preparing, and/or analyzing nucleic acid. Nucleic acid or a nucleic acid mixture used in the methods and compositions described herein can be isolated from a sample (e.g., a test sample) obtained from a plant cultivar. A plant cultivar can be any plant whose genome includes TPS synthase genes and/or that produces terpenes, including for example, angiosperms, any species of woody, ornamental or decorative, crop or cereal, fruit or vegetable, fruit plant or vegetable plant, flower or tree, macroalga or microalga, phytoplankton and photosynthetic algae (e.g., green algae Chlamydomonas reinhardtii). A plant also refers to a unicellular plant (e.g. microalga) and a plurality of plant cells that are largely differentiated into a colony (e.g. volvox) or a structure that is present at any stage of a plant’s development. Such structures include, but are not limited to, a fruit, a flower, a seed, a shoot, a stem, a leaf, a root, plant tissue sand the like. As used herein, the term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue can be in planta, in organ culture, tissue culture, or cell culture. Any of the foregoing plant cultivars, portions thereof or extracts thereof are contemplated for use in the methods provided herein.

A nucleic acid sample can be isolated, obtained or prepared from any type of suitable biological (e.g., plant) specimen or sample (e.g., a test sample). A nucleic acid sample can be isolated or obtained from a single plant cell, a plurality of plant cells (e.g., cultured plant cells), plant cell culture media, conditioned plant cell culture media, or plant tissue (e.g., leaves, roots, stems).

A sample can be heterogeneous. For example, a sample can include more than one cell type and/or one or more nucleic acid species. In embodiments, a sample can include plant nucleic acid from more than one plant cultivar. In embodiments, the more than one plant cultivar providing the nucleic acid belong to the same species, e.g., both can be Cannabis cultivars. In embodiments, a sample can include plant cells and/or nucleic acid from a single plant or can include plant cells and/or nucleic acid from multiple plants.

### Nucleic Acid

Provided herein are methods and compositions for processing, preparing, and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (IncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. The plant nucleic acid analyzed according to the methods provided herein can be from, a plant, a plasmid containing plant nucleic acid, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term “nucleic acid” includes nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions can be achieved by generating sequences in which the third position of one or more selected (or all) codons is substituted with mixed-base and/or deoxyinosine residues. The term nucleic acid can be used interchangeably herein with locus, gene, cDNA, and mRNA encoded by a gene. The term also can include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” also can refer to a section of DNA involved in producing a polypeptide chain, such as an exon or portion thereof; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size can be expressed as a number of bases.

Target nucleic acids, such as a TPS gene or a paralog thereof or a portion thereof containing a unique subsequence, can be any nucleic acids of interest. Nucleic acids can be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid can be single or double stranded. Single stranded DNA (ssDNA), for example, can be generated by denaturing double stranded DNA by heating or by treatment with alkali, for example. Accordingly, in some embodiments, ssDNA is derived from double-stranded DNA (dsDNA).

Nucleic acid (e.g., nucleic acid targets, polynucleotides, primers, polynucleotide primers, polynucleotide primer pairs, sequences, and subsequences) as described herein can be complementary to another nucleic acid, hybridize to another nucleic acid, and/or be capable of hybridizing to another nucleic acid. The terms “complementary” or “complementarity” or “hybridization” generally refer to a nucleotide sequence that base-pairs by non-covalent bonds to a region of a nucleic acid (e.g., a primer that hybridizes to a unique subsequence of a TPS gene or a paralog thereof). In the canonical Watson-Crick base pairing, adenine (A) forms a base pair with thymine (T), and guanine (G) pairs with cytosine (C) in DNA. In RNA, thymine (T) is replaced by uracil (U). Thus, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “hybridize” or “capable of hybridizing” refers to a nucleotide sequence that is at least partially complementary. These terms can also encompass duplexes that are fully complementary such that every nucleotide in one strand is complementary or hybridizes to every nucleotide in the other strand in corresponding positions.

In certain instances, a nucleotide sequence can be partially complementary to a target, wherein not all nucleotides of, e.g., a primer, are complementary to every nucleotide in the target nucleic acid (unique subsequence, e.g., exon of a TPS synthase gene or paralog thereof) in all the corresponding positions. For example, the primer can be perfectly (i.e., 100%) complementary to a unique subsequence of a TPS synthase gene or paralog thereof, or a primer can share some degree of complementarity to a unique subsequence of a TPS synthase gene or paralog thereof, e.g., 70%, 75%, 85%, 90%, 95%, 99%.

The percent identity of two nucleotide sequences can be determined by aligning the sequences for optimal comparison purposes (e.g., gaps can be introduced in the sequence of a first sequence for optimal alignment) The nucleotides at corresponding positions are then compared, and the percent identity between the two sequences can be determined as a function of the number of identical positions shared by the sequences (i.e., % identity= # of identical positions/total # of positions×100). When a position in one sequence is occupied by the same nucleotide as the corresponding position in the other sequence, then the molecules are identical at that position.

In certain embodiments, nucleic acids in a mixture of nucleic acids are analyzed. A mixture of nucleic acids can include two or more nucleic acid species having the same or different nucleotide sequences, different lengths, different origins (e.g., genomic origins, cDNA, cell or tissue origins, sample origins, subject origins, and the like), different amplification products (e.g., amplification products generated from different sets of primer pairs), or combinations thereof. In certain embodiments, a mixture of nucleic acids includes or can generate a plurality of amplification product species generated from different sets of primer pairs (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more, 150 or more, 200 or more, 250 or more, 300 or more, 350 or more, 400 or more, 450 or more, 500 or more, 550 or more, 600 or more, or 650 or more amplification product species). In embodiments, a mixture of nucleic acids includes single-stranded nucleic acid and double-stranded nucleic acid. In certain embodiments, a mixture of nucleic acids includes DNA and RNA. In certain embodiments, a mixture of nucleic acids includes ribosomal RNA (rRNA) and messenger RNA (mRNA).

Nucleic acids used in the methods provided herein can contain nucleic acid from one plant sample or from two or more plant samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more plant samples).

Nucleic acid can be derived from one or more plant sources by methods known in the art. Any suitable method can be used for isolating, extracting and/or purifying DNA from a plant sample, non-limiting examples of which include methods of DNA preparation (e.g., described by Sambrook and Russell, Molecular Cloning: A Laboratory Manual 3d ed., 2001), various commercially available reagents or kits, such as DNeasy^{®}, RNeasy^{®}, QlAprep^{®}, QlAquick^{®}, and QlAamp^{®}, nucleic acid isolation/purification kits by Qiagen, Inc. (Germantown, Md); DNAzol^{®}, ChargeSwitch^{®}, Purelink^{®}, GeneCatcher^{®} nucleic acid isolation/purification kits by Life Technologies, Inc. (Carlsbad, CA); NucleoMag^{®}, NucleoSpin^{®}, and NucleoBond^{®} nucleic acid isolation/purification kits by Clontech Laboratories, Inc. (Mountain View, CA), DNA/RNA extraction kits from Zymo Research (e.g., ZYMOBIOMICS DNA Mini Kit, ZYMOBIOMICS DNA/RNA Miniprep Kit, ZYMOCLEAN gel DNA recovery); the like or combinations thereof.

Nucleic acid can be provided for performing methods described herein with or without processing of the sample(s) containing the nucleic acid. In embodiments, nucleic acid is provided for performing methods provided herein after processing of the sample(s) containing the nucleic acid. For example, a nucleic acid can be extracted, isolated, purified, partially purified and/or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a test subject (e.g., a plant). An isolated nucleic acid can be provided with fewer non-nucleic acid components (e.g., protein, lipid) than the number of components present in a source sample. A composition containing isolated nucleic acid can be about 50% to greater than 99% free of non-nucleic acid components. A composition containing isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the number of non-nucleic acid components present prior to subjecting the nucleic acid to a purification and/or analysis procedure. A composition containing purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition containing purified nucleic acid may be about 90%, 91%, 92%, 93%, 94%, 95%, 96%, 97%, 98%, 99% or greater than 99% free of nucleic acid species other than the plant nucleic acid of interest.

In certain embodiments, nucleic acid for performing methods provided herein is used without prior processing of the sample(s) containing the nucleic acid. For example, nucleic acid can be analyzed directly from a plant sample without prior extraction, purification, partial purification, and/or amplification.

Nucleic acid also can be exposed to a process that modifies certain nucleotides in the nucleic acid before being analyzed or prepared according to the methods provided herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid can be provided in any form that is suitable for conducting an analysis (e.g., genotype analysis, sequence analysis).

### Primers

Primers useful for detection, amplification, quantification, sequencing and/or analysis of nucleic acid are provided. The term “primer” as used herein refers to a nucleic acid that includes a nucleotide sequence capable of hybridizing or annealing to a target nucleic acid, at or near (e.g., adjacent to) a specific region of interest. Primers can allow for specific determination of a target nucleic acid nucleotide sequence or detection of the target nucleic acid (e.g., presence or absence of a sequence), or feature thereof, for example. A primer typically is a synthetic sequence. The term “specific” or “specificity,” as used herein, refers to the binding or hybridization of one molecule to another molecule, such as a primer for a target polynucleotide. That is, “specific” or “specificity” refers to the recognition, contact, and formation of a stable complex between two molecules, as compared to substantially less recognition, contact, or complex formation of either of those two molecules with other molecules. As used herein, the terms “anneal” and “hybridize” refer to the formation of a stable complex between two molecules. The terms “primer,” “polynucleotide,” “oligo,” or “oligonucleotide” are used interchangeably herein, when referring to primers.

A primer nucleic acid can be designed and synthesized using methods known to those of skill in the art as well as those provided herein. The primers used in the methods provided herein can be of any length suitable for hybridizing to a nucleotide sequence of interest (e.g., where the nucleic acid is in liquid phase or is bound to a solid support) and performing methods of analyses described herein. Primers can be designed based on any target nucleotide sequence, such as a unique subsequence of a TPA gene or a paralog thereof. A primer, in embodiments, can be about 10 to about 100 nucleotides, about 10 to about 70 nucleotides, about 10 to about 50 nucleotides, about 15 to about 30 nucleotides, or about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 nucleotides in length. A primer can include naturally occurring and/or non-naturally occurring nucleotides (e.g., labeled nucleotides), or a mixture thereof. Primers suitable for use in the methods provided herein can be synthesized and labeled using known techniques. For example, primers can be chemically synthesized according to the solid phase phosphoramidite triester method first described by Beaucage and Caruthers, Tetrahedron Lett., 22:1859-1862, 1981, using an automated synthesizer, as described in Needham-VanDevanter et al., Nucleic Acids Res. 12:6159-6168, 1984. Purification of primers can be achieved by native acrylamide gel electrophoresis or by anion-exchange high-performance liquid chromatography (HPLC), for example, as described in Pearson and Regnier, J. Chrom., 255:137-149, 1983.

All or a portion of a primer sequence can be complementary or substantially complementary to a target nucleic acid. As referred to herein, “substantially complementary” with respect to sequences refers to nucleotide sequences that will hybridize with each other. The stringency of the hybridization conditions can be altered to tolerate varying amounts of sequence mismatch. Included are target and primer sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other.

Primers that are substantially complimentary to a target nucleic acid sequence are also substantially identical to the compliment of the target nucleic acid sequence. That is, primers are substantially identical to the anti-sense strand of the nucleic acid. As referred to herein, “substantially identical” with respect to sequences refers to nucleotide sequences that are 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more identical to each other. One test for determining whether two nucleotide sequences are substantially identical is to determine the percent of identical nucleotide sequences shared.

Primer sequences and length can affect hybridization to target nucleic acid sequences. Depending on the degree of mismatch between the primer and target nucleic acid, low, medium or high stringency conditions may be used to effect primer/target annealing. s used herein, the term “stringent conditions” refers to conditions for hybridization and washing. Methods for hybridization reaction temperature condition optimization are known, and can be found, e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6X sodium chloride/sodium citrate (SSC) at about 45° C., followed by one or more washes in 0.2X SSC, 0.1% SDS at 50° C. Another example of stringent hybridization conditions includes hybridization in 6X sodium chloride/sodium citrate (SSC) at about 45° C., followed by one or more washes in 0.2X SSC, 0.1% SDS at 55° C. A further example of stringent hybridization conditions includes hybridization in 6X sodium chloride/sodium citrate (SSC) at about 45° C., followed by one or more washes in 0.2X SSC, 0.1% SDS at 60° C. Often, stringent hybridization conditions are hybridization in 6X sodium chloride/sodium citrate (SSC) at about 45° C., followed by one or more washes in 0.2X SSC, 0.1% SDS at 65° C. More often, stringency conditions can include 0.5 M sodium phosphate, 7% SDS at 65° C., followed by one or more washes at 0.2X SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids. Features of primers described herein also can apply to probes such as, for example, the qPCR probes provided herein.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, to a nucleic acid molecule having a sequence complementary to the primer compared to hybridization to a nucleic acid molecule not having a complementary sequence. For example, specific hybridization includes the hybridization of a primer to a target nucleic acid sequence that is complementary to the primer.

In certain embodiments, primers can include a nucleotide subsequence that is complementary to a solid phase nucleic acid primer hybridization sequence or substantially complementary to a solid phase nucleic acid primer hybridization sequence (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% identical to the primer hybridization sequence complement when aligned). A primer can contain a nucleotide subsequence not complementary to or not substantially complementary to a solid phase nucleic acid primer hybridization sequence (e.g., a sequence at the 3′ or 5′ end of the nucleotide subsequence in the primer complementary to or substantially complementary to the solid phase primer hybridization sequence, which sequence can hybridize to a unique subsequence in a TPS gene or paralog thereof).

A primer, in certain embodiments, can contain a modification such as one or more nonstandard nucleotides, non-natural nucleotides, universal bases, degenerate nucleotides, inosines, abasic sites, locked nucleic acids, minor groove binders, duplex stabilizers (e.g., acridine, spermidine), Tm modifiers or any modifier that changes the binding properties of the primers or probes. A primer, in certain embodiments, can contain a detectable molecule or entity (e.g., a fluorophore, radioisotope, colorimetric agent, particle, enzyme, and the like).

A primer also can refer to a polynucleotide sequence that, when hybridized to a subsequence of a target nucleic acid or another primer, facilitates the detection of a primer, a target nucleic acid or both, as with molecular beacons, for example. The term “molecular beacon,” as used herein, refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

### Amplification

Nucleic acids can be amplified under amplification conditions. The terms “amplify,” “amplification,” “amplification reaction,” “amplifying,” “amplified,” or “amplification conditions” as used herein refer to subjecting a target nucleic acid in a plant sample (e.g., TPS genes or paralogs thereof in a plant cultivar genome, or plant cDNA) to a process that linearly or exponentially generates amplicon nucleic acids having the same or substantially the same nucleotide sequence as the target nucleic acid or a portion thereof. In certain embodiments, the term “amplified” or “amplification” or “amplification conditions” refers to a method that includes a polymerase chain reaction (PCR). Nucleic acid can be amplified using a suitable amplification process. Nucleic acid amplification typically involves enzymatic synthesis of nucleic acid amplicons (copies), which contain a sequence complementary to a nucleotide sequence being amplified.

In certain embodiments, a limited amplification reaction, also known as pre-amplification, can be performed (e.g., of gDNA). Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification can limit inaccuracies associated with depleted reactants in standard PCR reactions, for example, and also can reduce amplification biases due to nucleotide sequence or species abundance of the target. In embodiments, a one-time primer extension can be performed as a prelude to linear or exponential amplification.

Any suitable amplification technique can be utilized. Amplification methods include, but are not limited to, polymerase chain reaction (PCR); ligation amplification (or ligase chain reaction (LCR)); amplification methods based on the use of Q-beta replicase or template-dependent polymerase (e.g., U.S. Pat. Publication No. US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence-based amplification (3SR or NASBA), and transcription-associated amplification (TAA). Non-limiting examples of PCR amplification methods include standard PCR, AFLP-PCR, allele-specific PCR, Alu-PCR, asymmetric PCR, colony PCR, hot start PCR, inverse PCR (IPCR), in situ PCR (ISH), intersequence-specific PCR (ISSR-PCR), long PCR, multiplex PCR, nested PCR, quantitative PCR (qPCR), touchdown PCR, reverse transcriptase PCR (RT-PCR), reverse transcriptase quantitative PCR (RT-qPCR), TAQMAN qPCR, real time PCR, single cell PCR, solid phase PCR, combinations thereof, and the like. Reagents and hardware for conducting PCR are commercially available.

It is understood by those of skill in the art that modifications to these PCR protocols can be made to achieve the same or similar results. For example, the temperatures for the various steps in the can be modified by between about 1-5° C., or touchdown PCR can be performed, i.e., the annealing temperature is adjusted based on the cycle number.

A generalized description of an amplification process is as follows. Primers and target nucleic acid are contacted, and complementary sequences hybridize to one another, for example. Primers can hybridize to a target nucleic acid, at or near (e.g., adjacent to, abutting, and the like) a sequence of interest. A reaction mixture, containing components necessary for enzymatic functionality, is added to the primer-target nucleic acid hybrid, and amplification can occur under suitable conditions. Components of an amplification reaction can include, but are not limited to, e.g., primers (e.g., individual primers, primer pairs, a plurality of primer pairs, and the like) a polynucleotide template (e.g., target nucleic acid), polymerase, nucleotides, dNTPs and the like. In embodiments, non-naturally occurring nucleotides or nucleotide analogs, such as analogs containing a detectable label (e.g., fluorescent or colorimetric label), can be used for example.

Any suitable polymerase can be selected, which can include polymerases for thermocycle amplification (e.g., Taq DNA Polymerase; Q-Bio™ Taq DNA Polymerase (recombinant truncated form of Taq DNA Polymerase lacking 5′-3′exo activity); SurePrime™ Polymerase (chemically modified Taq DNA polymerase for “hot start” PCR); Arrow™ Taq DNA Polymerase (high sensitivity and long template amplification)) and polymerases for thermostable amplification (e.g., RNA polymerase for transcription-mediated amplification (TMA) described at World Wide Web URL “gen-probe.com/pdfs/tma_whiteppr.pdf”). Other enzyme components can be added, such as reverse transcriptase for transcription mediated amplification (TMA) reactions, for example.

PCR conditions can be dependent upon primer sequences, target abundance, and the desired amount of amplification, and therefore, any suitable PCR protocol may be selected. PCR is typically carried out as an automated process with a thermostable enzyme. In this process, the temperature of the reaction mixture is cycled through a denaturing step, a primer-annealing step, and an extension reaction step automatically. Some PCR protocols also include an activation step and a final extension step. Machines specifically adapted for this purpose are commercially available. A non-limiting example of a PCR protocol that may be suitable for embodiments described herein is as follows: treating the sample at 95° C. for 2 minutes; repeating 40 cycles of 95° C. for 15 seconds and 60° C. for 30 seconds. Additional examples of suitable PCR protocols are provided in the working examples herein. A completed PCR reaction can optionally be kept at 4° C. until further action is desired. Multiple cycles frequently are performed using a commercially available thermal cycler. Suitable isothermal amplification processes also can be applied, in certain embodiments.

In certain embodiments, an amplification product can include naturally occurring nucleotides, non-naturally occurring nucleotides, nucleotide analogs and the like and combinations of the foregoing. An amplification product often has a nucleotide sequence that is identical to or substantially identical to a sample nucleic acid nucleotide sequence or complement thereof. A “substantially identical” nucleotide sequence in an amplification product will generally have a high degree of sequence identity to the nucleotide sequence species being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of infidelity of the polymerase used for extension and/or amplification, or additional nucleotide sequence(s) added to the primers used for amplification.

In embodiments, where a target nucleic acid is RNA, prior to the amplification step, a DNA copy (cDNA) of the RNA transcript of interest may be synthesized. A cDNA can be synthesized by reverse transcription, which can be carried out as a separate step, or in a homogeneous reverse transcription-polymerase chain reaction (RT-PCR), a modification of the polymerase chain reaction for amplifying RNA.

Amplification also can be accomplished using digital PCR, in certain embodiments. Digital PCR takes advantage of nucleic acid (DNA, cDNA or RNA) amplification on a single molecule level and offers a highly sensitive method for quantifying low copy number nucleic acid. Systems for digital amplification and analysis of nucleic acids are available (e.g., Fluidigm® Corporation).

Amplification reactions can be performed as individual amplification reactions, where one primer pair is used for each reaction and the presence or absence of one amplification product is detected. In certain embodiments, multiple individual amplification reactions may be performed (i.e., carried out in separate containers) using a different set of primers for each reaction, and the presence or absence of an amplification product is detected for each individual reaction. In embodiments, amplification reactions are performed as multiplex amplification reactions (i.e., a plurality of amplification reactions performed in a single container), where a plurality of primer pairs is used for the multiplex reaction, and the presence or absence of more than one amplification product is detected. Both individual amplification reactions and multiplex amplification reactions are contemplated for the primers provided herein.

In certain embodiments, a method provided herein includes generating nucleic acid amplification products from a plant sample. Such methods include contacting nucleic acid of a plant sample with a pair of polynucleotide primers under conditions wherein the pair of polynucleotide primers hybridize to and amplify a unique subsequence, when present, in a TPS gene or a paralog thereof in the genome (or cDNA, e.g., for obtaining an expression profile) of a plant cultivar.

### Quantitative PCR

In certain embodiments, an amplification method includes a quantifiable amplification method. For example, levels of expression of a TPS synthase gene or a paralog thereof can be measured using a quantitative PCR (qPCR) approach (e.g., on cDNA generated from RNA from a plant sample), or a reverse transcriptase quantitative PCR (RT-qPCR) approach (e.g., on RNA from a plant sample). Quantitative PCR (qPCR), which also can be referred to a real-time PCR, monitors the amplification of a targeted nucleic acid molecule during a PCR reaction (i.e., in real time). This method can be used quantitatively (quantitative real-time PCR) and semi-quantitatively (i.e., above/below a certain amount of nucleic acid molecules; semi-quantitative real-time PCR). The primers can be gene-specific probes that quantitate each amplicon (i.e., individual TPS genes), or they can be class-specific probes, e.g., to quantitate all monoterpene synthases, all diterpene synthases, all sesquiterpene synthases or combinations thereof in the TPS gene profile of the plant cultivar.

Methods for qPCR include use of non-specific fluorescent dyes that intercalate with double-stranded DNA, and sequence-specific DNA probes labelled with a fluorescent reporter, which generally allows detection after hybridization of the probe with its complementary sequence. Quantitative PCR methods typically are performed in a thermal cycler with the capacity to illuminate each sample with a beam of light of at least one specified wavelength and detect the fluorescence emitted by an excited fluorophore.

For non-specific detection, a DNA-binding dye can bind to all double-stranded (ds) DNA during PCR. An increase in DNA product during PCR therefore leads to an increase in fluorescence intensity measured at each cycle. For qPCR using dsDNA dyes, the reaction typically is prepared like a basic PCR reaction, with the addition of fluorescent dsDNA dye. Then the reaction is run in a real-time PCR instrument, and after each cycle, the intensity of fluorescence is measured with a detector (the dye only fluoresces when bound to the dsDNA (i.e., the PCR product)). In certain applications, multiple target sequences can be monitored in a tube by using different types of dyes. For specific detection, fluorescent reporter probes detect only the DNA containing the sequence complementary to the probe. Accordingly, use of the reporter probe can, in embodiments, increase specificity and facilitate performing the technique even in the presence of other dsDNA. Using different types of labels, fluorescent probes can be used in multiplex assays for monitoring several target sequences in the same tube. This method typically uses a DNA-based probe with a fluorescent reporter at one end and a quencher of fluorescence at the opposite end of the probe. The close proximity of the reporter to the quencher prevents detection of its fluorescence. During PCR, the probe is broken down by the 5′ to 3′ exonuclease activity of the polymerase, which breaks the reporter-quencher proximity and thus permits unquenched emission of fluorescence, which can be detected after excitation with a laser. An increase in the product targeted by the reporter probe at each PCR cycle therefore causes a proportional increase in fluorescence due to the breakdown of the probe and release of the reporter.

In certain embodiments, a method provided herein includes contacting nucleic acid of a plant sample with one or more primer pairs and one or more quantitative PCR probes. For example, certain primers provided herein (e.g., primers provided in Table B, in Primer Groups 1-19 as set forth in Tables 1-16 and in B3/F3 polynucleotide primer pairs set forth in Tables 17-35) can be used in combination with certain qPCR probes.

### Loop Mediated Isothermal Amplification (LAMP)

In certain embodiments, an amplification method includes loop mediated isothermal amplification (LAMP). Loop-mediated isothermal amplification (LAMP) is a single-tube technique useful for nucleic acid amplification. Reverse transcription loop-mediated isothermal amplification (RT-LAMP) combines LAMP with a reverse transcription step for the detection of RNA. LAMP is typically performed under isothermal conditions. In contrast to a polymerase chain reaction (PCR) technology, which is typically performed using a series of alternating temperature cycles, isothermal amplification is performed at a constant temperature, and does not require a thermal cycler.

In LAMP, a target sequence is amplified at a constant temperature (e.g., between about 60° C. to about 65° C.) using a plurality of primer pairs (e.g., two primer pairs, three primer pairs) and a polymerase (e.g., a polymerase with high strand displacement activity). In certain applications, four different primers can be used to amplify six distinct regions on a target sequence, for example, which can increase specificity. An additional pair of loop primers can further accelerate the reaction.

The amplification product can be detected via photometry (i.e., measuring the turbidity caused by magnesium pyrophosphate precipitate in solution as a byproduct of amplification). This generally allows for visualization by the naked eye or by photometric detection approaches (e.g., for small volumes). In certain applications, the reaction can be followed in real-time either by measuring turbidity or by fluorescence using intercalating dyes (e.g., SYTO 9, SYBR green). Certain dyes can be used to create a visible color change that can be seen with the naked eye without the need for specialized equipment. Dye molecules intercalate or directly label the DNA, and in turn can be correlated with the number of copies initially present. Accordingly, certain variations of LAMP can be quantitative. Detection of LAMP amplification products also can be achieved using manganese loaded calcein, which starts fluorescing upon complexation of manganese by pyrophosphate during in vitro DNA synthesis. Another method for visual detection of LAMP amplification products by the naked eye is based on the ability of the products to hybridize with complementary gold-bound single-stranded DNA, which prevents a red to purple-blue color change that would otherwise occur during salt-induced aggregation of the gold particles.

A number of LAMP visualization technologies are known to those of skill in the art (see, e.g., Fischbach et al., Biotechniques, 58(4):189-194 (2015), the contents of which are incorporated in their entirety by reference herein). Examples of such visualization reagents, summarized in the Table below from Fischbach et al., include magnesium pyrophosphate, hydroxynaphthol blue (HNB), calcein, SYBR Green I, EvaGreen and the nucleic acid-specific dye, berberine, which emits a fluorescent signal under UV light after a positive LAMP reaction.

| | | | | | | |
|---|---|---|---|---|---|---|
| | Turbidity | Hydroxynaphthol blue | Calcein | SYBR Green I | EvaGreen | Berberine |
| Substance | Mg-pyrophosphate (Mg-PPi) | Hydroxynaphthol blue (HNB) | Calcein AM + MnCl₂ | SYBR Green I | EvaGreen | Berberine-SO₄ |
| Origin | Amplification product | Synthetic | Synthetic | Synthetic | Synthetic | Natural |
| Toxicity | None | May cause eye irritation | May be harmful to skin and eyes | Mutation enhancer | Possible carcinogen | May be toxic in high concentrations |
| Detection Mechanism | Insoluble complex; precipitation | Decrease of free Mg²⁺ | Decrease of free Mn²⁺ | dsDNA intercalation | dsDNA intercalation | Small groove intercalation |
| Readout | Turbidity Absorbance: 400 nm | Absorption Absorbance: 650 nm | Fluorescence Excitation: 495 nm Emission: 515 nm | Fluorescence Excitation: 494 nm Emission: 521 nm | Fluorescence Excitation: 500 nm Emission: 530 nm | Fluorescence Excitation: 450 nm Emission: 530 nm |
| Effect on amplification | None | None | Manganese may inhibit reaction | Not inhibiting when used 0.5-1x | Not inhibiting when used 0.5-1x | Not inhibiting (≤180 µM) |
| One-pot real-time assay | ++ | - | +++ | ++++ | ++++ | ++++ |
| Equipment for real-time detection | Turbidometer | - | Fluorometer with FAM filter | Fluorometer with FAM filter | Fluorometer with FAM filter | Fluorometer with FAM filter |
| One-pot end point assay | +++ | ++++ | ++++ | - | - | +++ (UV light with low background signal) |
| Equipment for end point detection | None (cordless centrifuge) | None | UV lamp (optional) | Not applicable | Not applicable | UV lamp |
| Evaluation of results | + Turbid - Clear | + Sky blue - Violet | + Green - Orange | + Fluorescence signal - No signal | + Fluorescence signal - No signal | + Fluorescence signal - No signal |
| Percentage of overall costs * | 0 | <0.1 | <0.1 | 26.01 | 1.18 | 0.82 |
| Relative sensitivity ** | +++ | +++ | +++ | +++ | +++ | +++ |
| Field applicability *** | +++ | ++++ | ++++ | ++ | ++ | ++++ |

Summary of features relevant for in-the-field loop-mediated isothermal amplification (LAMP) assays tested for detection of potato spindle tuber viroid (PSTVd).
Number of “+” describes the applicability/relevance of the feature.
* Overall costs represent the basic chemicals of common suppliers for one LAMP reaction, depending on reaction volume.
** In our assays. Analytical sensitivity may depend on LAMP setup and has to be optimized separately.
*** Depending on visualization.

In embodiments, a method herein includes contacting nucleic acid of a plant sample with a set of loop mediated isothermal amplification (LAMP) primers. For example, Cannabis plant cultivars, or offspring thereof, containing particular TPS genes can be identified via a LAMP assay. In aspects, the LAMP assay can be a colorimetric assay. Examples of LAMP primer sets that can be used to identify Cannabis plant cultivars, or offspring thereof, containing a terpinolene producing TPS gene (csTPS37FN) are shown in Table C below:

TABLE 5

5 LAMP primer sets for Terpinolene-producing gene, csTPS37FN

| Set 1 | | 1381-2005 | dimer(minimum) dG=-2.36 | | | | | |
|---|---|---|---|---|---|---|---|---|
| label | 5′pos | 3′pos | len | Tm | 5′dG | 3′dG | GC rate | Sequence (SEQ ID NO) |
| F3 | 134 | 151 | 18 | 57.55 | -4.41 | -4.16 | 0.5 | TCAGTTGGGGGACCAATT (1285) |
| B3 | 332 | 350 | 19 | 56.29 | -5.68 | -4.27 | 0.53 | CATCCGACGATGTTCCTAG (1286) |
| FIP | | | 47 | | | | | GGATCATCATAACCTTCTTCCAAGA-TCTTTTGCATGCTTATTTTGCT (1287) |
| BIP | | | 47 | | | | | TGAAGGATCCCTGGAAATATCAAAT-TCTTCAAGTCGTAAAAGTATGG (1288) |
| F2 | 154 | 175 | 22 | 57.4 | -3.52 | -4.91 | 0.32 | TCTTTTGCATGCTTATTTTGCT (1289) |
| F1c | 210 | 234 | 25 | 60.23 | -4.76 | -4.86 | 0.4 | GGATCATCATAACCTTCTTCCAAGA (1290) |
| B2 | 306 | 327 | 22 | 55.41 | -4.27 | -4.23 | 0.36 | TCTTCAAGTCGTAAAAGTATGG (1291) |
| B1c | 250 | 274 | 25 | 60.09 | -4.86 | -3.57 | 0.36 | TGAAGGATCCCTGGAAATATCAAAT (1292) |
| LF | 178 | 202 | 25 | 60.48 | -5.7 | -4.33 | 0.4 | GGAAGCTTTTTCTAAGGGATTTGTG (1293) |
| F3 | 233 | 257 | 25 | 56.76 | -5.3 | -4.24 | 0.32 | CCTTCCATAAATATTCATGAAGGAT (1294) |
| B3 | 425 | 442 | 18 | 55.77 | -2.39 | -7.2 | 0.44 | AAATTTGATGTGCTCGCG (1295) |
| FIP | | | 47 | | | | | TCAAGTCGTAAAAGTATGGATCCAA-CCTGGAAATATCAAATGATGGT (1296) |
| BIP | | | 43 | | | | | CTAGGAACATCGTCGGATGAGA-CAGAAACACCTGTATCATTCA (1297) |
| F2 | 259 | 280 | 22 | 55.91 | -5.7 | -4.9 | 0.36 | CCTGGAAATATCAAATGATGGT (1298) |
| F1c | 300 | 324 | 25 | 60.07 | -4.41 | -4.41 | 0.36 | TCAAGTCGTAAAAGTATGGATCCAA (1299) |
| B2 | 393 | 413 | 21 | 55.12 | -4.02 | -4.07 | 0.38 | CAGAAACACCTGTATCATTCA (1300) |
| B1c | 332 | 353 | 22 | 60.75 | -4.27 | -4.15 | 0.5 | CTAGGAACATCGTCGGATGAGA (1301) |
| LB | 359 | 383 | 25 | 60.89 | -4.94 | -3.67 | 0.4 | AGAGGAGATGTTCCGAAATCAATTC (1302) |
| F3 | 210 | 231 | 22 | 56.36 | -4.86 | -4.32 | 0.36 | TCTTGGAAGAAGGTTATGATGA (1303) |
| B3 | 425 | 442 | 18 | 55.77 | -2.39 | -7.2 | 0.44 | AAATTTGATGTGCTCGCG (1304) |
| FIP | | | 48 | | | | | TCAAGTCGTAAAAGTATGGATCCAA-TAAATATTCATGAAGGATCCCTG (1305) |
| BIP | | | 43 | | | | | CTAGGAACATCGTCGGATGAGA-CAGAAACACCTGTATCATTCA (1306) |
| F2 | 240 | 262 | 23 | 55.42 | -1.98 | -5.7 | 0.35 | TAAATATTCATGAAGGATCCCTG (1307) |
| F1c | 300 | 324 | 25 | 60.07 | -4.41 | -4.41 | 0.36 | TCAAGTCGTAAAAGTATGGATCCAA (1308) |
| B2 | 393 | 413 | 21 | 55.12 | -4.02 | -4.07 | 0.38 | CAGAAACACCTGTATCATTCA (1309) |
| B1c | 332 | 353 | 22 | 60.75 | -4.27 | -4.15 | 0.5 | CTAGGAACATCGTCGGATGAGA (1310) |
| LB | 359 | 383 | 25 | 60.89 | -4.94 | -3.67 | 0.4 | AGAGGAGATGTTCCGAAATCAATTC (1311) |
| F3 | | 21 | 21 | 56.35 | -6.3 | -5.08 | 0.38 | GGGACCAATTATTCTTTTGCA (1312) |
| B3 | 191 | 209 | 19 | 56.29 | -5.68 | -4.27 | 0.53 | CATCCGACGATGTTCCTAG (1313) |
| FIP | | | 48 | | | | | GGATCATCATAACCTTCTTCCAAGA-GCTTATTTTGCTTTCACAAATCC (1314) |
| BIP | | | 47 | | | | | ATGAAGGATCCCTGGAAATATCAAA-TCTTCAAGTCGTAAAAGTATGG (1315) |
| F2 | 23 | 45 | 23 | 57.04 | -3.97 | -4.01 | 0.35 | GCTTATTTTGCTTTCACAAATCC (1316) |
| F1c | 69 | 93 | 25 | 60.23 | -4.76 | -4.86 | 0.4 | GGATCATCATAACCTTCTTCCAAGA (1317) |
| B2 | 165 | 186 | 22 | 55.41 | -4.27 | -4.23 | 0.36 | TCTTCAAGTCGTAAAAGTATGG (1318) |
| B1c | 108 | 132 | 25 | 60.09 | -3.9 | -3.57 | 0.36 | ATGAAGGATCCCTGGAAATATCAAA (1319) |
| F3 | 38 | 58 | 21 | 55.06 | -3.71 | -4.74 | 0.33 | ACAAATCCCTTAGAAAAAGCT (1320) |
| B3 | 206 | 227 | 22 | 55.62 | -4.25 | -4.25 | 0.41 | CATCTCCTCTTTTCATCTCATC (1321) |
| FIP | | | 49 | | | | | TTCCAGGGATCCTTCATGAATATTT-CCATAAAATTCTTGGAAGAAGGTT (1322) |
| BIP | | | 44 | | | | | ACCCTACCATATTTCATCTTGGATC-CGACGATGTTCCTAGGTCA (1323) |
| F2 | 60 | 83 | 24 | 57.31 | -3.74 | -4.5 | 0.33 | CCATAAAATTCTTGGAAGAAGGTT (1324) |
| F1c | 100 | 124 | 25 | 60.09 | -4.86 | -2.28 | 0.36 | TTCCAGGGATCCTTCATGAATATTT (1325) |
| B2 | 187 | 205 | 19 | 57.8 | -6.37 | -5.25 | 0.53 | CGACGATGTTCCTAGGTCA (1326) |
| B1c | 141 | 165 | 25 | 60.11 | -4.92 | -4.76 | 0.4 | ACCCTACCATATTTCATCTTGGATC (1327) |

### Detection of Amplification Products

Amplification products generated by a method provided herein can be detected by a suitable detection process. Non-limiting examples of methods of detection include electrophoresis, nucleic acid sequencing, mass spectrometry, mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, coded microspheres, template-directed incorporation (TDI), fluorescence polarization, colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), digital PCR, nanopore sequencing, chips, and combinations thereof.

In certain embodiments, amplification products are detected using electrophoresis. Any suitable electrophoresis method, whereby amplified nucleic acids are separated by size, can be used in conjunction with the methods provided herein, which include, but are not limited to, standard electrophoretic techniques and specialized electrophoretic techniques, such as, for example capillary electrophoresis (e.g., Capillary Zone Electrophoresis (CZE), also known as free-solution CE (FSCE), Capillary Isoelectric Focusing (CIEF), Isotachophoresis (ITP), Electrokinetic Chromatography (EKC), Micellar Electrokinetic Capillary Chromatography (MECC OR MEKC), Micro Emulsion Electrokinetic Chromatography (MEEKC), Non-Aqueous Capillary Electrophoresis (NACE), and Capillary Electrochromatography (CEC)).

Non-limiting standard electrophoresis example is presented as follows. After running an amplified nucleic acid sample in an agarose or polyacrylamide gel, the gel can be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which can then be compared to the control based on the intensity of the band, thus detecting and quantifying the target sequence of interest. In embodiments, where a plurality of primer pairs is used in an amplification reaction, multiple amplification products of varying size can be detected using electrophoresis.

### High Resolution Melting (HRM)

In certain embodiments, nucleic acid is analyzed in the methods provided herein using a high-resolution melting (HRM) endpoint assay. In embodiments, an analysis includes performing a high-resolution melting (HRM) endpoint assay on amplification products (e.g., amplification products generated using primers provided herein). In embodiments, an analysis includes performing a high-resolution melting (HRM) endpoint assay on nucleic acid in a mixture (e.g., a mixture of amplification products generated using a plurality of primer pairs).

High resolution melt or high-resolution melting (HRM) analysis is a technique useful for the detection of mutations, polymorphisms, and epigenetic differences in double-stranded DNA. Typically, amplification (e.g., a polymerase chain reaction (PCR)) is performed prior to HRM analysis to amplify a DNA region in which a mutation or other variant of interest is located. The HRM process involves a precise warming of the amplification product from around 50° C. up to around 95° C. At some point during this process, the melting temperature of the amplicon is reached and the two strands of DNA separate (i.e., melt apart).

The separation of strands can be monitored in real-time (e.g., using a fluorescent dye). Dyes that can be used for HRM include intercalating dyes, which specifically bind to double-stranded DNA and emit fluorescence when bound to DNA. At the start of an HRM analysis there is a high level of fluorescence in the sample because of the billions of copies of the amplicon. However, as the sample is heated up and the two strands of the DNA melt apart, presence of double stranded DNA decreases, and thus the fluorescence is reduced. In certain configurations, an HRM machine has a camera that monitors this process by measuring the fluorescence. The machine can plot the data (e.g., as a graph sometimes referred to as a melt curve), showing the level of fluorescence vs. temperature.

The melting temperature of an amplification product at which the two DNA strands come apart is a predictable parameter, and typically is dependent on the DNA sequence of the amplicon. When comparing two samples from two different plants containing the same TPS gene, for example, amplification products from both samples should have the same shaped melt curve. However, if one of the plants contains a TPS gene variant, this will alter the temperature at which the DNA strands melt apart. Accordingly, the two melt curves will be different. The difference can be subtle, but because HRM machines typically are capable of monitoring the HRM process in high resolution, it generally is possible to accurately document these changes and therefore identify if a mutation or variant is present or absent.

In certain embodiments, an analysis includes detecting one or more genetic variations (e.g., single nucleotide substitutions) in a TPS gene or paralog thereof according to results obtained from a high-resolution melting (HRM) endpoint assay. In embodiments, an analysis includes detecting two or more genetic variations (e.g., single nucleotide substitutions) in a in a TPS gene or paralog thereof, according to results obtained from a high-resolution melting (HRM) endpoint assay. In certain embodiments, an analysis includes detecting three or more genetic variations (e.g., single nucleotide substitutions) in a TPS gene or paralog thereof, according to results obtained from a high-resolution melting (HRM) endpoint assay. In certain embodiments, an analysis includes detecting four or more genetic variations (e.g., single nucleotide substitutions) in a TPS gene or paralog thereof, according to results obtained from a high-resolution melting (HRM) endpoint assay. In certain embodiments, an analysis includes detecting five or more genetic variations (e.g., single nucleotide substitutions) in a TPS gene or paralog thereof, according to results obtained from a high-resolution melting (HRM) endpoint assay. In certain embodiments, an analysis includes detecting six or more genetic variations (e.g., single nucleotide substitutions) in a TPS gene or paralog thereof, according to results obtained from a high-resolution melting (HRM) endpoint assay. In certain embodiments, an analysis includes detecting seven or more genetic variations (e.g., single nucleotide substitutions) in a TPS gene or paralog thereof, according to results obtained from a high-resolution melting (HRM) endpoint assay. In certain embodiments, an analysis includes detecting eight or more genetic variations (e.g., single nucleotide substitutions) in a TPS gene or paralog thereof, according to results obtained from a high-resolution melting (HRM) endpoint assay. In certain embodiments, an analysis includes detecting nine or more genetic variations (e.g., single nucleotide substitutions) in a TPS gene or paralog thereof, according to results obtained from a high-resolution melting (HRM) endpoint assay. In certain embodiments, an analysis includes detecting ten or more genetic variations (e.g., single nucleotide substitutions) in a TPS gene or paralog thereof, according to results obtained from a high-resolution melting (HRM) endpoint assay.

### Nucleic Acid Sequencing

In certain embodiments of the methods provided herein, the nucleic acid is sequenced. In embodiments, amplified subsequences of a TPS gene or a paralog thereof are sequenced by a sequencing process. In embodiments, the sequencing process generates sequence reads (or sequencing reads). In certain embodiments, a method herein comprises determining the sequence of a unique subsequence, such as an exon or a portion thereof, of a TPS gene or a paralog thereof, based on the sequence reads. In certain embodiments, a method provided herein includes determining the TPS gene profile, and/or the TPS gene expression profile, of a plant cultivar based on the sequence reads. In embodiments, the methods provided herein include determining one or TPS gene profiles of one or more plant cultivars based on the sequence reads.

Nucleic acid can be sequenced using any suitable sequencing platform, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In embodiments, a high-throughput sequencing method can be used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In embodiments, MPS sequencing methods utilize a targeted approach, where specific chromosomes, genes or regions of interest are sequenced. For example, a targeted approach can include targeting specific TPS genes, or specific unique subsequences of a TPS gene, for sequencing. In certain embodiments, a non-targeted approach is used where most or all nucleic acids in a sample are sequenced, amplified and/or captured randomly.

Non-limiting examples of sequencing platforms include a sequencing platform provided by Illumina® (e.g., HiSeq™, HiSeq™ 2000, MiSeq™, Genome Analyzer™, and Genome Analyzer™ II sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLiD sequencing system); Roche (e.g., 454 GS FLX+ and/or GS Junior sequencing systems); Helicos True Single Molecule Sequencing; Ion semiconductor-based sequencing (e.g., as developed by Life Technologies), WildFire, 5500, 5500xl W and/or 5500xl W Genetic Analyzer based technologies (e.g., as developed and sold by Life Technologies, U.S. Pat. Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing; or any other suitable sequencing platform. Other sequencing methods that can be used to conduct methods herein include digital PCR, sequencing by hybridization, nanopore sequencing, chromosome-specific sequencing (e.g., using DANSR (digital analysis of selected regions) technology).

In certain embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In embodiments, a sequencing process generates short sequencing reads or “short reads.” In embodiments, the nominal, average, mean or absolute length of short reads sometimes is about 10 continuous nucleotides to about 250 or more contiguous nucleotides. In certain embodiments, the nominal, average, mean or absolute length of short reads sometimes is about 50 continuous nucleotides to about 150 or more contiguous nucleotides.

The length of a sequence read often is associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments, sequence reads are of a mean, median, average or absolute length of about 100 bp to about 200 bp.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid.

In certain embodiments, “obtaining” nucleic acid sequence reads of a sample from a plant and/or “obtaining” nucleic acid sequence reads from one or more amplification products can involve directly sequencing nucleic acid to obtain the sequence information. In some embodiments, “obtaining” can involve receiving sequence information obtained directly from a nucleic acid by another.

In certain embodiments, some or all nucleic acids in a sample are enriched and/or amplified (e.g., non-specifically, or specifically using amplification primers described herein) prior to or during sequencing. In certain embodiments, specific nucleic acid species or subsets in a sample are enriched and/or amplified prior to or during sequencing. In some embodiments, nucleic acid from a pathogen may be enriched and/or amplified prior to or during sequencing, while nucleic acid from a host plant is not enriched and/or amplified prior to or during sequencing. For example, nucleic acid from the genome of the plant cultivar can be enriched and/or amplified prior to or during sequencing, while nucleic acid from the Cannabis genome is not enriched and/or amplified prior to or during sequencing. In embodiments, nucleic acids in a sample are not enriched and/or amplified prior to or during sequencing.

In certain embodiments, one nucleic acid sample from one plant is sequenced. In certain embodiments, nucleic acids from each of two or more samples are sequenced, where samples are from one plant or from different plants. In certain embodiments, nucleic acid samples from two or more biological samples are pooled, where each biological sample is from one plant or two or more plants, and the pool is sequenced. In the latter embodiments, a nucleic acid sample from each biological sample often is identified by one or more unique identifiers.

A sequencing method can utilize identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes and use of identifiers can facilitate analyzing a number of samples in each lane A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina’s multiplexing sample preparation oligonucleotide kit and multiplexing sequencing primers and PhiX control kit (e.g., Illumina’s catalog numbers PE-400-1001 and PE-400-1002, respectively).

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. For example, primers specific for the unique subsequences in the TPS gene profile of a plant genome can be used for a targeted enrichment, amplification and/or sequencing approach. Any suitable method or combination of methods can be used for enrichment, amplification and/or sequencing of one or more subsets of targeted nucleic acids. In certain embodiments, targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets (e.g., primers provided herein). Sequence specific anchors often can be used as sequence-specific primers.

In embodiments, nucleic acid is sequenced and the sequencing product (e.q., a collection of sequence reads) is processed prior to, or in conjunction with, an analysis of the sequenced nucleic acid. For example, sequence reads can be processed according to one or more of the following: aligning, mapping, filtering, counting, normalizing, weighting, generating a profile, and the like, and combinations thereof. Certain processing steps may be performed in any order and certain processing steps may be repeated.

### Solid Supports

Provided herein are solid supports that include the primers provided herein. The primers can directly be attached to the solid support, such as by covalent linkage, or can otherwise be associated with the solid support. For example, the primers can include, in addition to a sequence complementary to a unique subsequence of a TPS gene or paralog thereof in the genome of a plant cultivar of interest, a sequence that is complementary to a nucleic acid sequence that is directly attached to the solid support. The solid supports that include the primers provided herein can be contacted with nucleic acid from a sample obtained from a plant cultivar, under conditions that facilitate hybridization of a primer to a corresponding unique subsequence of a TPS gene or paralog thereof in the genome of a plant cultivar of interest. The resulting hybrids can directly be analyzed, such as by a signal or a label, for the presence or absence of hybridized product containing one or more primers specifically bound to a unique subsequence of a TPS gene in the nucleic acid. Alternately, the resulting hybrids can be subjected to polymerase-based extension reaction conditions using, e.g., one or more labeled nucleotides that can be incorporated into an extension product, thereby identifying, based on the presence or absence of a label in the extension product, whether a TPS gene or paralog thereof is present in the genome of a plant cultivar of interest.

The term “solid support” or “solid phase” as used herein refers to a wide variety of materials including solids, semi-solids, gels, films, membranes, meshes, felts, composites, particles, and the like typically used to sequester molecules, and more specifically refers to an insoluble material with which nucleic acid can be associated. A solid support for use with processes described herein sometimes is selected in part according to size: solid supports having a size smaller than the size a microreactor sometimes are selected. Examples of solid supports for use with processes described herein include, without limitation, beads (e.g., microbeads, nanobeads), particles (e.g., microparticles, nanoparticles) and chips.

The terms “beads” and “particles” as used herein refer to solid supports suitable for associating with biomolecules, and more specifically nucleic acids. Beads may have a regular (e.g., spheroid, ovoid) or irregular shape (e.g., rough, jagged), and sometimes are non-spherical (e.g., angular, multi-sided). Particles or beads having a nominal, average or mean diameter less than the nominal, average, mean or minimum diameter of a microreactor can be utilized. Particles or beads having a nominal, average or mean diameter of about 1 nanometer to about 500 micrometers can be utilized, such as those having a nominal, mean or average diameter, for example, of about 10 nanometers to about 100 micrometers; about 100 nanometers to about 100 micrometers; about 1 micrometer to about 100 micrometers; about 10 micrometers to about 50 micrometers; about 1, 5, 10, 15, 20, 25, 30, 35, 40, 45, 50, 55, 60, 65, 70, 75, 80, 85, 90, 95, 100, 200, 300, 400, 500, 600, 700, 800 or 900 nanometers; or about 1, 5, 10, 15, 20, 25, 30, 35, 40, 45, 50, 55, 60, 65, 70, 75, 80, 85, 90, 95, 100, 200, 300, 400, 500 micrometers.

A bead or particle can be made of virtually any insoluble or solid material. For example, the bead or particle can comprise or consist essentially of silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidenedifluoride (PVDF)) and the like. Beads or particles may be swellable (e.g., polymeric beads such as Wang resin) or non-swellable (e.g., CPG). Commercially available examples of beads include without limitation Wang resin, Merrifield resin and Dynabeads®. Beads may also be made as solid particles or particles that contain internal voids.

The solid supports can be provided in a collection of solid supports. A solid support collection can include two or more different solid support species. The term “solid support species” as used herein refers to a solid support in association with one particular primer or primer pair provided herein, or a combination of different primers or primer pairs. In certain embodiments, a solid support includes about 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 650 or 700 or more primers that specifically bind to unique subsequences of one or more TPS genes or paralogs thereof in one or more plant cultivars of interest. The solid supports (e.g., beads) in the collection of solid supports can be homogeneous (e.g., all are Wang resin beads) or heterogeneous (e.g., some are Wang resin beads, and some are magnetic beads). In certain embodiments, one or more primers selected from among SEQ ID NOS:1-1284, 1398, 1399, in Primer Groups 1-19 as set forth in Tables 1-16, and in B3/F3 polynucleotide primer pairs set forth in Tables 17-35, are attached or otherwise associated with a solid support, or a collection of solid supports. In embodiments, one or more primers selected from among those set forth in SEQ ID NOS: 1-1284, 1398, 1399, in Primer Groups 1-19 as set forth in Tables 1-16, and in B3/F3 polynucleotide primer pairs set forth in Tables 17-35, or sequences that share 90%, 91%, 92%, 93%, 94%, 95%, 96%, 97%, 98%, 99% or more identity with any of the sequences set forth in SEQ ID NOS: 1-1284, are attached or otherwise associated with a solid support, or a collection of solid supports.

The primers attached to the solid supports generally are single-stranded and are of any type suitable for hybridizing sample nucleic acid (e.g., DNA, RNA, analogs thereof (e.g., peptide nucleic acid (PNA)), chimeras thereof (e.g., a single strand comprises RNA bases and DNA bases) and the like). The primers can be associated with the solid support in any manner suitable for hybridization of the primers to nucleic acid from the plant cultivar. The primers can be in association with a solid support by a covalent linkage or a non-covalent interaction. Non-limiting examples of non-covalent interactions include hydrophobic interactions (e.g., C18 coated solid support and tritylated nucleic acid), polar interactions (e.g., “wetting” association between nucleic acid/polyethylene glycol), pair interactions including without limitation, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA) and the like.

The primers provided herein also can be associated with a solid support by different methodology, which include, without limitation (i) sequentially synthesizing nucleic acid directly on a solid support, and (ii) synthesizing nucleic acid, providing the nucleic acid in solution phase and linking the nucleic acid to a solid support. The primers can be linked covalently at various sites in the nucleic acid to the solid support, such as (i) at a 1′, 2′, 3′, 4′ or 5′ position of a sugar moiety or (ii) a pyrimidine or purine base moiety, of a terminal or non-terminal nucleotide of the nucleic acid, for example. The 5′ terminal nucleotide of the primer can be linked to the solid support, in certain embodiments.

Methods for sequentially synthesizing nucleic acid directly on a solid support are known. For example, the 3′ end of nucleic acid can be linked to the solid support (e.g., phosphoramidite method described in Caruthers, Science 230: 281-286 (1985)) or the 5′ end of the nucleic acid can be linked to the solid support (e.g., Claeboe et al, Nucleic Acids Res. 31(19): 5685-5691 (2003)).

Methods for linking solution phase nucleic acid to a solid support also are known (e.g., U.S. Pat. No. 6,133,436, naming Koster et al. and entitled “Beads bound to a solid support and to nucleic acids” and WO 91/08307, naming Van Ness and entitled “Enhanced capture of target nucleic acid by the use of oligonucleotides covalently attached to polymers”). Examples include, without limitation, thioether linkages (e.g., thiolated nucleic acid); disulfide linkages (e.g., thiol beads, thiolated nucleic acid); amide linkages (e.g., Wang resin, amino-linked nucleic acid); acid labile linkages (e.g., glass beads, tritylated nucleic acid) and the like. Nucleic acid can be linked to a solid support without a linker or with a linker (e.g., S. S. Wong, “Chemistry of Protein Conjugation and Cross-Linking,” CRC Press (1991), and G. T. Hermanson, “Bioconjugate Techniques,” Academic Press (1995). A homo or hetero-biofunctional linker reagent, can be selected, and examples of linkers include without limitation N-succinimidyl(4-iodoacetyl) aminobenzoate (SlAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl)cyclohexane-1-carboxylate (SMCC), 6-hydrazinonicotimide (HYNIC), 3-amino-(2-nitrophenyl)propionic acid and the like.

Nucleic acid can be synthesized using standard methods and equipment, such as the ABl®3900 High Throughput DNA Synthesizer and EXPEDITE®8909 Nucleic Acid Synthesizer, both of which are available from Applied Biosystems (Foster City, CA). Analogs and derivatives are described in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing nucleic acids containing such analogs or derivatives are disclosed, for example, in the patent publications cited above and in U.S. Pat. Nos. 5,614,622; 5,739,314; 5,955,599; 5,962,674; 6,117,992; in WO 00/75372 and in related publications. In certain embodiments, analog nucleic acids include inosines, abasic sites, locked nucleic acids, minor groove binders, duplex stabilizers (e.g., acridine, spermidine) and/or other melting temperature modifiers (e.g., target nucleic acid, solid phase nucleic acid, and/or primer nucleic acid may comprise an analog).

The density of solid phase-bound primer molecules per solid support unit (e.g., one bead or one sample location of a chip) can be selected. A maximum density can be selected that allows for hybridization of sample nucleic acid from the plant cultivar to solid phase-bound primers. In certain embodiments, solid phase-bound primer density per solid support unit (e.g., nucleic acid molecules per bead) is about 5 nucleic acids to about 10,000 nucleic acids per solid support. The density of the solid phase-bound primer per unit solid support in some embodiments can be about 5, 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 nucleic acids per solid support. In certain embodiments the density of the solid phase-bound primer per unit solid support is about 1 to 1 (e.g., one molecule of solid phase nucleic acid to one bead).

In certain embodiments, the solid supports can include any number of primer species useful for carrying out the analysis methods provided herein. Solid supports having primers attached or otherwise attached thereto can be provided in any convenient form for contacting a sample nucleic acid from a plant cultivar, such as solid or liquid form, for example. In certain embodiments, a solid support can be provided in a liquid form optionally containing one or more other components, which include without limitation one or more buffers or salts. Solid supports of a collection can be provided in one container or can be distributed across multiple containers.

Solid supports can be provided in an array in certain embodiments, or instructions can be provided to arrange solid supports in an array on a substrate. The term “array” as used herein can refer to an arrangement of sample locations (for nucleic acid samples from plant cultivars) on a single two-dimensional solid support, or an arrangement of solid supports across a two-dimensional surface. An array can be of any convenient general shape (e.g., circular, oval, square, rectangular). An array can be referred to as an “X by Y array” for square or rectangular arrays, where the array includes X number of sample locations or solid supports in one dimension and Y number of sample locations or solid supports in a perpendicular dimension. An array can be symmetrical (e.g., a 16 by 16 array) or non-symmetrical (e.g., an 8 by 16 array). An array may include any convenient number of sample locations or solid supports in any suitable arrangement. For example, X or Y independently can be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 in some embodiments.

An array can contain one solid support species or multiple solid support species from a collection. The array can be arranged on any substrate suitable for sequence analysis or manufacture processes described herein. Examples of substrates include without limitation flat substrates, filter substrates, wafer substrates, etched substrates, substrates having multiple wells or pits (e.g., microliter (about 1 microliter, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900 and up to about 999 microliter volume), nanoliter (1 nanoliter, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900 and up to about 999 nanoliter volume), picoliter (1 picoliter, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900 and up to about 999 picoliter volume) wells or pits; wells having filter bottoms), substrates having one or more channels, substrates having one or more electrodes, chips and the like, and combinations thereof. Wells or pits of multiple well and pit substrates can contain one or more solid support units (e.g., each unit being a single bead or particle). Substrates can include a suitable material for conducting sequence analysis or nucleic acid manufacture processes described herein, including without limitation, fiber (e.g., fiber filters), glass (e.g., glass surfaces, fiber optic surfaces), metal (e.g., steel, gold, silver, aluminum, silicon and copper; metal coating), plastic (e.g., polyethylene, polypropylene, polyamide, polyvinylidenedifluoride), silicon and the like. In certain embodiments, the array can be a microarray or a nanoarray. A “nanoarray,” often is an array in which solid support units are separated by about 0.1 nanometers to about 10 micrometers, for example from about 1 nanometer to about 1 micrometer (e.g. about 0.1 nanometers, 0.5, 1, 2, 3, 4, 5, 10, 20, 30, 40, 50, 60, 70, 80, 90, 100, 200, 300, 400, 500, 600, 700, 800, 900 nanometers, 1 micrometer, 2, 3, 4, 5, 6, 7, 8, 9, and up to about 10 micrometers). A “microarray” is an array in which solid support units are separated by more than 1 micrometer. The density of solid support units on arrays often is at least 100/cm², and can be 100/cm² to about 10,000/cm², 100/cm² to about 1,000/cm² or about 150, 200, 300, 400, 500, 600, 700, 800, 900, 1000, 2000, 3000, 4000, 5000, 6000, 7000, 8000, 9000 or 10000 solid support units/cm².

### Applications / Uses

The methods provided herein can provide an outcome indicative of one or more characteristics of a plant cultivar, including, but not limited to, a TPS gene profile, a terpene profile, a cannabinoid profile, a flavonoid profile, and the presence of a genetic variation in a TPS gene or paralog thereof.

This information in turn permits identifying and selecting plants of desired genotype or phenotype for agricultural, industrial or medicinal applications based on desired characteristics, such as lineage, resistance or affinity for an organism or condition, or therapeutic activity, and using the selected plants or portions or extracts thereof in methods provided herein, such as methods of breeding, methods of cultivating a crop, and methods of treatment.

Provided herein are plant cultivars identified as containing at least one terpene synthase gene having at least one subsequence, such as an exon, that is amplified by the methods provided herein. In embodiments, the plant cultivars are Cannabis plant cultivars. Examples of plant cultivars selected for desirable properties (e.g., medicinal uses, insecticidal properties) according to the methods provided herein include, but are not limited to:

(a)- A plant that would produce a non-volatile extract to preserve the smell, taste, and aroma of the plant material or an extract thereof by selecting parent cultivars to breed offspring expressing terpene synthase genes that provide such properties, e.g., one or more of the genes designated as follows, or genes that are similar thereto in sequence, structure and/or function, and/or products thereof:
Sesquiterpene Synthases, aka “TPS-a” gene list. Examples include:
   TPS4-like
   TPS9-like1
   TPS9-like2
   TPS50
   TPS18
   TPS14
   TPS7
   TPS4
   TPS32
   TPS9
   TPS20
   TPS8-like
   TPS8
   TPS23
   TPS44
   TPS59
   TPS55
   TPS58
   TPS69
For example, TPS4-likeJL, TPS9-like1JL, TPS9-like2JL, TPS50JL, TPS18JL, TPS14JL, TPS7JL, TPS4JL, TPS32JL, TPS9JL, TPS20JL, TPS8-likeJL, TPS8JL, TPS23JL, TPS44JL, TPS59JL, TPS55JL, TPS58JL, TPS69JL.

(b)- A plant that would produce a volatile smell profile to produce an aromatic and fragrant extract and/or have anti-pathogenic properties by selecting parent cultivars to breed offspring expressing terpene synthase genes that provide such properties, e.g., one or more of the genes designated as follows, or genes that are similar thereto in sequence, structure and/or function, and/or products thereof:
Monoterpene Synthases, aka “TPS-b” gene list. Examples include:
   TPS13-like2
   TPS13
   TPS17
   TPS30
   TPS64
   TPS6-like
   TPS6
   TPS11-like
   TPS51
   TPS30-like
   TPS3
   TPS52
   TPS5
   TPS13-like1
   TPS42
   TPS1
   TPS53
   TPS12
   TPS40
   TPS63
   TPS33
   TPS61
   TPS12-like
   TPS62
   TPS2
   TPS43
   TPS11
   TPS38
   TPS36
   TPS37
For example, TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPS5JL, TPS13-like1JL, TPS42JL, TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37JL.

(c)- A plant having the absence of one or more monoterpene synthase (TPS-b) genes that use GPP as a precursor to allow for greater cannabinoid production by selecting parent cultivars to breed to breed offspring not expressing or having reduced expression of terpene synthase genes that interfere with cannabinoid production, e.g., one or more of the genes listed in (b) above, or genes that are similar thereto in sequence, structure and/or function, and/or products thereof.

(d)- A plant that would contain one or more root specifically expressed terpene synthases to increase resistance against pests in the soil and/or respond favorably to beneficial microorganisms in the soil such as beneficial insects, mycorrhizal fungi and beneficial bacteria by selecting parent cultivars to breed offspring expressing terpene synthase genes and/or products that provide such properties, e.g., one or more of the genes designated as follows, or genes that are similar thereto in sequence, structure and/or function, and/or products thereof:
TPS11
TPS49
TPS41
TPS12
TPS11-like
TPS36
TPS6
TPS37
TPS64
For example, TPS11JL, TPS49JL, TPS41JL, TPS12JL, TPS11-likeJL, TPS36JL, TPS6JL, TPS37JL, TPS64JL.

(e)- A plant that would contain one or more predominantly stem specifically expressed terpene synthases to increase resistance against pests that are stem-hosted, e.g., stem-hosted insects, by selecting parent cultivars to breed offspring expressing terpene synthase genes and/or products that provide such properties, e.g., one or more of the one or more of the genes designated as follows, or genes that are similar thereto in sequence, structure and/or function, and/or products thereof:
TPS63
TPS43
TPS6-like
TPS33
TPS24
For example, TPS63JL, TPS43JL, TPS41JL, TPS6-likeJL, TPS33JL, TPS24JL.

The cultivars listed in (f) through (r) below are identified and/or selected for the production of desired terpene product profiles for the indicated applications. Examples of enzymes that can generate all or part of the terpene product profiles for Cannabis are listed (“cs” TPS enzymes). It is understood that one of skill in the art can identify, for any given plant cultivar, TPS enzymes that are similar in sequence, structure and/or function as the indicated Cannabis TPS enzymes and can obtain specialized cultivars having similar terpene product profiles. In is understood that TPS enzymes that are similar in function to the indicated “Cs” (Cannabis Sativa) enzymes are contemplated in the cultivars and methods provided herein.

(f)- A plant for terpene dominance that changes the smell and/or therapeutic/physiologic effect of the plant and/or extract thereof, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| CsTPS9FN | β-caryophyllene |
| CsTPS3FN, csTPS5PK, csTPS5FN, csTPS15CT, csTPS17AK, csTPS23Choc, csTPS30PK (csTPS32PK, csTPS7PK, csTPS1SK, csTPS2SK) | Myrcene |
| CsTPS31PK, csTPS37FN (csTPS32PK, csTPS24Choc, csTPS1 SK) | Terpinolene |
| csTPS1SK, csTPS14CT (csTPS5FN, csTPS5PK, csTPS7FN, csTPS23Choc, csTPS30PK, csTPS32PK) | Limonene |
| CsTPS25LS (CsTPS32PK) | β-Farnescene |
| csTPS2SK (csTPS1SK, csTPS5FN, csTPS30PK) | Alpha-Pinene |

(g)- A plant for producing an energetic effect when the plant or an extract thereof is ingested and/or vaporized (e.g., as a spray or for inhalation), or selecting parent cultivars to breed offspring expressing or not expressing terpene synthase genes and/or products that result in such properties, e.g., presence or lack thereof of one or more of the following genes and/or products:

| | |
|---|---|
| Genes: Major Product (Minor Product) | Terpene Product to Breed For: |
| CsTPS18VF (CsTPS19BL) | S-linalool |
| CsTPS31PK, csTPS37FN (csTPS32PK, csTPS24Choc, csTPS1SK) | Terpinolene |
| csTPS6PK, csTPS13PK, and csTPS38FN (csTPS7AK) | β-Ocimene |
| CsTPS2FN, csTPS5FN (CsTPS32PK | α-Pinene >> β-pinene |
| CsTPS18Choc (CsTPS19BL) | Lack of R-linalool |
| (csTPS5PK, csTPS17AK, csTPS31PK, csTPS32PK) | Lack of α-terpineol |
| | Lack of fenchol |
| csTPS37LPA5 | 3-carene |

(h)- A plant for producing a sedative effect when the plant or an extract thereof is ingested and/or vaporized (e.g., as a spray or for inhalation), or selecting parent cultivars to breed offspring expressing or not expressing terpene synthase genes and/or products that result in such properties, e.g., presence or lack thereof of one or more of the following genes and/or products:

| | |
|---|---|
| Genes: Major Product (Minor Product) | Terpene Product to Breed For: |
| CsTPS31PK, csTPS2SK (csTPS1SK, csTPS5FN, csTPS5PK, csTPS30PK) | β-pinene = α-Pinene (equal amounts) |
| CsTPS18Choc (CsTPS19BL) | R-linalool |
| csTPS1SK, csTPS14CT (csTPS5FN, csTPS5PK, csTPS7FN, csTPS23Choc, csTPS30PK, csTPS32PK) | Limonene |
| csTPS18VF, csTPS19BL, csTPS35LS (csTPS22PK, csTPS25LS, csTPS32PK) | Trans-nerolidol |
| (csTPS5PK, csTPS17AK, csTPS31PK, csTPS32PK) | Terpineol |
| CsTPS32PK | Camphene |
| csTPS6PK, csTPS13PK, and csTPS38FN (csTPS7AK) | Lack of β-ocimene |
| CsTPS18VF (CsTPS19BL) | Lack of S-Linalool |
| CsTPS31PK, csTPS37FN (csTPS32PK, csTPS24Choc, csTPS1SK) | Lack of Terpinolene |

(i)- A plant that would produce a cognitive-enhancing effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2FN, csTPS5FN, and csTPS32PK | α-Pinene >> β-pinene |
| csTPS6PK, csTPS13PK, and csTPS38FN (csTPS7AK) | β-ocimene |
| | Eucalyptol |

(j)- A plant that would produce an appetite-suppressing effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| CsTPS9FN (csTPS4FN, csTPS22PK) | Humulene |

(k)- A plant that would produce an anti-inflammatory effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2SK (csTPS1SK, csTPS5FN, csTPS30PK) | α-Pinene |
| CsTPS9FN (csTPS4FN, csTPS22PK) | Humulene |
| CsTPS9FN | β-caryophyllene |

(I)- A plant that would produce an anxiolytic (anti-anxiety) effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |

| | |
|---|---|
| csTPS2SK (csTPS1SK, csTPS5FN, csTPS30PK) | α-Pinene |
| CsTPS9FN (csTPS4FN, csTPS22PK) | Humulene |
| CsTPS9FN | β-caryophyllene |
| csTPS17AK, csTPS18VF, csTPS18Choc, csTPS19BL, csTPS29BC, csTPS35LS (csTPS31PK) | Linalool |
| csTPS18VF, csTPS19BL, csTPS35LS (csTPS22PK, csTPS25LS, csTPS32PK) | Nerolidol |
| csTPS1SK, csTPS14CT (csTPS5FN, csTPS5PK, csTPS7FN, csTPS23Choc, csTPS30PK, csTPS32PK) | Limonene |

(m)- A plant that would produce an antinociceptive effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Products) | Terpene Product to Breed For: |
| csTPS5PK (csTPS31PK, csTPS32PK) | α-bisabolol |
| (csTPS5PK, csTPS17AK, csTPS31PK, csTPS32PK) | α -terpineol |
| csTPS18VF, csTPS19BL, csTPS35LS (csTPS22PK, csTPS25LS, csTPS32PK) | trans nerolidol |
| (CsTPS2SK, csTPS32PK, TPS37LPA5) | α-phellandrene |
| | Eucalyptol |

(n)- A plant that would produce a body relaxing effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS5PK (csTPS31PK, csTPS32PK) | α-bisabolol |
| (csTPS5PK, csTPS17AK, csTPS31PK, csTPS32PK) | α -terpineol |
| csTPS18VF, csTPS19BL, csTPS35LS (csTPS22PK, csTPS25LS, csTPS32PK) | trans nerolidol |
| (CsTPS2SK, csTPS32PK, TPS37LPA5) | α-phellandrene |

(o)- A plant that would produce an anti-depressant effect when the plant, or extract thereof, is ingested and/or vaporized, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| CsTPS31PK, csTPS2SK (csTPS1SK, csTPS5FN, csTPS5PK, csTPS30PK) | b-pinene = a-Pinene |
| csTPS1SK, csTPS14CT (csTPS5FN, csTPS5PK, csTPS7FN, csTPS23Choc, csTPS30PK, csTPS32PK) | Limonene |
| csTPS18VF, csTPS19BL, csTPS35LS (csTPS22PK, csTPS25LS, csTPS32PK) | nerolidol |
| csTPS17AK, csTPS18VF, csTPS18Choc, csTPS19BL, csTPS29BC, csTPS35LS (csTPS31PK) | Linalool |

(p)- A plant that contains acetyl cholinesterase-inhibitor (AChEI) terpenes, or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2SK (csTPS1SK, csTPS5FN, csTPS30PK) | α-pinene |
| CsTPS31PK, csTPS37FN (csTPS32PK, csTPS24Choc, csTPS1SK) | Terpinolene |
| csTPS6PK, csTPS13PK, and csTPS38FN (csTPS7AK) | β-ocimene |
| TPS37LPA5 | (3-Carene) |
| csTPS33PK and TPS37LPA5 | α- and γ-terpinene |
| (csTPS5FN, csTPS7FN, csTPS30PK) | Sabinene |

(q)- A plant that contain one or more of the Herbivore-induced Plant Volatiles (HIPV) terpene synthases (see, e.g., Booth et al., Plant Physiol., 184(1):130-147 (2020), the contents of which are incorporated in their entirety by reference herein), or selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS6PK, csTPS13PK, csTPS38FN (csTPS7AK) | Ocimene |
| csTPS17AK, csTPS18VF, csTPS18Choc, csTPS19BL, csTPS29BC, csTPS35LS (csTPS31PK) | Linalool |
| csTPS5PK (csTPS31PK, csTPS32PK) | Bisabolol |
| csTPS18VF, csTPS19BL, csTPS35LS (csTPS22PK, csTPS25LS, csTPS32PK) | Nerolidol |
| csTPS2SK (csTPS1SK, csTPS5FN, csTPS30PK) | α-pinene |
| (csTPS2SK, csTPS1SK, csTPS5FN, csTPS5PK, csTPS30PK) | β-pinene |
| csTPS1SK, csTPS14CT (csTPS5FN, csTPS5PK, csTPS7FN, csTPS23Choc, csTPS30PK, csTPS32PK) | Limonene |
| (csTPS5PK, csTPS17AK, csTPS31 PK, csTPS32PK) | α-terpineol |
| (csTPS5PK, csTPS17AK, csTPS31 PK, csTPS32PK) | Y-elemene |
| (csTPS5PK, csTPS31PK, csTPS32PK) | Bergamotene |
| (csTPS8FN, csTPS16CC, csTPS22PK, csTPS28PK) | Eudesmol |
| csTPS16CC | Germacrene B |
| N/A | Guiaol |
| (csTPS5PK, csTPS17AK, csTPS31PK, csTPS32PK) | Fenchol |
| N/A | Eugenol |

(r)- A plant that produces, in the plant or in an extract thereof, one or more of the following properties, or selecting parent cultivars to breed offspring expressing terpene synthase genes and/or products that provide such properties, e.g., one or more of the following genes and/or gene products:

Antibacterial Properties:

| | |
|---|---|
| Genes: Major Product (Minor Product) | Terpene Product to Breed For: |
| CsTPS4FN | Aromadendrene |
| | Carvacrol |
| csTPS9FN | β-Caryophyllene |
| TPS-b and/or other | Eucalyptol (1,8-Cineole) |
| TPS-b and/or other | Fenchol |
| csTPS16CC | Germacrene D |
| CsTPS32PK (CsTPS17AK) | Nerol (cis-Geraniol) |
| TPS-b and/or other | Pulegone |
| (csTPS5FN, csTPS7FN, csTPS30PK) | Sabinene |
| CsTPS32PK (CsTPS17AK) | Geraniol |

Antimicrobial Properties:

| | |
|---|---|
| Genes: Major Product (Minor Product) | Terpene Product to Breed For: |
| (CsTPS32PK) | Camphor |
| (csTPS30PK, CsTPS7FN) | Sabinene Hydrate |
| csTPS33PK | Thymol |

Fungicidal Properties:

| | |
|---|---|
| Genes: Major Product (Minor Product) | Terpene Product to Breed For: |
| (CsTPS35PK) | Citronellol |
| csTPS33PK | para-Cymene |
| TPS-b and/or other | Pulegone |
| CsTPS32PK | Geraniol |

Herbicidal Properties:

| | |
|---|---|
| Genes: Major Product (Minor Product) | Terpene Product to Breed For: |
| CsTPS32PK | Geraniol |
| TPS-b and/or other | Pulegone |
| (CsTPS35PK) | Citronellol |
| TPS-b and/or other | Borneol |
| csTPS33PK | para-Cymene |

Pesticidal Properties:

| | |
|---|---|
| Genes: Major Product (Minor Product) | Terpene Product to Breed For: |
| CsTPS4FN - TPS9-like2JL, TPS4-likeJL | Aromadendrene |
| CsTPS5PK (CsTPS32PK - TPS5JL) | α-Bisabolol |
| TPS-a | Cedrol |
| CsTPS18VF, csTPS19BL, csTPS35LS, (csTPS22PK, csTPS25LS, csTPS32PK) | Nerolidol |
| CsTPS18VF, csTPS19BL, csTPS35LS, (csTPS22PK, csTPS25LS, csTPS32PK) | trans-Nerolidol |
| TPS-a | Guaiol |

Pheromone: Insect attractant (e.g., for pollination; to attract insects that are predators of other insects or other pathogens that cause plant damage) or insecticidal (e.g., contact or fumigant toxicities)

| | |
|---|---|
| Genes: Major Product (Minor Product) | Terpene Product to Breed For: |
| | endo-Borneol |
| | Isoborneol |
| TPS37LPA5 | (3-Carene) |
| | Carveol |
| csTPS16CC | Germacrene B |
| CsTPS21AK | Hedycaryol |
| | Menthol |
| CsTPS18VF, csTPS19BL, csTPS35LS, (csTPS22PK, csTPS25LS, csTPS32PK) | cis-Nerolidol |
| CsTPS6FN, csTPS13PK, csTPS38FN, (csTPS17AK) | cis-β-Ocimene |
| CsTPS6FN, csTPS13PK, csTPS38FN, (csTPS17AK) | trans-β-Ocimene |
| (csTPS30PK, CsTPS7FN) | Sabinene Hydrate |
| csTPS33PK and TPS37LPA5 | α-Terpinen |
| csTPS33PK | Thymol |
| CsTPS25LS (CsTPS32PK) | β-Farnesene |
| CsTPS25LS | α-Farnesene |
| CsTPS8FN, (csTPS16CC, csTPS22PK, csTPS28PK) | γ-Eudesmol |
| CsTPS4FN | Alloaromadendrene |
| (CsTPS8FN, csTPS4FN) | Valencene |
| TPS-b and/or other | Pulegone |

Expectorant Properties:

| | |
|---|---|
| Genes: Major Product (Minor Product) | Terpene Product to Breed For: |
| CsTPS32PK | Camphene |
| CsTPS32PK | Geraniol |
| (csTPS30PK, CsTPS7FN) | Sabinene Hydrate |

Non-irritant properties: (Breed for reduced production or absence of, e.g., one or more of the following terpene products that are irritants, which can be useful, e.g., when breeding plants such as Cannabis for dermatological uses in salves, creams, ointment and transdermal applications)

| | |
|---|---|
| Genes: Major Product (Minor Product) | Terpene Product to Breed for Absence/Reduced Amount: |
| TPS-b and/or other | Borneol |
| | α-Cedrene |
| (CsTPS35PK) | Citronellol |
| csTPS33PK | para-Cymene |
| | Fenchone (Presence — Counterirritant) |

The methods of preparing and/or analyzing nucleic acids provided herein, and the primers provided herein for such analysis, permit the identification and select for plants that contain the TPS gene(s) and/or variants of the gene(s) that have desirable characteristics such as desired terpene profiles, ratios of monoterpenes to sesquiterpenes, and/or terpenes that confer agronomic or pathogenesis-related traits (such as insect, pest, mold, mildew, fungus, bacterial, or environmental resistance, as well as attract certain predator or beneficial organisms). The selection can, in embodiments, be used to identify desired parental lines for breeding daughter cultivars that contain desired combinations of these TPS genes or variants of these genes. In addition, the primers provided herein can be used to identify offspring/daughter cultivars that contain the desired gene(s)/variants of these gene(s) from one or both parent cultivars. In addition, the methods of preparing and/or analyzing nucleic acids provided herein, and the primers provided herein for such analysis, can be used for lineage- specific analysis to identify related and distant cultivars to in-breed or out-cross plant cultivars, such as Cannabis cultivars, based on the genetic profiling of unique subsequences (e.g., exons) of TPS genes. Other applications include, but are not limited to:
(1) Using the TPS gene-specific primers provided herein to increase or decrease terpene production, concentration and bioaccumulation in a plant cultivar, such as Cannabis, including, but not limited to the following terpenes:
   α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Farnesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, _{Y}-Terpinene, α-Terpineol, Terpinolene, Valencene, _{Y}-Elemene, Z-Ocimene, E-Ocimene, α-Thujone, Thujene, _{Y}-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, _{Y}-gurjunene, α-bulnesene, Bulnesol, α-eudesmol, β-eudesmol, Hedycaryol, _{Y}-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A, Artemisinin

Identify more or less active variants of terpene synthase genes for transgenic experiments including CRISPR, Cre-Lox, and other genetic modification applications to transfer the more or less active variant to another Cannabis cultivar via breeding methods strategies while using the primers provided herein to track the inheritance of that gene variant.

Identify the sub-cellular localization of the TPS genes through identifying the amplicons generated in the methods provided.

Selecting for terpene genes with tissue-specific expression behavior, such as root, flower, stem, or leaf specific terpene synthase genes.

Using the TPS gene-specific primers provided herein in a microassay based presence/absence variation (PAV) identification screening tool, to identify the presence or absence of a TPS gene, including whether a genetic variant is present. In embodiments, a panel of information about several or all TPS genes in a plant cultivar can be obtained, and this information can be related to overall terpene production and accumulation in the plant cultivar.

Using the TPS gene-specific primers provided herein in a cDNA microassay based expression screening tool to identify the level of expression of each gene in the TPS family of a plant cultivar and, in embodiments, relating the level of expression of this panel of genes to overall terpene production and accumulation.

Using the TPS gene-specific primers provided herein to identify and select for gene variants of monoterpene synthases that would deplete the pre-cursor pool of GPP to lower overall cannabinoid and flavonoid concentrations and, in embodiments, breeding these genes into a higher cannabinoid producing cultivar to lower overall cannabinoid content.

Using the TPS gene-specific primers provided herein to identify gene variants of monoterpene synthases that would deplete the pre-cursor pool of GPP to raise the overall cannabinoid and flavinoid concentrations and, in embodiments, breeding this genetic profile into another low cannabinoid producing cultivar to higher overall cannabinoid content using these molecular markers.

Using the TPS gene-specific primers provided herein to select TPS gene combinations that provide specific terpene concentration/production profiles in plants of varying cannabinoid concentration, to decrease the cytotoxicity of the plant extract for medicinal application.

Using the TPS gene-specific primers provided herein, select TPS gene variants that are linked to higher or lower cannabinoid producing cultivars.

In certain aspects, the TPS gene-specific primers provided herein, or subsets thereof, can be used, e.g., in genetic testing and/or amplicon sequencing, to identify plants having a TPS gene profile, TPS gene expression profile, one or more TPS gene variants, a terpene profile, a cannabinoid profile, a flavonoid profile or other characteristics or combinations thereof that impart certain properties to the plant including, but not limited to: pathogen resistance (e.g., insect resistance, fungus resistance), adaptability to regional geographic or environmental features that would make the plant less prone to diseases or predators in a certain region or environment (e.g., resistant to certain diseases or predators at the humidity level in the environment in which the plant is grown), or a desired medicinal use or medical effect. In certain embodiments, the medicinal uses / medical effects are selected from among one or more of antioxidant, anti-inflammatory, antibacterial, antiviral, anti-anxiety, antinociceptive, analgesic, antihypertensive, sedative, antidepressant, acetylcholine esterase inhibition (AChEI), neuro-protective and gastro-protective effects. In embodiments, at least one therapeutic effect is AChEl and in certain embodiments, the analytes are terpenes and the terpenes that are scored include one or more terpenes selected from among alpha pinene, eucalyptol, 3 carene, alpha terpinene, gamma terpinene, cis ocimene, trans ocimene and beta caryophyllene oxide. In certain embodiments, at least one therapeutic effect is analgesic and in embodiments, the analytes are terpenes and the terpenes that are scored comprise one or more terpenes selected from among alpha bisabolol, alpha terpineol, alpha phellandrene and nerolidol.

For example, subsets of these primers can be applied in various specific tests to classify a strain’s effect on its user through genetic testing and/or amplicon sequencing. In aspects, sets of between 1-50, 1-45, 1-40, 1-35, 1-30, 1-25, 1-20, 1-15, 1-10, 1-5, 1-4, 1-3, 2 or 1 TPS genes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, up to 100 or more TPS genes can be assigned as imparting one or more desired property to a plant cultivar. For example, sets of 1-10 TPS genes, when present in a Cannabis cultivar, can be characterized as involved in a specific feeling achieved from administration, e.g., by inhalation or ingestion of a product derived from the Cannabis cultivar. For example, the primers could be used for exon-specific genotyping on genomic DNA for a specific subset of genes to identify genotypes that lead to a change in the presence or level of certain terpenes that are known to be associated with medical/physiological effects such as energy, sedation, mental clarity, mental and physical impairments, appetite stimulation or suppression, and/or the other common effects that are associated with products derived from Cannabis or other plant cultivars, or that are known to be associated with pathogen resistance in a Cannabis or other plant cultivar. In aspects, the TPS genes or portions thereof identified by the methods provided herein and used in the methods provided herein include any TPS gene or combinations of TPS genes that produce one or more of: one or more terpenes selected from among α-Bisabolol, endo-Borneol, Camphene, Camphor, 3-Carene, Caryophyllene, Caryophyllene Oxide, α-Cedrene, Cedrol, Citronellol, Eucalyptol (1,8 Cineole), α-Farnesene, β-Famesene, Fenchol, Fenchone, Geraniol, Geranyl Acetate, Guaiol, Humulene, Isoborneol, Isopulegol, D-Limonene, Linalool, Menthol, β-Myrcene, Nerol, trans-Nerolidol, cis-Nerolidol, trans-Ocimene, cis-Ocimene, α-Phellandrene, Phytol 1, Phytol 2, α-Pinene, β-Pinene, Pulegone, Sabinene, Sabinene Hydrate, α-Terpinene, _{Y}-Terpinene, α-Terpineol, Terpinolene, Valencene, _{Y}-Elemene, Z-Ocimene, E-Ocimene, α-Thujone, Thujene, _{Y}-Muurolene, 2-Norpinene, α-Santalene, α-Selinene, Germacrene D, Eudesma-3,7(11)-diene, δ-Cadinol, trans-α-Beramotene, trans-2-pinanol, p-cymen-8-ol, Sativene, Cyclosativene, α-guaiene, _{Y}-gurjunene, α-bulnesene, Bulnesol, α-eudesmol, β-eudesmol, Hedycaryol, _{Y}-eudesmol, Alloaromadendrene, p-cymene, α-Copaene, β-Elemene, α-Cubebene, Linalyl acetate, Bornyl acetate, Heptacosane, Tricosane, S-Limonene, (-)-Thujopsene, Hashenene 5,5-dimethyl-1-vinylbicyclo[2.1.1]hexane, (-)-englerin A and Artemisinin.

Examples of such breeding methods for a plant cultivar include, but are not limited to those described below (TPS gene nomenclature is characterized in part in Allen et al, PLoS ONE, 14(9):e0222363 (2019), the contents of which are expressly incorporated in their entirety by reference herein):

(a)- A method of breeding a plant that would produce a non-volatile extract to preserve the smell, taste, and aroma of the plant material or an extract thereof by selecting parent cultivars to breed offspring expressing terpene synthase genes that provide such properties, e.g., one or more of the genes designated as follows, or genes that are similar thereto in sequence, structure and/or function, and/or products thereof:
Sesquiterpene Synthases, aka “TPS-a” gene list. Examples include:
   TPS4-like
   TPS9-like1
   TPS9-like2
   TPS50
   TPS18
   TPS14
   TPS7
   TPS4
   TPS32
   TPS9
   TPS20
   TPS8-like
   TPS8
   TPS23
   TPS44
   TPS59
   TPS55
   TPS58
   TPS69
For example, TPS4-likeJL, TPS9-like1JL, TPS9-like2JL, TPS50JL, TPS18JL, TPS14JL, TPS7JL, TPS4JL, TPS32JL, TPS9JL, TPS20JL, TPS8-likeJL, TPS8JL, TPS23JL, TPS44JL, TPS59JL, TPS55JL, TPS58JL, TPS69JL.

(b)- A method of breeding a plant that would produce a volatile smell profile to produce an aromatic and fragrant extract and/or have anti-pathogenic properties by selecting parent cultivars to breed offspring expressing terpene synthase genes that provide such properties, e.g., one or more of the genes designated as follows, or genes that are similar thereto in sequence, structure and/or function, and/or products thereof:
Monoterpene Synthases, aka “TPS-b” gene list. Examples include:
   TPS13-like2
   TPS13
   TPS17
   TPS30
   TPS64
   TPS6-like
   TPS6
   TPS11-like
   TPS51
   TPS30-like
   TPS3
   TPS52
   TPS5
   TPS13-like1
   TPS42
   TPS1
   TPS53
   TPS12
   TPS40
   TPS63
   TPS33
   TPS61
   TPS12-like
   TPS62
   TPS2
   TPS43
   TPS11
   TPS38
   TPS36
   TPS37
For example, TPS13-like2JL, TPS13JL, TPS17JL, TPS30JL, TPS64JL, TPS6-likeJL, TPS6JL, TPS11-likeJL, TPS51JL, TPS30-likeJL, TPS3JL, TPS52JL, TPS5JL, TPS13-like1JL, TPS42JL,TPS1JL, TPS53JL, TPS12JL, TPS40JL, TPS63JL, TPS33JL, TPS61JL, TPS12-likeJL, TPS62JL, TPS2JL, TPS43JL, TPS11JL, TPS38JL, TPS36JL, TPS37JL.

(c)- A method of breeding a plant for the absence of one or more monoterpene synthase (TPS-b) genes that use GPP as a precursor to allow for greater cannabinoid production by selecting parent cultivars to breed to breed offspring not expressing or having reduced expression of terpene synthase genes that interfere with cannabinoid production, e.g., one or more of the genes listed in (b) above, or genes that are similar thereto in sequence, structure and/or function, and/or products thereof.

(d)- A method of breeding a plant that would contain one or more root specifically expressed terpene synthases to increase resistance against pests in the soil and/or respond favorably to beneficial microorganisms in the soil such as beneficial insects, mycorrhizal fungi and beneficial bacteria by selecting parent cultivars to breed offspring expressing terpene synthase genes and/or products that provide such properties, e.g., one or more of the genes designated as follows, or genes that are similar thereto in sequence, structure and/or function, and/or products thereof:
● TPS11
● TPS49
● TPS41
● TPS12
● TPS11-like
● TPS36
● TPS6
● TPS37
● TPS64
For example, TPS11JL, TPS49JL, TPS41JL, TPS12JL, TPS11-likeJL, TPS36JL, TPS6JL, TPS37JL, TPS64JL.

(e)- A method of breeding a plant that would contain one or more predominantly stem specifically expressed terpene synthases to increase resistance against pests that are stem-hosted, e.g., stem-hosted insects, by selecting parent cultivars to breed offspring expressing terpene synthase genes and/or products that provide such properties, e.g., one or more of the one or more of the genes designated as follows, or genes that are similar thereto in sequence, structure and/or function, and/or products thereof:
TPS63
TPS43
TPS6-like
TPS33
TPS24
For example, TPS63JL, TPS43JL, TPS41JL, TPS6-likeJL, TPS33JL, TPS24JL.

The methods listed in (f) through (r) below are for the production of desired terpene product profiles for the indicated applications. Examples of enzymes that can generate all or part of the terpene product profiles for Cannabis are listed (“cs” TPS enzymes). It is understood that one of skill in the art can identify, for any given plant cultivar, TPS enzymes that are similar in sequence, structure and/or function as the indicated Cannabis TPS enzymes and can obtain specialized cultivars having similar terpene product profiles.

(f)- A method of breeding a plant for terpene dominance that changes the smell and/or therapeutic/physiologic effect In the methods listed in (f) through (r) below, it is understood that TPS enzymes that are similar in function to the indicated “Cs” (Cannabis Sativa) enzymes

(f)- A method of breeding a plant for terpene dominance that changes the smell and/or therapeutic/physiologic effect of the plant and/or extract thereof, by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| CsTPS9FN | β-caryophyllene |
| CsTPS3FN, csTPS5PK, csTPS5FN, csTPS15CT, csTPS17AK, csTPS23Choc, csTPS30PK (csTPS32PK, csTPS7PK, csTPS1SK, csTPS2SK) | Myrcene |
| CsTPS31PK, csTPS37FN (csTPS32PK, csTPS24Choc, csTPS1SK) | Terpinolene |
| csTPS1SK, csTPS14CT (csTPS5FN, csTPS5PK, csTPS7FN, csTPS23Choc, csTPS30PK, csTPS32PK) | Limonene |
| CsTPS25LS (CsTPS32PK) | β-Famescene |
| csTPS2SK (csTPS1SK, csTPS5FN, csTPS30PK) | Alpha-Pinene |

(g)- A method of breeding a plant for producing an energetic effect when the plant or an extract thereof is ingested and/or vaporized (e.g., as a spray or for inhalation), by selecting parent cultivars to breed offspring expressing or not expressing terpene synthase genes and/or products that result in such properties, e.g., presence or lack thereof of one or more of the following genes and/or products:

| | |
|---|---|
| Genes: Major Product (Minor Product) | Terpene Product to Breed For: |
| CsTPS18VF (CsTPS19BL) | S-linalool |
| CsTPS31PK, csTPS37FN (csTPS32PK, csTPS24Choc, csTPS1SK) | Terpinolene |
| csTPS6PK, csTPS13PK, and csTPS38FN (csTPS7AK) | β-Ocimene |
| CsTPS2FN, csTPS5FN (CsTPS32PK | α-Pinene >> β-pinene |
| CsTPS18Choc (CsTPS19BL) | Lack of R-linalool |
| (csTPS5PK, csTPS17AK, csTPS31PK, csTPS32PK) | Lack of α-terpineol |
| | Lack of fenchol |
| csTPS37LPA5 | 3-carene |

(h)- A method of breeding a plant for producing a sedative effect when the plant or an extract thereof is ingested and/or vaporized (e.g., as a spray or for inhalation), by selecting parent cultivars to breed offspring expressing or not expressing terpene synthase genes and/or products that result in such properties, e.g., presence or lack thereof of one or more of the following genes and/or products:

| | |
|---|---|
| Genes: Major Product (Minor Product) | Terpene Product to Breed For: |
| CsTPS31PK, csTPS2SK (csTPS1SK, csTPS5FN, csTPS5PK, csTPS30PK) | β-pinene = α-Pinene (equal amounts) |
| CsTPS18Choc (CsTPS19BL) | R-linalool |
| csTPS1SK, csTPS14CT (csTPS5FN, csTPS5PK, csTPS7FN, csTPS23Choc, csTPS30PK, csTPS32PK) | Limonene |
| csTPS18VF, csTPS19BL, csTPS35LS (csTPS22PK, csTPS25LS, csTPS32PK) | Trans-nerolidol |
| (csTPS5PK, csTPS17AK, csTPS31PK, csTPS32PK) | Terpineol |
| CsTPS32PK | Camphene |
| csTPS6PK, csTPS13PK, and csTPS38FN (csTPS7AK) | Lack of β-ocimene |
| CsTPS18VF (CsTPS19BL) | Lack of S-Linalool |
| CsTPS31PK, csTPS37FN (csTPS32PK, csTPS24Choc, csTPS1SK) | Lack of Terpinolene |

(i)- A method of breeding a plant that would produce a cognitive-enhancing effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2FN, csTPS5FN, and csTPS32PK | α-Pinene >> β-pinene |
| csTPS6PK, csTPS13PK, and csTPS38FN (csTPS7AK) | β-ocimene |
| | Eucalyptol |

(j)- A method of breeding a plant that would produce an appetite-suppressing effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| CsTPS9FN (csTPS4FN, csTPS22PK) | Humulene |

(k)- A method of breeding a plant that would produce an anti-inflammatory effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2SK (csTPS1SK, csTPS5FN, csTPS30PK) | α-Pinene |
| CsTPS9FN (csTPS4FN, csTPS22PK) | Humulene |
| CsTPS9FN | β-caryophyllene |

(l)- A method of breeding a plant that would produce an anxiolytic (anti-anxiety) effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2SK (csTPS1SK, csTPS5FN, csTPS30PK) | α-Pinene |
| CsTPS9FN (csTPS4FN, csTPS22PK) | Humulene |
| CsTPS9FN | β-caryophyllene |
| csTPS17AK, csTPS18VF, csTPS18Choc, csTPS19BL, csTPS29BC, csTPS35LS (csTPS31PK) | Linalool |
| csTPS18VF, csTPS19BL, csTPS35LS (csTPS22PK, csTPS25LS, csTPS32PK) | Nerolidol |
| csTPS1SK, csTPS14CT (csTPS5FN, csTPS5PK, csTPS7FN, csTPS23Choc, csTPS30PK, csTPS32PK) | Limonene |

(m)- A method of breeding a plant that would produce an antinociceptive effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Products) | Terpene Product to Breed For: |
| csTPS5PK (csTPS31PK, csTPS32PK) | α-bisabolol |
| (csTPS5PK, csTPS17AK, csTPS31PK, csTPS32PK) | α -terpineol |
| csTPS18VF, csTPS19BL, csTPS35LS (csTPS22PK, csTPS25LS, csTPS32PK) | trans nerolidol |
| (CsTPS2SK, csTPS32PK, TPS37LPA5) | α-phellandrene |
| | Eucalyptol |

(n)- A method of breeding a plant that would produce a body relaxing effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS5PK (csTPS31PK, csTPS32PK) | α-bisabolol |
| (csTPS5PK, csTPS17AK, csTPS31PK, csTPS32PK) | α -terpineol |
| csTPS18VF, csTPS19BL, csTPS35LS (csTPS22PK, csTPS25LS, csTPS32PK) | trans nerolidol |
| (CsTPS2SK, csTPS32PK, TPS37LPA5) | α-phellandrene |

(o)- A method of breeding a plant that would produce an anti-depressant effect when the plant, or extract thereof, is ingested and/or vaporized by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| CsTPS31PK, csTPS2SK (csTPS1SK, csTPS5FN, csTPS5PK, csTPS30PK) | b-pinene = a-Pinene |
| csTPS1SK, csTPS14CT (csTPS5FN, csTPS5PK, csTPS7FN, csTPS23Choc, csTPS30PK, csTPS32PK) | Limonene |
| csTPS18VF, csTPS19BL, csTPS35LS (csTPS22PK, csTPS25LS, csTPS32PK) | nerolidol |
| csTPS17AK, csTPS18VF, csTPS18Choc, csTPS19BL, csTPS29BC, csTPS35LS (csTPS31PK) | Linalool |

(p)- A method of breeding a plant that contains acetyl cholinesterase-inhibitor (AChEI) terpenes by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS2SK (csTPS1SK, csTPS5FN, csTPS30PK) | α-pinene |
| CsTPS31PK, csTPS37FN (csTPS32PK, csTPS24Choc, csTPS1SK) | Terpinolene |
| csTPS6PK, csTPS13PK, and csTPS38FN (csTPS7AK) | β-ocimene |
| TPS37LPA5 | (3-Carene) |
| csTPS33PK and TPS37LPA5 | α- and γ-terpinene |
| (csTPS5FN, csTPS7FN, csTPS30PK) | Sabinene |

(q)- A method of breeding a plant that contain one or more of the Herbivore-induced Plant Volatiles (HIPV) terpene synthases (see, e.g., Booth et al., Plant Physiol., 184(1):130-147 (2020), the contents of which are incorporated in their entirety by reference herein), by selecting parent cultivars to breed offspring expressing terpene synthase genes and/or products that provide such properties, e.g., one or more of the following genes and/or products:

| | |
|---|---|
| Genes: Major Product (Minor Product) | Terpene Product to Breed For: |
| csTPS6PK, csTPS13PK, csTPS38FN (csTPS7AK) | Ocimene |
| csTPS17AK, csTPS18VF, csTPS18Choc, csTPS19BL, csTPS29BC, csTPS35LS (csTPS31PK) | Linalool |
| csTPS5PK (csTPS31 PK, csTPS32PK) | Bisabolol |
| csTPS18VF, csTPS19BL, csTPS35LS (csTPS22PK, csTPS25LS, csTPS32PK) | Nerolidol |
| csTPS2SK (csTPS1SK, csTPS5FN, csTPS30PK) | α-pinene |
| (csTPS2SK, csTPS1SK, csTPS5FN, csTPS5PK, csTPS30PK) | β-pinene |
| csTPS1SK, csTPS14CT (csTPS5FN, csTPS5PK, csTPS7FN, csTPS23Choc, csTPS30PK, csTPS32PK) | Limonene |
| (csTPS5PK, csTPS17AK, csTPS31PK, csTPS32PK) | α-terpineol |
| (csTPS5PK, csTPS17AK, csTPS31PK, csTPS32PK) | Y-elemene |
| (csTPS5PK, csTPS31PK, csTPS32PK) | Bergamotene |
| (csTPSBFN, csTPS16CC, csTPS22PK, csTPS28PK) | Eudesmol |
| csTPS16CC | Germacrene B |
| N/A | Guiaol |
| (csTPS5PK, csTPS17AK, csTPS31PK, csTPS32PK) | Fenchol |
| N/A | Eugenol |

(r)- A method of breeding a plant that produces, in the plant or in an extract thereof, one or more of the following properties by selecting parent cultivars to breed offspring expressing terpene synthase genes and/or products that provide such properties, e.g., one or more of the following genes and/or gene products:

Antibacterial Properties:

| | |
|---|---|
| Genes: Major Product (Minor Product) | Terpene Product to Breed For: |
| CsTPS4FN | Aromadendrene |
| | Carvacrol |
| csTPS9FN | β-Caryophyllene |
| TPS-b and/or other | Eucalyptol (1,8-Cineole) |
| TPS-b and/or other | Fenchol |
| csTPS16CC | Germacrene D |
| CsTPS32PK (CsTPS17AK) | Nerol (cis-Geraniol) |
| TPS-b and/or other | Pulegone |
| (csTPS5FN, csTPS7FN, csTPS30PK) | Sabinene |
| CsTPS32PK (CsTPS17AK) | Geraniol |

Antimicrobial Properties:

| | |
|---|---|
| Genes: Major Product (Minor Product) | Terpene Product to Breed For: |
| (CsTPS32PK) | Camphor |
| (csTPS30PK, CsTPS7FN) | Sabinene Hydrate |
| csTPS33PK | Thymol |

Fungicidal Properties:

| | |
|---|---|
| Genes: Major Product (Minor Product) | Terpene Product to Breed For: |
| (CsTPS35PK) | Citronellol |
| csTPS33PK | para-Cymene |
| TPS-b and/or other | Pulegone |
| CsTPS32PK | Geraniol |

Herbicidal Properties:

| | |
|---|---|
| Genes: Major Product (Minor Product) | Terpene Product to Breed For: |
| CsTPS32PK | Geraniol |
| TPS-b and/or other | Pulegone |
| (CsTPS35PK) | Citronellol |
| TPS-b and/or other | Borneol |
| csTPS33PK | para-Cymene |

Pesticidal Properties:

| | |
|---|---|
| Genes: Major Product (Minor Product) | Terpene Product to Breed For: |
| CsTPS4FN - TPS9-like2JL, TPS4-likeJL | Aromadendrene |
| CsTPS5PK (CsTPS32PK - TPS5JL) | α-Bisabolol |
| TPS-a | Cedrol |
| CsTPS18VF, csTPS19BL, csTPS35LS, (csTPS22PK, csTPS25LS, csTPS32PK) | Nerolidol |
| CsTPS18VF, csTPS19BL, csTPS35LS, (csTPS22PK, csTPS25LS, csTPS32PK) | trans-Nerolidol |
| TPS-a | Guaiol |

Pheromone: Insect attractant (e.g., for pollination; to attract insects that are predators of other insects or other pathogens that cause plant damage) or insecticidal (e.g., contact or fumigant toxicities)

| | |
|---|---|
| Genes: Major Product (Minor Product) | Terpene Product to Breed For: |
| | endo-Borneol |
| | Isoborneol |
| TPS37LPA5 | (3-Carene) |
| | Carveol |
| csTPS16CC | Germacrene B |
| CsTPS21AK | Hedycaryol |
| | Menthol |
| CsTPS18VF, csTPS19BL, csTPS35LS, (csTPS22PK, csTPS25LS, csTPS32PK) | cis-Nerolidol |
| CsTPS6FN, csTPS13PK, csTPS38FN, (csTPS17AK) | cis-β-Ocimene |
| CsTPS6FN, csTPS13PK, csTPS38FN, (csTPS17AK) | trans-β-Ocimene |
| (csTPS30PK, CsTPS7FN) | Sabinene Hydrate |
| csTPS33PK and TPS37LPA5 | α-Terpinen |
| csTPS33PK | Thymol |
| CsTPS25LS (CsTPS32PK) | β-Famosene |
| CsTPS25LS | α-Farnesene |
| CsTPS8FN, (csTPS16CC, csTPS22PK, csTPS28PK) | γ-Eudesmol |
| CsTPS4FN | Alloaromadendrene |
| (CsTPS8FN, csTPS4FN) | Valencene |
| TPS-b and/or other | Pulegone |

Expectorant Properties:

| | |
|---|---|
| Genes: Major Product (Minor Product) | Terpene Product to Breed For: |
| CsTPS32PK | Camphene |
| CsTPS32PK | Geraniol |
| (csTPS30PK, CsTPS7FN) | Sabinene Hydrate |

Non-irritant properties: (Breed for reduced production or absence of, e.g., one or more of the following terpene products that are irritants, which can be useful, e.g., when breeding plants such as Cannabis for dermatological uses in salves, creams, ointment and transdermal applications)

| | |
|---|---|
| Genes: Major Product (Minor Product) | Terpene Product to Breed for Absence/Reduced Amount: |
| TPS-b and/or other | Borneol |
| | α-Cedrene |
| (CsTPS35PK) | Citronellol |
| csTPS33PK | para-Cymene |
| | Fenchone (Presence - Counterirritant) |

The methods provided herein can, in certain aspects, be used to identify plant genotypes, e.g., Cannabis cultivar genotypes, that produce greater HIPV terpene concentrations in response to a pest or pathogen, or in response to one or more signals emitted by a companion Cannabis cultivar, or in response to one or more signals emitted by a plant cultivar of a species other than Cannabis. It is understood by those of skill in the art that when breeding for certain TPS genes, the presence of a certain gene does not mean that it will always be expressed or produce a measurable product in a flower until it is triggered to express such gene and/or produce a product that is a direct or indirect result of the expression of the gene. This includes, for example, the production of a higher concentration of one or more HIPV terpene concentrations when a threshold pest or pathogen pressure is applied, which then induces the expression of the one or more HIPV terpenes. In addition, variants of the TPS family can have a greater or lesser response to these pest or pathogen pressures, which can be identified by barcoding as provided herein, since the barcode of a TPS gene variant can be indicative of the greater or lesser expression response to a given pest or pathogen.

Similarly, the production of a higher concentration of one or more HIPV terpene concentrations in response to one or more external signals produced by one or more companion Cannabis plants or by one or more companion plants of a species other than Cannabis can be dependent on a threshold signal, which then induces the expression of the one or more HIPV terpenes. In addition, variants of the TPS family can have a greater or lesser response to these external signals, which can be identified by barcoding as provided herein, since the barcode of a TPS gene variant can be indicative of the greater or lesser expression response to a given pest. This also includes, for example, the production of a higher concentration of one or more HIPV terpene concentrations when one or more external signals from a companion Cannabis species or other species of plant is applied, which then induces the expression of the one or more HIPV terpenes. In addition, variants of the TPS family can have a greater or lesser response to these external signals, which can be identified by barcoding as provided herein, since the barcode of a TPS gene variant can be indicative of the greater or lesser expression response to an external signal from a companion plant.

In aspects, provided herein are methods of identifying plant cultivars containing terpene synthase gene profiles that result in the expression of terpenoids associated with oviposition deterrence (deter an insect that is a pest from laying eggs on the plant), fumigant insect repellent activity, contact toxicity and/or insect herbivore predator attractant. In certain aspects, the insect oviposition deterrent is selected from among one or more of linalool, α-bisabolol, and trans-neridol or any combinations thereof. In aspects, the contact insecticide is guaiol. In aspects, the fumigant insect repellent is selected from among β-ocimene, α-bisabololor a combination thereof.

In certain aspects, provided herein are methods of breeding plants that produce terpenoids associated with oviposition deterrence, insect fumigant activity, contact toxicity and/or an insect herbivore predator attractant. In aspects, the breeding comprises creating a plant with an oviposition deterrence profile by crossing a plant that produces an amount of trans-nerolidiol that is at or above a threshold amount with a plant that produces an amount of α-bisabololthat is at or above a threshold amount. In aspects, the breeding comprises creating a plant with an insect fumigant terpene profile by crossing a plant that produces an amount of α-bisabololthat is at or above a threshold amount with a plant that produces β-ocimene in an amount that is at or above a threshold amount. In certain aspects, the breeding comprises creating a plant with both oviposition deterrence and insect fumigant terpene profiles by crossing a plant that produces an amount of α-bisabolol and β-ocimene that is at or above a threshold amount with a plant that produces trans-nerolidiol in an amount that is at or above a threshold amount.

In certain aspects, the methods provided herein can be used to amplify the entire coding sequence of a TPS gene for analyzing, e.g., its homology to other TPS gene sequences by sequencing and/or restriction digest analysis, its terpene production (e.g., in vitro) and/or for transgenic cloning to functionally characterize the gene and/or to create variant cultivars having a desired terpene synthase gene expression profile. For example, amplicons of a TPS gene can be generated using a forward primer closest to the 5′ end of the gene, and a reverse primer closest to the 3′ end of the gene that could amplify the full transcript gene sequence from a given plant cultivar’s gDNA, or cDNA library. The resulting amplicons could be subject to any genotyping application such as HRM, sequencing, microarray analysis, restriction enzyme digestion, or other common genotyping methods.

In aspects, the TPS gene of interest can be inserted into a cloning vector for expression in a compatible host cell. A large number of vector-host systems known in the art can be used. Possible vectors include, but are not limited to, plasmids or modified viruses. Such vectors include, but are not limited to, bacteriophages such as lambda derivatives, or plasmids such as pCMV4, pBR322 or pUC plasmid derivatives or the Bluescript vector (Stratagene, La Jolla, CA). Other expression vectors include the HZ24 expression vector exemplified herein (see e.g., SEQ ID NOS:4 and 5). The insertion into a cloning vector can, for example, be accomplished by ligating the DNA fragment into a cloning vector which has complementary cohesive termini. Insertion can be effected using TOPO cloning vectors (Invitrogen, Carlsbad, CA). Prokaryotic and eukaryotic host cells can be used to express a gene contained in a vector. Such cells include bacterial cells, yeast cells, fungal cells, Archea, plant cells, insect cells and animal cells. These include but are not limited to mammalian cell systems infected with virus (e.g., vaccinia virus, adenovirus and other viruses); insect cell systems infected with virus (e.g., baculovirus); microorganisms such as yeast containing yeast vectors; or bacteria transformed with bacteriophage, DNA, plasmid DNA, or cosmid DNA.

The host cells are used to produce a protein encoded by the TPS gene or by a vector containing the gene by growing them under conditions whereby the encoded protein is expressed by the cell. The encoded TPS enzyme can be studied/manipualted in the host cell, or can be recovered from the cell by proteoin isolation and purification methods known to those of skill in the art. In aspects, the host-nucleic acid (vector or gene) system can be enginnered, by methods known to those of skill in the art, to secrete the TPS enzyme into the medium.

In aspects, the TPS gene is involved in the production of terpinolene. In aspects, the primers are as shown below:

Terpinolene - csTPS37FN full mRNA cloning primers (including signal peptide sequences)

| | Sequence (5′->3′) | Template strand | Length | Start | Stop | Tm | GC % | Self complem entarity | Self 3′ complem entarity |
|---|---|---|---|---|---|---|---|---|---|
| Forward primer | ATGCAGTGCATGGCT TTTC (SEQ ID NO:1328) | Plus | 19 | 1 | 19 | 57.17 | 47.37 | 9.00 | 0.00 |
| Reverse primer | TTACATGGGAATAGG GTTAATAATCAAATC (SEQ ID NO:1329) | Minus | 30 | 1920 | 1891 | 58.38 | 30.00 | 5.00 | 3.00 |
| Product length | 1920 | | | | | | | | |

In certain aspects, the TPS genes are selected from among the following: 1) TPS9 LPA4 type, 2) TPS9 LPA21.3 type, 3) TPS37 Cleaved (lacking the signal peptide, i.e., does not encode the chloroplast import signal), 4) TPS16CC, and 5) TPS20CT, and the primers are as follows:

Beta-Caryophyllene and Humulene - TPS9L21 (TPS9 LPA21.3)

| | Sequence (5′->3′) | Template strand | Length | Start | Stop | Tm | GC % | Self complem entarity | Self 3′ complem entarity |
|---|---|---|---|---|---|---|---|---|---|
| Forward primer | AT GTC ATA TCA AGT TTT AGC (SEQ ID NO:1330) | Plus | 20 | 1 | 20 | 48.75 | 30.00 | 5.00 | 2.00 |
| Reverse primer | TGG GAT TTG ATC TAT AAG TAA CG (SEQ ID NO:1331) | Minus | 23 | 1668 | 1646 | 53.76 | 34.78 | 4.00 | 2.00 |
| Product length | 1668 | | | | | | | | |

Beta-Caryophyllene and Humulene - TPS9L4 (TPS9 LPA4)

| | Sequence (5′->3′) | Template strand | Length | Start | Stop | Tm | GC % | Self complem entarity | Self 3′ complem entarity |
|---|---|---|---|---|---|---|---|---|---|
| Forward primer | AT GTC ATA TCA AGT TTT AGC (SEQ ID NO:1330) | Plus | 20 | 1 | 20 | 48.75 | 30.00 | 5.00 | 2.00 |
| Reverse primer | CGG GAT TTG ATC TAT AAG TAA CG (SEQ ID NO:1332) | Minus | 23 | 1668 | 1646 | 54.82 | 39.13 | 4.00 | 2.00 |
| Product length | 1668 | | | | | | | | |

Germacrene B / gamma-eudesmol - TPS16CC

| | Sequence (5′->3′) | Template strand | Length | Start | Stop | Tm | GC % | Self complem entarity | Self 3′ complem entarity |
|---|---|---|---|---|---|---|---|---|---|
| Forward primer | AT GTC TAG TCA AGT GTT AGC (SEQ ID NO:1333) | Plus | 20 | 1 | 20 | 52.37 | 40.00 | 6.00 | 2.00 |
| Reverse primer | TAA TGG GAT GGG ATC TAT AAG C (SEQ ID NO:1334) | Minus | 22 | 1713 | 1692 | 54.59 | 40.91 | 4.00 | 2.00 |
| Product length | 1713 | | | | | | | | |

Hedycaryol - CsTPS20CT

| | Sequence (5′->3′) | Template strand | Length | Start | Stop | Tm | GC % | Self complem entarity | Self 3′ complem entarity |
|---|---|---|---|---|---|---|---|---|---|
| Forward primer | AT GTC AAA TAT TCA AGT CTT AGC (SEQ ID NO:1335) | Plus | 23 | 1 | 23 | 52.90 | 30.43 | 6.00 | 2.00 |
| Reverse primer | TAA TGG GAT GGG ATC TAT AAG C (SEQ ID NO:1336) | Minus | 26 | 1653 | 1628 | 55.68 | 30.77 | 7.00 | 0.00 |
| Product length | 1653 | | | | | | | | |

Terpinolene - csTPS37FN coding primers (cleaved; does not include signal peptide sequences)

| | Sequence (5′->3′) | Template strand | Length | Start | Stop | Tm | GC % | Self complem entarity | Self 3′ complem entarity |
|---|---|---|---|---|---|---|---|---|---|
| Forward primer | ACT GTG GTC GAT AAC CCT AGT TC (SEQ ID NO:1337) | Plus | 23 | 184 | 206 | 59.55 | 47.83 | 4.00 | 0.00 |
| Reverse primer | CAT GGG AAT AGG GTT AAT AAT CAA ATC (SEQ ID NO:1338) | Minus | 27 | 1917 | 1891 | 56.63 | 33.33 | 5.00 | 3.00 |
| Product length | 1734 | | | | | | | | |

In embodiments, the above-mentioned primers can be adapted for use in commercial cloning, expression and/or amplification kits. For example, if a directional cloning/amplification/expression system is performed, such as TOPO directional cloning/amplification/expression (Thermofisher Scientific, USA), using one or more of the above-mentioned sets of primers, a 5′ CACC 3′ sequence can be attached on the 5′ end of any of the forward primera to allow for an overhang to be created during the process of PCR, which then allows for subsequent cloning/amplification/expression protocols to be carried out. In embodiments, the 5′CACC3′ tag can be interchanged with overhang sequences designated by kits other than the TOPO kit, to accomplish the same goal of functional characterization. In embodiments, introduction of an artificial start codon (ATG) at the 5′ end of a forward primer can permit the coding mRNA sequence to be expressed and properly folded in a bacterial or non-eukaryotic expression system. In embodiments, the ATG start codon is added to the 5′ end of the primer of SEQ ID NO:1337.

Energetic Terpene Profile Methods:

A method (to increase/to select for) bioaccumulation of at least one of the following terpenes: terpinolene, (Z)/(E)-β-ocimene, α-pinene, 3-carene and S-Linalool and decreased bioaccumulation/absence of R-linalool through gene selection and genotyping.
Where α-pineneis greater than β-pinene through selection of at least one of the following genes CsTPS2SK, (csTPS5FN, csTPS30PKand CsTPS32PK) using at least one forward & reverse primer combination from primer groups 1, 2, 3, and 4, respectively and a gel-based, HRM, qPCR, RT-qPCR, restriction enzyme digestion, or sequencing endpoint.
Where (Z)/(E)-β-ocimene bioaccumulation is increased through selection of at least one of the following genes csTPS6FN, csTPS13PK, and csTPS38FN using at least one forward & reverse primer combination from primer groups 12, 13, and 14 respectively and a gel-based, HRM, qPCR, RT-qPCR, restriction enzyme digestion, or sequencing endpoint.
Where terpinolene bioaccumulation is increased through selection of at least one of the following genes CsTPS31 PK and CsTPS37FN using at least one forward & reverse primer combination from primer groups 5 and 6, respectively and a gel-based, HRM, qPCR, RT-qPCR, restriction enzyme digestion, or sequencing endpoint.
Where 3-carene bioaccumulation is increased through selection of CsTPS37FN and/or CsTPS37LPA5 using at least one forward & reverse primer combination from primer group 6 and a gel-based, HRM, qPCR, RT-qPCR, restriction enzyme digestion, or sequencing endpoint.
Where terpinene (alpha- and/or gamma-) bioaccumulation is increased through selection of the following gene, csTPS33PK and/or TPS37LPA5 using at least one forward & reverse primer combination from primer group 19, and a gel-based, HRM, qPCR, RT-qPCR, restriction enzyme digestion, or sequencing endpoint.
Where S-Linalool bioaccumulation is increased through selection of at least one of the following genes CsTPS18VF using at least one forward & reverse primer combination from primer group 9, and a gel-based, HRM, qPCR, RT-qPCR, restriction enzyme digestion, or sequencing endpoint.
Where the absence of R-linalool is achieved through selection for absence or non-functional genotypes of CsTPS18Choc using at least one forward & reverse primer combination from primer group 7 and a gel-based, HRM, qPCR, RT-qPCR, restriction enzyme digestion, or sequencing endpoint.

A Method of Breeding Plants Through Selecting Parental Lines and/or Offspring:
Where the parental line and/or offspring is selected through selection of the presence/absence of 1 or more terpene synthase genes to produce a terpene profile with a given effect and where the selection of more than one gene that produces the same terpene product increases the % of the terpene fraction that is the intended terpene.
   Specifically, where the terpene synthase gene encoding an alpha-pinene:beta-pinene ratigreater than 1:1 is selected via PCR using primer groups 1, 2, 3, and/or 4 that is crossed ta plant containing high in terpinolene selected via PCR using primer groups 5, and/or 6 tproduce a more energetic terpene profile than produced by either parental line containing 1 of the 2 TPS genes.
   Specifically, where the terpene synthase gene encoding an alpha-pinene:beta-pinene ratigreater than 1:1 is selected via PCR using primer groups 1, 2, 3, and/or 4 that is crossed ta plant containing high in beta-ocimene selected via PCR using primer groups 12, 13, and/or 14 tproduce a more energetic terpene profile than produced by either parental line containing 1 of the 2 TPS genes.
   Specifically, where the terpene synthase gene encoding an S-linalool production is selected via PCR using primer group 9 that is crossed ta plant containing high in beta-ocimene selected via PCR using primer groups 12, 13, and/or 14 tproduce a more energetic terpene profile than produced by either parental line containing 1 of the 2 TPS genes.
   Specifically, where the terpene synthase gene encoding an alpha-pinene:beta-pinene ratigreater than 1:1 is selected via PCR using primer groups 1, 2, 3, and/or 4 that is crossed ta plant containing high in beta-ocimene and/or terpinolene that is selected via PCR using primer groups 12, 13, and/or 14 and/or primer groups 5 and/or 6 respectively tproduce offspring with a combination of three terpenes that produce a greater energetic terpene profile than either parent containing 1-2 of the 3 TPS genes.
      This can be expanded to any combination of the 4-6 terpenes (terpinolene, trans-nerolidol, alpha-pinene/beta-pinene, and presence of S-linalool/absence of R-linalool) that can produce a combination effect that can be more effective than selecting for a single terpene.
      Similarly, having multiple versions of a given terpene synthase allows for a greater bioaccumulation/% of the terpene fraction to be a given terpene. (l.e. selecting primer groups 5 and 6 for terpinolene can produce a higher % of the terpene fraction as terpinolene than selecting 5 or 6 alone.
         An effective combination can have multiple synthases selected for or against each of the 4-6 terpenes listed above.

Anti-Nociceptive/Pain-Relieving Terpene Profile Methods:

A method to increase bioaccumulation of at least one of the following terpenes: a-bisabolol, a-terpineol, trans-nerolidol, and a-phellandrene through gene selection and genotyping.
Where a-bisabolol bioaccumulation is increased through selection of at least one of the following genes csTPS5PK (csTPS31 PK, csTPS32PK) using at least one forward & reverse primer combination from primer groups 8 (Groups 5 and 4) and a gel-based, HRM, qPCR, RT-qPCR, restriction enzyme digestion, or sequencing endpoint.
Where a-terpineol bioaccumulation is increased through selection of at least one of the following genes (csTPS5PK, csTPS31 PK, csTPS32PK) using at least one forward & reverse primer combination from primer groups 8, 5, and 4, respectively, and a gel-based, HRM, qPCR, RT-qPCR, restriction enzyme digestion, or sequencing endpoint.
Where trans-nerolidol bioaccumulation is increased through selection of at least one of the following genes csTPS18VF, csTPS19BL, and csTPS35LS using at least one forward & reverse primer combination from primer groups 9, 10, and 11, respectively, and a gel-based, HRM, qPCR, RT-qPCR, restriction enzyme digestion, or sequencing endpoint.
Where a-phellandrene bioaccumulation is increased through selection of at least one of the following genes (CsTPS2SK, csTPS32PK, TPS37LPA5) using primer groups 1, 4 and 6, respectively, and a gel-based, HRM, restriction enzyme, or sequencing endpoint.

A Method of Breeding Plants Through Selecting Parental Lines and/or Offspring:
Where the parental line and/or offspring is selected through selection of the presence of 1 or more terpene synthase genes to produce a terpene profile with a given effect and where the selection of more than one gene that produces the same terpene product increases the % of the terpene fraction that is the intended terpene.
   Specifically, where the terpene synthase gene encoding trans-nerolidol production is selected via PCR using primer groups 9, 10, and/or 11 that is crossed ta plant containing high in alpha-bisabolol selected via PCR using primer groups 4, 5, and/or 8 tproduce a more anti-nociceptive terpene profile than produced by either parental line containing 1 of the 2 TPS genes.
   Specifically, where the terpene synthase gene encoding alpha-phellandrene production is selected via PCR using primer groups 1 and/or 4 and/or 6 that is crossed ta plant containing high in alpha-bisabolol that is selected via PCR using primer groups 4, 5, and/or 8 tproduce a more anti-nociceptive terpene profile than produced by either parental line containing 1 of the 2 TPS genes.
   Specifically, where the terpene synthase gene encoding trans-nerolidol production is selected via PCR using primer groups 9, 10, and/or 11 that is crossed ta plant containing high in alpha-terpineol and/or alpha-bisabolol that is selected via PCR using primer groups 4, 5, and or 8 and/or 4, 5, and/or 6, respectively tproduce offspring with a combination of three terpenes that produce a greater anti-nociceptive terpene profile than either parent containing 1-2 genes of the 3 TPS genes.
      This can be expanded to any combination of the 4 terpenes (alpha-bisabolol, trans-nerolidol, alpha-phellandrene, and alpha-terpineol) that can produce a combination effect that can be more effective than selecting for a single terpene.
      Similarly, having multiple versions of a given terpene synthase allows for a greater bioaccumulation/% of the terpene fraction to be a given terpene. (l.e. selecting primer groups 4 and 6 for alpha-bisabolol can produce a higher % of the terpene fraction as alpha-bisbolol than selecting 4 or 6 alone.
         An effective combination can have multiple synthases selected for each of the 4 terpenes listed above.

A Method to Identify and Breed Cannabis/Hemp Plant Genotypes Containing Terpenoids that are Insecticidal and/or Attractants of certain predator or beneficial organisms, where the Terpenoids are Associated with Oviposition Deterrence, Insect Fumigant Activity, Contact Toxicity, And Insect Herbivore Predator Attractant.
Where the starting plant contains the contact insecticide Guiaol or is selected for by using at least one forward & reverse primer combination from primer group 16, and a gel-based, HRM, qPCR, RT-qPCR, restriction enzyme digestion, or sequencing endpoint.
Where the starting plant contains the insecticide 3-carene or is selected for by using at least one forward & reverse primer combination from primer group 6, and a gel-based, HRM, qPCR, RT-qPCR, restriction enzyme digestion, or sequencing endpoint
● Where the insect oviposition deterrent is linalool selected for by using at least one of the following genes csTPS18VF, csTPS19BL, csTPS35LS, csTPS18Choc, csTPS29BC, and/or csTPS17AK using at least one forward & reverse primer combination from primer groups 9, 10, 11, 7, 17 and 18, respectively, and a gel-based, HRM, qPCR, RT-qPCR, restriction enzyme digestion, or sequencing endpoint.
Where the herbivore predator attractant is β-Famescene selected for by using at least one of the following genes csTPS32PK and/or csTPS25LS using at least one forward & reverse primer combination from primer groups 4 and 15 respectively, and a gel-based, HRM, qPCR, RT-qPCR, restriction enzyme digestion, or sequencing endpoint.
Where the fumigant insect repellent is beta ocimene selected for by using at least one of the following genes csTPS6FN, csTPS13PK, and csTPS38FN using at least one forward & reverse primer combination from primer groups 12, 13, and 14 respectively, and a gel-based, HRM, qPCR, RT-qPCR, restriction enzyme digestion, or sequencing endpoint.
Where the oviposition deterrent is trans nerolidol selected for by using at least one of the following genes csTPS18VF, csTPS19BL, and csTPS35LS using at least one forward & reverse primer combination from primer groups 9, 10, and 11, respectively, and a gel-based, HRM, qPCR, RT-qPCR, restriction enzyme digestion, or sequencing endpoint.
   Wherein trans nerolidol impairs an insect’s neural function
Where the insect fumigant is alpha bisabolol selected for by using at least one of the following genes csTPS5PK (csTPS31 PK, csTPS32PK) using at least one forward & reverse primer combination from primer group 8 (Groups 5 and 4) respectively, and a gel-based, HRM, qPCR, RT-qPCR, restriction enzyme digestion, or sequencing endpoint.
Where the oviposition deterrent is alpha bisabolol selected for by using at least one of the following genes csTPS5PK (csTPS31 PK, csTPS32PK) using at least one forward & reverse primer combination from primer group 8 (Groups 5 and 4) respectively, and a gel-based, HRM, qPCR, RT-qPCR, restriction enzyme digestion, or sequencing endpoint.
Where the oviposition deterrent is a-terpineol selected for by using at least one of the following genes (csTPS5PK, csTPS31 PK, csTPS32PK) using at least one forward & reverse primer combination from primer groups 8, 5, and 4, respectively, and a gel-based, HRM, qPCR, RT-qPCR, restriction enzyme digestion, or sequencing endpoint.

A Method of Breeding Plants Through Selecting Parental Lines and/or Offspring:
Where the parental line and/or offspring is selected through selection of the presence of 1 or more terpene synthase genes to produce a terpene profile with a given effect and where the selection of more than one gene that produces the same terpene product increases the % of the terpene fraction that is the intended terpene.
   Specifically, where the terpene synthase gene encoding trans-nerolidol production is selected via PCR using primer groups 9, 10, and/or 11 that is crossed to a plant containing high in alpha-bisabolol that is selected via PCR using primer groups 4, 5, and/or 8 to produce an oviposition deterrence terpene profile.
   Specifically, where the terpene synthase gene encoding alpha-bisabolol production is selected via PCR using primer groups 4, 5, and/or 6 that is crossed to a plant containing high in beta-ocimene that is selected via PCR using primer groups 12, 13, and/or 14 to produce offspring with a combination of an insect fumigant and oviposition deterrence terpene profile.
   Specifically, where a plant with the greatest oviposition deterrence is produced through selecting for the terpene synthase genes encoding a-terpineol (primer groups 4, 5, and 8), alpha bisabolol (primer groups 4, 5, and 8), and linalool (primer groups 9, 10, 11, 7, 17 and 18) to be present via PCR using the respective primer groups for those terpene synthase genes.
   Specifically, where the terpene synthase gene encoding trans-nerolidol production is selected via PCR using primer groups 9, 10, and/or 11 that is crossed to a plant containing high in beta-ocimene and/or alpha-bisabolol that is selected via PCR using primer groups 12, 13, and/or 14 and/or 4, 5, and/or 6, respectively to produce offspring with a combination of an insect fumigant and oviposition deterrence terpene profile.
      This can be expanded to any combination of the 5 terpenes (alpha-bisabolol, trans-nerolidol, beta-ocimene, guaiol, and beta-farnescene) that can produce a combination effect that can be more effective than selecting for a single terpene.
      Similarly, having multiple versions of a given terpene synthase allows for a greater bioaccumulation/% of the terpene fraction to be a given terpene. (l.e. selecting primer groups 9, 10, and 11 for trans-nerolidol can produce a higher % of the terpene fraction as trans-nerolidol than selecting 9, 10, or 11 alone.
         An effective combination can have multiple synthases selected for each of the 5 terpenes listed above.

In embodiments, the terpene synthase genes that produce an insecticidal profile produce at least one terpene that acts as a neuromodulator that can paralyze and/or kill insects, e.g., by one or more of binding to octopamine receptors, inhibiting AChE (acetylcholine esterase) and modulating GABA.A.

### Octopamine Receptors

In aspects, the compositions and methods provided herein are for identifying/selecting plants that express at least one terpene synthase that produces at least one terpene that binds to an octopamine receptor. In aspects, binding of the at least one terpene to an octopamine receptor can exert an insecticidal effect, i.e., the plant identified and/or selected, e.g., for cultivating and/or breeding has an insecticidal TPS/terpene profile. Octopamine receptors (octopamine receptors) govern insect neural function. The structure of octopamine is similar to epinephrine and norepinephrine; in insects, octopamine operates analogously to epinephrine and norepinephrine in the vertebrates. Vertebrates do not appear to have octopamine receptors. Octopamine is found is much higher concentrations in the insect nervous system than in mammals. Binding to octopamine receptors can cause neural excitation that can include behavioral modification in invertebrates, including insects, such as a lack of desire to eat, or to reproduce. The oviposition deterrence observed for mono and sesquiterpene alcohols can, in aspects, be mediated through binding to octopamine receptors. Without being bound by theory, octopamine is an aromatic(ring) amine diol, so oviposition deterrent terpenes may mediate their activity by either a secondary or tertiary alcohol functional group, e.g., of tertiary substituted mono and sesquiterpene alcohols (including α-bisabolol, eugenol). Insect repellent and oviposition deterrence function can be caused by modulation of the octopamine receptor by terpenes in Cannabis (alpha bisabolol, alpha terpineol, linalool, trans nerolidol, guiaol) with tertiary alcohol structure (low polarity OH group) that can modulate the octopamine receptor which controls many neural functions like behavior, etc. Monoterpene octopamine modulators can include, but are not limited to, all tertiary substituted terpene alcohols, tertiary substituted monoterpenes such as alpha terpineol, eugenol, geraniol, and linalool, and tertiary substituted sesquiterpene alcohols such as t-nerolidol, a bisabolol, and guiaol.

### Acetylcholine Esterase (AChE) Inhibition

In aspects, the compositions and methods provided herein are for identifying/selecting plants that express at least one terpene synthase that produces at least one terpene that inhibits acetylcholine esterase. In aspects, inhibition of actylcholine esterase can exert an insecticidal effect, i.e., the plant identified and/or selected, e.g., for cultivating and/or breeding has an insecticidal TPS/terpene profile. In aspects, inhibition of actylcholine esterase can improve cognitive function in a subject, e.g., a human subject.

The AChE enzyme can, in aspects, serve as an insect-specific insecticidal target (or a human-specific target to improve cognitive function) because the insect AChE enzyme differs from the human enzyme by one amino acid residue. “Dual binding site” terpene inhibitors can interact with the AChE at both the catalytic site and at peripheral site(s); thus, they can act both as competitive and uncompetitive inhibitors. In aspects, AChEl (acetylcholinesterase inhibition) can be improved using pairs of terpenes, or groups of terpenes, that bind uncompetitively at two sites of the AChE enzyme (e.g., alpha pinene at the active site and beta pinene at a peripheral site). Alternately, in aspects, a poor competitive binder can reduce the binding and AChEl by an active site binder, or can block active site binders at the peripheral site (“entourage effects” between terpenes). The size, dimension and/or functional groups of the terpenes can all play a role in the efficacy of AChEl. AChEl function can, in aspects, be improved by a synergistic effect (entourage effect) involving catalytic and peripheral noncompetitive inhibition; in aspects, the strong AChEl effects of some terpenes can be modulated by competitive binding of low AChEl terpenes. Examples of terpenes that exhibit AChEl effects are described in Jankowska et al., Molecules, 23(34):1-20

, the contents of which are expressly incorporated by reference herein. Certain examples include, but are not limited to, α-pinene, β-pinene, eugenol, isoeugenol, ocimene, pulegon, α-terpinene, α-terpineoland Terpinen-4-ol.

### GABA Receptor Modulation

In aspects, the compositions and methods provided herein are for identifying/selecting plants that express at least one terpene synthase that produces at least one terpene that is a positive allosteric modulator of a GABA receptor. In aspects, the GABA receptor is GABA A. In aspects, positive allosteric modulation of a GABA receptor, such as GABA A, can exert an insecticidal effect, i.e., the plant identified and/or selected, e.g., for cultivating and/or breeding has an insecticidal TPS/terpene profile. In aspects, positive alloateric modulation of a GABA receptor, such as GABA A, can be used to manage anxiety, seizures, pain, alcohol withdrawal and the like in a subject, e.g., a human subject. Examples of terpenes that exhibit effects through positive allosteric modulation of a GABA receptor, such as GABA A, are described in Jankowska et al., Molecules, 23(34):1-20 (2018), the contents of which are expressly incorporated by reference herein. Certain examples include, but are not limited to, linalool, geraniol, α-terpineol, α/β-thujone, α-thujone, (-)-borneol and nerolidol.

### Terpene Synthase Genes Corresponding to Each of the Primer Groups

Primer Group 1 - CsTPS2SK
Primer Group 2 - CsTPS5FN
Primer Group 3 - CsTPS30PK
Primer Group 4 - CsTPS32PK
Primer Group 5 - CsTPS31 PK
Primer Group 6 - CsTPS37FN and TPS37LPA5
Primer Group 7 - CsTPS18Choc
Primer Group 8 - CsTPS5PK
Primer Group 9 - CsTPS18VF
Primer Group 10 - CsTPS19BL
Primer Group 11 - CsTPS35LS
Primer Group 12 - CsTPS6FN
Primer Group 13 - CsTPS13PK
Primer Group 14 - CsTPS38FN
Primer Group 15 - CsTPS25LS
Primer Group 16 - Guiaol Synthase QTL
Primer Group 17 - CsTPS29BC
Primer Group 18 - CsTPS1 7AK
Primer Group 19 - CsTPS33PK

### Use of Devices, Programs and Media

In certain embodiments, an outcome and/or classification obtained by the methods provided herein is provided using a suitable visual medium (e.g., a component of a machine, e.g., a printer or display). A classification and/or outcome may be provided in the form of a report. A report typically includes a display of an outcome and/or classification (e.g., a value, one or more characteristics of a sample, an assessment or probability of presence or absence of a genotype, phenotype or genetic variation; and/or an assessment or probability of a genotype, genetic variation, and/or genetic variation signature, e.g., of a TPS gene profile for a plant cultivar), sometimes includes an associated confidence parameter, and sometimes includes a measure of performance for a test used to generate the outcome and/or classification. A report sometimes includes a recommendation for a follow-up test (e.g., a test that confirms the outcome or classification).

A report can be displayed in a suitable format that facilitates determination of presence or absence of a genotype, phenotype, genetic variation or genetic variation signature. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing. In embodiments, the report can be in the form of a barcode, where each line/number in the barcode represents a TPS gene or paralog thereof that is present in the plant cultivar.

A report can be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, or any other medium described in the previous paragraph. A laboratory file may be in tangible form or electronic form (e.g., computer readable form), in certain embodiments. After a report is generated and transmitted, a report can be received by obtaining, via a suitable communication medium, a written and/or graphical representation of an outcome and/or classification, which upon review allows a qualified individual to make a determination as to one or more characteristics of a sample from a plant cultivar, such as the presence or absence of a genotype, phenotype or genetic variation in a test sample (e.g., a Cannabis plant sample).

An outcome and/or classification can be provided by and obtained from a laboratory (e.g., obtained from a laboratory file). A laboratory file can be generated by a laboratory that carries out one or more tests for determining one or more characteristics of a sample such as presence or absence of a genotype, phenotype or genetic variation for a test sample (e.g., a Cannabis plant sample). Laboratory personnel (e.g., a laboratory manager) can analyze information associated with test samples (e.g., test profiles, reference profiles, test values, reference values, level of deviation) underlying an outcome and/or classification. For calls pertaining to presence or absence of a genotype, phenotype or genetic variation that are close or questionable, laboratory personnel can re-run the same procedure using the same (e.g., aliquot of the same sample) or different test sample from a plant.

A laboratory can be in the same location or different location (e.g., in another town, city or country) as personnel assessing the presence or absence of a genotype, phenotype or genetic variation from the laboratory file. For example, a laboratory file can be generated in one location and transmitted to another location in which the information for a test sample therein is assessed by a qualified individual, and optionally, transmitted to the facility and/or grower from which the test sample was obtained. A laboratory sometimes generates and/or transmits a laboratory report containing a classification of presence or absence of a genotype, phenotype or a genetic variation for a test sample (e.g., a Cannabis plant sample).

### Machines, Software and Interfaces

Methods described herein (e.g., processing amplification results, processing high resolution melting (HRM) assay results, processing sequence read data, determining one or more characteristics of a plant cultivar based on sequence read data, associating one or more phenotypes of a plant cultivar (e.g., terpene, cannabinoid or flavonoid production profiles with one or more genotypes or genetic variants of the plant cultivar, and/or providing an outcome (e.g., indicated as desirable for breeding, or cultivating as a crop, or for therapeutic use, based on the specified selection criteria) can be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., processing amplification results, processing high resolution melting (HRM) assay results, processing sequence read data, determining one or more characteristics of a plant cultivar based on sequence read data, associating one or more phenotypes of a plant cultivar (e.g., a Cannabis plant) with one or more genotypes or genetic variations for the plant cultivar and/or providing an outcome are performed by automated methods. In embodiments, one or more steps of a method described herein are carried out by a microprocessor and/or computer, and/or carried out in conjunction with memory. In certain embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces can be used to conduct methods described herein. Using machines, software and interfaces, a user can enter, request, query or determine options for using particular information, programs or processes (e.g., processing amplification results, processing high resolution melting (HRM) assay results, processing sequence read data, determining one or more characteristics of a plant cultivar based on sequence read data, associating one or more phenotypes of a plant cultivar with one or more genotypes or genetic variations and/or providing an outcome, which can involve implementing statistical analysis algorithms, statistical significance algorithms, statistical algorithms, iterative steps, validation algorithms, and graphical representations, for example. In embodiments, a data set can be entered by a user as input information, a user may download one or more data sets by suitable hardware media (e.g., flash drive), and/or a user can send a data set from one system to another for subsequent processing and/or providing an outcome (e.g., send sequence read data from a sequencer to a computer system for sequence read processing; send processed sequence read data to a computer system for further processing and/or yielding an outcome and/or report).

A system typically includes one or more machines. Each machine includes one or more of memory, one or more microprocessors, and instructions. Where a system includes two or more machines, some or all of the machines can be located at the same location, some or all of the machines can be located at different locations, all of the machines can be located at one location and/or all of the machines may be located at different locations. Where a system includes two or more machines, some or all of the machines can be located at the same location as a user, some or all of the machines can be located at a location different than a user, all of the machines can be located at the same location as the user, and/or all of the machine can be located at one or more locations different than the user.

A system sometimes includes a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine an outcome from the sequence reads.

A user can, for example, place a query to software which then may acquire a data set via internet access, and in certain embodiments, a programmable microprocessor may be prompted to acquire a suitable data set based on given parameters. A programmable microprocessor also can prompt a user to select one or more data set options selected by the microprocessor based on given parameters. A programmable microprocessor can prompt a user to select one or more data set options selected by the microprocessor based on information found via the internet, other internal or external information, or the like. Options can be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein can include general components of computer systems, such as, for example, network servers, laptop systems, desktop systems, handheld systems, personal digital assistants, computing kiosks, and the like. A computer system can include one or more input means such as a keyboard, touch screen, mouse, voice recognition or other means to allow the user to enter data into the system. A system can further include one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components can be connected to a central processing unit which may comprise among other components, a microprocessor for executing program instructions and memory for storing program code and data. In embodiments, processes can be implemented as a single user system located in a single geographical site. In certain embodiments, processes can be implemented as a multi-user system. In the case of a multi-user implementation, multiple central processing units can be connected by means of a network. The network can be local, encompassing a single department in one portion of a building, an entire building, span multiple buildings, span a region, span an entire country or be worldwide. The network can be private, being owned and controlled by a provider, or it can be implemented as an internet-based service where the user accesses a web page to enter and retrieve information. Accordingly, in certain embodiments, a system includes one or more machines, which can be local or remote with respect to a user. More than one machine in one location or multiple locations can be accessed by a user, and data can be mapped and/or processed in series and/or in parallel. Thus, a suitable configuration and control can be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface can be used to receive signal information that can be detected by a signal detection module.

Data can be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

A system can include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can include a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules can be located in different machines in different physical locations.

A machine, in some embodiments, includes at least one microprocessor for carrying out the instructions in a module. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In embodiments, a machine includes a module one or more modules. A machine that includes a module often is capable of receiving and transferring one or more of data and/or information to and from other modules.

In certain embodiments, a machine includes peripherals and/or components. In certain embodiments, a machine can include one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magnetooptical discs, flash memory devices (e.g., flash drives), RAM, floppy discs, the like, and other such media on which the program instructions can be recorded. In online implementation, a server and web site maintained by an organization can be configured to provide software downloads to remote users, or remote users can access a remote system maintained by an organization to remotely access software. Software can obtain or receive input information. Software can include a module that specifically obtains or receives data and may include a module that specifically processes the data (e.g., a processing module that processes received data). The terms “obtaining” and “receiving” input information refers to receiving data by computer communication means from a local, or remote site, human data entry, or any other method of receiving data. The input information may be generated in the same location at which it is received, or it may be generated in a different location and transmitted to the receiving location. In embodiments, input information is modified before it is processed (e.g., placed into a format amenable to processing, e.g., tabulated).

Software can include one or more algorithms in certain embodiments. An algorithm can be used for processing data and/or providing an outcome or report according to a finite sequence of instructions. An algorithm often is a list of defined instructions for completing a task. Starting from an initial state, the instructions can describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In embodiments, an algorithm can be configured or modified to include margin of errors, statistical analysis, statistical significance, and/or comparison to other information or data sets (e.g., applicable when using a neural net or clustering algorithm).

In certain embodiments, several algorithms can be implemented for use in software. These algorithms can be trained with raw data in some embodiments. For each new raw data sample, the trained algorithms can produce a representative processed data set or outcome. A processed data set sometimes is of reduced complexity compared to the parent data set that was processed. Based on a processed set, the performance of a trained algorithm can be assessed based on sensitivity and specificity, in some embodiments. An algorithm with the highest sensitivity and/or specificity can be identified and utilized, in certain embodiments.

In certain embodiments, simulated (or simulation) data can aid data processing, for example, by training an algorithm or testing an algorithm. In embodiments, simulated data includes hypothetical various samplings of different groupings of sequence reads, genotypes, phenotypes, genetic variations, and/or genetic variation signatures. Simulated data can be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In embodiments, an empirical model may be assessed, in which it is assumed that at least one sample matches a reference sample (with or without resolved variations). In certain embodiments, another distribution, such as a Poisson distribution for example, can be used to define the probability distribution.

A system can include one or more microprocessors in certain embodiments. A microprocessor can be connected to a communication bus. A computer system can include a main memory, often random access memory (RAM), and can also include a secondary memory. Memory in some embodiments includes a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor can implement software in a system. In some embodiments, a microprocessor can be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample.

In certain embodiments, secondary memory can include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

### Compositions and Kits

Provided in certain embodiments are compositions. Compositions useful for carrying out any of the methods described herein are provided. For example, compositions that include any of the primers, primer pairs and sets of more than one primer pair described herein are provided. In certain embodiments, the compositions include one or more of primers or primer pairs for identifying monoterpene synthases, primers or primer pairs for identifying diterpene synthases and primers or primer pairs for identifying sesquiterpene synthases. In embodiments, the compositions include one or more of primers or primer pairs for identifying monoterpene synthases. In embodiments the compositions include one or more of primers or primer pairs for identifying diterpene synthases. In embodiments, the compositions include one or more of primers or primer pairs for identifying sesquiterpene synthases. In any of the compositions provided herein, in certain embodiments, the primers are selected from among those of SEQ ID NOS:1-1284, 1398 and 1399; from among the LAMP primers of SEQ ID NOS:1285-1327 or the LAMP primer sets of SEQ ID NOS:1285-1293; 1294-1302; 1303-1311; 1312-1319 and 1320-1327; or from among the full-length amplifying primers of SEQ ID NOS:1328-1338; in Primer Groups 1-19 as set forth in Tables 1-16 and in B3/F3 polynucleotide primer pairs set forth in Tables 17-35. In embodiments, the primers are selected from among those set forth in SEQ ID NOS: 1-1284, 1398 and 1399; from among the LAMP primers of SEQ ID NOS:1285-1327 or the LAMP primer sets of SEQ ID NOS:1285-1293; 1294-1302; 1303-1311; 1312-1319 and 1320-1327, or from among the full-length amplifying primers of SEQ ID NOS:1328-1338; in Primer Groups 1-19 as set forth in Tables 1-16 and in B3/F3 polynucleotide primer pairs set forth in Tables 17-35 and/or from among sequences that share 90%, 91%, 92%, 93%, 94%, 95%, 96%, 97%, 98%, 99% or more identity with any of the sequences set forth in SEQ ID NOS: 1-1284, 1398 and 1399; the LAMP primers of SEQ ID NOS:1-1327 or the LAMP primer sets of SEQ ID NOS:1285-1293; 1294-1302; 1303-1311; 1312-1319 and 1320-1327, or from among the full-length amplifying primers of SEQ ID NOS:1328-1338; in Primer Groups 1-19 as set forth in Tables 1-16 and in B3/F3 polynucleotide primer pairs set forth in Tables 17-35. In embodiments, any of the forward primers in the primer pairs provided in SEQ ID NOS: 1-1284, or in sequences that share 90%, 91%, 92%, 93%, 94%, 95%, 96%, 97%, 98%, 99% or more identity with any of the sequences set forth in SEQ ID NOS: 1-1284, 1398 and 1399; in Primer Groups 1-19 as set forth in Tables 1-16 and in B3/F3 polynucleotide primer pairs set forth in Tables 17-35 can be paired with any of the reverse primers of the primer pairs having the sequences set forth in SEQ ID NOS: 1-1284, 1398 and 1399; in Primer Groups 1-19 as set forth in Tables 1-16 and in B3/F3 polynucleotide primer pairs set forth in Tables 17-35 or with sequences that share 90%, 91%, 92%, 93%, 94%, 95%, 96%, 97%, 98%, 99% or more identity with any of the sequences set forth in SEQ ID NOS: 1-1284, 1398 and 1399, in Primer Groups 1-19 as set forth in Tables 1-16 and in B3/F3 polynucleotide primer pairs set forth in Tables 17-35.

In embodiments, the primer pairs of the compositions provided herein are complementary to a unique subsequence of a TPS gene or a paralog thereof, wherein the unique subsequence of the TPS gene or paralog thereof is different than the other subsequences of the TPS gene or paralog thereof and is different than the subsequences of other TPS genes or paralogs thereof. In certain embodiments, the TPS gene or a paralog thereof is of a Cannabis cultivar. In embodiments, the compositions provided herein include at least one primer or primer pair that is complementary to a genetically modified TPS gene or paralog thereof.

Also provided herein are compositions that include at least one amplicon generated by any of the polynucleotide primer pairs provided herein.

In aspects, one or more of the primers or primer pairs in the compositions provided herein can be synthesized using standard methods and equipment, such as the ABl®3900 High Throughput DNA Synthesizer and EXPEDITE®8909 Nucleic Acid Synthesizer, both of which are available from Applied Biosystems (Foster City, CA). Analogs and derivatives are described in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing nucleic acids containing such analogs or derivatives are disclosed, for example, in the patent publications cited above and in U.S. Pat. Nos. 5,614,622; 5,739,314; 5,955,599; 5,962,674; 6,117,992; in WO 00/75372 and in related publications. In certain embodiments, analog nucleic acids include inosines, abasic sites, locked nucleic acids, minor groove binders, duplex stabilizers (e.g., acridine, spermidine) and/or other melting temperature modifiers, containing base analogs, sugar analogs and/or a non-native backbone and the like, RNA/DNA hybrids and polyamide nucleic acids (PNAs). A primer or primer pair can, in certain aspects, contain a modification such as one or more nonstandard nucleotides, non-natural nucleotides, universal bases, degenerate nucleotides, inosines, abasic sites, locked nucleic acids, minor groove binders, duplex stabilizers (e.g., acridine, spermidine), Tm modifiers or any modifier that changes the binding properties of the primers. A primer or primer pair, in certain aspects, can contain a detectable molecule or entity (e.g., a fluorophore, radioisotope, colorimetric agent, particle, enzyme, and the like). A primer also can refer to a polynucleotide sequence that, when hybridized to a subsequence of a target nucleic acid or another primer, facilitates the detection of a primer, a target nucleic acid or both, as with molecular beacons, for example. The term “molecular beacon,” as used herein, refers to detectable molecule, where the detectable property of the molecule is detectable only under certain specific conditions, thereby enabling it to function as a specific and informative signal. Non-limiting examples of detectable properties are, optical properties, electrical properties, magnetic properties, chemical properties and time or speed through an opening of known size.

In the compositions provided herein, any of the modified primer or primer pairs provided herein can be used to generate amplicons containing the modifications. In aspects, the amplicons can directly be modified, e.g., under amplification conditions that include non-natural nucleotide analogs or by complexing/labeling the amplicon using a detectable label.

Modified nucleotides and nucleotide analogs in some aspects of the primer, primer pairs and/or amplicons of the compositions provided herein can include, in certain aspects and without limitation, dideoxynucleotides, acyclic nucleotide analogs, deazapurine nucleotides, e.g., 7-deaza-deoxyguanosine (7-deaza-dG) and 7-deaza-deoxyadenosine (7-deaza-dA) mono-, di- and triphosphates, deutero-deoxythymidine (deutero-dT) mon-, di- and triphosphates, methylated nucleotides e.g., 5-methyldeoxycytidine triphosphate, ¹³C/¹⁵N labeled nucleotides and deoxyinosine mono-, di- and triphosphate.

### Detectable Labels

Examples of detectable labels include, but are not limited to, radiolabels, chromophores, chemiluminescent moieties, bioluminescent moieties, fluorescent moieties and metals.

Examples of chemiluminescent materials include, but are not limited to, any selected from among oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others. Examples of chromophores include, but are not limited to, 3,3′-diaminobenzidine (DAB); 3-amino-9-ethyl carbazole (AEC); Fast Red; FD&C Yellow 5 (Tartrazine); Malachite Green Carbinol hydrochloride; Crocein Scarlet 7B (Dark Red); Erloglaucine (Dark Blue); Crystal Violet (Dark Purple); Bromophenol Blue; Cobalt(II) Chloride Hexahydrate (Red); Basic Violet 3; Acid Blue 9; Acid Red 71; FD&C Blue 1 (Brilliant Blue FCF); FD&C Red 3 (Erythrozine); and FD&C Red 40 (Allura Red AC).

Examples of fluorophores (fluorescent labels) include, but are not limited to, di-8-ANEPPS, di-4-ANEPPS, a carbocyanine dye (e.g., DiO, DiL), a PKH dye (exemplary of which are PKH-26 and PKH-67), Dylight488, Brilliant Violet, Pacific Blue, Chrome Orange, Brilliant Blue 515, phycoerythrin (PE), rhodamine, fluorescein, FITC, PE-Cy5.5, PE-Cy7, APC, Alexa647, APC-Alexa700 and APC-Alexa750, Oregon Green®, derivatives of rhodamine (e.g., Texas Red and tetrarhodimine isothiocynate (TRITC)), AMCA, Alexa Fluor®, Li-COR®, CyDyes® or DyLight® Fluors); tdTomato, mCherry, mPlum, Neptune, TagRFP, mKate2, TurboRFP and TurboFP635 (Katushka). Fluorescent “reporter” labels for labelling primers or amplicons also are known to those of skill in the art and can include, for example, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, Cy5 (cyanine 5), Cy3 (cyanine 3), FAM (6-carboxyfluorescein), TET (tetrachloro-6-carboxy-fluorescein), TEX (sulforhodamine 101), ROX (carboxy-X-rhodamine), JOE (4,5-dichlorocarboxyfluorescein), 6-JOE (6-carboxy-4′5′-dichloro-2′,7′-dimethoxy fluorescein), 6-JOE SE (6-carboxy-4′5′-dichloro-2′,7′-dimethoxy fluorescein, succinimidyl ester), 6-JOE NHS (6-carboxy-4′5′-dichloro-2′,7′-dimethoxy fluorescein, N-hydroxysuccinimide ester), HEX (hexacholoro-6-carboxy-fluorescein), FITC (fluorescein isothiocyanate), rhodamines, tetramethylrhodamine, TRITC (tetrarhodimine isothiocynate), BODIPY (N-[6-(2,2-difluoro-10,12-dimethyl-1-aza-3-azonia-2-boranuidatricyclo[7.3.0.0^{3,7}]dodeca-3,5,7,9,11-pentaen-4-yl)-1-(3-hydroxy-2,5-dioxopyrrolidin-1-yl)-1-oxohexan-2-yl]propenamide), xanthenes, fluoresceins, cyanines, carbocyanines, coumarins and derivatives thereof. Many equivalent detection labels, both proprietary and non-proprietary, are readily available and known to those of skill in the art.

Examples of non-specific dyes that emit a fluorescent signal when bound to double-stranded DNA (e.g., by intercalation) include, but are not limited to, ethidium bromide, thiazole orange, oxazole yellow, BOXTO (4-[6-(benzoxazole-2-yl-(3-methyl-)-2,3-dihydro-(benzo-1,3-thiazole)-2-methylidene)]-1-methyl-quinolinium chloride) and its positive divalent derivative BOXTO-PRO (4-[(3-methyl-6- (benzoxazole-2-yl)-2,3-dihydro-(benzo-1,3-thiazole)-2-methylidene)]-1-(3-trimethylammonium-propyl)-quinolinium dibromide), SYBR GREEN I (N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR GOLD [2-[N-(3-dimethylaminopropyl)-N-propylamino]-4-[2,3-dihydro-3-methyl-(benzo-1,3-thiazol-2-yl)-methylidene]-1-phenyl-quinolinium], YoYo-1 [1²(2)Z,16(17²)Z]-1³,7,7,11,11,17³-Hexamethyl-13H,173H-7,11-diaza-3¹λ⁵,15¹λ⁵-3(4,1),15(1,4)-diquinolina-1,17(2)-bis([1,3]benzoxazola)heptadecaphane-1²(2),16(17²)-diene-7,11-diium-3′,15′-bis(ylium) tetraiodide), Yo-Pro-1 (trimethyl-[3-[4-[(2)-(3-methyl-1,3-benzoxazol-2-ylidene)methyl]quinolin-1-ium-1-yl]propyl]azanium;diiodide), BEBO (4-[(3-methyl-6-(benzothiazol-2-yl)-2,3-dihydro- (benzo-1,3-thiazole)-2-methylidene)]-1-methyl-pyridinium iodide) and others such as those described, for example, in PCT Publication No. WO 2002/090443 A1 and in U.S. Pat. No. 7,378,240 B2. Many equivalent non-specific detection labels, both proprietary and non-proprietary, are readily available and known to those of skill in the art.

Fluorescence quencher labels also can be used to modify the primers, primer pairs and/or amplicons in the compositions provided herein; examples of these include, but are not limited to, 5-TAMRA (5-carboxytetramethylrhodamine), 6-TAMRA (6-carboxytetramethylrhodamine), a mixture of 5-TAMRA and 6-TAMRA, 5-TAMRA NHS (N-hydroxysuccinimide) ester, 6-TAMRA NHS ester, a mixture of 5-TAMRA NHS ester and 6-TAMRA NHS ester, DABSYL

(dimethylaminoazobenzenesulfonic acid), Black Hole Quencher molecules (BHQ), Iowa Black FQ / Iowa Black RQ and related fluorescence quenchers such as those described in U.S. Pat. Nos. 7,439,341, 7,803,536, 7,476,735, 7,605,243, 7,645,872, 8,030,460, 8,084,588, 8,114,979, 8,258,276 and 8,916,345, the contents of which are incorporated expressly by reference herein. Many equivalent fluorescence quencher labels, both proprietary and non-proprietary, are readily available and known to those of skill in the art.

### Kits

Provided in certain embodiments are kits. The kits can include any components and compositions described herein, e.g., primers, primer pairs, primer sets, including LAMP primer sets, reagents for hybridization or amplification of at least one TPS gene or paralog thereof, solid supports, collections of solid supports, one or more detection labels for detecting amplicons and instructions for use to, e.g., analyze the TPS gene profile of a plant cultivar of interest, or to identify a genetically modified plant cultivar of interest. A kit for amplifying nucleic acid from an RNA template can further include reagents for reverse transcription (e.g., for generating cDNA).

Components of a kit can be present in separate containers, or multiple components can be present in a single container. In embodiments, primers are provided such that each container contains a single primer pair (e.g., for individual amplification reactions). In certain embodiments, primers are provided such that one container contains a plurality of primer pairs (e.g., for multiplexed amplification reactions). Suitable containers include a single tube (e.g., vial), one or more wells of a plate (e.g., a 96-well plate, a 384-well plate, and the like), chips and the like.

Kits also can include instructions for performing one or more methods described herein and/or a description of one or more components described herein. For example, a kit can include instructions for using the amplification primers provided herein, to amplify nucleic acid (e.g., to amplify unique subsequences of a TPS gene or paralog thereof in a plant cultivar). In certain embodiments, a kit can include instructions or a guide for interpreting the results of an amplification reaction. Instructions and/or descriptions can be in printed form and can be included in a kit insert. In embodiments, instructions and/or descriptions are provided as an electronic storage data file present on a suitable computer readable storage medium, e.g., portable flash drive, DVD, CD-ROM, diskette, and the like. A kit also can include a written description of an internet location that provides such instructions or descriptions.

### EXAMPLES

The examples set forth below illustrate certain embodiments and do not limit the technology.

### Example 1: Representative PCR Protocol Using Polynucleotide Primer Pairs / Sets of Primer Pairs Provided Herein to Identify and/or Select Plant Cultivars With Desired Terpene Profiles

The primer sequences that were designed, and their corresponding target terpene synthase genes, are set forth in Table B, in Primer Groups 1-19 as set forth in Tables 1-16 and in B3/F3 polynucleotide primer pairs set forth in Tables 17-35. These primers specifically targeted the identified coding regions (exons) of the terpene synthase genes but could also be used to examine informative introns through using other combinations of the primers described herein.

These primers can be used to amplify their target sequences in traditional PCR on total DNA or purified genomic DNA, as well as in reverse-transcriptase PCR to identify variants and presence/absence variation of expressed DNA.

Table 36 depicts an example of parameters of a traditional PCR protocol in which the primers provided herein (e.g., as described in Table B, in Primer Groups 1-19 as set forth in Tables 1-16 and in B3/F3 polynucleotide primer pairs set forth in Tables 17-35) can be used:

TABLE 36

| | | | |
|---|---|---|---|
| Traditional Program: 30 cycles | | Temperature | Time |
| Initiation/Polymerase Activation: | | 95-97° C. | 2 mins |
| | Start of Cycling: | | |
| Denaturation: | | 95° C. | 10 secs |
| Step Down Annealing: | | 52-58° C. ** | 10 secs |
| Elongation: | | 72° C. | 30 secs |
| | Final Elongation Step: | 72° C. | 5-15 mins |

** Annealing temperature is specific for each primer set. For determining optimal T _{annealing}, the highest Tₘ of the two primers in a given set minus 5° C. results in the optimal annealing temperature.

Table 37 depicts an example of parameters of a step-down PCR protocol, using standard PCR reagents, in which the primers provided herein (e.g., as described in Table B) can be used:

TABLE 37

| | | | |
|---|---|---|---|
| Step-Down Program: 30 cycles | | Temperature | Time |
| Initiation/Polymerase Activation: | | 95-97° C. | 2 mins |
| | Start of Cycling: | | |
| Denaturation: | | 95° C. | 10 secs |
| Step Down Annealing: | | 60° C. (sec. target 50° C.) | 10 secs |
| Elongation: | | 72° C. | 30 secs |
| | Final Elongation Step: | 72° C. | 5-15 mins |

It is understood by those of skill in the art that modifications to these protocols can be made to achieve the same or similar results. For example, the temperatures for the various steps in the can be modified by between about 1-5° C., or touchdown PCR can be performed, i.e., the annealing temperature is adjusted based on the cycle number.

### Example 2: Use of Exon-Specific Primers to Obtain Terpene Synthase Fingerprints of a Plant Cultivar

### DNA Isolation

Genomic DNA is isolated from Cannabis samples using the Qiagen DNA Easy Plant genomic DNA isolation kits (Qiagen) or the Promega Wizard genomic DNA kit (Promega) using manufacturer’s instructions, FTA plant saver cards (Whatman’s Flinders Technology Associates, a technology developed by GE Healthcare for lysing cells and storing DNA on a piece of Whatman filter paper), or an in-house crude preparation of genomic DNA extracts. A crude DNA extract is prepared by Tris/Triton-X pre-treatment of 1 mm raw leaf or leaf imprinted FTA card sections, as modified from Klimyuk et al. (Plant J., 3(3):493-494 (1993)) in a modified 96 well format for high throughput processing. Leaf or FTA selections were placed aseptically in a 96 well microtiter plate, 100 uL 0.25 M Tris-HCI with 0.25% Triton-X-100 was added to each well, and the plates were incubated at 100° C. for 5 minutes on a Veriti thermocycler (ABI). 3 uL of crude genomic DNA extract was used as input for the pre-amplification PCR reaction.

### RNA Isolation

Plant material/tissue from Cannabis is flash frozen in liquid nitrogen or placed in a RNase inhibitory solution, such as RNALater, for in-grow collection of tissue for cDNA analysis. Total RNA is then isolated from Cannabis tissues using any plant RNA extraction kit or method available and/or known to those of skill in the art. Examples of such kits include: the Qiagen RNAeasy plant extraction kit, which can be used following manufacturer’s instructions or modifying the instructions by using RLC buffer to provide a higher quality extraction that yields greater concentrations of RNA, the Direct-zol RNA isolation kit and the Zymogen Quick-RNA Plant mini prep kit. An example of an RNA isolation protocol is as follows:

Total RNA is isolated from fresh Cannabis leaf tissue samples using the Direct-zol RNA isolation kit and Zymogen Quick-RNA Plant mini prep kit with DNAase digestion, using manufacturer’s instructions (Zymogen). Purified RNA is prepared for quantification using the QuantiFluor HS-ssRNA System (Promega) and quantified using a Quantus Fluorometer (Promega), as per the manufacturer’s instructions.

The quantified RNA is diluted to a final working concentration of 5 ng/uL and used as normalized input into either a First strand cDNA synthesis reaction or a one-step reverse transcriptase real-time qPCR reaction.

### cDNA Synthesis

The single-stranded RNA is then converted to double-stranded cDNA using any available cDNA-synthesis reverse transcriptase (RT-PCR) kit or method available and/or known to those of skill in the art. Example of kits that can be used are the High Capacity RNA-to-cDNA Kit or The SuperScript IV First-Strand Synthesis System (both from Thermofisher Scientific), or Qiagen’s FastLane Cell cDNA Kit. These provide double-stranded DNA that can be subjected to High Resolution Melt (HRM) analysis with or without a pre-amplification step, depending on the RNA extraction quality. Additionally, after the RNA extraction and before the cDNA synthesis, the sample can be subjected to ribo-depletion or mRNA amplification, to remove rRNA and obtain greater sensitivity for the detection of terpene synthase genes that are expressed at a low level. An example of a cDNA synthesis protocol is as follows:

Quantified RNA is used as input for cDNA synthesis using the SuperScript™ IV First-Strand Synthesis System (ThermoFisher). cDNA synthesis reactions are prepared as follows: 1 µL 50 µM Oligo d(T) 20 primer (SEQ ID NO:1400), 1 µL of 10 mM dNTP mix (10 mM each dNTP), 8 µLTemplate RNA (10 pg-5 µg total RNA or 10 pg-500 ng mRNA), up to 3 µL DEPC-treated water are mixed together for a 13 µL final volume. After mixing and briefly centrifuging, the RNA-primer mix reactionsawere heated at 65° C. for 5 minutes, and then incubated at 0° C. for 2 minutes on a Veriti thermocycler (ABI).

Following annealing, the plate is pierced using a plate piercer and 7 uL Reverse transcriptase (RT) reaction mix is added to each reaction for a final volume of 20 uL final volume for cDNA synthesis. The RT reaction mix is prepared by mixing together the following: 4 µL of 5× SSIV Buffer, 1 µL of 100 mM DTT, 1 µL of Ribonuclease Inhibitor, and 1 µL of SuperScript™ IV Reverse Transcriptase (200 U/µL). The plate is sealed and briefly centrifuged, then loaded onto a Veriti thermocycler for cDNA synthesis using the following protocol:
Incubate the combined reaction mixture at 50-55° C. for 10 minutes;
Inactivate the reaction mixture by incubation at 80° C. for 10 minutes, then store at 4° C.

The resulting products of cDNA synthesis are prepared for quantification using the QuantiFluor HS-dsDNA System (Promega) and quantified using a Quantus Fluorometer (Promega), as per the manufacturer’s instructions. The quantitated cDNA is diluted to a 2 ng/uL final working concentration and used as normalized input into either an end-point PCR reaction or a Taqman real-time qPCR reaction.

### Endpoint PCR With Gel Analysis

2.5 uL of normalized cDNA is used as input for a PCR Master Mix (total volume: 22.5 uL), as follows: 12.5 uL 2X Promega Colorless GoTaq (Promega), 0.1 uL of 100 uM Primer Mix (see Table B, in Primer Groups 1-19 as set forth in Tables 1-16 and in B3/F3 polynucleotide primer pairs set forth in Tables 17-35 for primer sequences); 100 uM of a single primer pair to detect one exon, or multiple primer pairs that each detects an exon of unique size in the set of TPS genes of the plant cultivar of interest), and 9.5 uL Nuclease free Water (Ambion).

The reactions are subjected to the following thermocycler protocol: 1 cycle of 95° C. for 10 mins; 35 cycles of 95° C. for 40 seconds, 60° C. for 2 mins, 72° C. for 2 mins; 1 cycle of 72° C. for 5 mins; 4° C. hold. The End point PCR reactions are analyzed by diluting 1:2 in nuclease free water and 20 ul is loaded into each well of one or more E-Gel™ EX Agarose Gels, 2%, depending on the number of samples, and run for 10 minutes on 1-2% gel settings for the E-gel system. The bands are analyzed for the presence of exons, based on the expected sizes of the amplicons. In addition, if the DNA is normalized, the intensities (e.g., fluorescence intensity) of the bands can provide information regarding the numbers of copies of the TPS genes and/or the ploidy (e.g., diploid, triploid, tetraploid, etc.).

### Pro-Amplification PCR

To reduce the effect of plant materials in subsequent reactions and analyses, for example when crude DNA or RNA extracts are used, plant pigments and potentially real time qPCR-inhibiting compounds often found in such extracts can optionally be removed by performing a pre-amplification PCR for 10 cycles. 2.5 uL of crude genomic DNA extract is transferred to a second PCR plate, with each well pre-loaded with 22.5 uL of Pre-amplification PCR master mix prepared per reaction as follows: 12.5 uL 2X Promega Colorless GoTaq (Promega), 3 uL of 4 uM of the desired primers to be analyzed (see Table B, in Primer Groups 1-19 as set forth in Tables 1-16 and in B3/F3 polynucleotide primer pairs set forth in Tables 17-35 for primer sequences), and 7 uL Nuclease free Water (Ambion). The reactions are subjected to the following thermocycler protocol: 1 cycle of 95° C. for 10 mins; 10 cycles of 95° C. for 40 sec, 60° C. for 2 mins, 72° C. for 2 mins; 1 cycle of 72° C. for 5 mins; 4° C. hold.

Pre-amplification reactions are diluted 1:5 with 100 uL Nuclease free water (Ambion). The diluted pre-amplification reactions are prepared for quantification using the QuantiFluor dsDNA System (Promega) and quantified using a Quantus Fluorometer (Promega), as per manufacturer’s instructions. Quantitated diluted pre-amplification reactions reveal a final working concentration of ~1 ng/uL, which is used as unnormalized input into the real time qPCR reactions.

### High Resolution Melt (HRM) Analysis

HRM analysis was performed in 10 uL reactions on a LightCycler 480 qPCR (Roche Applied Systems) using the following protocol: 1 pre-incubation cycle (95° C. for 10 mins), 45 amplification cycles (95° C. for 10 secs, 60° C. for 15 secs, 72C for 10 secs), 1 cycle of HRM (95° C. for 1 min, 40° C. for 1 min, 65° C. for 1 sec and heat to 95° C. with 25 continuous acquisitions per degree Celsius followed by a final cooling cycle (40° C. for 10 secs) (Vossen et al., Biochemica 4:10-11 (2007)). Each reaction contains: 5 uL of ~1 ng/uL of the diluted pre-amplified template that is the product of pre-amplification PCR. cDNA also can be amplified in an exon-specific manner to get a fingerprint of the terpene synthase expressosome, i.e., to see which of the TPS genes are expressed (using a single pair of primers or multiple pairs of primers that each amplify an exon of a unique size), or gDNA can be amplified (with or without pre-amplification) to get a fingerprint of the entire genome. 5 uL of HRM Master Mix (prepared per reaction as follows: 3.5 uL 2X High Resolution Melting Master Mix containing HRM dye (Roche Applied Systems), 0.6 uL of 4 uM Primer Mix (see Table B, in Primer Groups 1-19 as set forth in Tables 1-16 and in B3/F3 polynucleotide primer pairs set forth in Tables 17-35 for primer sequences; 4 uM of a single primer pair to detect one exon, or multiple primer pairs that each detects an exon of unique size in the set of TPS genes of the plant cultivar of interest), 0.8 uL of 25 mM MgCl₂, 1.125 uL of Nuclease free water). High Resolution Melting data was analyzed using the LightCycler 480 Melt Genotyping software. Fluorescence intensity as a function of temperature for each sample also was analyzed using R software and Matlab custom scripts to determine statistical variation of melt curves and statistical analysis

### Example 3: Analysis of gDNA and cDNA From Cannabis Plants by LAMP Assay

A representative LAMP assay is described herein (as applied to the LAMP primer sets depicted in Table C). This assay can be performed with any of the LAMP primer sets, with or without the loop primers, provided in Tables 17-35 herein.

Total RNA, total gDNA, crude FTA extract (nucleic acid from extract from filter paper, such as Whatman, and synthesized cDNA templates were prepared from three distinct genotype/chemotypes of Type I Cannabis plants (de Meijer et al., Genetics, 163(1):335-346 (2003)). The samples were isolated from mid-flower tissue of the plants, and the samples for each of the distinct genotypes/chemotypes are named LPA4, LPA5, and LPA21.3.

For each of the samples, gDNA, cDNA and FTA crude extracts were subjected to GoTaq PCR reaction (Promega, Madison, WI) using primers B3 and F3 from csTPS37FN LAMP Primer Sets 1, 2 and 3 for the detection of csTPS37FN. Using the B3 and F3 primers from LAMP Primer Set 1 in a GoTaq PCR reaction using LPA005 gDNA, LPA005 cDNA, LPA004 gDNA, LPA004 cDNA, LPA021.3 gDNA, LPA021.3 gDNA, gel electrophoresis analysis showed that the target amplicons of interest are present after PCR as a non-specfic uniform sized ~200 bp amplicon product whether amplified from gDNA, cDNA, or FTA extract input in the LPA005 sample. The amplicon product was absent in LPA004 and LPA021.3 samples from either cDNA or gDNA.

The csTPS37FN B3/F3 Primer Set 1 target amplicons of interest were each excised from the gel, and the amplicons purified and sequenced by Sanger sequencing; 133 bp of DNA sequence was recovered with a 99.2% consensus agreement (132 bp out of 133 bp) between bands labeled TPS37-1 gDNA (LPA005 gDNA), TPS37-1 cDNA (LPA005 cDNA), and TPS37-1 FTA (LPA005 FTA Extract) samples and the csTPS37FN published reference sequences MK614216.1 and Finola (GCA 003417725.2). A single SNP (C to A) in the alignment was observed in the labeled TPS37-1 gDNA (LPA005 gDNA), TPS37-1 cDNA (LPA005 cDNA), and TPS37-1 FTA (LPA005 FTA Extract) samples relative to the csTPS37FN published reference sequences MK614216.1 and Finola (GCA 003417725.2).

Using the B3 and F3 primers from LAMP Primer Set 2 in a GoTaq PCR reaction on LPA005 gDNA, LPA005 cDNA, LPA004 gDNA, LPA004 cDNA, LPA021.3 gDNA, LPA021.3 gDNA LPA005 FTA Extract and NTC, as revealed by gel electrophoresis analysis, showed specific detection of distinct sized amplicons at -480 bp for LPA005 gDNA, at ~200 bp for LPA005cDNA, and at ~480 bp for LPA005 FTA extract. Amplicons were absent in LPA004 and LPA021.3 samples from either cDNA or gDNA input. The csTPS37FN B3/F3 Set 2 target amplicons of interest were each excised from the gel, the amplicons purified and sequenced by Sanger sequencing; 415 bp and 414 bp of DNA sequence was recovered from the LPA005 gDNA and LPA005 FTA extract with a 98.5% and 98.3% nucleotide consensus agreement (409 bp and 408 bp out of 415 bp) between the LPA005 gDNA and LPA005 FTA extract bands labeled TPS37-2 gDNA (LPA005 gDNA), TPS37-2 FTA extract (LPA005 FTA extract) and the csTPS37FN published reference sequence from Finola (GCA_003417725.2), while 141 bp of DNA sequence was recovered from the LPA005 cDNA with a 100% nucleotide similarity consensus agreement (141 bp out of 141 bp) between the LPA005 cDNA bands labeled TPS37-2 cDNA (LPA005 cDNA) and the published reference sequence from Finola (GCA_003417725.2).

Using the B3 and F3 primers from LAMP Primer Set 3, in a GoTaq PCR reaction on LPA005 gDNA, LPA005 cDNA, LPA004 gDNA, LPA004 cDNA, LPA021.3 gDNA, LPA021.3 gDNA LPA005 FTA Extract, and NTC, gel electrophoresis analysis showed specific detection of distinct sized amplicons at ~500 bp for LPA005 gDNA, at ~250 for LPA005cDNA, and at ~500 bp for the LPA005 FTA extract. The target amplicons of interest are present after PCR whether amplified from gDNA, cDNA, or FTA extract from in the LPA005 sample. Amplicons were absent in LPA004 and LPA021.3 samples from either cDNA or gDNA input. The csTPS37FN B3/F3 Set 3 target amplicons of interest were each excised from the gel, the amplicons purified and sequenced by Sanger sequencing; 437 bp and 427 bp of DNA sequence was recovered from the LPA005 gDNA and LPA005 FTA extract with a 98.8% and 96.5% nucleotide similarity consensus agreement (432 bp and 422 bp out of 437 bp) between the LPA005 gDNA and LPA005 FTA extract bands labeled TPS37-3 gDNA (LPA005 gDNA), TPS37-3 FTA extract (LPA005 FTA extract) and the csTPS37FN published reference sequence from Finola (GCA_003417725.2), while 180 bp of DNA sequence was recovered from the LPA005 cDNA with a 100% nucleotide similarity consensus agreement (180 bp out of 180 bp) between the LPA005 cDNA bands labeled TPS37-2 cDNA (LPA005 cDNA) and the published reference sequence from Finola (GCA 003417725.2).

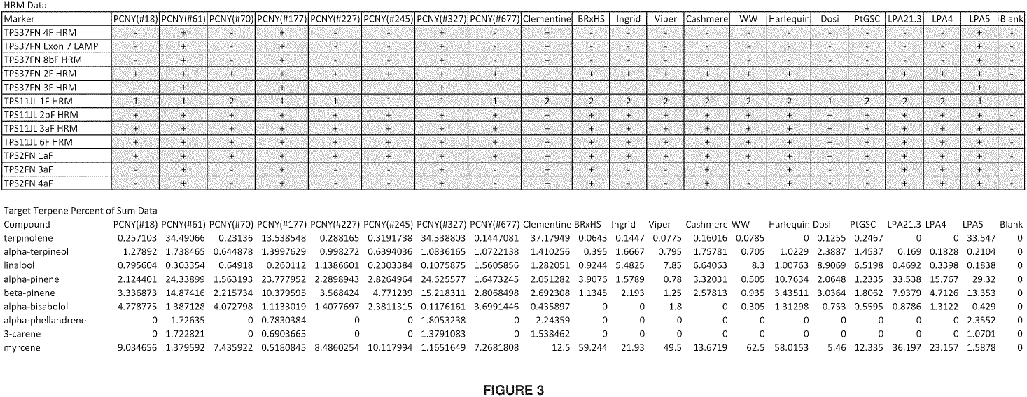

Using csTPS37FN LAMP Primer Sets 1, 2, and 3, 1 uL of the following input from sample numbers 1) LPA005 gDNA, 2) LPA005, 3) LPA004 gDNA, 4) LPA004 cDNA, 5) LPA021.3 gDNA, 6) LPA021.3 cDNA, 7) LPA005 FTA extract, and 8) NTC were analyzed. The sample set was loaded into a csTPS37FN LAMP based assay detection reaction prepared with NEB WarmStart Colorimetric LAMP Mastermix Mix (New England Biolabs, Ipswich, MA), csTPS37 LAMP Primer sets 1, 2, or 3, and nuclease free water. At time 0, all LAMP reactions are seen as dark grey. After a 45-minute reaction at 65° C., positive LAMP reactions are seen as a pale liquid and negative LAMP reactions are seen as dark grey (FIG. 3). LPA005 gDNA, LPA005 cDNA, LPA004 gDNA, LPA021.3 gDNA, and LPA005 FTA extract are seen as a pale liquid in the LAMP Primer Set 1 reactions and recorded positive; LPA004 cDNA, LPA021.3 cDNA and the negative control are seen as dark grey in the LAMP Primer Set 1 reactions and recorded negative. LPA005 cDNA are seen as pale grey in LAMP Primer Sets 2 and 3 reactions and are recorded positive; LPA005 gDNA, LPA004 gDNA, LPA004 cDNA, LPA021.3 gDNA, LPA004 cDNA, and FTA extract are seen as dark grey in LAMP Primer Sets 2 and 3 reactions and recorded negative. The results demonstrate specific detection of expressed csTPS37FN in LPA5 cDNA using csTPS37FN LAMP Primer Sets 2 and 3. Non-specific detection using csTPS37FN LAMP Primer set 1 was observed in cDNA and FTA extract for LPA005 and gDNA for LPA004, LPA005, and LPA021.3. Positive detection of accumulated amplicon is seen as pale samples rather than dark grey samples.

### Example 4: Correlation of Terpene Synthase Profiles With Chemical Phenotypes of Terpene Distribution in Plants

High Resolution Melting (HRM) experiments (and one LAMP experiment, see below) were performed using genomic DNA from various Cannabis cultivars. A Master Mix for the HRM reaction reaction was prepared with 2.75 uL LightCycler® 480 High Resolution Melting Master Mix, 0.8 uL of 25 mM MgCl₂, 0.6 uL of primer, and 5 uL of water, with 1 uL of 1 ng/uL Cannabis genomic DNA/water used as template. Samples were tested in duplicate. LPA5 genomic DNA was used as a positive control and water was used as a negative/no template control. The reactions were performed using the following protocol:

A PCR/HRM reaction was carried out with a ramp rate of at 1.6° C./s and was incubated at 50° C. for 2 minutes, ramped to 95° C. for 10 minutes for 1 time. Then the reaction was cycled through 95° C. for 15 seconds, 68° C. for 30 seconds, and 72° C. for 30 seconds for 40 times, where SYBR fluorescence measurements were captured. This was followed by a melting event at 95° C. for 15 seconds, 60° C. for 1 minute and ramping up at a ramp rate of 0.15° C./s with SYBR fluorescence measurements until reaching 95° C. for 1 second.

A Tm was measured for each reaction, the peak height was normalized against a threshold detection of Sybr fluorescence, and the data was analyzed and organized into an output based on well, well position, Cannabis sample, target gene/exon, Tm and Melt Peak Height. The results are summarized in FIG. 3.

Depicted in FIG. 3 is a top panel summarizing the results of the HRM terpene synthase analysis for each Cannabis cultivar, and a bottom panel showing the corresponding terpene chemical phenotypes for each cultivar. In the top panel summarizing the HRM analysis, the Cannabis cultivars/samples are listed along the x-axis at the top (“Blank” = negative control, i.e., water) and the primers tested are displayed along the y axis (under the “Marker” column; note that primer pairs were used to detect each terpene synthase exon indicated so, for example, where TPS37FN 4F is indicated, the 4F and 4R primers (forward and reverse, respectively) were used to detect exon 4 of TPS37FN, TPS2FN3aF means the 3aF and 3aR primers for detecting the 3a exon of TPS2FN were used, and the like). The sequences for each primer pair under the “Marker” column are provided in Table B, except for “TPS37FN Exon 7 LAMP primers,” where LAMP analysis was performed using the LAMP primer set as set forth in Table 17. Results are described as either presence (+) or absent (-) for each primer pair tested. Additionally, the TPS11JL 1F/1R primer pair detected genotype groups identified by their melting peak TPS2FN3aF and the results for that primer set are denoted as either group 1 (1) or group 2 (2).

The bottom panel of FIG. 3 shows the corresponding terpene distribution of each Cannabis cultivar, as measured by GC-MS; the amount of each terpene is displayed as a percent of sum for each target terpene from the Cannabis samples. The data demonstrates that analysis of the target terpene synthase (TPS) exons tested using primers TPS37 4F/R HRM, TPS37 Exon 7 LAMP, TPS37 8bF/R HRM, TPS37 3F/R HRM, and TPS11JL 1F/R HRM can predict terpinolene as a significant component of the chemical phenotype of plant cultivars having these exons. Additional exon analysis using primers TPS2FN 3aF/R and TPS2FN 4aF/R allow prediction of Cannabis sultivars having alpha pinene as a major component of their chemical phenotype.

The results further demonstrate the correlation between the specificity of detection of an exon from a functional terpene synthase allele and the prediction of its corresponding terpene profile. For example, Cannabis cultivars in which specific detection of TPS37FN exons 3 and 4 were obtained were found to have terpinolene as a major terpene component. Non-specific detection of TPS37FN exon 2 across all Cannabis cultivar samples tested, however, did not correlate to terpinolene as a major component of the cultivars. A summary of the results is provided below:

A. “Informative” Exons (i.e., detection of the exons is specific and can be correlated to chemical phenotype, i.e., used to predict terpene composition)

### TPS2FN 3aF/R HRM Validation

Specificity of functional TPS2FN allele detection was observed with this primer set. Samples with the functional allele were measured with a melting peak of around 76° C., while samples without this allele were not detected, i.e., no fluorescence was measured, nor amplicon produced. The positive control LPA5 cultivar demonstrated presence of the target allele for this genotype. No signal was observed in the negative/NTC (no template control, i.e., water and no plant sample).

### TPS2FN 4aF/R HRM Validation

Specificity of functional TPS2FN allele detection was observed with this primer set. Samples with the functional allele were measured with a melting peak of around 78° C., while samples without this allele were not detected, i.e., no fluorescence was measured, nor amplicon produced. The positive control LPA5 cultivar demonstrated presence of the target allele for this genotype. No signal was observed in the negative/NTC (no template control, i.e., water and no plant sample).

### TPS11JL 1F/R HRM Validation

While all Cannabis samples generated a positive signal with this primer set, the specificity of either two melting peaks or one melting peak based on genotypes of TPS11JL exon 1 were observed. Samples with a single melting peak (denoted as Group 1) were measured with a melting peak around 80° C. The positive control LPA5 cultivar showed the presence of the single melting peak, thereby corresponding to the target allele for the “Group 1” genotype. Samples in which the alternate allele was detected (Group 2) showed two melting peaks at around 76° C. and 80° C. No signal was observed in the negative/NTC (no template control, i.e., water and no plant sample).

### TPS37FN 3F/R HRM Validation

Specificity of functional TPS37FN allele detection was observed with this primer set. Samples with the functional allele were measured with a melting peak of around 75° C., while samples without this allele were not detected, i.e., no fluorescence was measured, nor amplicon produced. The positive control LPA5 cultivar demonstrated presence of the target allele for this genotype. No signal was observed in the negative/NTC (no template control, i.e., water and no plant sample).

### TPS37FN 4F/R HRM Validation

While most Cannabis samples generated a positive signal with this primer set, the specificity of either one melting peak or multiple melting peaks based on genotypes of the TPS37FN exon 4 were observed. Samples with a single melting peak (denoted as TPS37FN positive group) were measured with a melting peak around 75° C. The positive control LPA5 cultivar showed the presence of the single melting peak, thereby corresponding to the target allele for the genotype denoted as the TPS37FN positive group. Samples in which the alternate allele(s) were detected showed multiple melting peaks at around 73° C., 77° C., 81° C. or 83° C., or no melting peak. No signal was observed in the negative/NTC (no template control, i.e., water and no plant sample).

### TPS37FN Exon 7 LAMP Primers

Specificity of functional TPS37FN allele detection was observed with this primer set. Samples with the functional allele were measured as a positive colorimetric reaction (yellow), while samples without this allele were detected as a negative colorimetric reaction (pink). The positive control LPA5 cultivar demonstrated presence of the target allele for this genotype (yellow colorimetric reaction). No signal was observed in the negative/NTC (no template control, i.e., water and no plant sample).

### TPS37FN 8bF/R HRM Validation

Specificity of functional TPS37FN allele detection was observed with this primer set. Samples with the functional allele were measured with a melting peak of around 75° C., while samples without this allele were not detected, i.e., no fluorescence was measured, nor amplicon produced. The positive control LPA5 cultivar demonstrated presence of the target allele for this genotype. No signal was observed in the negative/NTC (no template control, i.e., water and no plant sample).

B. “Uninformative” Exons (i.e., detection of the exons is non-specific and not always correlated to chemical phenotype, i.e., terpene composition)

### TPS2FN 1aF/R HRM Validation

All Cannabis cultivar samples were detected with this exon primer set, with the measurement of a single melting peak at or around 75° C. for all samples tested. No specificity was observed for analysis of this allele, with the control LPA5 sample aligning with a conserved genotype for this allele. No signal was observed in the negative/NTC (no template control). No signal was observed in the negative/NTC (no template control, i.e., water and no plant sample).

### TPS11JL 2bF/R HRM Validation

All Cannabis cultivar samples were detected with this exon primer set, with the measurement of a single melting peak at or around 75° C. for all samples tested. No specificity was observed for analysis of this allele, with the control LPA5 sample aligning with a conserved genotype for this allele. No signal was observed in the negative/NTC (no template control). No signal was observed in the negative/NTC (no template control, i.e., water and no plant sample).

### TPS11JL 3aF/R HRM Validation

All Cannabis cultivar samples were detected with this exon primer set, with the measurement of a single melting peak at or around 78° C. for all samples tested. No specificity was observed for analysis of this allele, with the control LPA5 sample aligning with a conserved genotype for this allele. No signal was observed in the negative/NTC (no template control). No signal was observed in the negative/NTC (no template control, i.e., water and no plant sample).

### TPS11JL 6F/R HRM Validation

All Cannabis cultivar samples were detected with this exon primer set, with the measurement of a single melting peak at or around 77° C. for all samples tested. No specificity was observed for analysis of this allele, with the control LPA5 sample aligning with a conserved genotype for this allele. No signal was observed in the negative/NTC (no template control). No signal was observed in the negative/NTC (no template control, i.e., water and no plant sample).

### TPS37FN 2F/R HRM Validation

All Cannabis cultivar samples were detected with this exon primer set, with the measurement of a single melting peak at or around 77° C. for all samples tested. No specificity was observed for analysis of this allele, with the control LPA5 sample aligning with a conserved genotype for this allele. No signal was observed in the negative/NTC (no template control). No signal was observed in the negative/NTC (no template control, i.e., water and no plant sample).

The results demonstrate that allele-specific detection of terpene synthases using a multi-exon analysis can provide a robust correlation between the terpene synthase profile of a plant cultivar and its expected chemical phenotype, e.g., terpene composition.

### Example 5: Examples of Embodiments

A1. A method of identifying/selecting a plant cultivar comprising at least one terpene synthase gene that produces at least one terpene that has an energizing effect on a subject, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an energetic terpene product profile comprising at least one terpene that has an energizing effect on a subject, wherein the polynucleotide primer pair comprises at least one polynucleotide primer pair selected from among one or more of:
   primer group 1 as set forth in Table 1 and the B3/F3 primer pairs set forth in Table 18;
   primer group 2 as set forth in Table 2 and the B3/F3 primer pairs set forth in Tables 22 and 26;
   primer group 3 as set forth in Table 3 and the B3/F3 primer pairs set forth in Table 19;
   primer group 4 as set forth in Table 4 and the B3/F3 primer pairs set forth in Table 23;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 5 as set forth in Table 5 and the B3/F3 primer pairs set forth in Table 28;
   primer group 6 as set forth in Table 6 and the B3/F3 primer pairs set forth in Table 17;
   primer group 19 as set forth in Table 16 and the B3/F3 primer pairs set forth in Table 34; and
   primer group 9 as set forth in Table 9 and the B3/F3 primer pairs set forth in Table 30;
(c) amplifying the mixture, thereby obtaining an amplified mixture;
(d) analyzing the amplified mixture of (c), wherein the presence of at least one terpene synthase gene that produces an energetic terpene product profile comprising at least one terpene that has an energizing effect on a subject is identified in the amplified mixture based on the presence of at least one amplified subsequence of the terpene synthase gene; and
(e) identifying and/or selecting a plant cultivar based on the at least one terpene synthase gene that is identified in (d).

A1.1 A method of identifying/selecting a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, wherein the polynucleotide primer pair comprises at least one polynucleotide primer pair selected from among one or more of:
   primer group 1 as set forth in Table 1 and the B3/F3 primer pairs set forth in Table 18;
   primer group 2 as set forth in Table 2 and the B3/F3 primer pairs set forth in Tables 22 and 26;
   primer group 3 as set forth in Table 3 and the B3/F3 primer pairs set forth in Table 19;
   primer group 4 as set forth in Table 4 and the B3/F3 primer pairs set forth in Table 23;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 5 as set forth in Table 5 and the B3/F3 primer pairs set forth in Table 28;
   primer group 6 as set forth in Table 6 and the B3/F3 primer pairs set forth in Table 17;
   primer group 19 as set forth in Table 16 and the B3/F3 primer pairs set forth in Table 34; and
   primer group 9 as set forth in Table 9 and the B3/F3 primer pairs set forth in Table 30;
(c) amplifying the mixture, thereby obtaining an amplified mixture;
(d) analyzing the amplified mixture of (c), wherein the presence, absence and/or amount of at least one amplicon generated by at least one polynuceotide primer pair of (b) is identified in the amplified mixture; and
(e) identifying and/or selecting a plant cultivar based on the presence, absence and/or amount of at the least one amplicon analyzed in (d).

A1.2. The method of embodiment A1.1, wherein the presence and/or amount of at least one amplicon generated by at least one polynuceotide primer pair of (b) is identified in (d).

A1.3. The method of embodiment A1.2, wherein the amplicon comprises at least one amplified subsequence of a terpene synthase gene.

A1.4. The method of embodiment A1.3, wherein the terpene synthase gene produces an energetic terpene product profile comprising at least one terpene that has an energizing effect on a subject.

A2. A method of identifying/selecting a plant cultivar that does not comprise a terpene synthase gene that produces a terpene that has a sedative effect on a subject or that comprises a terpene synthase gene that produces a decreased amount of a terpene that has a sedative effect on a subject, whereby the terpene production profile has an energizing effect on the subject, the method comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces a sedative terpene product profile comprising at least one terpene that has an sedative effect on a subject, wherein the polynucleotide primer pair comprises at least one polynucleotide primer pair selected from primer group 7, as set forth in Table 7 and the B3/F3 primer pairs set forth in Tables 25 and 30;
(c) amplifying the mixture, thereby obtaining an amplified mixture;
(d) analyzing the amplified mixture of (c), wherein the absence of at least one terpene synthase gene that produces a sedative terpene product profile comprising at least one terpene that has a sedative effect on a subject, or the presence of at least one non-functional terpene synthase gene that inhibits or reduces the production of at least one terpene that has a sedative effect on a subject, is identified in the amplified mixture based on the absence of at least one amplified subsequence of the terpene synthase gene; and
(e) identifying and/or selecting a plant cultivar based on the absence of the least one terpene synthase gene identified in (d) or based on the presence of the at least one non-functional terpene synthase gene identified in (d).

A2.1. A method of identifying/selecting a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair comprises at least one polynucleotide primer pair selected from primer group 7, as set forth in Table 7 and the B3/F3 primer pairs set forth in Tables 25 and 30;
(c) amplifying the mixture, thereby obtaining an amplified mixture;
(d) analyzing the amplified mixture of (c), wherein the presence, absence and/or amount of at least one amplicon generated by at least one polynuceotide primer pair of (b) is identified in the amplified mixture; and
(e) identifying and/or selecting a plant cultivar based on the presence, absence and/or amount of at the least one amplicon analyzed in (d).

A2.2. The method of embodiment A2.1, wherein the presence, absence and/or amount of at least one amplicon generated by at least one polynuceotide primer pair of (b) is identified in (d).

A2.3. The method of embodiment A2.2, wherein the absence or a reduced amount of at least one amplicon generated by at least one polynuceotide primer pair of (b) is identified in (d).

A2.4. The method of embodiment A2.2 or A2.3, wherein the identifying and/or selecting in (e) is based on an absence or a reduced amount of at least one amplicon generated by at least one polynuceotide primer pair of (b) identified in (d).

A2.5. The method of embodiment A2.3 or A2.4, wherein the at least one amplicon is of a terpene synthase gene that produces a sedative terpene product profile comprising at least one terpene that has a sedative effect on a subject.

A3. A method of preparing nucleic acid from a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an energetic terpene product profile comprising at least one terpene that has an energizing effect on a subject, wherein the polynucleotide primer pair comprises at least one polynucleotide primer pair selected from among one or more of:
   primer group 1 as set forth in Table 1 and the B3/F3 primer pairs set forth in Table 18;
   primer group 2 as set forth in Table 2 and the B3/F3 primer pairs set forth in Tables 22 and 26;
   primer group 3 as set forth in Table 3 and the B3/F3 primer pairs set forth in Table 19;
   primer group 4 as set forth in Table 4 and the B3/F3 primer pairs set forth in Table 23;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 5 as set forth in Table 5 and the B3/F3 primer pairs set forth in Table 28;
   primer group 6 as set forth in Table 6 and the B3/F3 primer pairs set forth in Table 17;
   primer group 19 as set forth in Table 16 and the B3/F3 primer pairs set forth in Table 34; and
   primer group 9 as set forth in Table 9 and the B3/F3 primer pairs set forth in Table 30;
(c) amplifying the mixture, thereby obtaining an amplified mixture;
(d) analyzing the amplified mixture of (c), wherein the presence of at least one terpene synthase gene that produces an energetic terpene product profile comprising at least one terpene that has an energizing effect on a subject is identified in the amplified mixture based on the presence of at least one amplified subsequence of the terpene synthase gene; and
(e) identifying the amplified mixture analyzed in (d) as comprising prepared nucleic acid comprising at least one terpene synthase gene that produces an energetic terpene product profile comprising at least one terpene that has an energizing effect on a subject.

A3.1. A method of preparing nucleic acid from a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, wherein the polynucleotide primer pair comprises at least one polynucleotide primer pair selected from among one or more of:
   primer group 1 as set forth in Table 1 and the B3/F3 primer pairs set forth in Table 18;
   primer group 2 as set forth in Table 2 and the B3/F3 primer pairs set forth in Tables 22 and 26;
   primer group 3 as set forth in Table 3 and the B3/F3 primer pairs set forth in Table 19;
   primer group 4 as set forth in Table 4 and the B3/F3 primer pairs set forth in Table 23;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 5 as set forth in Table 5 and the B3/F3 primer pairs set forth in Table 28;
   primer group 6 as set forth in Table 6 and the B3/F3 primer pairs set forth in Table 17;
   primer group 19 as set forth in Table 16 and the B3/F3 primer pairs set forth in Table 34; and
   primer group 9 as set forth in Table 9 and the B3/F3 primer pairs set forth in Table 30; and
(c) amplifying the mixture, thereby obtaining an amplified mixture of prepared nucleic acid.

A3.2. The method of embodiment A3.1, further comprising analyzing the amplified mixture of (c), wherein the presence, absence and/or amount of at least one terpene synthase gene is determined.

A3.3. The method of embodiment A3.2, wherein the at least one terpene synthase gene produces an energetic terpene product profile comprising at least one terpene that has an energizing effect on a subject.

A3.4. The method of embodiment A3.3., wherein the presence and/or amount of at least one amplified subsequence of the terpene synthase gene is identified, and the terpene synthase gene produces an energetic terpene product profile comprising at least one terpene that has an energizing effect on a subject.

A4. A method of preparing nucleic acid from a plant cultivar that does not comprise a terpene synthase gene that produces a terpene that has a sedative effect on a subject or that comprises a terpene synthase gene that produces a decreased amount of a terpene that has a sedative effect on a subject, the method comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces a sedative terpene product profile comprising at least one terpene that has an sedative effect on a subject, wherein the polynucleotide primer pair comprises at least one polynucleotide primer pair selected from primer group 7, as set forth in Table 7 and the B3/F3 primer pairs set forth in Tables 25 and 30;
(c) amplifying the mixture, thereby obtaining an amplified mixture;
(d) analyzing the amplified mixture of (c), wherein the absence of at least one terpene synthase gene that produces a sedative terpene product profile comprising at least one terpene that has a sedative effect on a subject, or the presence of at least one non-functional terpene synthase gene that inhibits or reduces the production of at least one terpene that has a sedative effect on a subject, is identified in the amplified mixture based on the absence of at least one amplified subsequence of the terpene synthase gene; and
(e) identifying the amplified mixture analyzed in (d) as comprising prepared nucleic acid comprising the absence of at least one terpene synthase gene that produces at least one terpene that has s sedative effect or comprises a at least one non-functional terpene synthase gene that produces a reduced amount of at least one terpene that has a sedative effect.

A4.1. A method of preparing nucleic acid from a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair comprises at least one polynucleotide primer pair selected from primer group 7, as set forth in Table 7 and the B3/F3 primer pairs set forth in Tables 25 and 30; and
(c) amplifying the mixture, thereby obtaining an amplified mixture of prepared nucleic acid.

A4.2. The method of embodiment A4.1, further comprising analyzing the amplified mixture of (c), wherein the presence, absence and/or amount of at least one terpene synthase gene is determined.

A4.3. The method of embodiment A4.2, wherein the absence or a reduced amount of at least one terpene synthase gene produces a sedative terpene product profile comprising at least one terpene that has a sedative effect on a subject, is identified.

A4.4. The method of any one of embodiments A2, A2.4, A4, A4.2 or A4.3, wherein the at least one terpene that ha a sedative effect on the subject is R-linalool.

A5. The method of any one of embodiments A1 to A4.4, wherein the method comprises contacting the nucleic acid of the plant sample with a set of loop mediated isothermal amplification (LAMP) primers under the amplification conditions.

A6. The method of embodiment A5, wherein the LAMP primer set is selected from among one or more of the primer sets comprising B3, F3, BIP and FIP primers as set forth in each of the sets in Tables 17, 18, 19, 20, 21, 22, 23, 24, 25, 26, 28 and 30.

A7. The method of embodiment A6, wherein at least one LAMP primer set further comprises a LB loop primer, a LF loop primer, or both a LB loop primer and a LF loop primer.

A8. The method of embodiment A7, wherein at least one LB loop primer or at least one LF loop primer is set forth in Tables selected from among Tables 17, 18, 19, 20, 21, 22, 23, 24, 25, 26, 28 and 30.

A8.1. The method of any one of embodiments A1 to A8, wherein the plant is identified and/or selected based on the presence and/or amount of at least one of the following TPS genes: CsTPS2SK, CsTPSSFN, CsTPS30PK and CsTPS32PK, using at least one forward & reverse primer combination from primer groups 1, 2, 3, and 4, whereby the presence of α-pinene is greater than β-pinene.

A8.2. The method of any one of embodiments A1 to A8.1, wherein the plant is identified and/or selected based on the presence and/or amount of at least one of the following TPS genes: CsTPS6FN, CsTPS13PK, and CsTPS38FN, using at least one forward & reverse primer combination from primer groups 12, 13, and 14 respectively, whereby (Z)/(E)-β-ocimene bioaccumulation is present and/or increased.

A8.3. The method of any one of embodiments A1 to A8.2, wherein the plant is identified and/or selected based on the presence and/or amount of at least one of the following TPS genes: CsTPS31PK and CsTPS37FN using at least one forward & reverse primer combination from primer groups 5 and 6, respectively, whereby terpinolene bioaccumulation is present and/or increased.

A8.4. The method of any one of embodiments A1 to A8.3, wherein the plant is identified and/or selected based on the presence and/or amount of the following TPS gene: CsTPS33PK, using at least one forward & reverse primer combination from primer group 19, whereby terpinene (alpha- and gamma-) bioaccumulation is present and/or increased.

A8.4.1 The method of any one of embodiments A1 to A8.4, wherein the plant is identified and/or selected based on the presence and/or amount of the following TPS gene: CsTPS37LPA5, using at least one forward & reverse primer combination from primer group 6, whereby terpinene (alpha- and/or gamma-) bioaccumulation is present and/or increased.

A8.4.2 The method of embodiment A8.4.1, wherein the polypeptide encoded by the CsTPS37LPA5 comprises the sequence set forth in SEQ ID NO:1671.

A8.5. The method of any one of embodiments A1 to A8.4.2, wherein the plant is identified and/or selected based on the presence and/or amount of the following TPS gene: CsTPS18VF, using at least one forward & reverse primer combination from primer group 9, whereby S-Linalool bioaccumulation is present and/or increased.

A8.6. The method of any one of embodiments A1 to A8.5, wherein the plant is identified and/or selected based on the presence and/or amount of the following TPS gene: CsTPS18Choc, using at least one forward & reverse primer combination from primer group 7, whereby the absence of R-linalool is identified.

A9. A method of identifying/selecting a plant cultivar comprising at least one terpene synthase gene that produces at least one terpene that has an anti-nociceptive effect on a subject, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an anti-nociceptive terpene product profile comprising at least one terpene that has an anti-nociceptive effect on a subject, wherein the polynucleotide primer pair comprises at least one polynucleotide primer pair selected from among one or more of:
   primer group 1 as set forth in Table 1 and the B3/F3 primer pairs set forth in Table 18;
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29; and
   primer group 11 as set forth in Table 11 and the B3/F3 primer pairs set forth in Table 27;
(c) amplifying the mixture, thereby obtaining an amplified mixture;
(d) analyzing the amplified mixture of (c), wherein the presence of at least one terpene synthase gene that produces an anti-nociceptive terpene product profile comprising at least one terpene that has an anti-nociceptive effect on a subject is identified in the amplified mixture based on the presence of at least one amplified subsequence of the terpene synthase gene; and
(e) identifying and/or selecting a plant cultivar based on the at least one terpene synthase gene that is identified in (d).

A9.1 A method of identifying/selecting a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, wherein the polynucleotide primer pair comprises at least one polynucleotide primer pair selected from among one or more of:
   primer group 1 as set forth in Table 1 and the B3/F3 primer pairs set forth in Table 18;
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29; and
   primer group 11 as set forth in Table 11 and the B3/F3 primer pairs set forth in Table 27;
(c) amplifying the mixture, thereby obtaining an amplified mixture;
(d) analyzing the amplified mixture of (c), wherein the presence, absence and/or amount of at least one amplicon generated by at least one polynuceotide primer pair of (b) is identified in the amplified mixture; and
(e) identifying and/or selecting a plant cultivar based on the presence, absence and/or amount of at the least one amplicon analyzed in (d).

A9.2. The method of embodiment A9.1, wherein the presence and/or amount of at least one amplicon generated by at least one polynuceotide primer pair of (b) is identified in (d).

A9.3. The method of embodiment A9.2, wherein the amplicon comprises at least one amplified subsequence of a terpene synthase gene.

A9.4. The method of embodiment A9.3, wherein the terpene synthase gene produces an anti-nociceptive terpene product profile comprising at least one terpene that has an anti-nociceptive effect on a subject.

A10. A method of preparing nucleic acid from a plant cultivar comprising at least one terpene synthase gene that produces at least one terpene that has an anti-nociceptive effect on a subject, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an anti-nociceptive terpene product profile comprising at least one terpene that has an anti-nociceptive effect on a subject, wherein the polynucleotide primer pair comprises at least one polynucleotide primer pair selected from among one or more of:
   primer group 1 as set forth in Table 1 and the B3/F3 primer pairs set forth in Table 18;
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29; and
   primer group 11 as set forth in Table 11 and the B3/F3 primer pairs set forth in Table 27;
(c) amplifying the mixture, thereby obtaining an amplified mixture;
(d) analyzing the amplified mixture of (c), wherein the presence of at least one terpene synthase gene that produces an anti-nociceptive terpene product profile comprising at least one terpene that has an anti-nociceptive effect on a subject is identified in the amplified mixture based on the presence of at least one amplified subsequence of the terpene synthase gene; and
(e) identifying the amplified mixture analyzed in (d) as comprising prepared nucleic acid comprising at least one terpene synthase gene that produces an anti-nociceptive terpene product profile comprising at least one terpene that has an anti-nociceptive effect on a subject.

A10.1. A method of preparing nucleic acid from a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, wherein the polynucleotide primer pair comprises at least one polynucleotide primer pair selected from among one or more of:
   primer group 1 as set forth in Table 1 and the B3/F3 primer pairs set forth in Table 18;
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29; and
   primer group 11 as set forth in Table 11 and the B3/F3 primer pairs set forth in Table 27; and
(c) amplifying the mixture, thereby obtaining an amplified mixture of prepared nucleic acid.

A10.2. The method of embodiment A10.1, further comprising analyzing the amplified mixture of (c), wherein the presence, absence and/or amount of at least one terpene synthase gene is determined.

A10.3. The method of embodiment A10.2, wherein the at least one terpene synthase gene produces an anti-nociceptive terpene product profile comprising at least one terpene that has an anti-nociceptive effect on a subject.

A10.4. The method of embodiment A10.3., wherein the presence and/or amount of at least one amplified subsequence of the terpene synthase gene is identified, and the terpene synthase gene produces an anti-nociceptive terpene product profile comprising at least one terpene that has an anti-nociceptive effect on a subject.

A11. The method of any one or embodiments A9 to A10.4, wherein the method comprises contacting the nucleic acid of the plant sample with a set of loop mediated isothermal amplification (LAMP) primers under the amplification conditions.

A12. The method of embodiment A11, wherein the LAMP primer set is selected from among one or more of the primer sets comprising B3, F3, BIP and FIP primers as set forth in each of the sets in Tables 18, 23, 26, 27, 28, 29 and 30.

A13. The method of embodiment A12, wherein at least one LAMP primer set further comprises a LB loop primer, a LF loop primer, or both a LB loop primer and a LF loop primer.

A14. The method of embodiment A13, wherein at least one LB loop primer or at least one LF loop primer is set forth in Tables selected from among Tables 18, 23, 26, 27, 28, 29 and 30.

A14.1. The method of any one of embodiments A9 to A14, wherein the plant is identified and/or selected based on the presence and/or amount of at least one of the following TPS genes: CsTPS5PK, CsTPS31PK and CsTPS32PK, using at least one forward & reverse primer combination from primer groups 8, 5 and 4, whereby α-bisabolol accumulation is present and/or increased.

A14.2. The method of any one of embodiments A9 to A14.1, wherein the plant is identified and/or selected based on the presence and/or amount of at least one of the following TPS genes: CsTPS5PK, CsTPS31PK and CsTPS32PK, using at least one forward & reverse primer combination from primer groups 8, 5 and 4, respectively, whereby α-temineol accumulation is present and/or increased.

A14.3. The method of any one of embodiments A9 to A14.2, wherein the plant is identified and/or selected based on the presence and/or amount of at least one of the following TPS genes: CsTPS18VF, CsTPS19BL and CsTPS35LS, using at least one forward & reverse primer combination from primer groups 9, 10 and 11, respectively, whereby trans-nerolidol accumulation is present and/or increased.

A14.4. The method of any one of embodiments A9 to A14.3, wherein the plant is identified and/or selected based on the presence and/or amount of at least one of the following TPS genes: CsTPS2K and CsTPS32PK, using at least one forward & reverse primer combination from primer groups 1 and 4, respectively, whereby α-phellandrene accumulation is present and/or increased.

A14.5. The method of any one of embodiments A9 to A14.4, wherein the plant is identified and/or selected based on the presence and/or amount of the following TPS gene: TPS37LPA5, using at least one forward & reverse primer combination from primer group 6, whereby α-phellandrene accumulation is present and/or increased.

A14.5.1 The method of embodiment A14.5, wherein the polypeptide encoded by the CsTPS37LPA5 comprises the sequence set forth in SEQ ID NO:1671.

A15. A method of identifying/selecting a plant cultivar comprising at least one terpene synthase gene that produces at least one terpene that has an insecticidal effect and/or an insect predator attractant in the plant, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an insecticidal and/or an insect predator attractant terpene product profile comprising at least one terpene that has an insecticidal and/or an insect predator attractant effect in a plant, wherein the polynucleotide primer pair comprises at least one polynucleotide primer pair selected from among one or more of:
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29;
   primer group 11 as set forth in Table 11 and the B3/F3 primer pairs set forth in Table 27;
   primer group 16 as set forth in Table 13 and the B3/F3 primer pairs set forth in Table 35;
   primer group 7 as set forth in Table 7 and the B3/F3 primer pairs set forth in Table 25;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 15 as set forth in Table 12 and the B3/F3 primer pairs set forth in Table 31;
   primer group 17 as set forth in Table 14 and the B3/F3 primer pairs set forth in Table 32; and
   primer group 18 as set forth in Table 15 and the B3/F3 primer pairs set forth in Table 33;
(c) amplifying the mixture, thereby obtaining an amplified mixture;
(d) analyzing the amplified mixture of (c), wherein the presence of at least one terpene synthase gene that produces an insecticidal product profile and/or an insect predator attractant terpene product profile comprising at least one terpene that has an insecticidal effect and/or an insect predator attractant effect in a plant is identified in the amplified mixture based on the presence of at least one amplified subsequence of the terpene synthase gene; and
(e) identifying and/or selecting a plant cultivar based on the at least one terpene synthase gene that is identified in (d).

A15.1. A method of identifying/selecting a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair comprises at least one polynucleotide primer pair selected from among one or more of:
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29;
   primer group 11 as set forth in Table 11 and the B3/F3 primer pairs set forth in Table 27;
   primer group 16 as set forth in Table 13 and the B3/F3 primer pairs set forth in Table 35;
   primer group 7 as set forth in Table 7 and the B3/F3 primer pairs set forth in Table 25;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 15 as set forth in Table 12 and the B3/F3 primer pairs set forth in Table 31;
   primer group 17 as set forth in Table 14 and the B3/F3 primer pairs set forth in Table 32; and
   primer group 18 as set forth in Table 15 and the B3/F3 primer pairs set forth in Table 33;
(c) amplifying the mixture, thereby obtaining an amplified mixture;
(d) analyzing the amplified mixture of (c), wherein the presence, absence and/or amount of at least one amplicon generated by at least one polynuceotide primer pair of (b) is identified in the amplified mixture; and
(e) identifying and/or selecting a plant cultivar based on the presence, absence and/or amount of at the least one amplicon analyzed in (d).

A15.2. The method of embodiment 15.1, wherein the presence and/or amount of at least one amplicon generated by at least one polynuceotide primer pair of (b) is identified in (d).

A15.3. The method of embodiment 15.2, wherein the amplicon comprises at least one amplified subsequence of a terpene synthase gene.

A15.4. The method of embodiment 15.3, wherein the terpene synthase gene produces an insecticidal terpene product profile comprising at least one terpene that has an insectidal effect on a subject.

A16. A method of preparing nucleic acid from a plant cultivar comprising at least one terpene synthase gene that produces at least one terpene that has an insecticidal and/or an insect predator attractant effect in a plant, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an insecticidal and/or an insect predator attractant terpene product profile comprising at least one terpene that has an insecticidal and/or an insect predator attractant effect in a plant, wherein the polynucleotide primer pair comprises at least one polynucleotide primer pair selected from among one or more of:
   primer group 4 as set forth in Table 4 and the B3/F3 primer pairs set forth in Table 23;
   primer group 5 as set forth in Table 5 and the B3/F3 primer pairs set forth in Table 28;
   primer group 6 as set forth in Table 6 and the B3/F3 primer pairs set forth in Table 17;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29;
   primer group 11 as set forth in Table 11 and the B3/F3 primer pairs set forth in Table 27;
   primer group 16 as set forth in Table 13 and the B3/F3 primer pairs set forth in Table 35;
   primer group 7 as set forth in Table 7 and the B3/F3 primer pairs set forth in Table 25;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 15 as set forth in Table 12 and the B3/F3 primer pairs set forth in Table 31;
   primer group 17 as set forth in Table 14 and the B3/F3 primer pairs set forth in Table 32; and
   primer group 18 as set forth in Table 15 and the B3/F3 primer pairs set forth in Table 33;
(c) amplifying the mixture, thereby obtaining an amplified mixture;
(d) analyzing the amplified mixture of (c), wherein the presence of at least one terpene synthase gene that produces an insecticidal terpene product profile and/or an insect predator attractant terpene product profile comprising at least one terpene that has an insecticidal effect insect predator attractant effect in a plant is identified in the amplified mixture based on the presence of at least one amplified subsequence of the terpene synthase gene; and
(e) identifying the amplified mixture analyzed in (d) as comprising prepared nucleic acid comprising at least one terpene synthase gene that produces an insecticidal terpene product profile and/or an insect predator attractant terpene product profile comprising at least one terpene that has an insecticidal effect and/or an insect predator attractant effect in a plant.

A16.1 A method of preparing nucleic acid from a plant cultivar, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair comprises at least one polynucleotide primer pair selected from among one or more of:
   primer group 4 as set forth in Table 4 and the B3/F3 primer pairs set forth in Table 23;
   primer group 5 as set forth in Table 5 and the B3/F3 primer pairs set forth in Table 28;
   primer group 8 as set forth in Table 8 and the B3/F3 primer pairs set forth in Table 26;
   primer group 9 as set forth in Table 9 and the B3/F3 primer pairs set forth in Table 30;
   primer group 10 as set forth in Table 10 and the B3/F3 primer pairs set forth in Table 29;
   primer group 11 as set forth in Table 11 and the B3/F3 primer pairs set forth in Table 27;
   primer group 16 as set forth in Table 13 and the B3/F3 primer pairs set forth in Table 35;
   primer group 7 as set forth in Table 7 and the B3/F3 primer pairs set forth in Table 25;
   primer group 12 as set forth in the B3/F3 primer pairs set forth in Table 20;
   primer group 13 as set forth in the B3/F3 primer pairs set forth in Tables 21;
   primer group 14 as set forth in the B3/F3 primer pairs set forth in Table 24;
   primer group 15 as set forth in Table 12 and the B3/F3 primer pairs set forth in Table 31;
   primer group 17 as set forth in Table 14 and the B3/F3 primer pairs set forth in Table 32; and
   primer group 18 as set forth in Table 15 and the B3/F3 primer pairs set forth in Table 33; and
(c) amplifying the mixture, thereby obtaining an amplified mixture of prepared nucleic acid.

A16.2. The method of embodiment A16.1, further comprising analyzing the amplified mixture of (c), wherein the presence, absence and/or amount of at least one terpene synthase gene is determined.

A16.3. The method of embodiment A16.2, wherein the at least one terpene synthase gene produces an insecticidal terpene product profile comprising at least one terpene that has an insecticidal effect in a plant.

A16.4. The method of embodiment A16.3., wherein the presence and/or amount of at least one amplified subsequence of the terpene synthase gene is identified, and the terpene synthase gene produces an insecticidal terpene product profile comprising at least one terpene that has an insecticidal effect in a plant cultivar.

A16.5 The method of any one of embodiments A15 to A16.4, wherein the plant is identified and/or selected based on the presence and/or amount of the following TPS gene: CsTPS37LPA5, using at least one forward & reverse primer combination from primer group 6, whereby 3-carene bioaccumulation is present and/or increased.

A16.6 The method of embodiment A16.5, wherein the polypeptide encoded by the CsTPS37LPA5 comprises the sequence set forth in SEQ ID NO:1671.

A16.7 The method of any one of embodiments A15 to A16.6, wherein the plant is identified and/or selected based on the presence and/or amount of the following TPS gene: CsTPS20LPA5, using at least one forward & reverse primer combination from primer group 6, whereby guaiol bioaccumulation is present and/or increased.

A16.8 The method of embodiment A16.7, wherein the TPS20LPA5 polypeptide is encoded by the nucleotide sequence set forth in SEQ ID NO:1408.

A17. The method of any one of embodiments A15 to A16.4, wherein the method comprises contacting the nucleic acid of the plant sample with a set of loop mediated isothermal amplification (LAMP) primers under the amplification conditions.

A18. The method of embodiment A17, wherein the LAMP primer set is selected from among one or more of the primer sets comprising B3, F3, BIP and FIP primers as set forth in each of the sets in Tables 23, 28, 26, 30, 29, 27, 35, 25, 20, 21, 24, 31, 32 and 33.

A19. The method of embodiment A18, wherein at least one LAMP primer set further comprises a LB loop primer, a LF loop primer, or both a LB loop primer and a LF loop primer.

A20. The method of embodiment A19, wherein at least one LB loop primer or at least one LF loop primer is set forth in Tables selected from among Tables 23, 28, 26, 30, 29, 27, 35, 25, 20, 21, 24, 31, 32 and 33.

A20.1. The method of any one of embodiments A15 to A20, wherein the plant is identified and/or selected based on the presence and/or amount of the following TPS gene: a guiaol synthase, using at least one forward & reverse primer combination from primer group 16, whereby guaiol bioaccumulation is present and/or increased.

A20.2. The method of any one of embodiments A15 to A20.1, wherein the plant is identified and/or selected based on the presence and/or amount of at least one of the following TPS genes: CsTPS18VF, CsTPS19BL, CsTPS35LS, CsTPS18Choc, CsTPS29BC, and/or CsTPS17AK, using at least one forward & reverse primer combination from primer groups 9, 10, 11, 7, 17 and 18, respectively, whereby linalool bioaccumulation is present and/or increased.

A20.3. The method of any one of embodiments A15 to A20.2, wherein the plant is identified and/or selected based on the presence and/or amount of at least one of the following TPS genes: CsTPS32PK and/or CsTPS25LS, using at least one forward & reverse primer combination from primer groups 4 and 15 respectively, whereby β-Farnescene bioaccumulation is present and/or increased.

A20.4. The method of any one of embodiments A15 to A20.3, wherein the plant is identified and/or selected based on the presence and/or amount of at least one of the following TPS genes: CsTPS6FN, CsTPS13PK, and CsTPS38FN using at least one forward & reverse primer combination from primer groups 12, 13, and 14 respectively, whereby beta ocimene bioaccumulation is present and/or increased.

A20.5. The method of any one of embodiments A15 to A20.4, wherein the plant is identified and/or selected based on the presence and/or amount of at least one of the following TPS genes: CsTPS18VF, CsTPS19BL, and CsTPS35LS using at least one forward & reverse primer combination from primer groups 9, 10, and 11, respectively, whereby trans nerolidol bioaccumulation is present and/or increased.

A20.6. The method of any one of embodiments A15 to A20.5, wherein the plant is identified and/or selected based on the presence and/or amount of at least one of the following TPS genes: CsTPS5PK, CsTPS31PK and CsTPS32PK, using at least one forward & reverse primer combination from primer group 8, 5 and 4, whereby alpha bisabolol bioaccumulation is present and/or increased.

A20.7. The method of any one of embodiments A15 to A20.6, wherein the plant is identified and/or selected based on the presence and/or amount of at least one of the following TPS genes: CsTPS5PK, CsTPS31PK and CsTPS32PK, using at least one forward & reverse primer combination from primer group 8, 5 and 4, respectively, whereby α-terpineol bioaccumulation is present and/or increased.

A21. The method of any one of embodiments A1 to A20.7, wherein one or more plant cultivars is/are of the family Rosidae.

A22. The method of any one of embodiments A1 to A21, wherein one or more plant cultivars is/are a Cannabis cultivar.

A23. A plant cultivar, identified and/or selected by the method of any one of embodiments A1 to A22.

B1. A method of producing a daughter plant cultivar, comprising:
analyzing two or more parent plant cultivars by the method of any one of embodiments A1 to A22;
identifying and selecting two or more plant cultivars comprising at least one teroene synthase that produces at least one terpene that has one or more of: an energizing effect on a subject, an anti-nociceptive effect on a subject, or an insecticidal effect and/or an insect predator attractant effect in a plant; and
inbreeding or outcrossing the parent plant cultivars to produce a daughter plant cultivar. B2. The method of embodiment B1, wherein at least one parent plant cultivar is a Cannabis cultivar, or both parent plant cultivars are Cannabis cultivars.

B3. The method of embodiment B1, wherein at least one parent plant cultivar is of the family Rosidae, or both plant cultivars are of the family Rosidae.

C1. A method of treating a subject with one or more plant cultivars or a portion thereof or an extract thereof, comprising:
(i) obtaining one or more plant cultivars or samples therefrom;
(ii) preparing or analyzing nucleic acid from the one or more plant cultivars according to the method of any one of embodiments A1 to A22;
(iii) based on (ii), identifying one or more plant cultivars as desirable for treating a subject or as not desirable for treating a subject; and
(iv) if one or more plant cultivars are identified as desirable for treating a subject in (iii), treating the subject with the one or more plant cultivars identified according to (iii), or with a portion thereof, or with an extract thereof.

C2. The method of embodiment C1, wherein the treatment characteristic identified in (iii) is energetic or anti-nociceptive, or a combination thereof.

C3. A method of imparting insect resistance to one or more plant cultivars, comprising:
(i) obtaining one or more plant cultivars or samples therefrom;
(ii) preparing or analyzing nucleic acid from the one or more plant cultivars according to the method of any one of embodiments A15 to A22;
(iii) based on (ii), identifying one or more plant cultivars as desirable for imparting insect resistance; and
(iv) cultivating the one or more plant cultivars as a crop, or for breeding daughter cultivars that are insect resistant.

C4. The method of embodiment C3, wherein the insect resistance comprises contact resistance or fumigant properties, or a combination thereof.

D1. A composition, comprising at least one polynucleotide primer pair selected from among the primer pairs set forth in Tables 1-16 and/or from among the B3/F3 primer pairs set forth in Tables 17-35, and/or from among sequences that share 90%, 91%, 92%, 93%, 94%, 95%, 96%, 97%, 98%, 99% or more sequence identity with one or more of the primer sequences set forth in Tables 1-16 and/or the B3 and/or F3 primer sequences set forth in Tables 17-35.

D1.1. A composition, comprising an amplicon generated by amplification of a target sequence of at least one polynucleotide primer pair selected from among the primer pairs set forth in Tables 1-16, and/or an amplicon generated by amplification of a target sequence of at least one of the B3/F3 primer pairs set forth in Tables 17-35, and/or an amplicon generated by amplification of a target sequence of at least one primer pair wherein at least one primer of the primer pair is selected from among sequences that share 90%, 91%, 92%, 93%, 94%, 95%, 96%, 97%, 98%, 99% or more sequence identity with one or more of the primer sequences set forth in Tables 1-16 and/or the B3 and/or F3 primer sequences set forth in Tables 17-35.

D2. The composition of embodiment D1, comprising more than one polynucleotide primer pair, wherein the more than one polynucleotide primer pairs belong to the same primer group selected from among primer group 1 in Table 1, primer group 2 in Table 2, primer group 3 in Table 3, primer group 4 in Table 4, primer group 5 in Table 5, primer group 6 in Table 6, primer group 7 in Table 7, primer group 8 in Table 8, primer group 9 in Table 9, primer group 10 in Table 10, primer group 11 in Table 11, primer group 15 in Table 12, primer group 16 in Table 13, primer group 17 in Table 14, primer group 18 in Table 15 and primer group 19 in Table 16, and/or from among groups of sequences that share 90%, 91%, 92%, 93%, 94%, 95%, 96%, 97%, 98%, 99% or more sequence identity with one or more of the primer sequences of the groups set forth in Tables 1-16.

D2.1 The composition of embodiment D1.1, comprising one or more amplicons generated from more than one polynucleotide primer pair, the more than one polynucleotide primer pairs belong to the same primer group selected from among primer group 1 in Table 1, primer group 2 in Table 2, primer group 3 in Table 3, primer group 4 in Table 4, primer group 5 in Table 5, primer group 6 in Table 6, primer group 7 in Table 7, primer group 8 in Table 8, primer group 9 in Table 9, primer group 10 in Table 10, primer group 11 in Table 11, primer group 15 in Table 12, primer group 16 in Table 13, primer group 17 in Table 14, primer group 18 in Table 15 and primer group 19 in Table 16, and/or from among groups of sequences that share 90%, 91%, 92%, 93%, 94%, 95%, 96%, 97%, 98%, 99% or more sequence identity with one or more of the primer sequences of the groups set forth in Tables 1-16.

D2.2. The composition of any one of embodiments D1 to D2.1, further comprising at least one probe for qPCR or RT-qPCR.

D3. A composition, comprising at least one set of B3, F3, BIP and FIP polynucleotide primer pairs selected from among the primer pairs set forth in Tables 17-35 and/or from among sequences that share 90%, 91%, 92%, 93%, 94%, 95%, 96%, 97%, 98%, 99% or more sequence identity with one or more of the B3, F3, BIP and FIP primer sequences set forth in Tables 17-35.

D4. The composition of embodiment D3, further comprising a LF loop primer, a LB loop primer, or both a LF loop primer and a LB loop primer, and/or from among sequences that share 90%, 91%, 92%, 93%, 94%, 95%, 96%, 97%, 98%, 99% or more sequence identity with one or both of the LF loop primer and the LB loop primer.

D5. The composition of embodiment D4, wherein the LF loop primer, the LB loop primer, or both the LF loop primer and the LB loop primer are selected from among those set forth in one or more of Tables 17-35.

D6. The composition of any one of embodiments D1 to D5, wherein at least one primer or at least one amplicon comprises a nucleotide analog or a detectable label.

E1. A kit, comprising the composition of embodiment D1 or embodiment D2, reagents for nucleic acid amplification, and, optionally, instructions for use.

E2. The kit of embodiment E1, further comprising reagents for HRM.

E2.1. The kit of embodiment E1 or E2, further comprising a probe for qPCR or RT-qPCR.

E3. A kit, comprising the composition of any one of embodiments D3 to D5, reagents for nucleic acid amplification, and, optionally, instructions for use.

E4. The kit of embodiment E3, further comprising reagents for LAMP.

F1. An isolated terpene synthase from a Cannabis plant cultivar, wherein the terpene synthase produces guaiol.

F2. An isolated nucleic acid encoding the terpene synthase of embodiment F1.

F3. The isolated nucleic acid of embodiment F2, comprising the sequence set forth in SEQ ID NO:1408.

F4. A vector, comprising the isolated nucleic acid of embodiment F2 or embodiment F3.

F5. A cell comprising the vector of embodiment F4.

F6. The cell of embodiment F5, wherein the vector is an expression vector.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications can be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claims that follow:

What is claimed is:

1. A method of identifying/selecting a plant cultivar comprising at least one terpene synthase gene that produces at least one terpene that has an energizing effect on a subject, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an energetic terpene product profile comprising at least one terpene that has an energizing effect on a subject, wherein the polynucleotide primer pair comprises at least one polynucleotide primer pair selected from among one or more of the primer pairs of:
primer group 1 as set forth in Table 1, and the B3/F3 primer pairs set forth in Table 18;
primer group 2 as set forth in Table 2, and the B3/F3 primer pairs set forth in Tables 22 and 26;
primer group 3 as set forth in Table 3, and the B3/F3 primer pairs set forth in Table 19;
primer group 4 as set forth in Table 4, and the B3/F3 primer pairs set forth in Table 23;
primer group 12 as set forth in the B3/F3 primer pairs set forth in Table 20;
primer group 13 as set forth in the B3/F3 primer pairs set forth in Tables 21;
primer group 14 as set forth in the B3/F3 primer pairs set forth in Table 24;
primer group 5 as set forth in Table 5, and the B3/F3 primer pairs set forth in Table 28;
primer group 6 as set forth in Table 6, and the B3/F3 primer pairs set forth in Table 17;
primer group 19 as set forth in Table 16, and the B3/F3 primer pairs set forth in Table 34; and
primer group 9 as set forth in Table 9, and the B3/F3 primer pairs set forth in Table 30;
(c) amplifying the mixture, thereby obtaining an amplified mixture;
(d) analyzing the amplified mixture of (c), wherein the presence of at least one terpene synthase gene that produces an energetic terpene product profile comprising at least one terpene that has an energizing effect on a subject is identified in the amplified mixture based on the presence of at least one amplified subsequence of the terpene synthase gene; and
(e) identifying and/or selecting a plant cultivar based on the at least one terpene synthase gene that is identified in (d).

2. The method of claim 1, wherein the presence and/or amount of at least one amplicon generated by at least one polynuceotide primer pair of (b) is identified in (d).

3. The method of claim 1, wherein the terpene synthase gene produces an energetic terpene product profile comprising at least one terpene that has an energizing effect on a subject.

4. A plant cultivar identified and/or selected by the method of claim 1.

5. The method of claim 3, wherein the energetic terpene product profile comprises one or more of 3-carene, alpha-terpinene and gamma-terpinene.

6. The method of claim 5, wherein the energetic terpene product profile further comprises terpinolene.

7. The method of claim 5, wherein the terpene synthase gene has the polypeptide sequence set forth in SEQ ID NO:1671.

8. A method of identifying/selecting a plant cultivar comprising at least one terpene synthase gene that produces at least one terpene that has an anti-nociceptive effect on a subject, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an anti-nociceptive terpene product profile comprising at least one terpene that has an anti-nociceptive effect on a subject, wherein the polynucleotide primer pair comprises at least one polynucleotide primer pair selected from among one or more of the primer pairs of:
primer group 1 as set forth in Table 1, and the B3/F3 primer pairs set forth in Table 18;
primer group 4 as set forth in Table 4, and the B3/F3 primer pairs set forth in Table 23;
primer group 5 as set forth in Table 5, and the B3/F3 primer pairs set forth in Table 28;
primer group 6 as set forth in Table 6, and the B3/F3 primer pairs set forth in Table 17;
primer group 8 as set forth in Table 8, and the B3/F3 primer pairs set forth in Table 26;
primer group 9 as set forth in Table 9, and the B3/F3 primer pairs set forth in Table 30;
primer group 10 as set forth in Table 10, and the B3/F3 primer pairs set forth in Table 29; and
primer group 11 as set forth in Table 11, and the B3/F3 primer pairs set forth in Table 27;
(c) amplifying the mixture, thereby obtaining an amplified mixture;
(d) analyzing the amplified mixture of (c), wherein the presence of at least one terpene synthase gene that produces an anti-nociceptive terpene product profile comprising at least one terpene that has an anti-nociceptive effect on a subject is identified in the amplified mixture based on the presence of at least one amplified subsequence of the terpene synthase gene; and
(e) identifying and/or selecting a plant cultivar based on the at least one terpene synthase gene that is identified in (d).

9. The method of claim 8, wherein the presence and/or amount of at least one amplicon generated by at least one polynuceotide primer pair of (b) is identified in (d).

10. The method of claim 8, wherein the terpene synthase gene produces an anti-nociceptive terpene product profile comprising at least one terpene that has an anti-nociceptive effect on a subject.

11. A plant cultivar identified and/or selected by the method of claim 8.

12. The method of claim 10, wherein the anti-nociceptive terpene product profile comprises alpha phellandrene.

13. The method of claim 10, wherein the terpene synthase gene has the polypeptide sequence set forth in SEQ ID NO:1671.

14. A method of identifying/selecting a plant cultivar comprising at least one terpene synthase gene that produces at least one terpene that has an insecticidal effect and/or an insect predator attractant in the plant, comprising:
(a) obtaining a nucleic acid sample from the plant cultivar;
(b) contacting the nucleic acid sample with at least one polynucleotide primer pair comprising a forward primer and a reverse primer under amplification conditions, thereby preparing a mixture, wherein the polynucleotide primer pair hybridizes to a unique subsequence of at least one terpene synthase gene that produces an insecticidal and/or an insect predator attractant terpene product profile comprising at least one terpene that has an insecticidal and/or an insect predator attractant effect in a plant, wherein the polynucleotide primer pair comprises at least one polynucleotide primer pair selected from among one or more of the primer pairs of:
primer group 4 as set forth in Table 4, and the B3/F3 primer pairs set forth in Table 23;
primer group 5 as set forth in Table 5, and the B3/F3 primer pairs set forth in Table 28;
primer group 6 as set forth in Table 6, and the B3/F3 primer pairs set forth in Table 17;
primer group 8 as set forth in Table 8, and the B3/F3 primer pairs set forth in Table 26;
primer group 9 as set forth in Table 9, and the B3/F3 primer pairs set forth in Table 30;
primer group 10 as set forth in Table 10, and the B3/F3 primer pairs set forth in Table 29;
primer group 11 as set forth in Table 11, and the B3/F3 primer pairs set forth in Table 27;
primer group 16 as set forth in Table 13, and the B3/F3 primer pairs set forth in Table 35;
primer group 7 as set forth in Table 7, and the B3/F3 primer pairs set forth in Table 25;
primer group 12 as set forth in the B3/F3 primer pairs set forth in Table 20;
primer group 13 as set forth in the B3/F3 primer pairs set forth in Tables 21;
primer group 14 as set forth in the B3/F3 primer pairs set forth in Table 24;
primer group 15 as set forth in Table 12, and the B3/F3 primer pairs set forth in Table 31;
primer group 17 as set forth in Table 14, and the B3/F3 primer pairs set forth in Table 32; and
primer group 18 as set forth in Table 15 and the B3/F3 primer pairs set forth in Table 33;
(c) amplifying the mixture, thereby obtaining an amplified mixture;
(d) analyzing the amplified mixture of (c), wherein the presence of at least one terpene synthase gene that produces an insecticidal product profile and/or an insect predator attractant terpene product profile comprising at least one terpene that has an insecticidal effect and/or an insect predator attractant effect in a plant is identified in the amplified mixture based on the presence of at least one amplified subsequence of the terpene synthase gene; and
(e) identifying and/or selecting a plant cultivar based on the at least one terpene synthase gene that is identified in (d).

15. The method of claim 14, wherein the presence and/or amount of at least one amplicon generated by at least one polynuceotide primer pair of (b) is identified in (d).

16. The method of claim 1, wherein the terpene synthase gene produces an insecticidal terpene product profile and/or an insect predator attractant terpene product profile comprising at least one terpene that has an insecticidal effect and/or an insect predator attractant effect on a subject plant cultivar.

17. A plant cultivar identified and/or selected by the method of claim 14.

18. The method of claim 16, wherein the insecticidal terpene product profile and/or an insect predator attractant terpene product profile comprises one or both of 3-carene and guaiol.

19. The method of claim 18, wherein the insecticidal terpene product profile and/or an insect predator attractant terpene product profile comprises 3-carene and the terpene synthase gene has the polypeptide sequence set forth in SEQ ID NO:1671.

20. The method of claim 18, wherein the insecticidal terpene product profile and/or an insect predator attractant terpene product profile comprises guaiol and the terpene synthase gene has the polypeptide encoded by the nucleic acid sequence set forth in SEQ ID NO:1408.
